(19) United States
(12) Patent Application Publication
(10) Pub. No.: US 2023/0398148 A1
(43) Pub. Date: Dec. 14, 2023

(54) CELLS EXPRESSING A CHIMERIC RECEPTOR FROM A MODIFIED INVARIANT CD3 IMMUNOGLOBULIN SUPERFAMILY CHAIN LOCUS AND RELATED POLYNUCLEOTIDES AND METHODS

(71) Applicant: Juno Therapeutics, Inc., Seattle, WA (US)

(72) Inventors: Mateusz Pawel POLTORAK, Munich (DE); Lothar GERMEROTH, Munich (DE); Christian STEMBERGER, Munich (DE)

(73) Assignee: Juno Therapeutics, Inc., Seattle, WA (US)

(21) Appl. No.: 18/035,277

(22) PCT Filed: Nov. 3, 2021
(86) PCT No.: PCT/US2021/057937

§ 371 Date: May 3, 2023

Related U.S. Application Data
(60) Provisional application No. 63/109,858, filed on Nov. 4, 2020.

(51) Int. Cl. A61K 35/17; A61P 35/02; C07K 16/28; C07K 14/725; A61K 39/00

(52) U.S. Cl. CPC A61K 35/17; A61P 35/02; C07K 16/2809; C07K 14/7051; A61K 39/4611; A61K 39/4631; A61K 39/464411; A61K 2239/13

(57) ABSTRACT

Provided herein are engineered T cells, expressing a chimeric receptor comprising an antigen-binding domain fused to an endogenous invariant CD3 chain of the immunoglobulin superfamily (invariant CD3-IgSF). In some embodiments, the engineered T cells contain a modified invariant CD3-IgSF chain locus that encodes the chimeric receptor. Also provided are cell compositions containing the engineered T cells, nucleic acids for engineering cells, and methods, kits and articles of manufacture for producing the engineered cells, such as by targeting a transgene encoding a portion of a chimeric receptor for integration into an invariant CD3-IgSF chain genomic locus. In some embodiments, the engineered cells, e.g. T cells, can be used in connection with cell therapy, including in connection with cancer immunotherapy comprising adoptive transfer of the engineered cells.

### CROSS REFERENCE TO RELATED APPLICATIONS

This application claims priority from U.S. provisional application No. 63/109,858, filed Nov. 4, 2020, the contents of which are incorporated by reference in their entirety.

### SEQUENCE LISTING

The present application is being filed along with a Sequence Listing in electronic format. The Sequence Listing is provided as a file entitled 735042016940SeqList.txt, created Nov. 3, 2021, which is 60,967 bytes in size. The information in the electronic format of the Sequence Listing is incorporated by reference in its entirety.

### FIELD

The present disclosure relates to engineered T cells, expressing a chimeric receptor comprising an antigen-binding domain fused to an endogenous invariant CD3 chain of the immunoglobulin superfamily (invariant CD3-IgSF). In some embodiments, the engineered T cells contain a modified invariant CD3-IgSF chain locus that encodes the chimeric receptor. Also provided are cell compositions containing the engineered T cells, nucleic acids for engineering cells, and methods, kits and articles of manufacture for producing the engineered cells, such as by targeting a transgene encoding a portion of a chimeric receptor for integration into an invariant CD3-IgSF chain genomic locus. In some embodiments, the engineered cells, e.g. T cells, can be used in connection with cell therapy, including in connection with cancer immunotherapy comprising adoptive transfer of the engineered cells.

### BACKGROUND

Adoptive cell therapies that utilize chimeric receptors, such as chimeric receptors including a binding domain, to recognize antigens associated with a disease represent an attractive therapeutic modality for the treatment of cancers and other diseases. Improved strategies are needed for engineering T cells to express chimeric receptors, such as for use in adoptive immunotherapy, e.g., in treating cancer, infectious diseases and autoimmune diseases. Provided are methods, cells, compositions and kits for use in the methods that meet such needs.

### SUMMARY

Provided herein are engineered T cells, comprising a modified invariant CD3-immunoglobulin superfamily (invariant CD3-IgSF) chain locus comprising a nucleic acid sequence encoding a mini chimeric antigen receptor (miniCAR), wherein the miniCAR is a fusion protein comprising a heterologous antigen-binding domain and an endogenous invariant CD3 chain of the invariant CD3-IgSF chain, wherein the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain.

Provided herein are engineered T cells expressing a mini chimeric antigen receptor (miniCAR), wherein the miniCAR is a fusion protein comprising a heterologous antigen-binding domain and an endogenous invariant CD3 chain of the immunoglobulin superfamily (invariant CD3-IgSF chain). In some of any embodiments, the miniCAR is expressed from a modified invariant CD3-immunoglobulin superfamily (invariant CD3-IgSF) chain locus comprising a nucleic acid sequence encoding the miniCAR, wherein the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain.

Provided herein are engineered T cells comprising a transgene encoding an antigen-binding domain inserted in-frame with an open reading frame of a locus encoding an endogenous invariant CD3 chain of the immunoglobulin superfamily (invariant CD3-IgSF chain), wherein the engineered T cell expresses a miniCAR fusion protein comprising a heterologous antigen-binding domain and the endogenous invariant CD3-IgSF chain.

In some of any of the provided embodiments, the invariant CD3-IgSF chain is a CD3 epsilon (CD3e) chain. In some of any of the provided embodiments, the invariant CD3-IgSF chain is a CD3 delta (CD3d) chain. In some of any of the provided embodiments, the invariant CD3-IgSF chain is a CD3 gamma (CD3g) chain. In some of any of the provided embodiments, the modified invariant CD3-IgSF chain locus is a modified CD3 epsilon (CD3E) locus encoding a CD3e chain, a modified CD3 delta (CD3D) locus encoding a CD3d chain, or a modified CD3 gamma (CD3G) locus encoding a CD3g chain. In some of any of the provided embodiments, the modified invariant CD3-IgSF chain locus is a modified CD3E locus encoding a CD3e chain. In some of any of the provided embodiments, the modified invariant CD3-IgSF chain locus is a modified CD3D locus encoding a CD3d chain. In some of any of the provided embodiments, the modified invariant CD3-IgSF chain locus is a modified CD3G locus encoding a CD3g chain.

Provided herein are engineered T cells, comprising a modified CD3E locus comprising a nucleic acid sequence encoding a mini chimeric antigen receptor (miniCAR), wherein the miniCAR is a fusion protein comprising a heterologous antigen-binding domain and an endogenous CD3e chain, wherein the nucleic acid sequence comprises an in-frame fusion of (i) a transgene comprising a sequence encoding the antigen-binding domain and (ii) an open reading frame of the endogenous CD3E locus encoding the CD3e chain.

Provided herein are engineered T cells expressing a mini chimeric antigen receptor (miniCAR), wherein the miniCAR is a fusion protein comprising a heterologous antigen-binding domain and an endogenous CD3e chain.

In some of any of the provided embodiments, the miniCAR is expressed from a modified CD3E chain locus comprising a nucleic acid sequence encoding the miniCAR, wherein the nucleic acid sequence comprises an in-frame fusion of (i) a transgene comprising a sequence encoding the antigen-binding domain and (ii) an open reading frame of the endogenous CD3E locus encoding the CD3e chain.

Provided herein are engineered T cells comprising a transgene encoding an antigen-binding domain inserted in-frame with an open reading frame of a locus encoding an endogenous CD3e chain, wherein the engineered T cell expresses a miniCAR fusion protein comprising a heterologous antigen-binding domain and the endogenous CD3e chain.

In some of any of the provided embodiments, the antigen-binding domain is or comprises an antibody or an antigen-binding fragment thereof. In some of any of the provided embodiments, the antigen-binding domain is or comprises a Fab fragment, a Fab₂ fragment, a single domain antibody, or a single chain variable fragment (scFv). In some of any of the provided embodiments, the antigen-binding domain is an scFv. In some of any of the provided embodiments, the modified invariant CD3-IgSF chain locus comprises, in order from 5′ to 3′, a sequence of nucleotides encoding the heterologous antigen-binding domain and the endogenous invariant CD3-IgSF chain. In some of any of the provided embodiments, the modified CD3E locus comprises, in order from 5′ to 3′, a sequence of nucleotides encoding the heterologous antigen-binding domain and the endogenous CD3e chain. In some of any of the provided embodiments, the heterologous antigen-binding domain and the invariant CD3-IgSF chain are directly linked. In some of any of the provided embodiments, the heterologous antigen-binding domain and the invariant CD3-IgSF chain are linked indirectly via a linker. In some of any of the provided embodiments, the heterologous antigen-binding domain and the CD3e chain are directly linked. In some of any of the provided embodiments, the heterologous antigen-binding domain and the CD3e chain are linked indirectly via a linker. In some of any of the provided embodiments, the transgene further comprises a nucleic acid sequence encoding a linker. In some of any embodiments, the linker is positioned 3′ to the antigen-binding domain.

Provided herein are engineered T cells, comprising a modified CD3E locus comprising a nucleic acid sequence encoding a miniCAR, the miniCAR comprising a heterologous antigen-binding domain and an endogenous CD3e chain, wherein the nucleic acid sequence comprises an in-frame fusion of (i) a transgene comprising a sequence encoding the antigen-binding domain, wherein the antigen-binding domain is an scFv, and a sequence encoding a linker, and (ii) an open reading frame of an endogenous CD3E locus encoding the CD3e chain.

In some of any of the provided embodiments, the transgene sequence comprises, in order from 5′ to 3′, a sequence of nucleotides encoding the antigen-binding domain and a sequence of nucleotides encoding the linker. In some of any of the provided embodiments, the modified invariant CD3-IgSF chain locus comprises, in order from 5′ to 3′, a sequence of nucleotides encoding the antigen-binding domain, the linker, and the invariant CD3-IgSF chain.

In some of any of the provided embodiments, the linker is a polypeptide linker. In some of any of the provided embodiments, the linker is a polypeptide that is 2, 3, 4, 5, 6, 7, 8, 9, 10, 11, 12, 13, 14, 15, 16, 17, 18, 19 or 20 amino acids in length. In some of any of the provided embodiments, the linker is a polypeptide that is 3 to 18 amino acids in length. In some of any of the provided embodiments, the linker is a polypeptide that is 12 to 18 amino acids in length. In some of any of the provided embodiments, the linker is a polypeptide that is 15 to 18 amino acids in length. In some of any of the provided embodiments, the linker comprises GS, GGS, GGGGS (SEQ ID NO:122), GGGGGS (SEQ ID NO:128) and combinations thereof. In some of any of the provided embodiments, the linker comprises (GGS)n, wherein n is 1 to 10, (GGGGS)n (SEQ ID NO: 121), wherein n is 1 to 10, or (GGGGGS)n (SEQ ID NO:129), wherein n is 1 to 4. In some of any of the provided embodiments, the linker is selected from among a linker that is or comprises GGS, is or comprises GGGGS (SEQ ID NO: 122), is or comprises GGGGGS (SEQ ID NO: 128), is or comprises (GGS)2 (SEQ ID NO: 130), is or comprises GGSGGSGGS (SEQ ID NO: 131), is or comprises GGSGGSGGSGGS (SEQ ID NO:132), is or comprises GGSGGSGGSGGSGGS (SEQ ID NO:133), is or comprises GGGGGSGGGGGSGGGGGS (SEQ ID NO:134), is or comprises GGSGGGGSGGGGSGGGGS (SEQ ID NO: 135), is or comprises and GGGGSGGGGSGGGGS (SEQ ID NO:16). In some of any of the provided embodiments, the linker is or comprises GGGGSGGGGSGGGGS (SEQ ID NO:16).

In some of any of the provided embodiments, the transgene further comprises a nucleic acid sequence encoding one or more multicistronic elements, optionally wherein the one or more multicistronic elements are or comprise a ribosome skip sequence, optionally wherein the ribosome skip sequence is a T2A, a P2A, an E2A, or an F2A element. In some of any of the provided embodiments, the P2A element comprises the sequence set forth in SEQ ID NO: 3. In some of any of the provided embodiments, at least one of the one or more multicistronic elements is positioned 5′ to the antigen-binding domain. In some of any of the provided embodiments, the transgene sequence comprises, in order from 5′ to 3′, a sequence of nucleotides encoding the multicistronic element, optionally the P2A element; the antigen-binding domain; and the linker.

In some of any of the provided embodiments, the transgene further comprises a nucleic acid sequence encoding an affinity tag. In some of any of the provided embodiments, the affinity tag is a streptavidin binding peptide. In some of any of the provided embodiments, the streptavidin binding peptide is or comprises the sequence Trp-Ser-His-Pro-Gln-Phe-Glu-Lys (SEQ ID NO: 137), Trp-Arg-His-Pro-Gln-Phe-Gly-Gly (SEQ ID NO:136), Trp-Ser-His-Pro-Gln-Phe-Glu-Lys-(GlyGlyGlySer)3-Trp-Ser-His-Pro-Gln-Phe-Glu-Lys (SEQ ID NO: 146), Trp-Ser-His-Pro-Gln-Phe-Glu-Lys-(GlyGlyGlySer)2-Trp-Ser-His-Pro-Gln-Phe-Glu-Lys (SEQ ID NO: 147) and Trp-Ser-His-Pro-Gln-Phe-Glu-Lys-(GlyGlyGlySer)2Gly-Gly-Ser-Ala-Trp-Ser-His-Pro-Gln-Phe-Glu-Lys (SEQ ID NO: 148).

In some of any of the provided embodiments, the modified invariant CD3-IgSF chain locus comprises, in order from 5′ to 3′, a sequence of nucleotides encoding the multicistronic element, optionally a P2A element; the antigen-binding domain; the linker; and the invariant CD3-IgSF chain. In some of any of the provided embodiments, the modified CD3E locus comprises, in order from 5′ to 3′, a sequence of nucleotides encoding the multicistronic element, optionally a P2A element; the antigen-binding domain; the linker; and the CD3e chain. In some of any of the provided embodiments, the open reading frame of the endogenous invariant CD3-IgSF chain locus in (ii) encodes a full length mature invariant CD3-IgSF chain. In some of any of the provided embodiments, the modified invariant CD3-IgSF chain locus comprises the promoter and/or regulatory or control element of the endogenous locus operably linked to control expression the nucleic acid sequence encoding the miniCAR. In some of any of the provided embodiments, the modified invariant CD3-IgSF chain locus comprises one or more heterologous regulatory or control elements operably linked to control expression of the miniCAR or a portion thereof.

In some of any of the provided embodiments, the antigen-binding domain binds to a target antigen that is associated with, specific to, and/or expressed on a cell or tissue of a disease, disorder or condition. In some of any of the provided embodiments, the target antigen is a tumor antigen. In some of any of the provided embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some of any of the provided embodiments, the miniCAR assembles into a TCR/CD3 complex in place of the corresponding endogenous invariant CD3-IgSF chain of the TCR/CD3 complex. In some of any of the provided embodiments, the miniCAR assembles into a TCR/CD3 complex in place of the corresponding endogenous invariant CD3-IgSF CD3e chain of the TCR/CD3 complex. In some of any of the provided embodiments, binding of a target antigen by the heterologous antigen-binding domain of the miniCAR induces antigen-dependent signaling via the TCR/CD3 complex. In some of any of the provided embodiments, the miniCAR exhibits reduced tonic signaling via the TCR/CD3 complex compared to T cells engineered with a chimeric antigen receptor (CAR) that comprises the same antigen-binding domain. In some of any of the provided embodiments, the engineered T cell exhibits increased persistence compared to T cells engineered with a chimeric antigen receptor (CAR) that comprises the same antigen-binding domain and a heterologous CD3zeta (CD3z) signaling domain, and optionally a costimulatory signaling domain. In some of any of the provided embodiments, the engineered T cell exhibits increased cytolytic activity compared to T cells engineered with a chimeric antigen receptor (CAR) that comprises the same antigen-binding domain and a heterologous CD3zeta (CD3z) signaling domain, and optionally a costimulatory signaling domain.

In some of any of the provided embodiments, the T cell is a primary T cell derived from a subject. In some of any of the provided embodiments, the subject is a human. In some of any of the provided embodiments, the T cell is a CD8+ T cell or a subtype thereof, or a CD4+ T cell or a subtype thereof.

In some of any of the provided embodiments, the transgene sequence is integrated at the endogenous invariant CD3-IgSF chain locus of a T cell via homology directed repair (HDR).

Provided herein are polynucleotides, comprising (a) a nucleic acid sequence encoding an antigen-binding domain; and (b) one or more homology arms linked to the nucleic acid sequence, wherein the one or more homology arms comprise a sequence homologous to one or more regions of an open reading frame of an invariant CD3 chain of the immunoglobulin superfamily (invariant CD3-IgSF chain) locus of a T cell, wherein the invariant CD3-IgSFchain locus encodes an invariant CD3-IgSF chain.

In some of any of the provided embodiments, the one or more homology arms comprise a sequence homologous to one or more regions of an open reading frame of the invariant CD3-IgSF chain locus, wherein the invariant CD3-IgSF chain locus is a CD3E locus encoding a CD3e chain, a CD3D locus encoding a CD3d chain, or a CD3G locus encoding a CD3g chain.

In some of any of the provided embodiments, the invariant CD3-IgSF chain locus is a CD3E locus encoding a CD3e chain. In some of any of the provided embodiments, the invariant CD3-IgSF chain locus is a CD3D locus encoding a CD3d chain. In some of any of the provided embodiments, the invariant CD3-IgSF chain locus is a CD3G locus encoding a CD3g chain.

Provided herein are polynucleotides, comprising (a) a nucleic acid sequence encoding an antigen-binding domain; and (b) one or more homology arms linked to the nucleic acid sequence encoding the transgene, wherein the one or more homology arms comprise a sequence homologous to one or more regions of an open reading frame of a CD3E locus encoding a CD3e chain.

In some of any of the provided embodiments, the antigen-binding domain is or comprises an antibody or an antigen-binding fragment thereof. In some of any of the provided embodiments, the antigen-binding domain is or comprises a Fab fragment, a Fab₂ fragment, a single domain antibody, or a single chain variable fragment (scFv). In some of any of the provided embodiments, the antigen-binding domain is an scFv. In some of any of the provided embodiments, the nucleic acid sequence further comprises nucleotides encoding a linker operably connected to the encoded antigen-binding domain, wherein the linker is positioned 3′ to the antigen-binding domain.

Provided herein are polynucleotides, comprising (a) a nucleic acid sequence encoding a single chain variable fragment (scFv) and a sequence encoding a linker; and (b) one or more homology arms linked to the nucleic acid sequence, wherein the one or more homology arms comprise a sequence homologous to one or more regions of an open reading frame of a CD3E locus encoding a CD3e chain.

In some of any of the provided embodiments, the encoded linker is a polypeptide encoded linker. In some of any of the provided embodiments, the encoded linker is a polypeptide that is 2, 3, 4, 5, 6, 7, 8, 9, 10, 11, 12, 13, 14, 15, 16, 17, 18, 19 or 20 amino acids in length. In some of any of the provided embodiments, the encoded linker is a polypeptide that is 3 to 18 amino acids in length. In some of any of the provided embodiments, the encoded linker is a polypeptide that is 12 to 18 amino acids in length. In some of any of the provided embodiments, the encoded linker is a polypeptide that is 15 to 18 amino acids in length. In some of any of the provided embodiments, the encoded linker comprises GS, GGS, GGGGS (SEQ ID NO:122), GGGGGS (SEQ ID NO:128) and combinations thereof. In some of any of the provided embodiments, the encoded linker comprises (GGS)n, wherein n is 1 to 10, (GGGGS)n (SEQ ID NO: 121), wherein n is 1 to 10, or (GGGGGS)n (SEQ ID NO:129), wherein n is 1 to 4. In some of any of the provided embodiments, the encoded linker is selected from among a encoded linker that is or comprises GGS, is or comprises GGGGS (SEQ ID NO: 122), is or comprises GGGGGS (SEQ ID NO: 128), is or comprises (GGS)2 (SEQ ID NO: 130), is or comprises GGSGGSGGS (SEQ ID NO: 131), is or comprises GGSGGSGGSGGS (SEQ ID NO:132), is or comprises GGSGGSGGSGGSGGS (SEQ ID NO:133), is or comprises GGGGGSGGGGGSGGGGGS (SEQ ID NO:134), is or comprises GGSGGGGSGGGGSGGGGS (SEQ ID NO: 135), is or comprises and GGGGSGGGGSGGGGS (SEQ ID NO:16).

In some of any of the provided embodiments, the encoded linker is or comprises GGGGSGGGGSGGGGS (SEQ ID NO:16). In some of any of the provided embodiments, the nucleic acid sequence comprises, in order from 5′ to 3′, a sequence of nucleotides encoding the antigen-binding domain and a sequence of nucleotides encoding the linker. In some of any of the provided embodiments, the nucleic acid sequence further comprises nucleotides encoding one or more multicistronic elements, optionally wherein the one or more multicistronic elements are or comprise a ribosome skip sequence, optionally wherein the ribosome skip sequence is a T2A, a P2A, an E2A, or an F2A element. In some of any of the provided embodiments, the P2A element comprises the sequence set forth in SEQ ID NO: 3.

In some of any of the provided embodiments, the nucleic acid sequence comprises, in order from 5′ to 3′, a sequence of nucleotides encoding the multicistronic element, optionally the P2A element; the antigen-binding domain; and the linker. In some of any of the provided embodiments, the nucleic acid sequence further comprises a nucleic acid sequence encoding an affinity tag. In some of any of the provided embodiments, the affinity tag is a streptavidin binding peptide. In some of any of the provided embodiments, the streptavidin binding peptide is or comprises the sequence Trp-Ser-His-Pro-Gln-Phe-Glu-Lys (SEQ ID NO: 137), Trp-Arg-His-Pro-Gln-Phe-Gly-Gly (SEQ ID NO:136), Trp-Ser-His-Pro-Gln-Phe-Glu-Lys-(GlyGlyGlySer)3-Trp-Ser-His-Pro-Gln-Phe-Glu-Lys (SEQ ID NO: 146), Trp-Ser-His-Pro-Gln-Phe-Glu-Lys-(GlyGlyGlySer)2-Trp-Ser-His-Pro-Gln-Phe-Glu-Lys (SEQ ID NO: 147) and Trp-Ser-His-Pro-Gln-Phe-Glu-Lys-(GlyGlyGlySer)2Gly-Gly-Ser-Ala-Trp-Ser-His-Pro-Gln-Phe-Glu-Lys (SEQ ID NO: 148).

In some of any of the provided embodiments, the one or more homology arms comprise a 5′ homology arm and a 3′ homology arm and the polynucleotide comprises the structure [5′ homology arm]-[nucleic acid sequence of (a)]-[3′ homology arm]. In some of any of the provided embodiments, the 5′ homology arm and the 3′ homology arm independently are at or about 100, 200, 300, 400, 500, 600, 700 or 800 nucleotides in length, or any value between any of the foregoing, or are greater than at or about 100 nucleotides in length, optionally at or about 100, 200 or 300 nucleotides in length, or any value between any of the foregoing. In some of any of the provided embodiments, the 5′ homology arm comprises (i) the sequence set forth in SEQ ID NO: 4, or (ii) a sequence that exhibits at least 85%, 86%, 87%, 88%, 89%, 90%, 91%, 92%, 93%, 94%, 95%, 96%, 97%, 98%, 99% or more sequence identity to SEQ ID NO: 4 or (ii) a partial sequence of (i) or (ii). In some of any of the provided embodiments, the 3′ homology arm comprises (i) the sequence set forth in SEQ ID NO: 5, or (ii) a sequence that exhibits at least 85%, 86%, 87%, 88%, 89%, 90%, 91%, 92%, 93%, 94%, 95%, 96%, 97%, 98%, 99% or more sequence identity to SEQ ID NO: 5 or (iii) a partial sequence of (i) or (ii).

In some of any of the provided embodiments, the encoded antigen-binding domain binds to a target antigen that is associated with, specific to, and/or expressed on a cell or tissue of a disease, disorder or condition. In some of any of the provided embodiments, the target antigen is a tumor antigen. In some of any of the provided embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some of any of the provided embodiments, introduction of the polynucleotide into a genome of a T cell generates a modified invariant CD3-IgSF chain locus encoding a mini chimeric antigen receptor (miniCAR), wherein the miniCAR is a fusion protein comprising the antigen-binding domain encoded by the nucleic acid of the polynucleotide and an endogenous invariant CD3-IgSF chain, and wherein the modified invariant CD3-IgSF chain locus comprises the nucleic acid encoding the antigen-binding domain in-frame with an open reading frame of the endogenous invariant CD3-IgSF chain locus encoding the invariant CD3-IgSF chain.

In some of any of the provided embodiments, the endogenous invariant CD3-IgSF chain is a CD3e chain, a CD3d chain, or a CD3g chain. In some of any of the provided embodiments, the endogenous invariant CD3-IgSF chain is a CD3e chain. In some of any of the provided embodiments, the endogenous invariant CD3-IgSF chain is a CD3d chain. In some of any of the provided embodiments, the endogenous invariant CD3-IgSF chain is a CD3g chain. In some of any of the provided embodiments, the encoded miniCAR assembles into a TCR/CD3 complex in place of the corresponding endogenous invariant CD3-IgSF chain of the TCR/CD3 complex.

In some of any of the provided embodiments, the polynucleotide is a linear polynucleotide, optionally a double-stranded polynucleotide or a single-stranded polynucleotide. In some of any of the provided embodiments, the polynucleotide is comprised in a vector. In some of any of the provided embodiments, the polynucleotide is between at or about 500 and at or about 3000 nucleotides, at or about 1000 and at or about 2500 nucleotides, or at or about 1500 nucleotides and at or about 2000 nucleotides in length. In some of any of the provided embodiments, any of the provided polynucleotides comprise a vector. In some of any of the provided embodiments, the vector is a viral vector. In some of any of the provided embodiments, the viral vector is an AAV vector, optionally wherein the AAV vector is an AAV2 or AAV6 vector. In some of any of the provided embodiments, the viral vector is a retroviral vector, optionally a lentiviral vector.

In some of any of the provided embodiments, the method comprising introducing any of the provided polynucleotides into a population of T cells, where T cells of the population comprise a genetic disruption at an endogenous invariant CD3-IgSF chain locus, wherein the invariant CD3-IgSF chain locus encodes an invariant CD3-IgSF chain. In some of any of the provided embodiments, the method comprising introducing any of the provided vectors into a population of T cells, where T cells of the population comprise a genetic disruption at an endogenous invariant CD3-IgSF chain locus, wherein the invariant CD3-IgSF chain locus encodes an invariant CD3-IgSF chain.

Provided herein are methods of producing genetically engineered T cells, the method comprising (a) introducing, into a population of T cells, one or more agents capable of inducing a genetic disruption at a target site within an endogenous invariant CD3-IgSF chain locus of T cells in the population, wherein the invariant CD3-IgSF chain locus encodes an invariant CD3-IgSF chain; and (b) introducing any of the provided the polynucleotides into the population of T cells, wherein T cells in the population comprise a genetic disruption at the endogenous invariant CD3 IgSF chain locus.

Provided herein are methods of producing genetically engineered T cells, the method comprising (a) introducing, into a population of T cells, one or more agents capable of inducing a genetic disruption at a target site within an endogenous invariant CD3-IgSF chain locus of T cells in the population, wherein the invariant CD3-IgSF chain locus encodes an invariant CD3-IgSF chain; and (b) introducing any of the provided vectors into the population of T cells, wherein T cells in the population comprise a genetic disruption at the endogenous invariant CD3 IgSF chain locus.

In some of any of the provided embodiments, the nucleic acid sequence of the polynucleotide is integrated in the endogenous invariant CD3-IgSF chain locus via homology directed repair (HDR).

Provided herein are methods of producing genetically engineered T cells, the method comprising (a) introducing, into a population comprising T cells, one or more agents capable of inducing a genetic disruption at a target site within an endogenous CD3E locus; and (b) introducing any of the provided polynucleotides into the population comprising T cells, wherein T cells in the population comprise a genetic disruption at the endogenous CD3E locus.

Provided herein are methods of producing genetically engineered T cells, the method comprising (a) introducing, into a population comprising T cells, one or more agents capable of inducing a genetic disruption at a target site within an endogenous CD3E locus; and (b) introducing any of the provided vectors into the population comprising T cells, wherein T cells in the population comprise a genetic disruption at the endogenous CD3E locus.

Provided herein are methods of producing genetically engineered T cells, the method comprising introducing into a population comprising T cells any of the provided polynucleotides, wherein T cells of the population comprise a genetic disruption within an endogenous CD3E locus, wherein the transgene of the polynucleotide is integrated into the endogenous CD3E locus via homology directed repair (HDR).

Provided herein are methods of producing genetically engineered T cells, the method comprising introducing into a population comprising T cells any of the provided vectors wherein T cells of the population comprise a genetic disruption within an endogenous CD3E locus, wherein the transgene of the polynucleotide is integrated into the endogenous CD3E locus via homology directed repair (HDR).

In some of any of the provided embodiments, the genetic disruption is carried out by introducing, into the population of T cells, one or more agents to induce a genetic disruption at a target site within an endogenous invariant CD3-IgSF chain locus of the T cell. In some of any of the provided embodiments, the method produces a modified invariant CD3-IgSF chain locus in T cells of the population of T cells, said modified invariant CD3-IgSF chain locus comprising a nucleic acid sequence encoding a miniCAR, wherein the miniCAR is a fusion protein comprising the antigen-binding domain encoded by the introduced polynucleotide or vector and the endogenous invariant CD3-IgSF chain. In some of any of the provided embodiments, the encoded miniCAR assembles into a TCR/CD3 complex in place of the corresponding endogenous invariant CD3-IgSF chain of the TCR/CD3 complex.

In some of any of the provided embodiments, the one or more agents capable of inducing a genetic disruption comprises a DNA binding protein or DNA-binding nucleic acid, a fusion protein comprising a DNA-targeting protein and a nuclease, or an RNA-guided nuclease that specifically binds to or hybridizes to the target site, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site. In some of any of the provided embodiments, each of the one or more agents comprise a guide RNA (gRNA) having a targeting domain that is complementary to the at least one target site. In some of any of the provided embodiments, the one or more agents are introduced as a ribonucleoprotein (RNP) complex comprising the gRNA and a Cas9 protein, optionally wherein the RNP is introduced via electroporation, particle gun, calcium phosphate transfection, cell compression or squeezing, optionally via electroporation. In some of any of the provided embodiments, the concentration of the RNP is at or about 0.1, 0.2, 0.3, 0.4, 0.5, 0.6, 0.7, 0.8, 0.9, 1, 2, 2.2, 2.5, 3, 4, 5 μg/10⁶ cells, or a range defined by any two of the foregoing values, optionally wherein the concentration of the RNP is at or about 1 μg/10⁶ cells. In some of any of the provided embodiments, the molar ratio of the gRNA and the Cas9 molecule in the RNP is at or about at or about 5:1, 4:1, 3:1, 2:1, 1:1, 1:2, 1:3, 1:4 or 1:5, or a range defined by any two of the foregoing values, optionally wherein the molar ratio of the gRNA and the Cas9 molecule in the RNP is at or about 2:1. In some of any of the provided embodiments, the gRNA has a targeting domain sequence UUGACAUGCCCUCAGUAUCC (SEQ ID NO: 8).

In some of any of the provided embodiments, the population of T cells comprise primary T cells derived from a subject, optionally wherein the subject is a human. In some of any of the provided embodiments, the T cells comprise CD8+ T cell or subtypes thereof, or CD4+ T cells or subtypes thereof.

In some of any of the provided embodiments, the polynucleotide is a linear polynucleotide, optionally a double-stranded polynucleotide or a single-stranded polynucleotide. In some of any of the provided embodiments, the polynucleotide is comprised in a vector.

In some of any of the provided embodiments, the one or more agent(s) and the polynucleotide or vector are introduced simultaneously or sequentially, in any order. In some of any of the provided embodiments, the one or more agent(s) and the polynucleotide or vector are introduced simultaneously. In some of any of the provided embodiments, the polynucleotide or vector is introduced after the introduction of the one or more agents. In some of any of the provided embodiments, the polynucleotide or vector is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the one or more agents.

In some of any of the provided embodiments, prior to the introducing of the one or more agents and/or the introducing of the polynucleotide or vector, the method involves incubating the population of T cells, in vitro with one or more stimulatory agents under conditions to stimulate or activate one or more T cells of the population, optionally wherein the one or more stimulatory agent(s) comprises and anti-CD3 and/or anti-CD28 antibodies, optionally anti-CD3/anti-CD28 beads, optionally wherein the bead to cell ratio is or is about 1:1, or oligomeric particle reagent comprising anti-CD3 and/or anti-CD28 antibodies.

In some of any of the provided embodiments, the method further involves incubating the population of T cells prior to, during or subsequent to the introducing of the one or more agents and/or the introducing of the polynucleotide or vector with one or more recombinant cytokines, optionally wherein the one or more recombinant cytokines are selected from the group consisting of IL-2, IL-7, and IL-15, optionally wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL. In some of any of the provided embodiments, the incubation is carried out subsequent to the introducing of the one or more agents and the introducing of the polynucleotide or vector, and wherein the incubation is for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

In some of any of the provided embodiments, the method further involves cultivating the population of T cells under conditions for expansion, wherein the cultivating is subsequent to the introducing of the one or more agents and/or the introducing of the polynucleotide or vector. In some of any of the provided embodiments, the cultivating under conditions for expansion comprises incubating the population of T cells with the target antigen of the antigen-binding domain, target cells expressing the target antigen, or an anti-idiotype antibody that binds to the antigen-binding domain. In some of any of the provided embodiments, the cultivating under conditions for expansion is carried out for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

In some of any of the provided embodiments, the method results in at least or greater than at or about 75%, 80%, or 90% of the cells in the population of T cells comprise a genetic disruption of at least one target site within the invariant CD3-IgSF chain locus. In some of any of the provided embodiments, the method results in at least or greater than at or about 25%, 30%, 35%, 40%, 45%, 50%, 55%, 60%, 65%, 70%, 75%, 80%, or 90% or more of T cells in the population of T cells generated by the method express the miniCAR.

Provided herein are populations comprising engineered T cells produced by the method provided herein.

Provided herein are T cells comprising a TCR/CD3 complex comprising a mini chimeric antigen receptor (CAR), wherein the miniCAR is a fusion protein comprising a heterologous antigen-binding domain and an endogenous invariant CD3 chain of the immunoglobulin superfamily (invariant CD3-IgSF chain).of the TCR/CD3 complex.

In some of any of the provided embodiments, the miniCAR is expressed from a modified invariant CD3-IgSF chain locus of the T cell, the modified invariant CD3-IgSF chain locus comprising a nucleic acid sequence encoding the miniCAR. In some of any of the provided embodiments, the invariant CD3-IgSF chain locus is a CD3 epsilon (CD3E), a CD3 delta (CD3D), or a CD3 gamma (CD3G) locus. In some of any of the provided embodiments, the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain.

Provided herein are T cells comprising a TCR/CD3 complex comprising a mini chimeric antigen receptor (miniCAR), wherein the miniCAR is a fusion protein comprising a heterologous antigen-binding domain and an endogenous CD3e chain of the TCR/CD3 complex.

In some of any of the provided embodiments, the miniCAR is expressed from a modified CD3E locus comprising a nucleic acid sequence encoding the miniCAR.

Provided herein are compositions, comprising any of the genetically engineered T cells provided herein.

Also provided herein are any of the genetically engineered T cells produced by the provided methods.

In some of any of the provided embodiments, the composition comprises CD4+ T cells and/or CD8+ T cells. In some of any of the provided embodiments, the composition comprises CD4+ T cells and CD8+ T cells and the ratio of CD4+ to CD8+ T cells is from or from about 1:3 to 3:1, optionally 1:1. In some of any of the provided embodiments, the composition comprises a plurality of T cells expressing the miniCAR. In some of any of the provided embodiments, the composition comprises at or about 1×10⁶, 1.5×10⁶, 2.5×10⁶, 5×10⁶, 7.5×10⁶, 1×10⁷, 1.5×10⁷, 2×10⁷, 2.5×10⁷, 5×10⁷, 7.5×10⁷, 1×10⁸, 1.5×10⁸, 2.5×10⁸, or 5×10⁸ total T cells. In some of any of the provided embodiments, the composition comprises at or about 1×10⁵, 2.5×10⁵, 5×10⁵, 6.5×10⁵, 1×10⁶, 1.5×10⁶, 2×10⁶, 2.5×10⁶, 5×10⁶, 7.5×10⁶, 1×10⁷, 1.5×10⁷, 5×10⁷, 7.5×10⁷, 1×10⁸ or 2.5×10⁸ T cells expressing the miniCAR. In some of any of the provided embodiments, the frequency of T cells in the composition expressing the miniCAR is at or about 25%, 30%, 35%, 40%, 45%, 50%, 55%, 60%, 65%, 70%, 75%, 80%, or 90% or more of the total cells in the composition, or of the total CD4+ T cells or CD8+ T cells in the composition, or the total cells in the composition that comprises a genetic disruption within an endogenous invariant CD3-IgSF chain locus.

In some of any of the provided embodiments, the composition is a pharmaceutical composition. In some of any of the provided embodiments, the composition further comprises a pharmaceutically acceptable carrier. In some of any of the provided embodiments, the composition further comprises a cryoprotectant.

Provided herein are methods of treatment comprising administering any of the provided engineered T cells or a population comprising engineered T cells, any of the provided T cells, or any of the provided compositions to a subject having a disease or disorder. Also provided herein are uses of any of the provided engineered T cells or a population comprising engineered T cells, any of the provided T cells, or any of the provided compositions for the treatment of a disease or disorder. Provided herein are uses of any of the provided engineered T cells or a population comprising engineered T cells, any of the provided T cells, or any of the provided compositions in the manufacture of a medicament for treating a disease or disorder.

In some of any of the provided embodiments, the methods, the engineered T cells, a population comprising engineered T cells, T cells, or compositions are for use in the treatment of a disease or disorder. In some of any of the provided embodiments, cells or tissues associated with the disease or disorder express the target antigen recognized by the antigen binding domain.

In some of any of the provided embodiments, the disease or disorder is a cancer or a tumor. In some of any of the provided embodiments, the cancer or the tumor is a hematologic malignancy, optionally a lymphoma, a leukemia, or a plasma cell malignancy. In some of any of the provided embodiments, the cancer is a lymphoma and the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL). In some of any of the provided embodiments, the cancer is a leukemia and the leukemia is chronic lymphocytic leukemia (CLL), plasma cell leukemia or acute lymphocytic leukemia (ALL). In some of any of the provided embodiments, the cancer is a plasma cell malignancy and the plasma cell malignancy is multiple myeloma (MM). In some of any of the provided embodiments, the cancer or the tumor is a solid tumor, optionally wherein the solid tumor is a non-small cell lung cancer (NSCLC) or a head and neck squamous cell carcinoma (HNSCC).

Provided herein are kits comprising one or more agents capable of inducing a genetic disruption at a target site within an endogenous invariant CD3-IgSF chain locus of a T cell; and any of the provided polynucleotides.

Provided herein are kits comprising one or more agents capable of inducing a genetic disruption at a target site within an endogenous invariant CD3-IgSF chain locus of a T cell; and any of the provided polynucleotides, wherein the polynucleotide is targeted for integration at or near the target site via homology directed repair (HDR); and instructions for carrying out any of the provided methods.

In some of any of the provided embodiments, the endogenous invariant CD3-IgSF chain locus is a CD3E locus encoding a CD3e chain, a CD3D locus encoding a CD3d chain, or a CD3G locus encoding an CD3g chain. In some of any of the provided embodiments, the endogenous invariant CD3-IgSF chain locus is a CD3E locus encoding a CD3e chain. In some of any of the provided embodiments, the endogenous invariant CD3-IgSF chain locus is a CD3D locus encoding a CD3d chain. In some of any of the provided embodiments, the endogenous invariant CD3-IgSF chain locus is a CD3G locus encoding a CD3g chain.

Provided herein are kits comprising one or more agents capable of inducing a genetic disruption at a target site within a CD3E locus of a T cell; and any of the provided polynucleotides.

In some of any of the provided embodiments, the one or more agents capable of inducing a genetic disruption comprises a DNA binding protein or DNA-binding nucleic acid that specifically binds to or hybridizes to the target site, a fusion protein comprising a DNA-targeting protein and a nuclease, or an RNA-guided nuclease, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site. In some of any of the provided embodiments, each of the one or more agents comprise a guide RNA (gRNA) having a targeting domain that is complementary to the at least one target site. In some of any of the provided embodiments, the gRNA has a targeting domain sequence UUGACAUGCCCUCAGUAUCC (SEQ ID NO: 8).

### BRIEF DESCRIPTION OF THE DRAWINGS

FIGS. 1A-1C show a schematic representation of different TCR/CD3 complex modifications. FIG. 1A depicts an assembled TCR/CD3 complex including an exemplary miniCAR containing a heterologous single chain variable fragment (scFv) antigen-binding domain linked to a CD3e chain via a linker. FIG. 1B depicts an assembled TCR/CD3 complex including an exemplary miniCAR containing a heterologous scFv antigen-binding domain linked directly to a CD3e chain. FIG. 1C depicts an assembled TCR/CD3 complex including an exemplary TCR alpha or beta chain variable domain replaced with a heterologous scFv antigen-binding domain.

FIG. 2A depicts surface expression of CD3 and CD4, detected using anti-CD3e and anti-CD4 antibodies, respectively (top panel), and surface expression of CD3 and an exemplary anti-CD19 scFv, detected using anti-CD3e and anti-idiotype (aID) antibodies, respectively, (bottom panel) of mock electroporated T cells (Mock Cells; left panel), cells electroporated with RNPs containing a gRNA targeting a T cell receptor alpha constant (TRAC) gene (TRAC KO; middle panel), and cells electroporated with RNPs containing gRNA targeting CD3E only without template polynucleotides (CD3E KO; right panel). FIGS. 2B and 2C depict flow cytometric results assessed as described in FIG. 2A for cells electroporated with pre-assembled RNP complexes containing CD3E-targeting gRNA and a Cas9 protein (1 μg/1×10⁶ cells) and either 1.2 μg (FIG. 2B, left two panels), 0.7 μg (FIG. 2B, right two panels) or 1.4 μg (FIG. 2C) of an exemplary linear template polynucleotide set forth in SEQ ID NO: 6.

FIG. 3A shows the percentage of CD3-negative cells (detected using an anti-CD3e antibody) in the mock electroporated T cell group (Mock Cells), the cells electroporated with RNPs containing gRNA targeting CD3E only without template polynucleotides (CD3E KO), and the cells electroporated with pre-assembled RNP complexes containing CD3E-targeting gRNA and a Cas9 protein (1 μg/1×10⁶ cells) and either 1.2 μg, 0.7 μg, or 1.4 μg of an exemplary linear template polynucleotide set forth in SEQ ID NO: 6. FIG. 3B shows the percentage of anti-CD19 scFv-positive cells (detected using an anti-idiotype antibody, aID) for each group described in FIG. 3A.

FIGS. 4A and 4B depict the percentage of CD3-negative cells (detected using an anti-CD3e antibody) and the percentage of anti-CD19 scFv-positive cells (detected using an anti-idiotype antibody, aID), respectively, before and after five (5) days of co-culturing with irradiated CD19-expressing LCL cells at an effector to target ratio (E:T) of 1:3 to induce cell antigen-specific cell expansion. The cell groups assessed are the same as those described in FIGS. 3A and 3B.

FIG. 5 shows the change in impedance over time during co-culture of test and control cells with plate adherent target human embryonic kidney (HEK) cells expressing CD19 at an effector to target ratio (E:T) of 10:1. The cell groups assessed are the same as those described in FIGS. 1A-1C. Additional control groups included plated HEK-CD19 cells only and media only.

FIG. 6 shows the change in impedance over time during co-culture of cells electroporated with pre-assembled RNP complexes containing CD3E-targeting gRNA and a Cas9 protein and the exemplary linear template polynucleotide set forth in SEQ ID NO: 6 with HEK-CD19+ cells at E:T ratios of 10:1, 5:1, 2.5:1, and 1.25:1. Control groups included plated HEK-CD19 cells only and media only.

FIG. 7 shows the percentage of anti-CD19 scFv-positive cells (detected using an anti-idiotype antibody, aID) for cells electroporated with RNP complexes containing TRAC-targeting gRNA and exemplary linear template polynucleotides encoding an exemplary anti-CD19 scFv (SEQ ID NO: 1, cells expressing an exemplary full length anti-CD19 chimeric antigen receptor (CAR) containing an scFv, a spacer, a transmembrane domain, a 4-1BB costimulatory domain and a CD3z domain integrated via HDR at the endogenous TRAC locus, control cells electroporated with TRAC-targeting gRNA only, and control cells electroporated with the exemplary full length CAR template only.

FIG. 8A shows the percentage of anti-CD19 scFv-positive cells (detected using an anti-idiotype antibody) for cells electroporated with pre-assembled RNP complexes containing CD3E-targeting gRNA and a Cas9 protein and an exemplary linear template polynucleotide set forth in SEQ ID NO: 7, cells expressing an exemplary full length anti-CD19 chimeric antigen receptor (CAR) containing an scFv, a spacer, a transmembrane domain, a 4-1BB costimulatory domain and a CD3z domain integrated via HDR at the endogenous TRAC locus, and Mock electroporated cells (negative control). FIG. 8B shows representative histogram profiles of the exemplary full length CAR expressed from a modified TRAC locus as described in FIGS. 7 and 8A (right panel) and the expression of the exemplary miniCAR from a modified CD3E locus as described in FIG. 8A (left panel) detected using an anti-idiotype antibody (aID) against the exemplary anti-CD19 scFv.

### DETAILED DESCRIPTION

Provided herein are genetically engineered cells such as T cells, having a modified invariant CD3 chain of the immunoglobulin superfamily (invariant CD3-IgSF chain) locus encoding a chimeric receptor. In some aspects, the modified invariant CD3-IgSF chain locus encodes a chimeric receptor that is a fusion protein containing a heterologous binding domain, e.g., antigen-binding domain, and an endogenous invariant CD3 chain of the immunoglobulin superfamily (invariant CD3-IgSF chain) (hereinafter also called a mini chimeric antigen receptor (miniCAR).

Endogenous invariant CD3-IgSF chain loci, i.e., unmodified invariant CD3-IgSF chain loci, encode invariant CD3-IgSF chains that assemble as components of the T cell receptor (TCR)-cluster of differentiation 3 (CD3) complex (TCR/CD3 complex), which is involved in the adaptive immune response. The invariant CD3-IgSF chains of the TCR/CD3 complex include the CD3epsilon (CD3e) chain, the CD3delta (CD3d) chain, and the CD3gamma (CD3g) chain, which each contain an immunoglobulin-like extracellular domain and thus are structurally related members of the immunoglobulin superfamily. The CD3e chain, the CD3d chain, and the CD3g chain, together with the CD3zeta (CD3z) and T cell receptor (TCR) alpha/beta (TCRαβ) or TCR gamma/delta (TCRγδ) heterodimers form a TCR/CD3 complex present on the surface of a T cell.

Also provided are methods for producing genetically engineered cells containing a modified invariant CD3-IgSF chain locus expressing a chimeric receptor, e.g., a miniCAR as described herein. The provided embodiments involve specifically targeting transgene sequences encoding a portion of the miniCAR, such as a portion that includes an extracellular antigen-binding domain (e.g. scFv), to the endogenous invariant CD3-IgSF chain locus, thereby producing or generating a miniCAR. In some contexts, the provided embodiments involve inducing a targeted genetic disruption, e.g., generation of a DNA break, for example, using gene editing methods, and HDR for targeted integration of the transgene sequences encoding a portion of the miniCAR, e.g., a binding domain of a miniCAR at the endogenous invariant CD3-IgSF chain locus. Also provided are related cell compositions, nucleic acids and kits for use in generation of the engineered cells provided herein and/or the methods provided herein. In some embodiments, the genetically engineered cells or cell compositions thereof can be used in adoptive cell therapy methods.

In some embodiments, the modified invariant CD3-IgSF chain locus includes one or more transgene sequences (hereinafter also referred to interchangeably as “donor” sequence, for example, sequences that are exogenous or heterologous to the T cell) encoding a portion of the miniCAR, e.g., a binding domain of a miniCAR. In some embodiments, at least a portion of the miniCAR is encoded by the genomic sequences at the endogenous invariant CD3-IgSF chain locus (the genomic locus encoding invariant CD3-IgSF chain) or a partial sequence thereof, of the engineered cell such as a T cell. In some aspects, the integration of the transgene sequence into the endogenous invariant CD3-IgSF chain locus, e.g., by homology-directed repair (HDR), is carried out such that nucleic acid sequences encoding a portion of the miniCAR are fused, e.g., fused in-frame, with an open reading frame or a partial sequence thereof, such as an exon of the open reading frame, of the endogenous invariant CD3-IgSF chain locus.

T cell-based therapies, such as adoptive T cell therapies (including those involving the administration of engineered cells expressing recombinant, engineered or chimeric receptors specific for a disease or disorder of interest, such as a chimeric antigen receptor (CAR) or other recombinant, engineered or chimeric receptors) can be effective in the treatment of cancer and other diseases and disorders. In certain contexts, other approaches for generating engineered cells for adoptive cell therapy may not always be entirely satisfactory. In some contexts, optimal efficacy can depend on the ability of the administered cells to express the receptor, including with uniform, homogenous and/or consistent expression of the receptors among cells, such as a population of immune cells and/or cells in a therapeutic cell composition, and for the receptor to recognize and bind to a target, e.g., target antigen, within the subject, tumors, and environments thereof. In some contexts, certain receptors expressed on the T cell require additional stimulatory signal, such as a co-stimulatory signal, or can be activated by antigen-independent tonic signaling. In some aspects, the provided embodiments address these problems.

In some cases, modification of an endogenous invariant CD3-IgSF chain locus as described herein results in assembly of the expressed miniCAR into the TCR/CD3 complex as part of an invariant CD3-IgSF chain. Thus, in some embodiments, the miniCAR encoded by the modified invariant CD3-IgSF chain locus can engage canonical TCR/CD3 complex signaling pathways to stimulate or activate cells, e.g., T cells, in which the miniCARs are expressed. For example, the binding of the heterologous binding domain of the miniCAR, e.g., antigen-binding domain of a miniCAR, may engage the endogenous invariant CD3-IgSF chain to which it is fused, thereby inducing an activating or stimulating signals in a T cell via the TCR/CD3 complex. In some embodiments, the ability to engage canonical TCR/CD3 complex signaling pathways according the compositions and methods described herein affords the engineered cell increased persistence, improved expression of the miniCAR, reduced tonic signaling, improved target specific cytolytic activity and/or reduced toxicity.

In some cases, available methods for introducing a chimeric receptor, such as a CAR, into a cell, include random integration of sequences encoding the chimeric receptor, such as by viral transduction. In certain respects, such methods are not entirely satisfactory. In some aspects, random integration can result in possible insertional mutagenesis and/or genetic disruption of one more random genetic loci in the cell, including those that may be important for cell function and activity. In some aspects, the efficiency of the expression of the chimeric receptor is limited among certain cells or certain cell populations that are engineered using currently available methods. In some cases, the chimeric receptor is only expressed in certain cells among a population of cells, and the level of expression of the chimeric receptor can vary widely among cells in the population. In particular aspects, the level of expression of the chimeric receptor may be difficult to predict, control and/or regulate. In some cases, semi-random or random integration of a transgene encoding the receptor into the genome of the cell may, in some cases, result in adverse and/or unwanted effects due to integration of the nucleic acid sequence into an undesired location in the genome, e.g., into an essential gene or a gene critical in regulating the activity of the cell.

In some cases, random integration may result in variable integration of the sequences encoding the recombinant or chimeric receptor, which can result in inconsistent expression, variable copy number of the nucleic acids, and/or variability of receptor expression within cells of the cell composition, such as a therapeutic cell composition. In some cases, random integration of a nucleic acid sequence encoding the receptor can result in variegated, heterogeneous, non-uniform and/or suboptimal expression or antigen binding, oncogenic transformation and transcriptional silencing of the nucleic acid sequence, depending on the site of integration and/or nucleic acid sequence copy number. In some aspects, heterogeneous and non-uniform expression in a cell population can lead to inconsistencies or instability of expression and/or antigen binding by the recombinant or chimeric receptor, unpredictability of the function or reduction in function of the engineered cells and/or a non-uniform drug product, thereby reducing the efficacy of the engineered cells. In some aspects, use of particular random integration vectors, such as certain lentiviral vectors, requires confirmation that the engineered cells do not contain replication competent virus, such as by performance of replication competent lentivirus (RCL) assay. Improved strategies are needed to achieve consistent expression levels and function of the recombinant or chimeric receptors while minimizing random integration of nucleic acids and/or heterogeneous expression in a population.

In some aspects, the size of the payload (such as transgene sequences or heterologous sequences to be inserted) in a particular polynucleotide or vector used to deliver the nucleic acid sequences encoding the chimeric receptor can be limiting. In some cases, the limited size may impact expression and/or efficiency of introduction and expression in a cell. In some cases, use of vectors such as viral vectors and/or large transgene payload can lead to reduced expression and/or efficiency of introduction of the nucleic acids and/or toxicity to the transduced cells.

The provided embodiments relate to engineering a cell to have nucleic acids encoding a portion of a miniCAR to be integrated into the endogenous invariant CD3-IgSF chain locus of a cell, e.g., T cell, by homology-directed repair (HDR). In some aspects, HDR can mediate the site specific integration of transgene sequences (such as transgene sequences encoding a recombinant receptor or a chimeric receptor or a portion, a chain or a fragment thereof), at or near a target site for genetic disruption, such as an endogenous invariant CD3-IgSF chain locus. In some embodiments, the presence of a genetic disruption (for example, at a target site at the endogenous invariant CD3-IgSF chain locus) and a polynucleotide, e.g., a template polynucleotide containing one or more homology arms (e.g., containing nucleic acid sequences that are homologous to sequences surrounding the genetic disruption) can induce or direct HDR, with homologous sequences acting as a template for DNA repair. Based on homology between the endogenous gene sequence surrounding the genetic disruption and the homology arms included in the polynucleotide, e.g., a template polynucleotide, cellular DNA repair machinery can use the polynucleotide, e.g., a template polynucleotide to repair the DNA break and resynthesize genetic information at the target site of the genetic disruption, thereby effectively inserting or integrating the sequences between the homology arms (such as transgene sequences encoding a portion of a miniCAR) at or near the target site of the genetic disruption. The provided embodiments can generate cells containing a modified invariant CD3-IgSF chain locus encoding a miniCAR, where transgene sequences encoding a portion of the miniCAR, e.g., a binding domain, is integrated into the endogenous invariant CD3-IgSF chain locus by HDR.

In some aspects, the provided embodiments offer advantages in producing engineered cells with improved and/or more efficient targeting of the nucleic acids encoding a portion of the chimeric into the cell. In some cases, the methods minimize possible semi-random or random integration and/or heterogeneous or variegated expression and/or undesired expression from unintegrated nucleic acid sequences, and result in improved, uniform, homogeneous, consistent, predictable or stable expression of the chimeric or recombinant receptor or having reduced, low or no possibility of insertional mutagenesis.

In some aspects, the provided chimeric receptor miniCARs exhibit improved features compared to conventional chimeric antigen receptors (CARs). Typically, a CAR is a chimeric or recombinant receptor that contains an extracellular antigen-binding domain, a transmembrane domain, an intracellular region comprising a CD3zeta (CD3) signaling domain, and optionally comprising a co-stimulatory signaling domain, typically in which all domains of the CAR are part of the same polypeptide chain and/or are all exogenous to the engineered cell in which it is expressed. In some aspects, compared to other methods of producing genetically engineered T cells expressing such other conventional chimeric or recombinant receptors, e.g., CARs, the provided embodiments allow for a more stable, more physiological, more controllable or more uniform, consistent or homogeneous expression of the miniCAR chimeric receptor. In some cases, the methods result in the generation of more consistent and more predictable drug product, e.g. cell composition containing the engineered cells, which can result in a safer therapy for treated patients. In some aspects, the provided embodiments also allow predictable and consistent integration at a single gene locus or a multiple gene loci of interest. In some embodiments, the provided embodiments can also result in generating a cell population with consistent copy number (typically, 1 or 2) of the nucleic acids that are integrated in the cells of the population, which, in some aspects, provide consistency in chimeric receptor expression and expression of the endogenous receptor genes within a cell population. In some cases, the provided embodiments do not involve the use of a viral vector for integration and thus can reduce the need for confirmation that the engineered cells do not contain replication competent virus, thereby improving the safety of the cell composition, and reducing toxicity resulting from use of viral vectors in transduction.

The methods of integration described herein, e.g., HDR, provide further advantages compared to other methods of integration of such chimeric receptors, such as random or semi-random genomic insertion. For example, engineering cells to encode a miniCAR at an endogenous invariant CD3-IgSF chain locus prevents said locus from expressing the endogenous invariant CD3-IgSF chain, thereby decreasing the availability of endogenous invariant CD3-IgSF chains for assembly into the TCR/CD3 complex and increasing the probability of the miniCAR assembly into the TCR/CD3 complex. In some cases, alternative methods for expressing a chimeric receptor containing fusion of an antigen binding domain with a heterologous invariant CD3-IgSF domain may lead to increased variability in expression of the chimeric receptor in engineered cells, for example, due to competition with the endogenous invariant CD3-IgSF chains of the TCR/CD3 complex. For instance, such methods include those that utilize random genome insertion, which do not necessarily decrease the availability of endogenous invariant CD3-IgSF chains, resulting in competition between the endogenous invariant CD3-IgSF chain and the randomly inserted chain for assembly into the TCR/CD3 complex. Thus, in some cases, the compositions and methods provided herein increase the probability of the miniCAR being assembled into the TCR/CD3 complex.

It should also be appreciated that in some embodiments, the integration methods and compositions provided herein minimize the total size of the transgene to be integrated. For example, in contrast to integration of a typical or conventional CAR sequence, which comprises multiple domains to function, the transgenes provided herein may minimally include sequences encoding a binding domain, e.g., an antigen-binding domain. In some aspects, the transgenes can also include sequences encoding a linker. In some embodiments, the transgene may also include a multicistronic element, e.g., a 2A element. Thus, the total size of the transgene provided herein may be at least 75%, 70%, 65%, 60%, 55%, 50%, or more smaller than a CAR. This can reduce the time and costs necessary for preparing the nucleic acids encoding the chimeric receptor, and the time and costs needed for cell engineering. Further, because the transgene can be integrated using precise HDR techniques, expression of the transgene may be controlled by an endogenous promoter sequence or other regulatory element, thus circumventing the need to include such elements in the transgene construct. In some embodiments, the smaller transgene size, for example as provided herein, may reduce production costs; increase integration efficiency, e.g., transfection efficiency; reduce the cytotoxic effects of integration; and eliminate the need for transgene delivery via virus-derived vectors.

The chimeric receptors encoded from the modified invariant CD3-IgSF chain locus in engineered cells provided herein can be encoded under the control of endogenous or exogenous regulatory elements. In some aspects, the provided embodiments allow the chimeric receptor to be expressed under the control of the endogenous invariant CD3-IgSF chain regulatory elements, which, in some cases, can provide a more physiological level of expression. In some aspects, the provided embodiments allow the nucleic acids encoding the miniCAR to be expressed under the control of the endogenous regulatory or control elements, e.g., cis regulatory elements, such as the promoter, or the 5′ and/or 3′ untranslated regions (UTRs) of the endogenous invariant CD3-IgSF chain locus. Thus, in some aspects, the provided embodiments allow the miniCAR to be expressed and/or the expression is regulated at a similar level to the endogenous invariant CD3-IgSF chain.

In some aspects, the provided embodiments can reduce or minimize antigen-independent signaling or activity (also known as “tonic signaling”) through the miniCAR. In some cases, antigen-independent signaling can result from overexpression or uncontrolled activity of the expressed chimeric receptor, and can lead to undesirable effects, such as increased differentiation and/or exhaustion of T cells that express the chimeric receptor. In some embodiments, the provided engineered cells and cell compositions can reduce the effect of antigen-independent signaling by that may result from overexpression or uncontrolled activity of the expressed chimeric receptor. Thus, the provided embodiments can facilitate the production of engineered cells that exhibit improved expression, function and uniformity of expression and/or other desired feature or properties, and ultimately higher efficacy. In some embodiments, the provided polynucleotides, transgenes, and/or vectors, when delivered into T cells, result in the expression of chimeric receptors, e.g., miniCARs, that can modulate T cell activity, and, in some cases, can modulate T cell differentiation or homeostasis.

In some aspects, the provided embodiments allow the miniCAR to be expressed under the control of exogenous or heterologous regulatory or control elements, which, in some aspects, provides a more controllable level of expression.

In some aspects, the provided embodiments can prevent uncontrolled expression or expression from randomly integrated or unintegrated polynucleotides. In some embodiments, the introduced polynucleotide, e.g., template polynucleotide, does not contain the nucleic acid sequences encoding the full length functional receptor, since the miniCAR is partially encoded by endogenous components of the cell. Thus, typically, the full invariant CD3-IgSF chain is not encoded by the introduced polynucleotide, but rather is at least partially encoded by the endogenous invariant CD3-IgSF locus of the cell into which the provided polynucleotides are introduced. In some aspects, transcription from randomly integrated or unintegrated polynucleotides would not produce a functional receptor. In some aspects, only upon integration at the target locus, e.g., the endogenous invariant CD3-IgSF chain locus, can a functional receptor containing all of required signaling regions be generated. In some aspects, the provided embodiments can result in improved safety of the cell composition, for example, by preventing uncontrolled expression, e.g. from randomly integrated or unintegrated polynucleotides, such as unintegrated viral vector sequences.

As described above, the provided embodiments can also reduce the length of transgene sequences required to produce the miniCAR. In some embodiments, reducing the size of the transgene allows for sufficient space to package additional elements and/or transgenes within the same vector, e.g., viral vector. In some aspects, the provided embodiments also permit the use of a smaller nucleic acid sequence fragments for engineering compared to existing methods, by utilizing a portion or all of the open reading frame sequences of the endogenous gene encoding the invariant CD3-IgSF chain, to encode all or a portion of invariant CD3-IgSF chain of the miniCAR. In some aspects, the provided embodiments provide flexibility for engineering cells to express a miniCAR compared to existing methods, because the methods utilize a portion or all of the open reading frame sequences of the endogenous gene encoding the invariant CD3-IgSF chain, to encode the invariant CD3-IgSF chain or a portion thereof of the miniCAR. In some cases, this can reduce the payload space for sequences encoding the portion thereof of the miniCAR and leave space for sequences encoding other components, such as other transgene sequences, homology arms, regulatory elements, since the length requirement for nucleic acid sequences encoding the portion thereof of the miniCAR is reduced. In some aspects, the provided embodiments may allow accommodation of larger homology arms compared to conventional embodiments that require the entire length of the chimeric receptor, e.g., CAR, in the introduced polynucleotide, and/or allow accommodation of nucleic acid sequences encoding additional molecules, as the length requirement for nucleic acid sequences encoding a portion of the miniCAR is reduced. In some aspects, generation, delivery of the nucleic acid sequences, e.g., transgene sequences, and/or targeting efficiency by homology-directed repair (HDR), may be facilitated or improved using the provided embodiments. In other aspects, the provided embodiments allow accommodation of nucleic acid sequences encoding additional molecules for expression on or in the cell.

Also provided are methods for engineering, preparing, and producing the engineered cells, and kits and devices for generating or producing the engineered cells. Also provided are cells and cell compositions generated by the methods. Provided are polynucleotides, e.g., viral vectors, that contain a nucleic acid sequence encoding a portion of the miniCAR, and methods for introducing such polynucleotides into the cells, such as by transduction or by physical delivery, such as electroporation. Also provided are compositions containing the engineered cells, and methods, kits, and devices for administering the cells and compositions to subjects, such as for adoptive cell therapy. In some aspects, the cells are isolated from a subject, engineered, and administered to the same subject. In other aspects, they are isolated from one subject, engineered, and administered to another subject. The resulting genetically engineered cells or cell compositions can be used in adoptive cell therapy methods.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Method for Generating Cells Expressing a Minicar by Homology-Directed Repair

Provided herein are methods of generating or producing genetically engineered cells comprising a modified invariant CD3 chain of the immunoglobulin superfamily (invariant CD3-IgSF) chain locus, e.g., CD3E, CD3D or CD3G locus, in which the modified invariant CD3-IgSF chain locus, includes nucleic acid sequences encoding a chimeric receptor, such as a mini chimeric antigen receptor (miniCAR).

In some aspects, the modified invariant CD3-IgSF chain locus, e.g., CD3E, CD3D or CD3G locus, in the genetically engineered cell comprises a transgene sequence encoding a portion of a miniCAR, such as an extracellular antigen-binding domain, integrated into an endogenous invariant CD3-IgSF chain locus, e.g., CD3E, CD3D or CD3G locus, which normally encodes one of the invariant CD3 chains of the immunoglobulin superfamily. In some embodiments, the methods involve inducing a targeted genetic disruption and homology-dependent repair (HDR), using polynucleotides (for example, also called “template polynucleotides”) containing the transgene encoding a portion, such as an antigen-binding domain, of a miniCAR, thereby targeting integration of the transgene at the invariant CD3-IgSF chain locus. Also provided are cells and cell compositions generated by the methods. In some embodiments, also provided are compositions containing a population of cells that have been engineered to express a miniCAR, such that the cell population that exhibits more improved, uniform, homogeneous and/or stable expression and/or antigen-binding by the miniCAR, including genetically engineered T cells produced by any of the provided methods, and polynucleotides, e.g., template polynucleotides, and kits for use in the methods.

In some aspects, the expressed miniCAR, comprises all or a portion of an invariant CD3 chain of the immunoglobulin superfamily (invariant CD3-IgSF chain). For example, in some aspects, the expressed miniCAR is a fusion protein comprising an antigen-binding domain, encoded by an introduced heterologous sequence (e.g., transgene), and all or a portion of the invariant CD3-IgSF chain, such as the extracellular region or domain, transmembrane region or domain and intracellular region or domain of the invariant CD3-IgSF chain, encoded by the endogenous sequences of the invariant CD3-IgSF chain locus. In some aspects, the expressed miniCAR is a fusion protein comprising a heterologous antigen-binding domain and an endogenous invariant CD3-IgSF chain. In some aspects, after integration of the transgene sequences encoding a portion, e.g., an antigen-binding domain, of the miniCAR into the invariant CD3-IgSF chain locus, at least a portion of the invariant CD3-IgSF chain is encoded by an open reading frame or partial sequence thereof of the invariant CD3-IgSF chain locus, in the genome. In some aspects, the heterologous antigen-binding domain is in the N terminus of the fusion protein, and the endogenous invariant CD3-IgSF chain is in the C terminus of the fusion protein. In some aspects, the invariant CD3-IgSF chain is a CD3epsilon (CD3e or CD3ε) chain, a CD3delta (CD3d or CD3δ) chain or a CD3gamma (CD3g or CD3γ) chain.

In some embodiments, the methods employ HDR for targeted integration of the transgene sequences into the invariant CD3-IgSF chain locus. In some cases, the methods involve introducing one or more targeted genetic disruption(s), e.g., DNA break, at the endogenous invariant CD3-IgSF chain locus, by gene editing techniques, combined with targeted integration of transgene sequences encoding a portion of the miniCAR by HDR. In some embodiments, the HDR step entails a disruption or a break, e.g., a double-stranded break, in the DNA at the target genomic location. In some embodiments, the DNA break is induced by employing gene editing methods, e.g., targeted nucleases.

In some aspects, the provided methods involve introducing one or more agent(s) capable of inducing a genetic disruption of at a target site within an invariant CD3-IgSF chain locus, into a T cell; and introducing into the T cell a polynucleotide, e.g., a template polynucleotide, comprising a transgene and one or more homology arms. In some aspects, the transgene contains a sequence of nucleotides encoding a portion of a miniCAR. In some embodiments, the nucleic acid sequence, such as the transgene, is targeted for integration within the invariant CD3-IgSF chain locus, via homology directed repair (HDR). In some aspects, the provided methods involve introducing a polynucleotide comprising a transgene sequence encoding a portion, such as an antigen-binding domain, of a miniCAR into a T cell having a genetic disruption of within an invariant CD3-IgSF chain locus, wherein the genetic disruption has been induced by one or more agents capable of inducing a genetic disruption of one or more target site within the invariant CD3-IgSF chain locus, and wherein the nucleic acid sequence, such as the transgene, is targeted for integration within the invariant CD3-IgSF chain locus, via HDR.

In some aspects, the embodiments involve generating a targeted genomic disruption, such as a targeted DNA break, using gene editing methods and/or targeted nucleases, followed by HDR based on one or more polynucleotide(s), e.g., template polynucleotide(s) that contains homology sequences that are homologous to sequences at the endogenous invariant CD3-IgSF chain locus, linked to transgene sequences encoding a portion of the miniCAR and, in some embodiments, nucleic acid sequences encoding other molecules, to specifically target and integrate the transgene sequences at or near the DNA break. Thus, in some aspects, the methods involve a step of inducing a targeted genetic disruption (e.g., via gene editing) and introducing a polynucleotide, e.g., a template polynucleotide comprising transgene sequences, into the cell (e.g., via HDR).

In some embodiments, the targeted genetic disruption and targeted integration of the transgene sequences by HDR occurs at one or more target site(s) at the endogenous invariant CD3-IgSF chain locus, e.g., CD3E, CD3D or CD3G locus, which encode CD3e, CD3d or CD3g, respectively. In some aspects, the targeted integration occurs within an open reading frame sequence of the endogenous invariant CD3-IgSF chain locus. In some aspects, targeted integration of the transgene sequences results in an in-frame fusion of the coding portion of the transgene with one or more exons of the open reading frame of the endogenous invariant CD3-IgSF chain locus, e.g., in-frame with the adjacent exon at the integration site.

In some embodiments, a polynucleotide, e.g., template polynucleotide, is introduced into the engineered cell, prior to, simultaneously with, or subsequent to introduction of one or more agent(s) capable of inducing one or more targeted genetic disruption. In the presence of one or more targeted genetic disruption, e.g., DNA break, the polynucleotide can be used as a DNA repair template, to effectively copy and/or integrate the transgene, at or near the site of the targeted genetic disruption by HDR, based on homology between the endogenous gene sequence surrounding the genetic disruption and the one or more homology arms, such as the 5′ and/or 3′ homology arms, included in the template polynucleotide.

In some aspects, the two steps can be performed sequentially. In some embodiments, the gene editing and HDR steps are performed simultaneously and/or in one experimental reaction. In some embodiments, the gene editing and HDR steps are performed consecutively or sequentially, in one or consecutive experimental reaction(s). In some embodiments, the gene editing and HDR steps are performed in separate experimental reactions, simultaneously or at different times.

The immune cells can include a population of cells containing T cells. Such cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, the T cells are primary cells, such as primary T cells. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the polynucleotide (e.g., template polynucleotide) and the step of introducing the agent (e.g. Cas9/gRNA RNP) can occur simultaneously or sequentially in any order. In some embodiments, the polynucleotide is introduced simultaneously with the introduction of the one or more agents capable of inducing a genetic disruption (e.g. Cas9/gRNA RNP). In particular embodiments, the polynucleotide template is introduced into the T cells after inducing the genetic disruption by the step of introducing the agent(s) (e.g. Cas9/gRNA RNP). In some embodiments, prior to, during and/or subsequent to introduction of the polynucleotide template and one or more agents (e.g. Cas9/gRNA RNP), the cells are cultured or incubated under conditions to stimulate expansion and/or proliferation of cells.

In particular embodiments of the provided methods, the introduction of the template polynucleotide is performed after the introduction of the one or more agent capable of inducing a genetic disruption. Any method for introducing the one or more agent(s) can be employed as described, depending on the particular agent(s) used for inducing the genetic disruption. In some aspects, the disruption is carried out by gene editing, such as using an RNA-guided nuclease such as a clustered regularly interspersed short palindromic nucleic acid (CRISPR)-Cas system, such as CRISPR-Cas9 system, specific for the invariant CD3-IgSF chain locus, e.g., CD3E, CD3D or CD3G locus, being disrupted. In some aspects, the disruption is carried out using a CRISPR-Cas9 system specific for the invariant CD3-IgSF chain locus. In some embodiments, an agent containing a Cas9 and a guide RNA (gRNA) containing a targeting domain, which targets a region of the invariant CD3-IgSF chain locus, is introduced into the cell. In some embodiments, the agent is or comprises a ribonucleoprotein (RNP) complex of Cas9 and gRNA containing the invariant CD3-IgSF chain locus,-targeted targeting domain (Cas9/gRNA RNP). In some embodiment, the introduction includes contacting the agent or portion thereof with the cells, in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap. In some cases, electroporation of the cells to be modified includes cold-shocking the cells, e.g. at 32° C. following electroporation of the cells and prior to plating.

In such aspects of the provided methods, the polynucleotide, e.g., template polynucleotide, is introduced into the cells after introduction with the one or more agent(s), such as Cas9/gRNA RNP, e.g. that has been introduced via electroporation. In some embodiments, the polynucleotide, e.g., template polynucleotide, is introduced immediately after the introduction of the one or more agents capable of inducing a genetic disruption. In some embodiments, the polynucleotide, e.g., template polynucleotide, is introduced into the cells within at or about 30 seconds, within at or about 1 minute, within at or about 2 minutes, within at or about 3 minutes, within at or about 4 minutes, within at or about 5 minutes, within at or about 6 minutes, within at or about 6 minutes, within at or about 8 minutes, within at or about 9 minutes, within at or about 10 minutes, within at or about 15 minutes, within at or about 20 minutes, within at or about 30 minutes, within at or about 40 minutes, within at or about 50 minutes, within at or about 60 minutes, within at or about 90 minutes, within at or about 2 hours, within at or about 3 hours or within at or about 4 hours after the introduction of one or more agents capable of inducing a genetic disruption. In some embodiments, the polynucleotide, e.g., template polynucleotide, is introduced into cells at time between at or about 15 minutes and at or about 4 hours after introducing the one or more agent(s), such as between at or about 15 minutes and at or about 3 hours, between at or about 15 minutes and at or about 2 hours, between at or about 15 minutes and at or about 1 hour, between at or about 15 minutes and at or about 30 minutes, between at or about 30 minutes and at or about 4 hours, between at or about 30 minutes and at or about 3 hours, between at or about 30 minutes and at or about 2 hours, between at or about 30 minutes and at or about 1 hour, between at or about 1 hour and at or about 4 hours, between at or about 1 hour and at or about 3 hours, between at or about 1 hour and at or about 2 hours, between at or about 2 hours and at or about 4 hours, between at or about 2 hours and at or about 3 hours or between at or about 3 hours and at or about 4 hours. In some embodiments, the polynucleotide, e.g., template polynucleotide, is introduced into cells at or about 2 hours after the introduction of the one or more agents, such as Cas9/gRNA RNP, e.g. that has been introduced via electroporation.

Any method for introducing the polynucleotide, e.g., template polynucleotide, can be employed as described, depending on the particular methods used for delivery of the polynucleotide, e.g., template polynucleotide, to cells. Exemplary methods include those for transfer of nucleic acids encoding the receptors, including via viral, e.g., retroviral or lentiviral, transduction, transposons, and electroporation. In particular embodiments, viral transduction methods are employed. In some embodiments, the polynucleotides can be transferred or introduced into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557. In particular embodiments, the viral vector is an AAV such as an AAV2 or an AAV6.

In some embodiments, prior to, during or subsequent to contacting the agent with the cells and/or prior to, during or subsequent to effecting delivery (e.g. electroporation), the provided methods include incubating the cells in the presence of a cytokine, a stimulating agent and/or an agent that is capable of inducing proliferation, stimulation or activation of the T cells. In some embodiments, at least a portion of the incubation is in the presence of a stimulating agent that is or comprises an antibody specific for CD3 an antibody specific for CD28 and/or a cytokine, such as anti-CD3/anti-CD28 beads. In some embodiments, at least a portion of the incubation is in the presence of a cytokine, such as one or more of recombinant IL-2, recombinant IL-7 and/or recombinant IL-15. In some embodiments, the incubation is for up to 8 days before or after the introduction with the one or more agent(s), such as Cas9/gRNA RNP, e.g. via electroporation, and the polynucleotide, e.g., template polynucleotide, such as up to 24 hours, 36 hours or 48 hours or 3, 4, 5, 6, 7 or 8 days.

In some embodiments, the method includes activating or stimulating cells with a stimulating agent (e.g. anti-CD3/anti-CD28 antibodies) prior to introducing the agent, e.g. Cas9/gRNA RNP, and the polynucleotide template. In some embodiments, the incubation in the presence of a stimulating agent (e.g. anti-CD3/anti-CD28) is for 6 hours to 96 hours, such as 24 to 48 hours or 24 to 36 hours prior to the introduction with the one or more agent(s), such as Cas9/gRNA RNP, e.g. via electroporation. In some embodiments, the incubation with the stimulating agents can further include the presence of a cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). In some embodiments the stimulating agent(s) (e.g. anti-CD3/anti-CD28 antibodies) is washed or removed from the cells prior to introducing or delivering into the cells the agent(s) capable of inducing a genetic disruption Cas9/gRNA RNP and/or the polynucleotide template. In some embodiments, prior to the introducing of the agent(s), the cells are rested, e.g. by removal of any stimulating or activating agent. In some embodiments, prior to introducing the agent(s), the stimulating or activating agent and/or cytokines are not removed.

In some embodiments, subsequent to the introduction of the agent(s), e.g. Cas9/gRNA, and/or the polynucleotide template the cells are incubated, cultivated or cultured in the presence of a recombinant cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). The cells can be incubated or cultivated under conditions to induce proliferation or expansion of the cells. In some embodiments, the cells can be incubated or cultivated until a threshold number of cells is achieved for harvest, e.g. a therapeutically effective dose.

In some embodiments, the incubation during any portion of the process or all of the process can be at a temperature of 30° C.±2° C. to 39° C.±2° C., such as at least or about at least 30° C.±2° C., 32° C.±2° C., 34° C.±2° C. or 37° C.±2° C. In some embodiments, at least a portion of the incubation is at 30° C.±2° C. and at least a portion of the incubation is at 37° C.±2° C.

In some embodiments, upon targeted integration, the nucleic acid sequence present at the modified invariant CD3-IgSF chain locus, e.g., CD3E, CD3D or CD3G locus, comprises a fusion of a transgene (e.g. a portion of a miniCAR, as described herein), targeted by HDR, with an open reading frame or a partial sequence thereof of an endogenous invariant CD3-IgSF chain locus. In some aspects, the nucleic acid sequence present at the modified invariant CD3-IgSF chain locus, comprises a transgene, e.g. a portion of a miniCAR as described herein, that is integrated at an endogenous invariant CD3-IgSF chain locus, comprising an open reading frame encoding an invariant CD3-IgSF chain locus, e.g., CD3e, CD3d or CD3g, respectively (see Tables 1-5 herein for description of exemplary endogenous invariant CD3-IgSF chain loci). In some aspects, upon targeted integration or fusion, e.g., in-frame fusion, the heterologous sequence (e.g., encoding an antigen-binding domain) of the transgene and a portion of the open reading frame at the endogenous invariant CD3-IgSF chain locus, e.g., CD3E, CD3D or CD3G locus, together encode a chimeric receptor, e.g. miniCAR, containing a heterologous antigen-binding domain and an endogenous invariant CD3-IgSF chain. Thus, the provided embodiments utilize all or a portion of the open reading frame sequences of the endogenous invariant CD3-IgSF chain locus, to encode a portion of the miniCAR, for example, including the transmembrane and intracellular portions of the chimeric receptor. In some embodiments, upon targeted, in-frame integration of the transgene sequence, the modified invariant CD3-IgSF chain locus, contains a sequence encoding a whole, complete or full-length miniCAR containing an extracellular antigen-binding domain and all or a portion of the extracellular region of the invariant CD3-IgSF chain; a transmembrane region of the invariant CD3-IgSF chain and the intracellular region of the invariant CD3-IgSF chain.

Exemplary methods for carrying out genetic disruption at the endogenous invariant CD3-IgSF chain locus, and/or for carrying out HDR for targeted integration of the transgene sequences, such as a portion of a chimeric receptor, e.g. a portion of a miniCAR, into the invariant CD3-IgSF chain locus, are described in the following subsections.

A. Genetic Disruption

In some embodiments, one or more targeted genetic disruption is induced at the endogenous genomic locus encoding an invariant CD3 chain of the immunoglobulin superfamily (invariant CD3-IgSF chain), e.g., a CD3epsilon (CD3e or CD3ε) chain, a CD3delta (CD3d or CD3δ) chain or a CD3gamma (CD3g or CD3γ) chain. In some embodiments, one or more targeted genetic disruption is induced at an endogenous invariant CD3-IgSF chain locus, e.g., a CD3E (encoding CD3e), a CD3D (encoding CD3d) or a CD3G (encoding CD3g) locus. In some embodiments, one or more targeted genetic disruption is induced at one or more target sites at or near an endogenous invariant CD3-IgSF chain locus, e.g., CD3E, CD3D or CD3G locus. In some embodiments, the targeted genetic disruption is induced in an intron of an endogenous invariant CD3-IgSF chain locus. In some embodiments, the targeted genetic disruption is induced in an exon of an endogenous invariant CD3-IgSF chain locus. In some aspects, the presence of the one or more targeted genetic disruption and a polynucleotide, e.g., a template polynucleotide that contains a transgene comprising a sequence encoding an antigen-binding domain, can result in targeted integration of the transgene sequences at or near the one or more genetic disruption (e.g., target site) at an endogenous invariant CD3-IgSF chain locus.

In some embodiments, genetic disruption results in a DNA break, such as a double-strand break (DSB) or a cleavage, or a nick, such as a single-strand break (SSB), at one or more target site in the genome. In some embodiments, at the site of the genetic disruption, e.g., DNA break or nick, action of cellular DNA repair mechanisms can result in knock-out, insertion, missense or frameshift mutation, such as a biallelic frameshift mutation, deletion of all or part of the gene; or, in the presence of a repair template, e.g., a template polynucleotide, can alter the DNA sequence based on the repair template, such as integration or insertion of the nucleic acid sequences, such as a transgene encoding all or a portion of a miniCAR contained in the template. In some embodiments, the genetic disruption can be targeted to one or more exon of a gene or portion thereof. In some embodiments, the genetic disruption can be targeted near a desired site of targeted integration of heterologous sequences, e.g., transgene sequences encoding a portion, such as an antigen-binding domain, of a miniCAR.

In some embodiments, a DNA binding protein or DNA-binding nucleic acid, which specifically binds to or hybridizes to the sequences at a region near one of the at least one target site(s), is used for targeted disruption. In some embodiments, template polynucleotides, e.g., template polynucleotides that include nucleic acid sequences, such as a transgene encoding a portion of a chimeric receptor, and homology sequences, can be introduced for targeted integration by HDR of the chimeric receptor-encoding sequences at or near the site of the genetic disruption, such as described herein, for example, in Section I.A.

In some embodiments, the genetic disruption is carried out by introducing one or more agent(s) capable of inducing a genetic disruption. In some embodiments, such agents comprise a DNA binding protein or DNA-binding nucleic acid that specifically binds to or hybridizes to the gene. In some embodiments, the agent comprises various components, such as a fusion protein comprising a DNA-targeting protein and a nuclease or an RNA-guided nuclease. In some embodiments, the agents can target one or more target sites or target locations. In some aspects, a pair of single stranded breaks (e.g., nicks) on each side of the target site can be generated.

In provided embodiments, the term “introducing” encompasses a variety of methods of introducing a nucleic acid and/or a protein, such as DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors. Methods, such as electroporation, also can be used to introduce or deliver proteins or ribonucleoprotein (RNP), e.g. containing the Cas9 protein in complex with a targeting gRNA, to cells of interest.

In some embodiments, the genetic disruption occurs at a target site (also known as “target position,” “target DNA sequence” or “target location”), for example, at an endogenous invariant CD3-IgSF chain locus, e.g., CD3E, CD3D or CD3G locus. In some embodiments, the target site includes a site on a target DNA (e.g., genomic DNA) that is modified by the one or more agent(s) capable of inducing a genetic disruption, e.g., a Cas9 molecule complexed with a gRNA that specifies the target site. For example, the target site can include locations in the DNA at a endogenous invariant CD3-IgSF chain locus, e.g., CD3E, CD3D or CD3G locus, where cleavage or DNA breaks occur. In some aspects, integration of nucleic acid sequences, such as a transgene encoding an antigen-binding domain of a miniCAR by HDR can occur at or near the target site or target sequence. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence.

1. Target Site at an Endogenous Invariant CD3-IgSF Chain Locus, e.g., CD3E, CD3D or CD3G Locus

In some embodiments, the genetic disruption, and/or integration of the transgene encoding an antigen-binding domain via homology-directed repair (HDR), are targeted at an endogenous or genomic locus that encodes an invariant CD3 chain of the immunoglobulin superfamily (invariant CD3-IgSF chain). In some of any of the provided embodiments, the genetic locus into which the genetic disruption and/or integration of the transgene encoding an antigen-binding domain via homology-directed repair (HDR), are targeted, is a gene locus encoding an invariant CD3 chain of the immunoglobulin superfamily (invariant CD3-IgSF chain). In some aspects, the invariant CD3-IgSF chain is a CD3e, and the invariant CD3-IgSF chain locus is CD3E. In some aspects, the invariant CD3-IgSF chain is a CD3d, and the invariant CD3-IgSF chain locus is CD3D. In some aspects, the invariant CD3-IgSF chain is a CD3g, and the invariant CD3-IgSF chain locus is CD3G. In some aspects, the genetic disruption is targeted at a target site within the invariant CD3-IgSF chain locus, e.g., CD3E, CD3D or CD3G locus, containing an open reading frame encoding CD3e, CD3d or CD3g, such that targeted integration, fusion or insertion of transgene sequences occurs at or near the site of genetic disruption at the invariant CD3-IgSF chain locus. In some aspects, the genetic disruption is targeted at or near an exon of the open reading frame encoding the invariant CD3-IgSF chain, e.g., CD3e, CD3d or CD3g. In some aspects, the genetic disruption is targeted at or near an intron of the open reading frame encoding the invariant CD3-IgSF chain, e.g., CD3e, CD3d or CD3g.

The invariant CD3-IgSF chains are components of the T cell receptor (TCR)-cluster of differentiation 3 (CD3) complex present on the surface of the T cell which is involved in adaptive immune response. In some aspects, the invariant CD3-IgSF chain is a CD3epsilon (CD3e) chain, a CD3delta (CD3d) chain or a CD3gamma (CD3g) chain. CD3epsilon (also known as CD3e, CD3ε; CD3E, IMD18, T3E, TCRE, CD3e molecule) chain, CD3delta (also known as CD3d, CD36 CD3D, CD3-DELTA, IMD19, T3D, CD3d molecule) chain, and CD3gamma (also known as CD3g, CD3γ, CD3G, CD3-GAMMA, IMD17, T3G, CD3g molecule), together with CD3zeta (also known as CD3-zeta; CD3; T-cell receptor T3 zeta chain; CD3Z; T3Z; TCRZ; cluster of differentiation 247; CD247; IMD25) and T cell receptor (TCR) alpha/beta (TCRαβ) or TCR gamma/delta (TCRγδ) heterodimers, CD3gamma (CD3γ), CD3delta (CD3δ) and CD3epsilon (CDR), form the TCR-CD3 complex.

The TCR-CD3 complex is a protein complex that is involved in stimulating or activating both the cytotoxic T cells (CD8+ T cells) and helper T cells (CD4+ T cells). In some aspects, the complex contains a CD3g chain, a CD3d chain, and two CD3e chains, associated with the TCR and the CD3z to generate a stimulating or activating primary cytoplasmic or intracellular signal in T lymphocytes. The TCR/CD3 complex typically comprises CD3ge-CD3de-CD3zz chain hexamer, and the TCR alpha and TCR beta chains (see, e.g., Call et al., Mol Immunol. 2004 April; 40(18): 1295-1305). The TCR, CD3z and the invariant CD3-IgSF chains together constitute the TCR complex. Following binding of the antigen to the antigen-binding domain, the TCR-CD3 complex relays information from the antigen- or ligand-binding modules, e.g., a TCR, to the signaling modules, e.g., CD3 chains, including the invariant CD3-IgSF, and on to the intracellular signaling apparatus. The CD3 chains of the TCR/CD3 complex contains one or more immunoreceptor tyrosine-based activation motifs (ITAMs) in their intracellular or cytoplasmic domain. In some aspects, the invariant CD3 chains of the immunoglobulin superfamily (invariant CD3-IgSF chains), e.g., CD3e, CD3d or CD3g, are highly related cell-surface proteins of the immunoglobulin superfamily containing a single extracellular immunoglobulin domain, and contain a single conserved ITAM, to generate the stimulating or activating signal. The TCR/CD3 complex can couple antigen recognition to intracellular signal transduction pathways, by stimulating or activating primary cytoplasmic or intracellular signaling, e.g., via the ITAMs. Upon engagement of the TCR with its ligand (e.g., a peptide in the context of an MHC molecule; MHC-peptide complex), the ITAM motifs can be phosphorylated by kinases including Src family protein tyrosine kinases LCK and FYN, resulting in the stimulation of downstream signaling pathways. In some aspects, the phosphorylation of CD3 ITAM creates docking sites for the protein kinase ZAP70, leading to phosphorylation and activation of ZAP70, and a signaling cascade in the T cell.

An exemplary human CD3e precursor polypeptide sequence is set forth in SEQ ID NO:17 (isoform 1; mature polypeptide includes residues 23-207 of SEQ ID NO:17; see Uniprot Accession No. P07766; NCBI Reference Sequence: NP_000724.1; mRNA sequence set forth in SEQ ID NO:18, NCBI Reference Sequence: NM_000733) or SEQ ID NO:19 (isoform 2; mature polypeptide includes residues 22-201 of SEQ ID NO:18; see Uniprot Accession No. E9PSH8). Exemplary mature CD3e chain isoform 1 contains an extracellular region (including amino acid residues 23-126 of the human CD3e chain precursor sequence set forth in SEQ ID NO:17), a transmembrane region (including amino acid residues 127-152 of the human CD3e chain precursor sequence set forth in SEQ ID NO:17), and an intracellular region (including amino acid residues 153-207 of the human CD3e chain precursor sequence set forth in SEQ ID NO:17). The CD3e chain isoform 1 contains an immunoreceptor tyrosine-based activation motif (ITAM) domains, at amino acid residues 178-205 of the human CD3e chain precursor sequence set forth in SEQ ID NO:17.

In humans, an exemplary genomic locus of CD3E (encoding CD3e) comprises an open reading frame that contains 9 exons and 8 introns for the transcript variant that encodes isoform 1. An exemplary mRNA transcript of CD3E can span the sequence corresponding to Chromosome 11: 118,304,730-118,316,173, on the forward strand, with reference to Genome Reference Consortium Human Build 38 patch release 13 (GRCh38.p13). Table 1 sets forth the coordinates of the exons and introns of the open reading frames and the untranslated regions of the transcript of an exemplary human CD3E locus.

TABLE 1
Coordinates of exons and introns of exemplary human
CD3E locus for transcript variant encoding isoform
1 (GRCh38, Chromosome 11, forward strand).

| | Start (GrCh38) | End (GrCh38) | Length |
|---|---|---|---|
| 5′ UTR and Exon 1 | 118,304,730 | 118,304,776 | 47 |
| Intron 1-2 | 118,304,777 | 118,304,893 | 117 |
| Exon 2 | 118,304,894 | 118,305,001 | 108 |
| Intron 2-3 | 118,305,002 | 118,307,287 | 2,286 |
| Exon 3 | 118,307,288 | 118,307,308 | 21 |
| Intron 3-4 | 118,307,309 | 118,308,426 | 1,118 |
| Exon 4 | 118,308,427 | 118,308,441 | 15 |
| Intron 4-5 | 118,308,442 | 118,312,152 | 3,711 |
| Exon 5 | 118,312,153 | 118,312,170 | 18 |
| Intron 5-6 | 118,312,171 | 118,312,617 | 447 |
| Exon 6 | 118,312,618 | 118,312,866 | 249 |
| Intron 6-7 | 118,312,867 | 118,313,706 | 840 |
| Exon 7 | 118,313,707 | 118,313,874 | 168 |
| Intron 7-8 | 118,313,875 | 118,314,447 | 573 |
| Exon 8 | 118,314,448 | 118,314,494 | 47 |
| Intron 8-9 | 118,314,495 | 118,315,485 | 991 |
| Exon 9 and 3′ UTR | 118,315,486 | 118,316,173 | 688 |

In humans, several different mRNA and protein isoforms are present for CD3delta (CD3d or CD3δ). Exemplary human CD3d precursor polypeptide sequence is set forth in SEQ ID NO:20 (isoform 1; mature polypeptide includes residues 22-171 of SEQ ID NO:20; see Uniprot Accession No. P04234-1; NCBI Reference Sequence: NP_000723.1; mRNA sequence set forth in SEQ ID NO:21, NCBI Reference Sequence: NM_000732.4); SEQ ID NO:22 (isoform 2; mature polypeptide includes residues 22-127 of SEQ ID NO:22; see Uniprot Accession No. P04234-2; NCBI Reference Sequence: NP_001035741.1; mRNA sequence set forth in SEQ ID NO:23, NCBI Reference Sequence: NM_001040651.1); or SEQ ID NO:24 (isoform 3; mature polypeptide includes residues 23-98 of SEQ ID NO:24; see Uniprot Accession No. E9PMT5; mRNA sequence set forth in SEQ ID NO:25, NCBI Reference Sequence: JN392069.1). Exemplary mature CD3d chain isoform 1 contains an extracellular region (including amino acid residues 22-105 of the human CD3d chain precursor sequence set forth in SEQ ID NO:20), a transmembrane region (including amino acid residues 106-126 of the human CD3d chain precursor sequence set forth in SEQ ID NO:20), and an intracellular region (including amino acid residues 127-171 of the human CD3d chain precursor sequence set forth in SEQ ID NO:20). The CD3d chain isoform 1 contains an immunoreceptor tyrosine-based activation motif (ITAM) domains, at amino acid residues 136-166 of the human CD3d chain precursor sequence set forth in SEQ ID NO:20. Exemplary mature CD3d chain isoform 3 contains an extracellular region (including amino acid residues 23-30 of the human CD3d chain precursor sequence set forth in SEQ ID NO:24), a transmembrane region (including amino acid residues 31-53 of the human CD3d chain precursor sequence set forth in SEQ ID NO:24), and an intracellular region (including amino acid residues 54-98 of the human CD3d chain precursor sequence set forth in SEQ ID NO:24).

In humans, an exemplary genomic locus of CD3D (encoding CD3d) comprises an open reading frame that contains 5 exons and 4 introns for the transcript variant that encodes isoform 1. An exemplary mRNA transcript of CD3D can span the sequence corresponding to Chromosome 11: 118,339,075-118,342,705, on the reverse strand, with reference to Genome Reference Consortium Human Build 38 patch release 13 (GRCh38.p13). Table 2 sets forth the coordinates of the exons and introns of the open reading frames and the untranslated regions of the transcript variant that encodes isoform 1 of an exemplary human CD3D locus.

TABLE 2
Coordinates of exons and introns of exemplary human
CD3D locus for transcript variant encoding isoform
1 (GRCh38, Chromosome 11, reverse strand).

| | Start (GrCh38) | End (GrCh38) | Length |
|---|---|---|---|
| 5′ UTR and Exon 1 | 118,342,705 | 118,342,553 | 153 |
| Intron 1-2 | 118,342,552 | 118,340,594 | 1,959 |
| Exon 2 | 118,340,593 | 118,340,375 | 219 |
| Intron 2-3 | 118,340,374 | 118,339,907 | 468 |
| Exon 3 | 118,339,906 | 118,339,775 | 132 |
| Intron 3-4 | 118,339,774 | 118,339,495 | 280 |
| Exon 4 | 118,339,494 | 118,339,451 | 44 |
| Intron 4-5 | 118,339,450 | 118,339,228 | 223 |
| Exon 5 and 3′ UTR | 118,339,227 | 118,339,075 | 153 |

In humans, an exemplary genomic locus of CD3D (encoding CD3d) comprises an open reading frame that contains 4 exons and 3 introns for the transcript variant that encodes isoform 2. An exemplary mRNA transcript of CD3D can span the sequence corresponding to Chromosome 11: 118,339,094-118,342,631, on the reverse strand, with reference to Genome Reference Consortium Human Build 38 patch release 13 (GRCh38.p13). Table 3 sets forth the coordinates of the exons and introns of the open reading frames and the untranslated regions of the transcript variant that encodes isoform 2 of an exemplary human CD3D locus.

TABLE 3
Coordinates of exons and introns of exemplary human
CD3D locus for transcript variant encoding isoform
2 (GRCh38, Chromosome 11, reverse strand).

| | Start (GrCh38) | End (GrCh38) | Length |
|---|---|---|---|
| 5′ UTR and Exon 1 | 118,342,631 | 118,342,553 | 79 |
| Intron 1-2 | 118,342,552 | 118,340,594 | 1,959 |
| Exon 2 | 118,340,593 | 118,340,375 | 219 |
| Intron 2-3 | 118,340,374 | 118,339,495 | 880 |
| Exon 3 | 118,339,494 | 118,339,451 | 44 |
| Intron 3-4 | 118,339,450 | 118,339,228 | 223 |
| Exon 4 and 3′ UTR | 118,339,227 | 118,339,094 | 134 |

In humans, an exemplary genomic locus of CD3D (encoding CD3d) comprises an open reading frame that contains 4 exons and 3 introns for the transcript variant that encodes isoform 3. An exemplary mRNA transcript of CD3E can span the sequence corresponding to Chromosome 11: 118,339,077-118,342,647, on the reverse strand, with reference to Genome Reference Consortium Human Build 38 patch release 13 (GRCh38.p13). Table 4 sets forth the coordinates of the exons and introns of the open reading frames and the untranslated regions of the transcript variant that encodes isoform 3 of an exemplary human CD3D locus.

TABLE 4
Coordinates of exons and introns of exemplary human
CD3D locus for transcript variant encoding isoform
3 (GRCh38, Chromosome 11, reverse strand).

| | Start (GrCh38) | End (GrCh38) | Length |
|---|---|---|---|
| 5′ UTR and Exon 1 | 118,342,647 | 118,342,553 | 95 |
| Intron 1-2 | 118,342,552 | 118,339,907 | 2,646 |
| Exon 2 | 118,339,906 | 118,339,775 | 132 |
| Intron 2-3 | 118,339,774 | 118,339,495 | 280 |
| Exon 3 | 118,339,494 | 118,339,451 | 44 |
| Intron 3-4 | 118,339,450 | 118,339,228 | 223 |
| Exon 4 and 3′ UTR | 118,339,227 | 118,339,077 | 151 |

Exemplary human CD3g precursor polypeptide sequence is set forth in SEQ ID NO:26 (mature polypeptide includes residues 23-182 of SEQ ID NO:26; see Uniprot Accession No. P09693; NCBI Reference Sequence: NP_000064.1; mRNA sequence set forth in SEQ ID NO:27, NCBI Reference Sequence: NM_000073.2). Exemplary mature CD3g chain contains an extracellular region (including amino acid residues 23-116 of the human CD3g chain precursor sequence set forth in SEQ ID NO:26), a transmembrane region (including amino acid residues 117-137 of the human CD3g chain precursor sequence set forth in SEQ ID NO:26), and an intracellular region (including amino acid residues 138-182 of the human CD3g chain precursor sequence set forth in SEQ ID NO:26). The CD3g chain contains an immunoreceptor tyrosine-based activation motif (ITAM) domains, at amino acid residues 149-177 of the human CD3g chain precursor sequence set forth in SEQ ID NO:26.

In humans, an exemplary genomic locus of CD3G (encoding CD3g) comprises an open reading frame that contains 7 exons and 6 introns. An exemplary mRNA transcript of CD3G can span the sequence corresponding to Chromosome 11: 118,344,344-118,355,161, on the forward strand, with reference to Genome Reference Consortium Human Build 38 patch release 13 (GRCh38.p13). Table 5 sets forth the coordinates of the exons and introns of the open reading frames and the untranslated regions of the transcript of an exemplary human CD3G locus.

TABLE 5
Coordinates of exons and introns of exemplary human
CD3G locus (GRCh38, Chromosome 11, forward strand).

| | Start (GrCh38) | End (GrCh38) | Length |
|---|---|---|---|
| 5′ UTR and Exon 1 | 118,344,344 | 118,344,478 | 135 |
| Intron 1-2 | 118,344,479 | 118,349,026 | 4,548 |
| Exon 2 | 118,349,027 | 118,349,050 | 24 |
| Intron 2-3 | 118,349,051 | 118,349,742 | 692 |
| Exon 3 | 118,349,743 | 118,349,970 | 228 |
| Intron 3-4 | 118,349,971 | 118,350,551 | 581 |
| Exon 4 | 118,350,552 | 118,350,683 | 132 |
| Intron 4-5 | 118,350,684 | 118,351,627 | 944 |
| Exon 5 | 118,351,628 | 118,351,671 | 44 |
| Intron 5-6 | 118,351,672 | 118,352,403 | 732 |
| Exon 6 | 118,352,404 | 118,352,487 | 84 |
| Intron 6-7 | 118,352,488 | 118,353,118 | 631 |
| Exon 7 and 3′ UTR | 118,353,119 | 118,355,161 | 2,043 |

In some aspects, the target site of genetic disruption can be used as a guide to design template polynucleotides and/or homology arms used for HDR. In some aspects, the transgene (e.g., heterologous nucleic acid sequences) within the template polynucleotide can be used to guide the location of target sites and/or homology arms. In some embodiments, the genetic disruption can be targeted near a desired site of targeted integration of transgene sequences (e.g., encoding a portion, such as an antigen-binding domain, of a chimeric receptor). In some aspects, the genetic disruption is targeted based on the desired location for fusion of the transgene sequence encoding the antigen-binding domain and the invariant CD3-IgSF chain contained in the homology arm of the template polynucleotide. In some aspects, the genetic disruption is targeted based on the sequences encoding the invariant CD3-IgSF chain contained in the homology arm of the template polynucleotide. In some aspects, the target site is within an exon of the open reading frame of the endogenous invariant CD3-IgSF chain locus, e.g., CD3E, CD3D or CD3G locus. In some aspects, the target site is within an intron of the open reading frame of the invariant CD3-IgSF chain locus.

In certain embodiments, a genetic disruption is targeted at, near, or within an invariant CD3-IgSF chain locus. In particular embodiments, the genetic disruption is targeted at, near, or within an open reading frame of the invariant CD3-IgSF chain locus (such as the CD3E open reading frame described in Table 1 herein; the CD3D open reading frame described in Table 2, 3 or 4; or the CD3G open reading frame described in Table 5 herein). In certain embodiments, the genetic disruption is targeted at, near, or within an open reading frame that encodes an invariant CD3-IgSF chain, such as CD3e, CD3d or CD3g. In some embodiments, the genetic disruption is targeted at, near, or within the invariant CD3-IgSF chain locus (such as the CD3E open reading frame described in Table 1 herein; the CD3D open reading frame described in Table 2, 3 or 4; or he CD3G open reading frame described in Table 5 herein), or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the invariant CD3-IgSF chain locus (such as the CD3E open reading frame described in Table 1 herein; the CD3D open reading frame described in Table 2, 3 or 4; or he CD3G open reading frame described in Table 5 herein).

In some embodiments, the target site for a genetic disruption is selected such that after integration of the transgene sequences, the miniCAR encoded by the modified invariant CD3-IgSF chain locus, e.g., CD3E, CD3D or CD3G locus, contains a functional invariant CD3-IgSF chain, e.g., CD3e, CD3d or CD3g, such that the miniCAR is capable of being assembled into a TCR/CD3 complex and/or is capable of signaling via the invariant CD3-IgSF chain, e.g., CD3e, CD3d or CD3g contained in the encoded miniCAR. In some embodiments, the target site for a genetic disruption is selected such that after integration of the transgene sequences, the miniCAR encoded by the modified invariant CD3-IgSF chain locus, contains the antigen-binding domain fused to an extracellular portion of the invariant CD3-IgSF chain, e.g., CD3e, CD3d or CD3g. In some embodiments, the target site for a genetic disruption is selected such that after integration of the transgene sequences, the miniCAR encoded by the modified invariant CD3-IgSF chain locus, contains the antigen-binding domain fused to a full-length mature invariant CD3-IgSF chain, e.g., CD3e, CD3d or CD3g, in the extracellular portion of the invariant CD3-IgSF chain.

In some embodiments, the one or more homology arm sequences of the template polynucleotide is designed to surround the site of genetic disruption. In some aspects, the target site is placed within or near an exon of the endogenous invariant CD3-IgSF chain locus, so that the transgene encoding a portion of the chimeric receptor can be integrated in-frame with the coding sequence of the invariant CD3-IgSF chain locus. In some aspects, the target site is placed within or near an exon of the endogenous invariant CD3-IgSF chain locus, so that the transgene encoding a portion of the chimeric receptor can be integrated in-frame with the sequences encoding the extracellular portion of the invariant CD3-IgSF chain locus.

In some embodiments, the target site is selected such that targeted integration of the transgene generates a gene fusion of transgene and endogenous sequences of the invariant CD3-IgSF chain locus, which together encode a miniCAR comprising a heterologous (e.g., encoded by the transgene) antigen-binding domain and an endogenous (e.g., encoded by open reading frame of the genomic or endogenous sequence) invariant CD3-IgSF chain.

The endogenous sequence can, in some aspects, encode a functional invariant CD3-IgSF chain locus that a full length mature chain or a portion thereof that is capable of mediating, activating or stimulating primary cytoplasmic or intracellular signal, e.g., a cytoplasmic domain of the invariant CD3-IgSF chain, e.g., CD3e, CD3d or CD3g, which includes the immunoreceptor tyrosine-based activation motif (ITAM). In some aspects, the target site is placed at or near the beginning of the endogenous open reading frame sequences encoding the extracellular portion of the mature polypeptide of the invariant CD3-IgSF chain, e.g., CD3e, CD3d or CD3g. In some instances, the target site is placed at or near the open reading frame sequences that encode amino acid residues 23-207 of the human CD3e chain precursor sequence set forth in SEQ ID NO:17. In some instances, the target site is placed at or near the open reading frame sequences that encode amino acid residues 22-201 of the human CD3e chain precursor sequence set forth in SEQ ID NO:19. In some instances, the target site is placed at or near the open reading frame sequences that encode amino acid residues 22-171 of the human CD3d sequence set forth in SEQ ID NO:20. In some instances, the target site is placed at or near the open reading frame sequences that encode amino acid residues 22-127 of the human CD3d sequence set forth in SEQ ID NO:22. In some instances, the target site is placed at or near the open reading frame sequences that encode amino acid residues 23-98 of the human CD3d sequence set forth in SEQ ID NO:24. In some instances, the target site is placed at or near the open reading frame sequences that encode amino acid residues 23-182 of the human CD3g sequence set forth in SEQ ID NO:26.

In some aspects, the target site is within an exon of the endogenous invariant CD3-IgSF chain locus, e.g., CD3E, CD3D or CD3G locus. In some aspects, the target site is within an intron of the endogenous invariant CD3-IgSF chain locus. In some aspects, the target site is within a regulatory or control element, e.g., a promoter, 5′ untranslated region (UTR) or 3′ UTR, of the invariant CD3-IgSF chain locus. In some embodiments, the target site is within the CD3E genomic region sequence described in Table 1 herein or any exon or intron of the CD3E genomic region sequence contained therein; the CD3D genomic region sequence described in Table 2, 3 or 4 herein or any exon or intron of the CD3D genomic region sequence contained therein; or he CD3G genomic region sequence described in Table 5 herein or any exon or intron of the CD3G genomic region sequence contained therein.

In some embodiments, a genetic disruption, e.g., DNA break, is targeted within an exon of the invariant CD3-IgSF chain locus, or open reading frame thereof. In certain embodiments, the genetic disruption is within the first exon, second exon, third exon, or forth exon of the invariant CD3-IgSF chain locus, or open reading frame thereof. In some aspects, the target site is within an exon, such as exons corresponding to early coding regions. In some embodiments, the target site is within or in close proximity to exons corresponding to early coding region, e.g., exon 1, 2 or 3 of the open reading frame of the endogenous invariant CD3-IgSF chain locus, (such as the CD3E genomic region sequence described in Table 1 herein or any exon of the CD3E genomic region sequence contained therein; the CD3D genomic region sequence described in Table 2, 3 or 4 herein or any exon of the CD3D genomic region sequence contained therein; or he CD3G genomic region sequence described in Table 5 herein or any exon of the CD3G genomic region sequence contained therein), or including sequence immediately following a transcription start site, within exon 1, 2, or 3, or within less than 500, 450, 400, 350, 300, 250, 200, 150, 100 or 50 bp of exon 1, 2, or 3. In some aspects, the target site is at or near exon 1 of the endogenous invariant CD3-IgSF chain locus, e.g., CD3E, CD3D or CD3G locus, e.g., within less than 500, 450, 400, 350, 300, 250, 200, 150, 100 or 50 bp of exon 1. In some embodiments, the target site is at or near exon 2 of the endogenous invariant CD3-IgSF chain locus, e.g., CD3E, CD3D or CD3G locus, or within less than 500, 450, 400, 350, 300, 250, 200, 150, 100 or 50 bp of exon 2. In some aspects, the target site is at or near exon 3 of the endogenous invariant CD3-IgSF chain locus, e.g., CD3E, CD3D or CD3G locus, e.g., within less than 500, 450, 400, 350, 300, 250, 200, 150, 100 or 50 bp of exon 3. In some aspects, the target site is within a regulatory or control element, e.g., a promoter, of the invariant CD3-IgSF chain locus, e.g., CD3E, CD3D or CD3G locus.

In some instances, the target site is placed at or near exons 1, 2 or 3 of an exemplary genomic locus of CD3E, for example, with genomic coordinates as described in Table 1 herein. In some instances, the target site is placed at or near exons 1, 2 or 3 of an exemplary genomic locus of CD3D, for example, with genomic coordinates as described in Table 2, 3 or 4 herein. In some instances, the target site is placed at or near exons 1, 2 or 3 of an exemplary genomic locus of CD3G, for example, with genomic coordinates as described in Table 5 herein.

In particular embodiments, the genetic disruption is within the first exon of the invariant CD3-IgSF chain locus, e.g., CD3E, CD3D or CD3G locus, or open reading frame thereof. In some embodiments, the genetic disruption is within 500 base pairs (bp) downstream from the 5′ end of the first exon in the invariant CD3-IgSF chain locus, e.g., CD3E, CD3D or CD3G locus, or open reading frame thereof. In particular embodiments, the genetic disruption is between the 5′ nucleotide of exon 1 and upstream of the 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the first exon in the invariant CD3-IgSF chain locus, e.g., CD3E, CD3D or CD3G locus, or open reading frame thereof. In particular embodiments, the genetic disruption is between 1 bp and 400 bp, between 50 and 300 bp, between 100 bp and 200 bp, or between 100 bp and 150 bp downstream from the 5′ end of the first exon in the invariant CD3-IgSF chain locus, e.g., CD3E, CD3D or CD3G locus, or open reading frame thereof, each inclusive. In certain embodiments, the genetic disruption is between 100 bp and 150 bp downstream from the 5′ end of the first exon in the invariant CD3-IgSF chain locus, e.g., CD3E, CD3D or CD3G locus, or open reading frame thereof, inclusive.

In particular embodiments, the genetic disruption is within the second exon of the invariant CD3-IgSF chain locus, e.g., CD3E, CD3D or CD3G locus, or open reading frame thereof. In some embodiments, the genetic disruption is within 500 base pairs (bp) downstream from the 5′ end of the second exon in the invariant CD3-IgSF chain locus, e.g., CD3E, CD3D or CD3G locus, or open reading frame thereof. In particular embodiments, the genetic disruption is between the 5′ nucleotide of exon 1 and upstream of the 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the second exon in the invariant CD3-IgSF chain locus, e.g., CD3E, CD3D or CD3G locus, or open reading frame thereof. In particular embodiments, the genetic disruption is between 1 bp and 400 bp, between 50 and 300 bp, between 100 bp and 200 bp, or between 100 bp and 150 bp downstream from the 5′ end of the second exon in the invariant CD3-IgSF chain locus, e.g., CD3E, CD3D or CD3G locus, or open reading frame thereof, each inclusive. In certain embodiments, the genetic disruption is between 100 bp and 150 bp downstream from the 5′ end of the second exon in the invariant CD3-IgSF chain locus, e.g., CD3E, CD3D or CD3G locus, or open reading frame thereof, inclusive.

In some aspects, the target site is placed before, or upstream of, the endogenous open reading frame sequences encoding the transmembrane region of the invariant CD3-IgSF chain, e.g., CD3e, CD3d or CD3g. In some aspects, the target site is placed within the endogenous open reading frame sequences encoding the extracellular portion of the invariant CD3-IgSF chain, e.g., CD3e, CD3d or CD3g. In some instances, the target site is placed at or near the open reading frame sequences that encode amino acid residues 23-126 of the human CD3e chain precursor sequence set forth in SEQ ID NO:17. In some instances, the target site is placed at or near the open reading frame sequences that encode amino acid residues 22-105 of the human CD3d sequence set forth in SEQ ID NO:20. In some instances, the target site is placed at or near the open reading frame sequences that encode amino acid residues 23-30 of the human CD3d sequence set forth in SEQ ID NO:24. In some instances, the target site is placed at or near the open reading frame sequences that encode amino acid residues 23-116 of the human CD3g sequence set forth in SEQ ID NO:26.

2. Methods of Genetic Disruption

In some aspects, the methods for generating the genetically engineered cells involve introducing a genetic disruption at one or more target site(s), e.g., one or more target sites at an invariant CD3-IgSF chain locus, e.g., CD3E, CD3D or CD3G locus. Methods for generating a genetic disruption, including those described herein, can involve the use of one or more agent(s) capable of inducing a genetic disruption, such as engineered systems to induce a genetic disruption, a cleavage and/or a double strand break (DSB) or a nick (e.g., a single strand break (SSB)) at a target site or target position in the endogenous or genomic DNA such that repair of the break by an error born process such as non-homologous end joining (NHEJ) or repair by HDR using repair template can result in the insertion of a sequence of interest (e.g., heterologous nucleic acid sequences or transgene encoding a portion of a chimeric receptor) at or near the target site or position. Also provided are one or more agent(s) capable of inducing a genetic disruption, for use in the methods provided herein. In some aspects, the one or more agent(s) can be used in combination with the template nucleotides provided herein, for homology directed repair (HDR) mediated targeted integration of the transgene sequences.

In some embodiments, the one or more agent(s) capable of inducing a genetic disruption comprises a DNA binding protein or DNA-binding nucleic acid that specifically binds to or hybridizes to a particular site or position in the genome, e.g., a target site or target position. In some aspects, the targeted genetic disruption, e.g., DNA break or cleavage, at the endogenous invariant CD3-IgSF chain locus, e.g., CD3E, CD3D or CD3G locus, is achieved using a protein or a nucleic acid is coupled to or complexed with a gene editing nuclease, such as in a chimeric or fusion protein. In some embodiments, the one or more agent(s). capable of inducing a genetic disruption comprises an RNA-guided nuclease, or a fusion protein comprising a DNA-targeting protein and a nuclease.

In some embodiments, the agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system (including Cas and/or Cfp1). In some embodiments, the targeted genetic disruption is carried using agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

In some embodiments, an engineered zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO WO 01/60970; WO 01/88197 and WO 02/099084.

Zinc finger and TALE DNA-binding domains can be engineered to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Pub. No. 20110301073.

In some embodiments, the one or more agent(s) specifically targets the at least one target site(s) at or near an invariant CD3-IgSF chain locus, e.g., CD3E, CD3D or CD3G locus. In some embodiments, the agent comprises a ZFN, TALEN or a CRISPR/Cas9 combination that specifically binds to, recognizes, or hybridizes to the target site(s). In some embodiments, the CRISPR/Cas9 system includes an engineered crRNA/tracr RNA (“single guide RNA”) to guide specific cleavage. In some embodiments, the agent comprises nucleases based on the Argonaute system (e.g., from T. thermophilus, known as ‘TtAgo’ (Swarts et al., (2014) Nature 507(7491): 258-261). Targeted cleavage using any of the nuclease systems described herein can be exploited to insert the nucleic acid sequences, e.g., transgene sequences encoding a portion of a chimeric receptor, into a specific target location at an endogenous invariant CD3-IgSF chain locus, using either HDR or NHEJ-mediated processes.

In some embodiments, a “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269: 978-982. Some gene-specific engineered zinc fingers are available commercially. For example, a platform called CompoZr, for zinc-finger construction is available that provides specifically targeted zinc fingers for thousands of targets. See, e.g., Gaj et al., Trends in Biotechnology, 2013, 31(7), 397-405. In some cases, commercially available zinc fingers are used or are custom designed. In some embodiments, the one or more target site(s), e.g., within the invariant CD3-IgSF chain locus, e.g., CD3E, CD3D or CD3G locus, can be targeted for genetic disruption by engineered ZFNs.

Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from different bacterial species. In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a “TALE-nuclease” (TALEN) is a fusion protein comprising a nucleic acid binding domain typically derived from a Transcription Activator Like Effector (TALE) and a nuclease catalytic domain that cleaves a nucleic acid target sequence. The catalytic domain comprises a nuclease domain or a domain having endonuclease activity, like for instance I-TevI, ColE7, NucA and Fok-I. In a particular embodiment, the TALE domain can be fused to a meganuclease like for instance I-CreI and I-OnuI or functional variant thereof. In some embodiments, the TALEN is a monomeric TALEN. A monomeric TALEN is a TALEN that does not require dimerization for specific recognition and cleavage, such as the fusions of engineered TAL repeats with the catalytic domain of I-TevI described in WO2012138927. TALENs have been described and used for gene targeting and gene modifications (see, e.g., Boch et al. (2009) Science 326(5959): 1509-12; Moscou and Bogdanove (2009) Science 326(5959): 1501; Christian et al. (2010) Genetics 186(2): 757-61; Li et al. (2011) Nucleic Acids Res 39(1): 359-72). In some embodiments, one or more sites in an invariant CD3-IgSF chain locus, e.g., CD3E, CD3D or CD3G locus, can be targeted for genetic disruption by engineered TALENs.

In some embodiments, a “TtAgo” is a prokaryotic Argonaute protein thought to be involved in gene silencing. TtAgo is derived from the bacteria Thermus thermophilus. See, e.g. Swarts et al., (2014) Nature 507(7491): 258-261, G. Sheng et al., (2013) Proc. Natl. Acad. Sci. U.S.A. 111, 652). A “TtAgo system” is all the components required including e.g. guide DNAs for cleavage by a TtAgo enzyme.

In some embodiments, the one or more target site(s), e.g., within an invariant CD3-IgSF chain locus, e.g., CD3E, CD3D or CD3G locus, can be targeted for genetic disruption by using clustered regularly interspaced short palindromic repeats (CRISPR) and CRISPR-associated (Cas) proteins. See Sander and Joung, (2014) Nature Biotechnology, 32(4): 347-355. In some embodiments, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9), with nuclease functionality.

In some embodiments, gene editing results in an insertion or a deletion at the targeted locus, or a “knock-out” of the targeted locus and elimination of the expression of the encoded protein. In some embodiments, the gene editing is achieved by non-homologous end joining (NHEJ) using a CRISPR/Cas9 system. In some embodiments, one or more guide RNA (gRNA) molecule can be used with one or more Cas9 nuclease, Cas9 nickase, enzymatically inactive Cas9 or variants thereof. Exemplary features of the gRNA molecule(s) and the Cas9 molecule(s) are described below.

In some embodiments, the CRISPR/Cas nuclease system comprises at least one of: a guide RNA (gRNA) having a targeting domain that is complementary with a target site of an invariant CD3-IgSF chain locus, e.g., CD3E, CD3D or CD3G locus; a gRNA having a targeting domain that is complementary with the one or more target site(s), e.g., within an invariant CD3-IgSF chain locus; or at least one nucleic acid encoding the gRNA.

In some embodiments, a guide sequence, e.g., guide RNA, is any polynucleotide sequences comprising at least a sequence portion, e.g., targeting domain, that has sufficient complementarity with a target site sequence, such as the one or more target site(s), e.g., within an invariant CD3-IgSF chain locus in humans, to hybridize with the target sequence at the target site and direct sequence-specific binding of the CRISPR complex to the target sequence. In some embodiments, in the context of formation of a CRISPR complex, “target site” (also known as “target position,” “target DNA sequence” or “target location”) can refer to a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. In some embodiments, a guide sequence is selected to reduce the degree of secondary structure within the guide sequence. Secondary structure may be determined by any suitable polynucleotide folding algorithm.

In some aspects, a CRISPR enzyme (e.g. Cas9 nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a guide RNA (gRNA) specific to the target site (within an invariant CD3-IgSF chain locus, e.g., CD3E, CD3D or CD3G locus, can be targeted for genetic disruption by in humans) is used with RNA-guided nucleases, e.g., Cas, to introduce a DNA break at the target site or target position. Methods for designing gRNAs and exemplary targeting domains can include those described in, e.g., International PCT Publication No. WO2015/161276. Targeting domains can be incorporated into the gRNA that is used to target Cas9 nucleases to the target site or target position.

Methods for selection and validation of target sequences as well as off-target analyses are described, e.g., in Mali et al., 2013 Science 339(6121): 823-826; Hsu et al. Nat Biotechnol, 31(9): 827-32; Fu et al., 2014 Nat Biotechnol, 2014 March; 32(3):279-284; Heigwer et al., 2014 Nat Methods 11(2):122-3; Bae et al., 2014 Bioinformatics 2014 May 15; 30(10):1473-Xiao A et al., 2014 Bioinformatics 2014 Apr. 15; 30(8):1180-1182. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

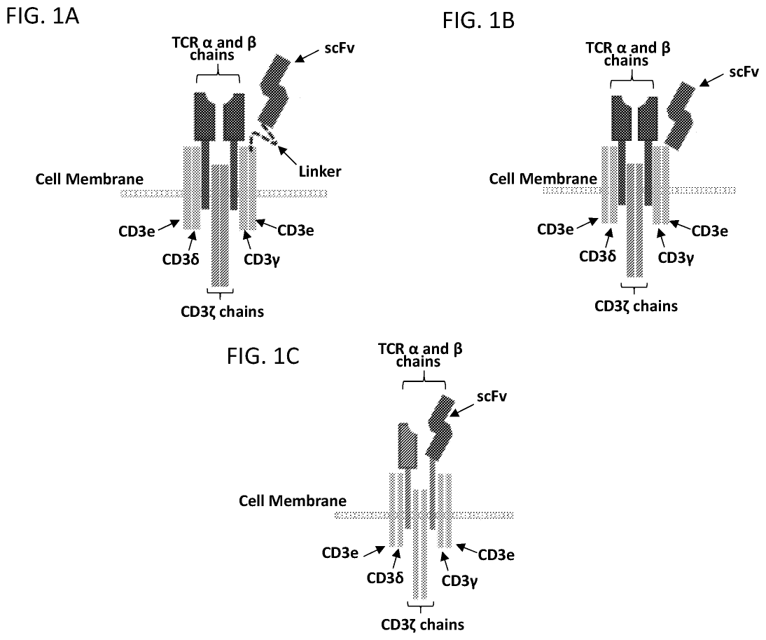

The targeting domain comprises a nucleotide sequence that is complementary, e.g., at least 80%, 85%, 90%, 95%, 98% or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting domains can be found, e.g., in Fu et al., Nat Biotechnol 2014 March; 32(3):279-284 and Sternberg et al., Nature 2014, 507:62-67. Examples of the placement of targeting domains include those described in WO2015/161276, e.g., in FIGS. 1A-1G therein.

The targeting domain is part of an RNA molecule and will therefore comprise the base uracil (U), while any DNA encoding the gRNA molecule will comprise the base thymine (T). While not wishing to be bound by theory, In some embodiments, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In some embodiments, the target domain itself comprises in the 5′ to 3′ direction, an optional secondary domain, and a core domain. In some embodiments, the core domain is fully complementary with the target sequence. In some embodiments, the targeting domain is 5 to 50 nucleotides in length. The strand of the target nucleic acid with which the targeting domain is complementary is referred to herein as the complementary strand. Some or all of the nucleotides of the domain can have a modification, e.g., to render it less susceptible to degradation, improve bio-compatibility, etc. By way of non-limiting example, the backbone of the target domain can be modified with a phosphorothioate, or other modification(s). In some cases, a nucleotide of the targeting domain can comprise a 2′ modification, e.g., a 2-acetylation, e.g., a 2′ methylation, or other modification(s).

In various embodiments, the targeting domain is 16-26 nucleotides in length (i.e. it is 16 nucleotides in length, or 17 nucleotides in length, or 18, 19, 20, 21, 22, 23, 24, 25 or 26 nucleotides in length.

In some embodiments, gRNA sequences that is or comprises a targeting domain sequence targeting the target site in a particular gene, such an invariant CD3-IgSF chain locus, e.g., CD3E, CD3D or CD3G locus, designed or identified. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the target sequence (target domain) is at or near an invariant CD3-IgSF chain locus, e.g., CD3E, CD3D or CD3G locus. In some embodiments, the target nucleic acid complementary to the targeting domain is located at an early coding region of a gene of interest, such as an invariant CD3-IgSF chain locus. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80%, 85%, 90%, 95%, 98% or 99% complementary, e.g., fully complementary, to the target sequence on the target nucleic acid, such as the target nucleic acid in an invariant CD3-IgSF chain locus. In some embodiments, the targeting domain is located downstream of and/or near the endogenous the endogenous transcriptional regulatory element, e.g., a promoter, of an invariant CD3-IgSF chain locus.

In some embodiments, the gRNA can target a site at an invariant CD3-IgSF chain locus, e.g., CD3E, CD3D or CD3G locus near a desired site of targeted integration of a transgene, e.g., encoding a portion, such as an antigen binding domain, of a miniCAR. In some aspects, the gRNA can target a site based on the amount of sequences encoding an invariant CD3-IgSF chain locus that is desired for regulation of expression of the portion, such as an antigen binding domain, of a miniCAR in a manner, time and extent similar to the regulation of the endogenous invariant CD3-IgSF chain locus. In some aspects, the gRNA can target a site based on the amount of sequences encoding an invariant CD3-IgSF chain locus that is desired for expression in the cell expressing the portion, such as an antigen binding domain, of a miniCAR. In some aspects, the gRNA can target a site such that upon integration of the transgene, e.g., encoding a portion, such as an antigen binding domain, of a miniCAR the resulting invariant CD3-IgSF chain locus retains expression of the full length endogenous mature gene product (e.g., mature polypeptide without the signal peptide) encoded by an invariant CD3-IgSF chain locus. In some aspects, the gRNA can target a site within an exon of the open reading frame of the endogenous invariant CD3-IgSF chain locus. In some aspects, the gRNA can target a site within an intron of the open reading frame of an invariant CD3-IgSF chain locus. In some aspects, the gRNA can target a site within or downstream of a regulatory or control element, e.g., a promoter, of an invariant CD3-IgSF chain locus. In some aspects, the target site at an invariant CD3-IgSF chain locus that is targeted by the gRNA can be any target sites described herein. In some embodiments, the gRNA can target a site within or in close proximity to exons corresponding to early coding region, e.g., exon 1, 2, 3, 4 or 5 of the open reading frame of the endogenous invariant CD3-IgSF chain locus, or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some embodiments, the gRNA can target a site at or near exon 2 of the endogenous invariant CD3-IgSF chain locus, or within less than 500, 450, 400, 350, 300, 250, 200, 150, 100 or 50 bp of exon 2.

Exemplary target sequence for CD3E locus include the sequence set forth in any of SEQ ID NO: 8 and 28-38. Exemplary gRNAs can include a sequence of ribonucleic acids that can bind to or target or is complementary to or can bind to the complimentary strand sequence of the target site sequences set forth in any of SEQ ID NO: 8 and 28-38. Exemplary CD3E gRNA sequences includes the sequence set forth in any of SEQ ID NO: 9 and 39-49. An exemplary CD3E gRNA sequence includes the sequence set forth in SEQ ID NO: 9. Any of the known methods can be used to target and generate a genetic disruption of the endogenous CD3E can be used in the embodiments provided herein. Exemplary target sequences or targeting domains contained within the gRNA for targeting the genetic disruption of the human CD3E locus include those described in, e.g., Chan et al., European Radiology (2020) 30:3538-3548 and Shifruit et al., Cell. 2018 Dec. 13; 175(7): 1958-1971.e15, which are incorporated by reference herein.

Exemplary target sequence for CD3D locus include the sequence set forth in any of SEQ ID NO: 50-57. Exemplary gRNAs can include a sequence of ribonucleic acids that can bind to or target or is complementary to or can bind to the complimentary strand sequence of the target site sequences set forth in any of SEQ ID NO:50-57. Exemplary CD3D gRNA sequences includes the sequence set forth in any of SEQ ID NO:58-65. An exemplary CD3D gRNA sequence includes the sequence set forth in SEQ ID NO: 58. Any of the known methods can be used to target and generate a genetic disruption of the endogenous CD3D can be used in the embodiments provided herein. Exemplary target sequences or targeting domains contained within the gRNA for targeting the genetic disruption of the human CD3D locus include those described in, e.g., Shifruit et al., Cell. 2018 Dec. 13; 175(7): 1958-1971.e15, which is incorporated by reference herein.

Exemplary target sequence for CD3G locus include the sequence set forth in any of SEQ ID NO: 66-74. Exemplary gRNAs can include a sequence of ribonucleic acids that can bind to or target or is complementary to or can bind to the complimentary strand sequence of the target site sequences set forth in any of SEQ ID NO: 66-74. Exemplary CD3G gRNA sequences includes the sequence set forth in any of SEQ ID NO:75-83. An exemplary CD3G gRNA sequence includes the sequence set forth in SEQ ID NO: 75. Any of the known methods can be used to target and generate a genetic disruption of the endogenous CD3G can be used in the embodiments provided herein. Exemplary target sequences or targeting domains contained within the gRNA for targeting the genetic disruption of the human CD3G locus include those described in, e.g., Shifruit et al., Cell. 2018 Dec. 13; 175(7): 1958-1971.e15, which is incorporated by reference herein.

3. Delivery of Agents for Genetic Disruption

In some embodiments, the targeted genetic disruption, e.g., DNA break, of an endogenous invariant CD3-IgSF chain locus, e.g., CD3E, CD3D or CD3G locus in humans is carried out by delivering or introducing one or more agent(s) capable of inducing a genetic disruption, e.g., Cas9 and/or gRNA components, to a cell, using any of a number of known delivery method or vehicle for introduction or transfer to cells, for example, using viral, e.g., lentiviral, delivery vectors, or any of the known methods or vehicles for delivering Cas9 molecules and gRNAs. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequences encoding one or more components of one or more agent(s) capable of inducing a genetic disruption, e.g., DNA break, is introduced into the cells, e.g., by any methods for introducing nucleic acids into a cell described herein or known. In some embodiments, a vector encoding components of one or more agent(s) capable of inducing a genetic disruption such as a CRISPR guide RNA and/or a Cas9 enzyme can be delivered into the cell.

In some embodiments, the one or more agent(s) capable of inducing a genetic disruption, e.g., one or more agent(s) that is a Cas9/gRNA, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more agent(s) capable of inducing genetic disruption, e.g., CRISPR/Cas9, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the integration of transgene can be inherited by the progeny cells, but without the agent itself, which may further introduce off-target genetic disruptions, being passed on to the progeny cells.

Agent(s) and components capable of inducing a genetic disruption, e.g., a Cas9 molecule and gRNA molecule, can be introduced into target cells in a variety of forms using a variety of delivery methods and formulations, as set forth in Tables 6 and 7, or methods described in, e.g., WO 2015/161276; US 2015/0056705, US 2016/0272999, US 2017/0211075; or US 2017/0016027. As described further herein, the delivery methods and formulations can be used to deliver template polynucleotides and/or other agents to the cell (such as those required for engineering the cells) in prior or subsequent steps of the methods described herein. When a Cas9 or gRNA component is encoded as DNA for delivery, the DNA may typically but not necessarily include a control region, e.g., comprising a promoter, to effect expression. Useful promoters for Cas9 molecule sequences include, e.g., CMV, EF-1α, EFS, MSCV, PGK, or CAG promoters. Useful promoters for gRNAs include, e.g., H1, EF-1α, tRNA or U6 promoters. Promoters with similar or dissimilar strengths can be selected to tune the expression of components. Sequences encoding a Cas9 molecule may comprise a nuclear localization signal (NLS), e.g., an SV40 NLS. In some embodiments a promoter for a Cas9 molecule or a gRNA molecule may be, independently, inducible, tissue specific, or cell specific. In some embodiments, an agent capable of inducing a genetic disruption is introduced RNP complexes.

TABLE 6
Exemplary Delivery Methods

| Elements | | |
|---|---|---|
| Cas9 | gRNA | |
| Molecule(s) | molecule(s) | Comments |
| DNA | DNA | In this embodiment, a Cas9 molecule and a gRNA are |
| | | transcribed from DNA. In this embodiment, they are |
| | | encoded on separate molecules. |
| DNA | | In this embodiment, a Cas9 molecule and a gRNA are |
| | | transcribed from DNA, here from a single molecule. |
| DNA | RNA | In this embodiment, a Cas9 molecule is transcribed from |
| | | DNA, and a gRNA is provided as in vitro transcribed or |
| | | synthesized RNA |
| mRNA | RNA | In this embodiment, a Cas9 molecule is translated from in |
| | | vitro transcribed mRNA, and a gRNA is provided as in vitro |
| | | transcribed or synthesized RNA. |
| mRNA | DNA | In this embodiment, a Cas9 molecule is translated from in |
| | | vitro transcribed mRNA, and a gRNA is transcribed from |
| | | DNA. |
| Protein | DNA | In this embodiment, a Cas9 molecule is provided as a |
| | | protein, and a gRNA is transcribed from DNA. |
| Protein | RNA | In this embodiment, a Cas9 molecule is provided as a |
| | | protein, and a gRNA is provided as transcribed or |
| | | synthesized RNA. |

TABLE 7
Comparison of Exemplary Delivery Methods

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic |
| particle gun, Calcium Phosphate | | | | | Acids and |
| transfection, cell compression or | | | | | Proteins |
| squeezing) | | | | | |
| Viral | Retrovirus | NO | Stable | YES | RNA |
| | Lentivirus | YES | Stable | YES/NO | RNA |
| | | | | with | |
| | | | | modifications | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic Liposomes | YES | Transient | Depends on | Nucleic |
| | | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated | YES | Transient | NO | Nucleic |
| Non-Viral | Bacteria | | | | Acids |
| Delivery | Engineered | YES | Transient | NO | Nucleic |
| Vehicles | Bacteriophages | | | | Acids |
| | Mammalian Virus- | YES | Transient | NO | Nucleic |
| | like Particles | | | | Acids |
| | Biological | YES | Transient | NO | Nucleic |
| | liposomes: | | | | Acids |
| | Erythrocyte Ghosts | | | | |
| | and Exosomes | | | | |

In some embodiments, DNA encoding Cas9 molecules and/or gRNA molecules, or RNP complexes comprising a Cas9 molecule and/or gRNA molecules, can be delivered into cells by known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof. In some embodiments, the polynucleotide containing the agent(s) and/or components thereof is delivered by a vector (e.g., viral vector/virus or plasmid). The vector may be any described herein.

In some aspects, a CRISPR enzyme (e.g. Cas9 nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target locus (e.g., an endogenous invariant CD3-IgSF chain locus, e.g., CD3E, CD3D or CD3G locus) are introduced into cells.

In some embodiments, the polynucleotide containing the agent(s) and/or components thereof or RNP complex is delivered by a non-vector based method (e.g., using naked DNA or DNA complexes). For example, the DNA or RNA or proteins or combination thereof, e.g., ribonucleoprotein (RNP) complexes, can be delivered, e.g., by organically modified silica or silicate (Ormosil), electroporation, transient cell compression or squeezing (such as described in Lee et al. (2012) Nano Lett 12: 6322-27, Kollmannsperger et al. (2016) Nat Comm 7, 10372), gene gun, sonoporation, magnetofection, lipid-mediated transfection, dendrimers, inorganic nanoparticles, calcium phosphates, or a combination thereof.

In some embodiments, delivery via electroporation comprises mixing the cells with the Cas9- and/or gRNA-encoding DNA or RNP complex in a cartridge, chamber or cuvette and applying one or more electrical impulses of defined duration and amplitude. In some embodiments, delivery via electroporation is performed using a system in which cells are mixed with the Cas9- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the delivery vehicle is a non-viral vector. In some embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In some embodiments, the non-viral vector is an organic nanoparticle. Exemplary organic nanoparticles include, e.g., SNALP liposomes that contain cationic lipids together with neutral helper lipids which are coated with polyethylene glycol (PEG), and protamine-nucleic acid complexes coated with lipid. Exemplary lipids for gene transfer include those described in, e.g., WO2015/161276, WO2017/193107, WO2017/093969, US2016/272999 and US2015/056705.

In some embodiments, the vehicle has targeting modifications to increase target cell update of nanoparticles and liposomes, e.g., cell specific antigens, monoclonal antibodies, single chain antibodies, aptamers, polymers, sugars, and cell penetrating peptides. In some embodiments, the vehicle uses fusogenic and endosome-destabilizing peptides/polymers. In some embodiments, the vehicle undergoes acid-triggered conformational changes (e.g., to accelerate endosomal escape of the cargo). In some embodiments, a stimulus-cleavable polymer is used, e.g., for release in a cellular compartment. For example, disulfide-based cationic polymers that are cleaved in the reducing cellular environment can be used.

In some embodiments, the delivery vehicle is a biological non-viral delivery vehicle. In some embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific cells, bacteria having modified surface proteins to alter target cell specificity). In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue-specificity. In some embodiments, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject-derived membrane-bound nanovescicles (30-100 nm) of endocytic origin (e.g., can be produced from various cell types and can therefore be taken up by cells without the need for targeting ligands).

In some embodiments, RNA encoding Cas9 molecules and/or gRNA molecules, can be delivered into cells, e.g., target cells described herein, by known methods or as described herein. For example, Cas9-encoding and/or gRNA-encoding RNA can be delivered, e.g., by microinjection, electroporation, transient cell compression or squeezing (such as described in Lee et al. (2012) Nano Lett 12: 6322-27), lipid-mediated transfection, peptide-mediated delivery, e.g., cell-penetrating peptides, or a combination thereof.

In some embodiments, delivery via electroporation comprises mixing the cells with the RNA encoding Cas9 molecules and/or gRNA molecules in a cartridge, chamber or cuvette and applying one or more electrical impulses of defined duration and amplitude. In some embodiments, delivery via electroporation is performed using a system in which cells are mixed with the RNA encoding Cas9 molecules and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, Cas9 molecules can be delivered into cells by known methods or as described herein. For example, Cas9 protein molecules can be delivered, e.g., by microinjection, electroporation, transient cell compression or squeezing (such as described in Lee et al. (2012) Nano Lett 12: 6322-27), lipid-mediated transfection, peptide-mediated delivery, or a combination thereof. Delivery can be accompanied by DNA encoding a gRNA or by a gRNA.

In some embodiments, the one or more agent(s) capable of introducing a cleavage, e.g., a Cas9/gRNA system, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, calcium phosphate transfection, cell compression or squeezing.

In some embodiments, delivery via electroporation comprises mixing the cells with the Cas9 molecules with or without gRNA molecules in a cartridge, chamber or cuvette and applying one or more electrical impulses of defined duration and amplitude. In some embodiments, delivery via electroporation is performed using a system in which cells are mixed with the Cas9 molecules with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, delivery via electroporation comprises mixing the cells with the Cas9 molecules with or without gRNA molecules in a cartridge, chamber or cuvette and applying one or more electrical impulses of defined duration and amplitude. In some embodiments, delivery via electroporation is performed using a system in which cells are mixed with the Cas9 molecules.

In some embodiments, the polynucleotide containing the agent(s) and/or components thereof is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, more than one agent(s) or components thereof are delivered to the cell. For example, in some embodiments, agent(s) capable of inducing a genetic disruption of two or more locations in the genome, such as at two or more sites within an endogenous invariant CD3-IgSF chain locus, e.g., CD3E, CD3D or CD3G locus, are delivered to the cell. In some embodiments, agent(s) and components thereof are delivered using one method. For example, in some embodiments, agent(s) for inducing a genetic disruption of an endogenous invariant CD3-IgSF chain locus are delivered as polynucleotides encoding the components for genetic disruption. In some embodiments, one polynucleotide can encode agents that target an endogenous invariant CD3-IgSF chain locus. In some embodiments, two or more different polynucleotides can encode the agents that target an endogenous invariant CD3-IgSF chain locus. In some embodiments, the agents capable of inducing a genetic disruption can be delivered as ribonucleoprotein (RNP) complexes, and two or more different RNP complexes can be delivered together as a mixture, or separately.

In some embodiments, the one or more agent(s) is or comprises a ribonucleoprotein (RNP) complex. In some embodiments, the concentration of the RNP incubated with, added to or contacted with the cells for engineering is at a concentration of at or about 0.1, 0.2, 0.3, 0.4, 0.5, 0.6, 0.7, 0.8, 0.9, 1, 2, 2.2, 2.5, 3, 4, 5, 6, 7, 7.5, 8, 9, 10, 11, 12, 13, 14, 15, 16, 17, 18, 19, or 20 μg/10⁶ cells, or a range defined by any two of the foregoing values. In some embodiments, the concentration of the RNP incubated with, added to or contacted with the cells for engineering is at a concentration of at or about 0.1, 0.2, 0.3, 0.4, 0.5, 0.6, 0.7, 0.8, 0.9, 1, 2, 2.2, 2.5, 3, 4, 5 μg/10⁶ cells, or a range defined by any two of the foregoing values. In some embodiments, the concentration of RNPs is 1 μg/10⁶ cells. In some embodiments, in the RNP complex, the ratio, e.g. the molar ratio, of the gRNA and the Cas9 molecule or other nucleases is at or about 5:1, 4:1, 3:1, 2:1, 1:1, 1:2, 1:3, 1:4 or 1:5, or a range defined by any two of the foregoing values. In some embodiments, in the RNP complex, the ratio, e.g., molar ratio, of the gRNA and the Cas9 molecule or other nucleases is at or about 3:1, 2.9:1, 2.8:1, 2.7:1, 2.6:1, 2.5:1, 2.4:1, 2.3:1, 2.2:1, 2.1:1, 2:1 or 1:1, or a range defined by any two of the foregoing values. In some embodiments, in the RNP complex, the molar ratio of the gRNA and the Cas9 molecule or other nucleases is at or about 2:1.

In some embodiments, one or more nucleic acid molecules other than the one or more agent(s) capable of inducing a genetic disruption and/or component thereof, e.g., the Cas9 molecule component and/or the gRNA molecule component, such as a template polynucleotide for HDR-directed integration (such as any template polynucleotide described herein, e.g., in Section I.B.2), are delivered. In some embodiments, the nucleic acid molecule, e.g., template polynucleotide, is delivered at the same time as one or more of the components of the Cas system. In some embodiments, the nucleic acid molecule is delivered before or after (e.g., less than about 1 minute, 5 minutes, 10 minutes, 15 minutes, 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In some embodiments, the nucleic acid molecule, e.g., template polynucleotide, is delivered by a different means from one or more of the components of the Cas system, e.g., the Cas9 molecule component and/or the gRNA molecule component. The nucleic acid molecule, e.g., template polynucleotide, can be delivered by any of the delivery methods described herein. For example, the nucleic acid molecule, e.g., template polynucleotide, can be delivered by a viral vector, e.g., a retrovirus or a lentivirus, and the Cas9 molecule component and/or the gRNA molecule component can be delivered by electroporation. In some embodiments, the nucleic acid molecule, e.g., template polynucleotide, includes one or more heterologous sequences, e.g., sequences that encode a portion, such as an antigen binding domain, of a miniCAR and/or other heterologous gene nucleic acid sequences.

B. Targeted Integration via Homology-directed Repair (HDR)

In some aspects, the provided embodiments involve targeted integration of a specific part of a polynucleotide, such as the part of a template polynucleotide containing a transgene encoding a portion, such as an antigen-binding domain, of a miniCAR at a particular location (such as target site or target location) in the genome at the endogenous invariant CD3-IgSF chain locus. In some aspects, homology-directed repair (HDR) can mediate the site specific integration of the transgene at the target site. In some embodiments, the presence of a genetic disruption (e.g., a DNA break, such as described in Section I.A) and a template polynucleotide containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the 5′ and/or 3′ homology arms included in the template polynucleotide, cellular DNA repair machinery can use the template polynucleotide to repair the DNA break and resynthesize genetic information at the site of the genetic disruption, thereby effectively inserting or integrating the transgene in the template polynucleotide at or near the site of the genetic disruption. In some embodiments, the genetic disruption at an endogenous invariant CD3-IgSF chain locus, e.g., CD3E, CD3D or CD3G locus, can be generated by any of the methods for generating a targeted genetic disruption described herein.

Also provided are polynucleotides, e.g., template polynucleotides described herein, and kits that include such polynucleotides. In some embodiments, the provided polynucleotides and/or kits can be employed in the methods described herein, e.g., involving HDR, to target the transgene encoding a portion, such as an antigen-binding domain, of a miniCAR at the endogenous invariant CD3-IgSF chain locus.

In some embodiments, the template polynucleotide is or comprises a polynucleotide containing a transgene, such as exogenous or heterologous nucleic acid sequences, encoding a portion, a region or a domain, such as an antigen binding domain, of a miniCAR and homology sequences (e.g., homology arms) that are homologous to sequences at or near the endogenous genomic site at the endogenous invariant CD3-IgSF chain locus. In some aspects, the transgene in the template polynucleotide comprise sequence of nucleotides encoding a portion, such as an antigen-binding domain, of a miniCAR. In some aspects, upon targeted integration of the transgene, the invariant CD3-IgSF chain locus in the engineered cell is modified such that the modified invariant CD3-IgSF chain locus contains the transgene encoding a portion, such as an antigen-binding domain, of a miniCAR.

In some aspects, the template polynucleotide is introduced as a linear DNA fragment or comprised in a vector. In some aspects, the step for inducing genetic disruption and the step for targeted integration (e.g., by introduction of the template polynucleotide) are performed simultaneously or sequentially.

1. Homology-Directed Repair (HDR)

In some embodiments, homology-directed repair (HDR) can be utilized for targeted integration or insertion of one or more nucleic acid sequences, e.g., a transgene, at one or more target site(s) at an endogenous invariant CD3-IgSF chain locus, e.g., CD3E, CD3D or CD3G locus. In some embodiments, the nuclease-induced HDR can be used to alter a target sequence, integrate the transgene at a particular target location, and/or to edit or repair a mutation in a particular target gene.

Alteration of nucleic acid sequences at the target site can occur by HDR with an heterologously provided template polynucleotide (also referred to as “donor polynucleotide” or “template sequence”). For example, the template polynucleotide provides for alteration of the target sequence, such as insertion of the transgene contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Template polynucleotide-effected alteration of a target sequence depends on cleavage by a nuclease, e.g., a targeted nuclease such as CRISPR/Cas9. Cleavage by the nuclease can comprise a double strand break or two single strand breaks.

In some embodiments, “recombination” includes a process of exchange of genetic information between two polynucleotides. In some embodiments, “homologous recombination (HR)” includes a specialized form of such exchange that takes place, for example, during repair of double-strand breaks in cells via homology-directed repair mechanisms. This process requires nucleotide sequence homology, uses a template polynucleotide to template repair of a target DNA (i.e., the one that experienced the double-strand break, such as target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide.

In some embodiments, a template polynucleotide, e.g., polynucleotide containing transgene, is integrated into the genome of a cell via homology-independent mechanisms. The methods comprise creating a double-stranded break (DSB) in the genome of a cell and cleaving the template polynucleotide molecule using a nuclease, such that the template polynucleotide is integrated at the site of the DSB. In some embodiments, the template polynucleotide is integrated via non-homology dependent methods (e.g., NHEJ). Upon in vivo cleavage the template polynucleotides can be integrated in a targeted manner into the genome of a cell at the location of a DSB. The template polynucleotide can include one or more of the same target sites for one or more of the nucleases used to create the DSB. Thus, the template polynucleotide may be cleaved by one or more of the same nucleases used to cleave the endogenous gene into which integration is desired. In some embodiments, the template polynucleotide includes different nuclease target sites from the nucleases used to induce the DSB. As described herein, the genetic disruption of the target site or target position can be created by any know methods or any methods described herein, such as ZFNs, TALENs, CRISPR/Cas9 system, or TtAgo nucleases.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break. In some embodiments, a single-stranded template polynucleotide is used and the target site can be altered by alternative HDR.

Template polynucleotide-effected alteration of a target site depends on cleavage by a nuclease molecule. Cleavage by the nuclease can comprise a nick, a double strand break, or two single strand breaks, e.g., one on each strand of the DNA at the target site. After introduction of the breaks on the target site, resection occurs at the break ends resulting in single stranded overhanging DNA regions.

In canonical HDR, a double-stranded template polynucleotide is introduced, comprising homologous sequence to the target site that will either be directly incorporated into the target site or used as a template to insert the transgene or correct the sequence of the target site. After resection at the break, repair can progress by different pathways, e.g., by the double Holliday junction model (or double strand break repair, DSBR, pathway) or the synthesis-dependent strand annealing (SDSA) pathway.

In the double Holliday junction model, strand invasion by the two single stranded overhangs of the target site to the homologous sequences in the template polynucleotide occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the target site, e.g., insertion of the transgene in template polynucleotide. Crossover with the template polynucleotide may occur upon resolution of the junctions.

In the SDSA pathway, only one single stranded overhang invades the template polynucleotide and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

In alternative HDR, a single strand template polynucleotide, e.g., template polynucleotide, is introduced. A nick, single strand break, or double strand break at the target site, for altering a desired target site, is mediated by a nuclease molecule, and resection at the break occurs to reveal single stranded overhangs. Incorporation of the sequence of the template polynucleotide to correct or alter the target site of the DNA typically occurs by the SDSA pathway, as described herein.

“Alternative HDR”, or alternative homology-directed repair, in some embodiments, refers to the process of repairing DNA damage using a homologous nucleic acid (e.g., an endogenous homologous sequence, e.g., a sister chromatid, or an heterologous nucleic acid, e.g., a template polynucleotide). Alternative HDR is distinct from canonical HDR in that the process utilizes different pathways from canonical HDR, and can be inhibited by the canonical HDR mediators, RAD51 and BRCA2. Also, alternative HDR uses a single-stranded or nicked homologous nucleic acid for repair of the break. “Canonical HDR”, or canonical homology-directed repair, in some embodiments, refers to the process of repairing DNA damage using a homologous nucleic acid (e.g., an endogenous homologous sequence, e.g., a sister chromatid, or an heterologous nucleic acid, e.g., a template nucleic acid). Canonical HDR typically acts when there has been significant resection at the double strand break, forming at least one single stranded portion of DNA In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2 and the homologous nucleic acid is typically double-stranded. Unless indicated otherwise, the term “HDR” in some embodiments encompasses canonical HDR and alternative HDR.

In some embodiments, double strand cleavage is effected by a nuclease, e.g., a Cas9 molecule having cleavage activity associated with an HNH-like domain and cleavage activity associated with a RuvC-like domain, e.g., an N-terminal RuvC-like domain, e.g., a wild type Cas9. Such embodiments require only a single gRNA.

In some embodiments, one single strand break, or nick, is effected by a nuclease molecule having nickase activity, e.g., a Cas9 nickase. A nicked DNA at the target site can be a substrate for alternative HDR.

In some embodiments, two single strand breaks, or nicks, are effected by a nuclease, e.g., Cas9 molecule, having nickase activity, e.g., cleavage activity associated with an HNH-like domain or cleavage activity associated with an N-terminal RuvC-like domain. Such embodiments usually require two gRNAs, one for placement of each single strand break. In some embodiments, the Cas9 molecule having nickase activity cleaves the strand to which the gRNA hybridizes, but not the strand that is complementary to the strand to which the gRNA hybridizes. In some embodiments, the Cas9 molecule having nickase activity does not cleave the strand to which the gRNA hybridizes, but rather cleaves the strand that is complementary to the strand to which the gRNA hybridizes. In some embodiments, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HNH activity and will cut on the strand to which the gRNA hybridizes (e.g., the complementary strand, which does not have the NGG PAM on it). In some embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In some embodiments, the Cas9 molecule is an N-terminal RuvC-like domain nickase, e.g., the Cas9 molecule comprises a mutation at N863, e.g., N863A.

In some embodiments, in which a nickase and two gRNAs are used to position two single strand nicks, one nick is on the + strand and one nick is on the − strand of the target DNA. The PAMs are outwardly facing. The gRNAs can be selected such that the gRNAs are separated by, from about 0-50, 0-100, or 0-200 nucleotides. In some embodiments, there is no overlap between the target sequences that are complementary to the targeting domains of the two gRNAs. In some embodiments, the gRNAs do not overlap and are separated by as much as 100, or 200 nucleotides. In some embodiments, the use of two gRNAs can increase specificity, e.g., by decreasing off-target binding (Ran et al., Cell. 2013 Sep. 12; 154(6):1380-9).

In some embodiments, a single nick can be used to induce HDR, e.g., alternative HDR. It is contemplated herein that a single nick can be used to increase the ratio of HR to NHEJ at a given cleavage site, such as target site. In some embodiments, a single strand break is formed in the strand of the DNA at the target site to which the targeting domain of said gRNA is complementary. In some embodiments, a single strand break is formed in the strand of the DNA at the target site other than the strand to which the targeting domain of said gRNA is complementary.

In some embodiments, other DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

Targeted integration results in the transgene, e.g., sequences between the homology arms, being integrated into an invariant CD3-IgSF chain locus in the genome, to produce a modified invariant CD3-IgSF chain locus encoding a miniCAR. The transgene may be integrated anywhere at or near one of the at least one target site(s) or site in the genome. In some embodiments, the transgene is integrated at or near one of the at least one target site(s), for example, within 300, 250, 200, 150, 100, 50, 10, 5, 4, 3, 2, 1 or fewer base pairs upstream or downstream of the site of cleavage, such as within 100, 50, 10, 5, 4, 3, 2, 1 base pairs of either side of the target site, such as within 50, 10, 5, 4, 3, 2, 1 base pairs of either side of the target site. In some embodiments, the integrated sequence comprising the transgene does not include any vector sequences (e.g., viral vector sequences). In some embodiments, the integrated sequence includes a portion of the vector sequences (e.g., viral vector sequences).

The double strand break or single strand break (such as target site) in one of the strands should be sufficiently close to the target integration site, e.g., site for targeted integration, such that an alteration is produced in the desired region, such as insertion of transgene or correction of a mutation occurs. In some embodiments, the distance is not more than 10, 25, 50, 100, 200, 300, 350, 400 or 500 nucleotides. In some embodiments, it is believed that the break should be sufficiently close to the target integration site such that the break is within the region that is subject to exonuclease-mediated removal during end resection. In some embodiments, the targeting domain is configured such that a cleavage event, e.g., a double strand or single strand break, is positioned within 1, 2, 3, 4, 5, 10, 15, 20, 25, 30, 35, 40, 45, 50, 70, 80, 90, 100, 150, 200, 300, 350, 400 or 500 nucleotides of the region desired to be altered, e.g., site for targeted insertion. The break, e.g., a double strand or single strand break, can be positioned upstream or downstream of the region desired to be altered, e.g., site for targeted insertion. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of target integration site.

In some embodiments, a single strand break is accompanied by an additional single strand break, positioned by a second gRNA molecule. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 350, 400 or 500 nucleotides of a target integration site. In some embodiments, the first and second gRNA molecules are configured such, that when guiding a Cas9 nickase, a single strand break will be accompanied by an additional single strand break, positioned by a second gRNA, sufficiently close to one another to result in alteration of the desired region. In some embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In some embodiments, the two gRNA molecules are configured to position cuts at the same position, or within a few nucleotides of one another, on different strands, e.g., essentially mimicking a double strand break.

In some embodiments, in which a gRNA (unimolecular (or chimeric) or modular gRNA) and Cas9 nuclease induce a double strand break for the purpose of inducing HDR-mediated insertion of transgene or correction, the cleavage site, such as target site, is between 0-200 bp (e.g., 0-175, 0 to 150, 0 to 125, 0 to 100, 0 to 75, 0 to 50, 0 to 25, 25 to 200, to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target integration site. In some embodiments, the cleavage site, such as target site such as target site, is between 0-100 bp (e.g., 0 to 75, 0 to 50, 0 to 25, 25 to 100, 25 to 75, 25 to 50, 50 to 100, 50 to 75 or 75 to 100 bp) away from the site for targeted integration.

In some embodiments, one can promote HDR by using nickases to generate a break with overhangs. In some embodiments, the single stranded nature of the overhangs can enhance the cell's likelihood of repairing the break by HDR as opposed to, e.g., NHEJ.

Specifically, in some embodiments, HDR is promoted by selecting a first gRNA that targets a first nickase to a first target site, and a second gRNA that targets a second nickase to a second target site which is on the opposite DNA strand from the first target site and offset from the first nick. In some embodiments, the targeting domain of a gRNA molecule is configured to position a cleavage event sufficiently far from a preselected nucleotide, e.g., the nucleotide of a coding region, such that the nucleotide is not altered. In some embodiments, the targeting domain of a gRNA molecule is configured to position an intronic cleavage event sufficiently far from an intron/exon border, or naturally occurring splice signal, to avoid alteration of the exonic sequence or unwanted splicing events. In some embodiments, the targeting domain of a gRNA molecule is configured to position in an early exon, to allow in-frame integration of the transgene at or near one of the at least one target site(s).

In some embodiments, a double strand break can be accompanied by an additional double strand break, positioned by a second gRNA molecule. In some embodiments, a double strand break can be accompanied by two additional single strand breaks, positioned by a second gRNA molecule and a third gRNA molecule. In some embodiments, two gRNAs, e.g., independently, unimolecular (or chimeric) or modular gRNA, are configured to position a double-strand break on both sides of a target integration site, e.g., site for targeted integration.

2. Template Polynucleotide

In some embodiments, a template polynucleotide, e.g., a polynucleotide containing a transgene, such as exogenous or heterologous nucleic acid sequences, that includes a sequence of nucleotides encoding a portion, such as an antigen-binding domain, of a miniCAR and homology sequences (e.g., homology arms) that are homologous to sequences at or near the endogenous genomic site for targeted integration, can be employed molecules and machinery involved in cellular DNA repair processes, such as homologous recombination, as a repair template. In some aspects, a template polynucleotide having homology with sequences at or near one or more target site(s) in the endogenous DNA can be used to alter the structure of a target DNA, such as target site at the endogenous invariant CD3-IgSF chain locus, for targeted insertion of the transgenic, heterologous or exogenous sequences, e.g., heterologous nucleic acid sequences encoding a portion, such as an antigen-binding domain, of a miniCAR. Also provided are polynucleotides, e.g., template polynucleotides, for use in the methods provided herein, e.g., as templates for homology directed repair (HDR) mediated targeted integration of the transgene. In some embodiments, the polynucleotide includes a nucleic acid sequence encoding a portion, such as an antigen-binding domain, of a miniCAR; and one or more homology arm(s) linked to the nucleic acid sequence, wherein the one or more homology arm(s) comprise a sequence homologous to one or more region(s) of an open reading frame of an endogenous invariant CD3-IgSF chain locus, e.g., CD3E, CD3D or CD3G locus.

In some embodiments, the template polynucleotide contains one or more homology sequences (e.g., homology arms) linked to and/or flanking the transgene (heterologous or exogenous nucleic acids sequences) that includes a transgene comprising a sequence encoding the antigen-binding domain. In some embodiments, the homology sequences are used to target the heterologous sequences at the endogenous invariant CD3-IgSF chain locus. In some embodiments, the template polynucleotide includes nucleic acid sequences, such as a transgene, between the homology arms, for insertion or integration into the genome of a cell. The transgene in the template polynucleotide may comprise one or more sequences encoding a functional polypeptide (e.g., a cDNA), with or without a promoter or other regulatory elements.

In some embodiments, a template polynucleotide is a nucleic acid sequence which can be used in conjunction with one or more agent(s) capable of introducing a genetic disruption (e.g., a CRISPR-Cas9 combination targeting an invariant CD3-IgSF chain locus), to alter the structure of a target site. In some embodiments, the template polynucleotide alters the structure of the target site, e.g., insertion of transgene, by a homology directed repair event.

In some embodiments, the template polynucleotide alters the sequence of the target site, e.g., results in insertion or integration of the transgene between the homology arms, into the genome of the cell. In some aspects, targeted integration results in an in-frame integration of the coding portion of the transgene with one or more exons of the open reading frame of the endogenous invariant CD3-IgSF chain locus, e.g., in-frame with the adjacent exon at the integration site. For example, in some cases, the in-frame integration results in a portion of the endogenous open reading frame and the miniCAR to be expressed. Thus, the modified invariant CD3-IgSF chain locus can express a fusion protein comprising the polypeptide encoded by the integrated transgene and a polypeptide encoded by the endogenous invariant CD3-IgSF chain locus.

In some embodiments, the template polynucleotide includes sequences that correspond to or is homologous to a site on the target sequence that is cleaved, e.g., by one or more agent(s) capable of introducing a genetic disruption. In some embodiments, the template polynucleotide includes sequences that correspond to or is homologous to both, a first site on the target sequence that is cleaved in a first agent capable of introducing a genetic disruption, and a second site on the target sequence that is cleaved in a second agent capable of introducing a genetic disruption.

In some embodiments, a template polynucleotide comprises the following components: [5′ homology arm]-[a transgene (heterologous or exogenous nucleic acid sequences, e.g., encoding a portion, such as an antigen-binding domain, of a miniCAR)]-[3′ homology arm]. The homology arms provide for recombination into the chromosome, thus effectively inserting or integrating the transgene, e.g., that encodes an antigen-binding domain of a miniCAR into the genomic DNA at or near the cleavage site, such as target site(s). In some embodiments, the homology arms flank the sequences at the target site of genetic disruption.

In some embodiments, the template polynucleotide is double stranded. In some embodiments, the template polynucleotide is single stranded. In some embodiments, the template polynucleotide comprises a single stranded portion and a double stranded portion. In some embodiments, the template polynucleotide is comprised in a vector. In some embodiments, the template polynucleotide is DNA. In some embodiments, the template polynucleotide is RNA. In some embodiments, the template polynucleotide is double stranded DNA. In some embodiments, the template polynucleotide is single stranded DNA. In some embodiments, the template polynucleotide is double stranded RNA. In some embodiments, the template polynucleotide is single stranded RNA. In some embodiments, the template polynucleotide comprises a single stranded portion and a double stranded portion. In some embodiments, the template polynucleotide is comprised in a vector.

In certain embodiments, the polynucleotide, e.g., template polynucleotide contains and/or includes a transgene encoding a portion, such as an antigen-binding domain, of a miniCAR. In some of any embodiments, the transgene is targeted at a target site(s) that is within an endogenous gene, locus, or open reading frame that encodes the invariant CD3-IgSF gene product, e.g., CD3e, CD3d or CD3g. In some embodiments, the transgene is targeted for integration within the endogenous invariant CD3-IgSF chain locus open reading frame, such as to result in the expression of all or a portion of the encoded invariant CD3-IgSF chain gene product, e.g., CD3e, CD3d or CD3g.

Polynucleotides for insertion can also be referred to as “transgene,” “heterologous sequences,” “exogenous sequences” or “donor” polynucleotides or molecules. The template polynucleotide can be DNA, single-stranded and/or double-stranded and can be introduced into a cell in linear or circular form. The template polynucleotide can be DNA, single-stranded and/or double-stranded and can be introduced into a cell in linear or circular form. The template polynucleotide can be RNA single-stranded and/or double-stranded and can be introduced as a RNA molecule (e.g., part of an RNA virus). See also, U.S. Patent Pub. Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by known methods. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting heterologous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Pub. No. 20130326645.

In some embodiments, the double-stranded template polynucleotide includes sequences (also referred to as transgene) greater than 1 kb in length, for example between 2 and 200 kb, between 2 and 10 kb (or any value therebetween).

In some embodiments, the template polynucleotide is a single stranded nucleic acid. In some embodiments, the template polynucleotide is a double stranded nucleic acid. In some embodiments, the template polynucleotide comprises a nucleotide sequence, e.g., of one or more nucleotides, that will be added to or will template a change in the target DNA. In some embodiments, the template polynucleotide comprises a nucleotide sequence that may be used to modify the target site, e.g., copying or inserting the transgene in the template polynucleotide into the genome of the cell. In some embodiments, the template polynucleotide comprises a nucleotide sequence, e.g., of one or more nucleotides, that corresponds to wild type sequence of the target DNA, e.g., of the target site.

In some embodiments, the template polynucleotide is linear double stranded DNA. The length may be, e.g., about 200-5000 base pairs, e.g., about 200, 300, 400, 500, 600, 700, 800, 900, 1000, 1200, 1400, 1600, 1800, 2000, 2500, 3000, 4000 or 5000 base pairs. The length may be, e.g., at least 200, 300, 400, 500, 600, 700, 800, 900, 1000, 1200, 1400, 1600, 1800, 2000, 2500, 3000, 4000 or 5000 base pairs. In some embodiments, the length is no greater than 200, 300, 400, 500, 600, 700, 800, 900, 1000, 1200, 1400, 1600, 1800, 2000, 2500, 3000, 4000 or 5000 base pairs. In some embodiments, a double stranded template polynucleotide has a length of more than at or about 160 base pairs, e.g., about 200-4000, 300-3500, 400-3000, 500-2500, 600-2000, 700-1900, 800-1800, 900-1700, 1000-1600, 1100-1500 or 1200-1400 base pairs.

In some embodiments, the template polynucleotide is at or about 1000, 1250, 1500, 1750, 2000, 2250, 2500, 2750, 3000, 3250, 3500, 3750 or 4000 nucleotides in length, or any value between any of the foregoing. In some embodiments, the polynucleotide is between at or about 1500 and at or about 2500 nucleotides or at or about 1750 and at or about 2250 nucleotides in length. In some embodiments, the template polynucleotide is about 2000±250, 2000±200, 2000±150, 2000±100 or 2000±50 nucleotides in length.

The transgene contained on the template polynucleotide described herein may be isolated from plasmids, cells or other sources using known standard techniques such as PCR. Template polynucleotide for use can include varying types of topology, including circular supercoiled, circular relaxed, linear and the like. Alternatively, they may be chemically synthesized using standard oligonucleotide synthesis techniques. In addition, template polynucleotides may be methylated or lack methylation. Template polynucleotides may be in the form of bacterial or yeast artificial chromosomes (BACs or YACs).

The template polynucleotide can be linear single stranded DNA In some embodiments, the template polynucleotide is (i) linear single stranded DNA that can anneal to the nicked strand of the target DNA, (ii) linear single stranded DNA that can anneal to the intact strand of the target DNA, (iii) linear single stranded DNA that can anneal to the transcribed strand of the target DNA, (iv) linear single stranded DNA that can anneal to the non-transcribed strand of the target DNA, or more than one of the preceding.

The length may be, e.g., about 200-5000 nucleotides, e.g., about 200, 300, 400, 500, 600, 700, 800, 900, 1000, 1200, 1400, 1600, 1800, 2000, 2500, 3000, 4000 or 5000 nucleotides. The length may be, e.g., at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a single stranded template polynucleotide has a length of about 160 nucleotides, e.g., about 200-4000, 300-3500, 400-3000, 500-2500, 600-2000, 700-1900, 800-1800, 900-1700, 1000-1600, 1100-1500 or 1200-1400 nucleotides.

In some embodiments, the template polynucleotide is circular double stranded DNA, e.g., a plasmid. In some embodiments, the template polynucleotide comprises about 500 to 1000 base pairs of homology on either side of the transgene and/or the target site. In some embodiments, the template polynucleotide comprises about 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 10, 20, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene.

a. Transgene

In some embodiments, the template polynucleotide contains a transgene encoding a portion, such as a binding domain, of any miniCAR described herein, e.g., in Section III.B, or one or more regions, domains or chains of such miniCAR.

In some aspects, the transgene can encode all or a portion of the miniCAR. In some embodiments, the transgene encodes any miniCAR or portion thereof described herein, for example in Section III.B, or a one or more regions, domains or chains thereof, such as the antigen-binding domain of the miniCAR, e.g., miniCAR. In some aspects, upon integration of the transgene into the endogenous invariant CD3-IgSF chain locus, the resulting modified invariant CD3-IgSF chain locus encodes a miniCAR, such as any miniCAR described herein, for example, in Section III.B. For example, the transgene can include sequence of nucleotides encoding an extracellular antigen-binding domain. In some aspects, the transgene contains sequence of nucleotides encoding different regions or domains or portions of the miniCAR, that can be from different genes, coding sequences or exons or portions thereof, that are joined or linked.

In some aspects, the transgene, which are nucleic acid sequences of interest encoding a portion, such as an antigen-binding domain, of a miniCAR, including coding and/or non-coding sequences and/or partial coding sequences thereof, that are inserted or integrated at the target location in the genome can also be referred to as “transgene,” “transgene sequences,” “heterologous sequences,” “exogenous nucleic acids sequences,” or “donor sequences.” In some aspects, the transgene is a nucleic acid sequence that is exogenous or heterologous to an endogenous genomic sequences, such as the endogenous genomic sequences at a specific target locus or target location in the genome, of a T cell, e.g., a human T cell. In some aspects, the transgene is a sequence that is modified or different compared to an endogenous genomic sequence at a target locus or target location of a T cell, e.g., a human T cell. In some aspects, the transgene is a nucleic acid sequence that originates from or is modified compared to nucleic acid sequences from different genes, species and/or origins. In some aspects, the transgene is a sequence that is derived from a sequence from a different locus, e.g., a different genomic region or a different gene, of the same species. In some aspects, exemplary miniCARs include any described herein, e.g., in Section III.B.

In some embodiments, nuclease-induced HDR results in an insertion of a transgene (also called “heterologous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

In some aspects, the transgene is a sequence that is exogenous or heterologous to an open reading frame of the endogenous genomic invariant CD3-IgSF chain locus, e.g., CD3E, CD3D or CD3G locus, optionally a human T cell. In some aspects, HDR in the presence of a template polynucleotide containing a transgene linked to one or more homology arm(s) that are homologous to sequences near a target site at an endogenous invariant CD3-IgSF chain locus, e.g., CD3E, CD3D or CD3G locus, results in a modified invariant CD3-IgSF chain locus encoding a miniCAR.

In some embodiments, the transgene encodes all or a portion of the various regions, domains or chains of a miniCAR and regions, domains or chains, such as the binding domain, described in Section III.B herein.

In some aspects, the transgene is a chimeric sequence, comprising a sequence generated by joining different nucleic acid sequences from different genes, species and/or origins. In some aspects, the transgene contains sequence of nucleotides encoding different regions or domains or portions thereof, from different genes, coding sequences or exons or portions thereof, that are joined or linked. In some aspects the transgene for targeted integration encode a polypeptide or a fragment thereof.

In some embodiments, the transgene can encode a portion of, such as a domain or region thereof, for example, an extracellular region, such as an extracellular antigen-binding domain, of a chimeric receptor, such as a mini chimeric antigen receptor (miniCAR). In some embodiments, the transgene encodes a portion of a miniCAR, for example, an antigen-binding domain of the miniCAR. Exemplary miniCARs include those described in Section III.B below.

In some aspects, the transgene also contains non-coding, regulatory or control sequences, e.g., sequences required for permitting, modulating and/or regulating expression of the encoded polypeptide or fragment thereof or sequences required to modify a polypeptide. In some embodiments, the transgene does not comprise an intron or lacks one or more introns as compared to a corresponding nucleic acid in the genome if the transgene is derived from a genomic sequence. In some embodiments, the transgene does not comprise an intron. In some of embodiments, the transgene contains sequences encoding a portion, such as an antigen binding domain, of a miniCAR wherein all or a portion of the transgene are codon-optimized, e.g., for expression in human cells.

In some embodiments, the length of the transgene, including coding and non-coding regions, is between or between about 100 to about 10,000 base pairs, such as about 100, 200, 300, 400, 500, 600, 700, 800, 900, 1000, 1500, 2000, 2500, 3000, 3500, 4000, 4500, 5000, 6000, 7000, 8000, 9000 or 10000 base pairs. In some embodiments, the length of the transgene is limited by the maximum length of polynucleotide that can be prepared, synthesized or assembled and/or introduced into the cell or the capacity of the viral vector. In some aspects, the length of the transgene can vary depending on the maximum length of the template polynucleotide and/or the length of the one or more homology arm(s) required.

In some embodiments, genetic disruption-induced HDR results in an insertion or integration of the transgene at a target location in the genome. The template polynucleotide sequence is typically not identical to the genomic sequence where it is targeted. A template polynucleotide sequence can contain a transgene flanked by two regions of homology to allow for efficient HDR at the location of interest. A template polynucleotide sequence can contain several, discontinuous regions of homology to the genomic DNA. For example, for targeted insertion of sequences not normally present in a region of interest, said sequences can be present in a transgene and flanked by regions of homology to sequence in the region of interest. In some embodiments, the transgene encodes a portion, such as an antigen binding domain, of a miniCAR.

In some aspects, upon targeted integration of the transgene by HDR, the genome of the cell contains a modified invariant CD3-IgSF chain locus, comprising a nucleic acid sequence encoding a functional miniCAR. In some aspects, the transgene also contain sequence of nucleotides encoding other molecules and/or regulatory or control elements, e.g., heterologous promoter, and/or multicistronic elements.

In some embodiments, the transgene also includes a signal sequence encoding a signal peptide, a regulatory or control elements, such as a promoter, and/or one or more multicistronic elements, e.g., a ribosome skip element or an internal ribosome entry site (IRES). In some embodiments, the signal sequence can be placed 5′ of the sequence of nucleotides encoding the a portion, such as an antigen binding domain, of a miniCAR.

Exemplary regions, domains or chains encoded by the transgene are described below, and also can be any region or domain described in Section III.B herein.

(i) Signal Sequence

In some embodiments, the transgene includes a signal sequence encoding a signal peptide. In some aspects, the signal sequence may encode a heterologous or non-native signal peptide, e.g., a signal peptide from a different gene or species or a signal peptide that is different from the signal peptide of the endogenous invariant CD3-IgSF chain locus. In some aspects, exemplary signal sequence includes signal sequence of the GMCSFR alpha chain set forth in SEQ ID NO:84 or 87 and encoding the signal peptide set forth in SEQ ID NO:85; or the CD8 alpha signal peptide set forth in SEQ ID NO:86. The encoded precursor polypeptide, e.g., a precursor miniCAR, can include the signal peptide sequence, typically at the N-terminal of the encoded polypeptide. In the mature form of an expressed polypeptide, the signal sequence is cleaved from the remaining portions of the polypeptide. In some aspects, the signal sequence is placed 3′ of a heterologous regulatory or control element, if present, e.g., a promoter, such as a heterologous promoter, e.g., a promoter not derived from the invariant CD3-IgSF chain locus. In some aspects, the signal sequence is placed 3′ of one or more multicistronic element(s), if present, e.g., a sequence of nucleotides encoding a ribosome skip sequence and/or an internal ribosome entry site (IRES). In some aspects, the signal sequence can be placed 5′ of the sequence of nucleotides encoding the one or more components of the extracellular region (e.g., antigen-binding domain) in the transgene. In some embodiments, the signal sequence the most 5′ region present in the transgene, and is linked to one of the homology arms.

(ii) Binding Domain

In some aspects, the transgene contains sequences encoding an extracellular region of a chimeric receptor, such as a miniCAR. In some embodiments, the transgene sequences encode extracellular binding domain, such as a binding domain that specifically binds an antigen or a ligand, for example, an extracellular antigen-binding domain.

Exemplary extracellular region or binding domains, e.g., antigen-binding domains, of the miniCAR encoded by the transgene are described below, and can include any extracellular regions or binding domains, e.g., antigen-binding domains of exemplary miniCARs described in Sections III.B 0.1 below.

In some embodiments, the transgene encodes a portion of a miniCAR with specificity for a particular antigen or ligand, such as an antigen expressed on the surface of a particular cell type. In some embodiments, the antigen is selectively expressed or overexpressed on cells of the disease or condition, e.g., a tumor or pathogenic cells, as compared to normal or non-targeted cells or tissues, e.g., in healthy cells or tissues. In some embodiments, the binding domain is capable of binding to a target antigen that is associated with, specific to, and/or expressed on a cell or tissue of a disease, disorder or condition. In some embodiments, the disease, disorder or condition is an infectious disease or disorder, an autoimmune disease, an inflammatory disease, or a tumor or a cancer. In some embodiments, the target antigen is a tumor antigen. In some aspects, the transgene contains sequences encoding an antigen-binding domain of a miniCAR. In some embodiments, the transgene encodes an extracellular binding domain, such as a binding domain that specifically binds an antigen or a ligand.

In some embodiments, the antigen-binding domain is or comprises a polypeptide, a ligand, a receptor, a ligand-binding domain, a receptor-binding domain, an antigen, an epitope, an antibody, an antigen-binding domain, an epitope-binding domain, an antibody-binding domain, a tag-binding domain or a fragment of any of the foregoing. In other embodiments, the antigen is expressed on normal cells and/or is expressed on the engineered cells. In some aspects, the antigen is recognized by a binding domain, such as a ligand binding domain or an antigen binding domain. In some aspects, the transgene encodes an extracellular region containing one or more antigen-binding domain(s). In some embodiments, exemplary binding domain encoded by the transgene include antibodies and antigen-binding fragments thereof, including scFv or sdAb. In some embodiments, an antigen-binding fragment comprises antibody variable regions joined by a flexible linker. In some embodiments, the binding domain is or comprises a single chain variable fragment (scFv). In some embodiments, the binding domain is or comprises a single domain antibody (sdAb).

Exemplary antigens and antigen- or ligand-binding domains encoded by the transgene include those described in Section III.B.1 herein. In some aspects, the encoded miniCAR contains a binding domain that is or comprises a TCR-like antibody or a fragment thereof, such as an scFv that specifically recognizes an intracellular antigen, such as a tumor-associated antigen, presented on the cell surface as a major histocompatibility complex (MHC)-peptide complex. In some aspects, the transgene can encode a binding domain that is a TCR-like antibody or fragment thereof. In some embodiments, the binding domain is a multi-specific, such as a bi-specific, binding domain.

In some aspects, in the transgene, the sequence of nucleotides encoding the antigen-binding domain is placed 3′ of the signal sequence and 5′ of the 3′ homology arm (i.e., the sequence of nucleotides encoding the antigen-binding domain is the most 3′ sequence of the transgene). In some aspects, in the transgene, the sequence of nucleotides encoding the antigen-binding domain is placed between the signal sequence and the nucleotides encoding the linker, if present in the transgene. In some aspects, the sequence of nucleotides encoding the linker is placed between the sequence of nucleotides encoding the binding domain and the 3′ homology arm.

In some aspects, sequence of nucleotides encoding the one or more binding domain(s) can be placed 3′ of a signal sequence, if present, in the transgene. In some aspects, sequence of nucleotides encoding the one or more binding domain(s) can be placed 3′ of the sequence of nucleotides encoding one or more regulatory or control element(s), in the transgene. In some aspects, sequence of nucleotides encoding the one or more binding domain(s) can be placed 5′ of the sequence of nucleotides encoding the linker, if present, in the transgene.

In some embodiments, the transgene also comprises one or more multicistronic element(s), e.g., a ribosome skip sequence and/or an internal ribosome entry site (IRES). In some aspects, the transgene also includes regulatory or control elements, such as a promoter, typically at the most 5′ portion of the transgene, e.g., 5′ of the signal sequence. In some aspects, sequence of nucleotides encoding one or more additional molecule(s) or additional domains or regions can be included in the transgene portion of the polynucleotide. In some aspects, the sequence of nucleotides encoding one or more additional molecule(s) or additional domains or regions can be placed 5′ of the sequence of nucleotides encoding an antigen-binding domain. In some aspects, the sequence of nucleotides encoding the one or more additional molecule(s) or additional domains, regions or chains is upstream of the sequence of nucleotides encoding the antigen-binding domain.

(iii) Linker

In some embodiments, the transgene includes sequences encoding a linker. In some embodiments, the extracellular region, e.g., antigen-binding domain, of the encoded miniCAR comprises a linker, optionally wherein the linker is operably linked between the extracellular antigen-binding domain and the transmembrane region of the miniCAR, e.g., from the endogenous invariant CD3-IgSF chain locus, e.g., CD3E, CD3D or CD3G locus. In some aspects, the linker can link the extracellular portion containing the antigen-binding domain (e.g., encoded by the transgene) and other regions or domains of the miniCAR, such as all or a portion of the extracellular region, transmembrane region and intracellular region of the receptor, e.g., encoded by endogenous invariant CD3-IgSF chain locus, e.g., CD3E, CD3D or CD3G locus. In some aspects, the transgene includes a sequence encoding a linker. In some aspects, the transgene sequence does not include a sequence encoding a linker.

Exemplary linkers that can be encoded by the transgene sequence include flexible peptide linkers, and any linkers that can be contained in the exemplary miniCARs described in Section III. B 0.2 below.

In some aspects, the sequence of nucleotides encoding the linker can be placed 3′ of the sequence of nucleotides encoding the antigen-binding domain in the transgene. In some aspects, the sequence of nucleotides encoding the linker can be placed 5′ of the 3′ homology arm, i.e., the sequence of nucleotides encoding the linker is the 3′ most sequence of the transgene and is directly adjacent to the 3′ homology arm.

(iv) Affinity Tag

In some embodiments, the transgene includes sequences encoding an affinity tag. In some embodiments, the extracellular region, e.g., antigen-binding domain, of the encoded miniCAR comprises an affinity tag, optionally wherein the affinity tag is positioned between the extracellular antigen-binding domain and the transmembrane region of the miniCAR, e.g., from the endogenous invariant CD3-IgSF chain locus, e.g., CD3E, CD3D or CD3G locus. In some embodiments, the affinity tag is positioned between the extracellular antigen-binding domain and the linker. In some embodiments, the affinity tag is positioned between the linker and the extracellular region of the invariant CD3-IgSF chain locus, e.g., CD3E, CD3D or CD3G locus. In some aspects, the affinity tag, in addition to the linker or in lieu of the linker, can be positioned between the extracellular portion containing the antigen-binding domain (e.g., encoded by the transgene) and other regions or domains of the miniCAR, such as all or a portion of the extracellular region, transmembrane region and intracellular region of the receptor, e.g., encoded by endogenous invariant CD3-IgSF chain locus, e.g., CD3E, CD3D or CD3G locus. In some aspects, the transgene includes a sequence encoding an affinity tag. In some aspects, the transgene sequence does not include a sequence encoding an affinity tag.

Exemplary affinity tags that can be encoded by the transgene sequence include streptavidin-binding peptides, and any affinity tags that can be contained in the exemplary miniCARs described in Section III.B.3 below.

In some aspects, the sequence of nucleotides encoding the affinity tag can be placed 5′ of the sequence of nucleotides encoding the antigen-binding domain in the transgene. In some aspects, the sequence of nucleotides encoding the affinity tag can be placed 3′ of the sequence of nucleotides encoding the antigen-binding domain in the transgene. In some aspects, the sequence of nucleotides encoding the affinity tag can be placed 5′ of the sequence of nucleotides encoding the linker in the transgene. In some aspects, the sequence of nucleotides encoding the affinity tag can be placed 3′ of the sequence of nucleotides encoding the linker in the transgene. In some aspects, the sequence of nucleotides encoding the affinity tag can be placed 5′ of the 3′ homology arm, i.e., the sequence of nucleotides encoding the affinity tag is the 3′ most sequence of the transgene and is directly adjacent to the 3′ homology arm.

(v) Additional Molecules, e.g., Markers

In some embodiments, the transgene also includes a sequence of nucleotides encoding one or more additional molecules, such as an antibody, an antigen, a transduction marker or a surrogate marker (e.g., truncated cell surface marker), an enzyme, an factors, a transcription factor, an inhibitory peptide, a growth factor, a nuclear receptor, a hormone, a lymphokine, a cytokine, a chemokine, a soluble receptor, a soluble cytokine receptor, a soluble chemokine receptor, a reporter, an additional miniCAR, functional fragments or functional variants of any of the foregoing and combinations of the foregoing. In some aspects, such sequence of nucleotides encoding one or more additional molecules can be placed 5′ of the sequence of nucleotides encoding the extracellular antigen-binding domain of the miniCAR. In some aspects, the sequences encoding one or more other molecules and the sequence of nucleotides encoding regions or domains of the miniCAR are separated by regulatory sequences, such as a 2A ribosome skipping element and/or promoter sequences.

In some embodiments, the transgene also includes a sequence of nucleotides encoding one or more additional molecules. In some aspects, one or more additional molecules include one or more marker(s). In some embodiments, the one or more marker(s) includes a transduction marker, a surrogate marker and/or a selection marker. In some embodiments, the transgene also includes nucleic acid sequences that can improve the efficacy of therapy, such as by promoting viability and/or function of transferred cells; nucleic acid sequences to provide a genetic marker for selection and/or evaluation of the cells, such as to assess in vivo survival or localization; nucleic acid sequences to improve safety, for example, by making the cell susceptible to negative selection in vivo as described by Lupton et al., Mol. and Cell Biol., 11:6 (1991); and Riddell et al., Human Gene Therapy 3:319-338 (1992); see also WO 1992008796 and WO 1994028143 describing the use of bifunctional selectable fusion genes derived from fusing a dominant positive selectable marker with a negative selectable marker, and U.S. Pat. No. 6,040,177. In some aspects, the markers include any markers described herein, for example, in this section or Sections II or III.B, or any additional molecules and/or receptor polypeptides described herein, for example, in Section III.B.1. In some embodiments, the additional molecule is a surrogate marker, optionally a truncated receptor, optionally wherein the truncated receptor lacks an intracellular signaling domain and/or is not capable of mediating intracellular signaling when bound by its ligand.

In some embodiments, the marker is a transduction marker or a surrogate marker. A transduction marker or a surrogate marker can be used to detect cells that have been introduced with the polynucleotide, e.g., a polynucleotide encoding a miniCAR. In some embodiments, the transduction marker can indicate or confirm modification of a cell. In some embodiments, the surrogate marker is a protein that is made to be co-expressed on the cell surface with the miniCAR. In some of any embodiments, such a surrogate marker is a surface protein that has been modified to have little or no activity. In certain embodiments, the surrogate marker is encoded on the same polynucleotide that encodes the miniCAR. In some embodiments, the nucleic acid sequence encoding the miniCAR is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the transgene includes sequences encoding one or more additional molecule that is an immunomodulatory agent. In some embodiments, the immunomodulatory molecule is selected from an immune checkpoint modulator, an immune checkpoint inhibitor, a cytokine or a chemokine. In some embodiments, the immunomodulatory agent is an immune checkpoint inhibitor capable of inhibiting or blocking a function of an immune checkpoint molecule or a signaling pathway involving an immune checkpoint molecule. In some embodiments, the immune checkpoint molecule is selected from among PD-1, PD-L1, PD-L2, CTLA-4, LAG-3, TIM3, VISTA, an adenosine receptor or extracellular adenosine, optionally an adenosine 2A Receptor (A2AR) or adenosine 2B receptor (A2BR), or adenosine or a pathway involving any of the foregoing. Other exemplary additional molecules include epitope tags, detectable molecules such as fluorescent or luminescent proteins, or molecules that mediate enhanced cell growth and/or gene amplification (e.g., dihydrofolate reductase). Epitope tags include, for example, one or more copies of FLAG, His, myc, Tap, HA or any detectable amino acid sequence. In some embodiments, additional molecules can include non-coding sequences, inhibitory nucleic acid sequences, such as antisense RNAs, RNAi, shRNAs and micro RNAs (miRNAs), or nuclease recognition sequences.

(vi) Multicistronic Elements and Regulatory or Control Elements

In some aspects, the transgene, including the transgene encoding a portion of a miniCAR, e.g., antigen-binding domain of a miniCAR, can be inserted so that its expression is driven by the endogenous promoter at the integration site, namely the promoter that drives expression of the endogenous invariant CD3-IgSF locus gene. In some embodiments in which the polypeptide encoding sequences are promoterless, the expression of the integrated transgene is then ensured by transcription driven by an endogenous promoter or other control element in the region of interest. For example, the transgene encoding a portion, such as an antigen-binding domain, of the miniCAR can be inserted without a promoter, but in-frame with the coding sequence of the endogenous invariant CD3-IgSF locus, such that expression of the integrated transgene is controlled by the transcription of the endogenous promoter and/or other regulatory elements at the integration site. In some embodiments, a multicistronic element such as a ribosome skipping element/self-cleavage element (e.g., a 2A element or an internal ribosome entry site (IRES)), is placed upstream of the transgene encoding a portion of the miniCAR such that the multicistronic element is placed in-frame with one or more exons of the endogenous open reading frame at the invariant CD3-IgSF locus, such that the expression of the transgene encoding the miniCAR is operably linked to the endogenous invariant CD3-IgSF locus promoter. In some embodiments, the transgene does not comprise a sequence encoding a 3′ UTR. In some embodiments, upon integration of the transgene into the endogenous invariant CD3-IgSF locus, the transgene is integrated upstream of the 3′ UTR of the endogenous invariant CD3-IgSF locus, such that the message encoding the miniCAR contains a 3′ UTR of the endogenous invariant CD3-IgSF locus, e.g., from the open reading frame or partial sequence thereof of the endogenous invariant CD3-IgSF locus. In some embodiments, the open reading frame or a partial sequence thereof encoding the remaining portion of the miniCAR comprises a 3′ UTR of the endogenous invariant CD3-IgSF locus.

In some embodiments, a “tandem” cassette is integrated into the selected site. In some embodiments, one or more of the “tandem” cassettes encode one or more polypeptide or factors, each independently controlled by a regulatory element or all controlled as a multi-cistronic expression system. In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows co-expression of gene products by a message from a single promoter. Alternatively, in some cases, a single promoter may direct expression of an RNA that contains, in a single open reading frame (ORF), two or three polypeptides separated from one another by sequences encoding a self-cleavage peptide (e.g., 2A sequences) or a protease recognition site (e.g., furin), as described herein. The ORF thus encodes a single polypeptide, which, either during (in the case of 2A) or after translation, is processed into the individual proteins. In some embodiments, the “tandem cassette” includes the first component of the cassette comprising a promoterless sequence, followed by a transcription termination sequence, and a second sequence, encoding an autonomous expression cassette or a multi-cistronic expression sequence. In some embodiments, the tandem cassette encodes two or more different polypeptides or factors, e.g., an antigen-binding domain of a miniCAR and one or more additional molecules. In some embodiments, nucleic acid sequences encoding an antigen-binding domain of a miniCAR and one or more additional molecules are introduced as tandem expression cassettes or bi- or multi-cistronic cassettes, into one target DNA integration site.

In some embodiments, the transgene (e.g., exogenous nucleic acid sequences) also contains one or more heterologous or exogenous regulatory or control elements, e.g., cis-regulatory elements, that are not, or are different from the regulatory or control elements of the endogenous invariant CD3-IgSF chain locus. In some embodiments, the heterologous or exogenous regulatory or control element is operably linked to nucleic acid sequences encoding an additional component of the transgene, e.g., a nucleic acid sequence encoding an additional polypeptide, apart from the nucleic acid sequence encoding the miniCAR.

In some aspects, the heterologous regulatory or control elements include such as a promoter, an enhancer, an intron, an insulator, a polyadenylation signal, a transcription termination sequence, a Kozak consensus sequence, a multicistronic element (e.g., internal ribosome entry sites (IRES), a 2A sequence), sequences corresponding to untranslated regions (UTR) of a messenger RNA (mRNA), and splice acceptor or donor sequences, such as those that are not, or are different from the regulatory or control element at the invariant CD3-IgSF chain locus. In some embodiments, the heterologous regulatory or control elements include a promoter, an enhancer, an intron, a polyadenylation signal, a Kozak consensus sequence, a splice acceptor sequence and/or a splice donor sequence. In some embodiments, the transgene comprises a promoter that is heterologous and/or not typically present at or near the target site, for example, to control the expression of additional components in the transgene.

In some cases, the multicistronic element, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004); also referred to as a self-cleavage element). This allows the inserted transgene to be controlled by the transcription of the endogenous promoter at the integration site such as an invariant CD3-IgSF chain locus promoter. Exemplary multicistronic element include 2A sequences from the foot-and-mouth disease virus (F2A, e.g., SEQ ID NO: 93), equine rhinitis A virus (E2A, e.g., SEQ ID NO: 92), Thosea asigna virus (T2A, e.g., SEQ ID NO: 88 or 89), and porcine teschovirus-1 (P2A, e.g., SEQ ID NO: 90, 91 or 94) as described in U.S. Patent Pub. No. 20070116690. In some embodiments, the template polynucleotide includes a P2A ribosome skipping element (sequence set forth in SEQ ID NO:3) upstream of the transgene, e.g., nucleic acids encoding a portion, such as an antigen-binding domain, of the miniCAR.

In some embodiments, the transgene encoding the antigen-binding domain of the miniCAR and/or the sequences encoding an additional molecule independently comprises one or more multicistronic element(s). In some embodiments, the one or more multicistronic element(s) are upstream of the transgene encoding the antigen-binding domain of the miniCAR and/or the sequences encoding an additional molecule. In some embodiments, the multicistronic element(s) is positioned between the transgene encoding the antigen-binding domain of the miniCAR and/or the sequences encoding an additional molecule.

In some embodiments, the sequence encoding an additional molecule is operably linked to a heterologous regulatory or control element. In some aspects, the heterologous regulatory or control element comprises a heterologous promoter. In some embodiments, the heterologous promoter is selected from among a constitutive promoter, an inducible promoter, a repressible promoter, and/or a tissue-specific promoter. In some embodiments, regulatory or control element is a promoter and/or enhancer, for example a constitutive promoter or an inducible or tissue-specific promoter. In some embodiments, the promoter is selected from among an RNA pol I, pol II or pol III promoter. In some embodiments, the promoter is recognized by RNA polymerase II (e.g., a CMV, SV40 early region or adenovirus major late promoter). In some embodiments, the promoter is recognized by RNA polymerase III (e.g., a U6 or H1 promoter). In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the heterologous promoter is or comprises a human elongation factor 1 alpha (EF1α) promoter or an MND promoter or a variant thereof.

In some embodiments, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence or is an analog thereof or is capable of being bound by or recognized by a Lac repressor or an analog thereof. In some embodiments, the promoter is a tissue-specific promoter. In some instances, the promoter is only expressed in a specific cell type (e.g., a T cell or B cell or NK cell specific promoter).

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In some instances, the promoter drives expression only in a specific cell type (e.g., a T cell or B cell or NK cell specific promoter).

In some embodiments, the promoter is a viral promoter. In some embodiments, the promoter is a non-viral promoter. In some cases, the promoter is selected from among human elongation factor 1 alpha (EF1α) promoter or a modified form thereof (EF1α promoter with HTLV1 enhancer) or the MND promoter. In some embodiments, the polynucleotide does not include a heterologous or exogenous regulatory element, e.g., a promoter. In some embodiments, the promoter is a bidirectional promoter (see, e.g., WO2016/022994).

In some embodiments, the transgene may also include splice acceptor sequences. Exemplary known splice acceptor site sequences include, e.g., CTGACCTCTTCTCTTCCTCCCACAG (SEQ ID NO:95) (from the human HBB gene) and TTTCTCTCCACAG (SEQ ID NO:96) (from the human IgG gene).

(vii) Exemplary Transgenes

In some embodiments, an exemplary transgene includes, in 5′ to 3′ order, a signal sequence, a sequence of nucleotides encoding an antigen-binding domain. In some embodiments, an exemplary transgene includes, in 5′ to 3′ order, a multicistronic element (e.g., 2A element), a signal sequence, a sequence of nucleotides encoding an antigen-binding domain. In some embodiments, the transgene includes, in 5′ to 3′ order, a multicistronic element (e.g., 2A element), a signal sequence, a sequence of nucleotides encoding an antigen-binding domain, and a sequence of nucleotides encoding a linker.

In some embodiments, an exemplary transgene includes, in 5′ to 3′ order, a multicistronic element (e.g., 2A element), a sequence of nucleotides encoding one or more additional molecules, optionally a multicistronic element (e.g., 2A element), a signal sequence, and a sequence of nucleotides encoding an antigen-binding domain. In some embodiments, an exemplary transgene includes, in 5′ to 3′ order, a multicistronic element (e.g., 2A element), a sequence of nucleotides encoding one or more additional molecules, a multicistronic element (e.g., 2A element), a signal sequence, a sequence of nucleotides encoding an antigen-binding domain, and a sequence of nucleotides encoding a linker.

In some embodiments, an exemplary transgene includes, in 5′ to 3′ order, a heterologous regulatory element (e.g., a heterologous promoter), optionally a multicistronic element (e.g., 2A element), a signal sequence, a sequence of nucleotides encoding an antigen-binding domain. In some embodiments, an exemplary transgene includes, in 5′ to 3′ order, a heterologous regulatory element (e.g., a heterologous promoter), a sequence of nucleotides encoding one or more additional molecules, optionally a multicistronic element (e.g., 2A element), a signal sequence, and a sequence of nucleotides encoding an antigen-binding domain. In some embodiments, an exemplary transgene includes, in 5′ to 3′ order, a heterologous regulatory element (e.g., a heterologous promoter), a sequence of nucleotides encoding one or more additional molecules, optionally a multicistronic element (e.g., 2A element), a signal sequence, a sequence of nucleotides encoding an antigen-binding domain, and a sequence of nucleotides encoding a linker.

In some aspects, exemplary sequence of nucleotides encoding an antigen-binding domain, sequence of nucleotides encoding linker, signal sequence, heterologous regulatory element (e.g., a heterologous promoter), multicistronic element, and one or more additional molecules include any described herein.

b. Homology Arms

In some embodiments, the template polynucleotide contains one or more homology sequences (also called “homology arms”) on the 5′ and 3′ ends, linked to or surrounding the transgene encoding a portion, a region or a domain of a miniCAR, such as the extracellular antigen-binding domain of the miniCAR. In some aspects, the transgene is linked directly to the homology arm(s). The homology arms allow the DNA repair mechanisms, e.g., homologous recombination machinery, to recognize the homology and use the template polynucleotide as a template for repair, and the nucleic acid sequence between the homology arms are copied into the DNA being repaired, effectively inserting or integrating the transgene into the target site of integration in the genome between the location of the homology.

In some embodiments, the transgene comprises a sequence of nucleotides that is in-frame with one or more exons of the open reading frame of the invariant CD3-IgSF locus comprised in the one or more homology arm(s). In some aspects, the a portion of the miniCAR such as the antigen-binding domain, is encoded by the transgene, and the remaining portion of the miniCAR is encoded by the endogenous invariant CD3-IgSF locus.

In some embodiments, the homology arm sequences include sequences that are homologous to the genomic sequences surrounding the genetic disruption, e.g., a target site within the invariant CD3-IgSF locus. In some embodiments, the template polynucleotide comprises the following components: [5′ homology arm]-[a transgene (heterologous or exogenous nucleic acid sequences, e.g., encoding a portion, such as an antigen-binding domain, of a miniCAR)]-[3′ homology arm]. In some embodiments, the 5′ homology arm sequences include contiguous sequences that are homologous to sequences located near the genetic disruption on the 5′ side. In some embodiments, the 3′ homology arm sequences include contiguous sequences that are homologous to sequences located near the genetic disruption on the 3′ side. In some aspects, the target site is determined by targeting of the one or more agent(s) capable of introducing a genetic disruption, e.g., Cas9 and gRNA targeting a specific site within an invariant CD3-IgSF locus, e.g., CD3E, CD3D or CD3G locus.

In some aspects, the transgene within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of the transgene. In some aspects, the homology arms are designed to target integration within an exon of the open reading frame of the endogenous invariant CD3-IgSF locus, and the homology arm sequences are determined based on the desired location of integration surrounding the genetic disruption, including exon and intron sequences surrounding the genetic disruption. In some embodiments, the location of the target site, relative location of the one or more homology arm(s), and the transgene (heterologous nucleic acid sequence) for insertion can be designed depending on the requirement for efficient targeting and the length of the template polynucleotide or vector that can be used. In some aspects, the homology arms are designed to target integration within an intron of the open reading frame of the invariant CD3-IgSF locus. In some aspects, the homology arms are designed to target integration within an exon of the open reading frame of the invariant CD3-IgSF locus.

In some aspects, the target integration site (site for targeted integration) within the invariant CD3-IgSF locus is located within an open reading frame at the endogenous invariant CD3-IgSF locus. In some embodiments, the target integration site is at or near any of the target sites described herein, e.g., in Section I.A. In some aspects, the target location for integration is at or around the target site for genetic disruption, e.g., within less than 500, 450, 400, 350, 300, 250, 200, 150, 100 or 50 bp of the target site for genetic disruption.

In some aspects, the target integration site is within an exon of the open reading frame of the endogenous invariant CD3-IgSF locus, e.g., CD3E, CD3D or CD3G locus. In some aspects, the target integration site is within an intron of the open reading frame of the invariant CD3-IgSF locus. In some aspects, the target integration site is within a regulatory or control element, e.g., a promoter, of the invariant CD3-IgSF locus. In some embodiments, the target integration site is within or in close proximity to exons corresponding to early coding region, e.g., exon 1, 2, 3, 4 or 5 of the open reading frame of the endogenous invariant CD3-IgSF locus, or including sequence immediately following a transcription start site, within exon 1, 2, 3, 4 or 5 (such as described in Tables 1-5 herein), or within less than 500, 450, 400, 350, 300, 250, 200, 150, 100 or 50 bp of exon 1, 2, 3, 4 or 5. In some embodiments, the integration is targeted at or near exon 2 of the endogenous invariant CD3-IgSF locus, or within less than 500, 450, 400, 350, 300, 250, 200, 150, 100 or 50 bp of exon 2. In some aspects, the target integration site is at or near exon 1 of the endogenous invariant CD3-IgSF locus, e.g., within less than 500, 450, 400, 350, 300, 250, 200, 150, 100 or 50 bp of exon 1. In some embodiments, the target integration site is at or near exon 2 of the endogenous invariant CD3-IgSF locus, or within less than 500, 450, 400, 350, 300, 250, 200, 150, 100 or 50 bp of exon 2. In some aspects, the target integration site is at or near exon 3 of the endogenous invariant CD3-IgSF locus, e.g., within less than 500, 450, 400, 350, 300, 250, 200, 150, 100 or 50 bp of exon 3. In some aspects, the target integration site is at or near exon 4 of the endogenous invariant CD3-IgSF locus, e.g., within less than 500, 450, 400, 350, 300, 250, 200, 150, 100 or 50 bp of exon 4. In some aspects, the target integration site is at or near exon 5 of the endogenous invariant CD3-IgSF locus, e.g., within less than 500, 450, 400, 350, 300, 250, 200, 150, 100 or 50 bp of exon 5. In some aspects, the target integration site is within a regulatory or control element, e.g., a promoter, of the invariant CD3-IgSF locus.

In some embodiments, the 5′ homology arm sequences include contiguous sequences of approximately 10, 20, 30, 40, 50, 100, 200, 300, 400, 500, 600, 700, 800, 900, 1000, 1500, 2000, 3000, 4000, or 5000 base pairs 5′ of the target site for genetic disruption, starting near the target site at the endogenous invariant CD3-IgSF locus. In some embodiments, the 3′ homology arm sequences include contiguous sequences of approximately 10, 20, 30, 40, 100, 200, 300, 400, 500, 600, 700, 800, 900, 1000, 1500, 2000, 3000, 4000, or 5000 base pairs 3′ of the target site for genetic disruption, starting near the target site at the endogenous invariant CD3-IgSF locus. Thus, upon integration via HDR, the transgene is targeted for integration at or near the target site for genetic disruption, e.g., a target site within an exon or intron of the endogenous invariant CD3-IgSF locus.

In some aspects, the homology arms contain sequences that are homologous to a portion of an open reading frame sequence at the endogenous invariant CD3-IgSF locus. In some aspects, the homology arm sequences contain sequences homologous to contiguous portion of an open reading frame sequence, including exons and introns, at the endogenous invariant CD3-IgSF locus. In some aspects, the homology arm contains sequences that are identical to a contiguous portion of an open reading frame sequence, including exons and introns, at the endogenous invariant CD3-IgSF locus.

In some embodiments, the template polynucleotide contains homology arms for targeting integration of the transgene at the endogenous invariant CD3-IgSF locus (exemplary genomic locus sequence described in Tables 1-5 herein; exemplary human mRNA sequence described in Section II.A.1 above) In some embodiments, the genetic disruption is introduced using any of the agents for genetic disruption, e.g., targeted nucleases and/or gRNAs described herein. In some embodiments, the template polynucleotide comprises about 500 to 1000, e.g., 500 to 900 or 600 to 700, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequences, which is homologous to 500, 600, 700, 800, 900 or 1000 base pairs of sequences 5′ of the genetic disruption at an invariant CD3-IgSF locus, the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption at an invariant CD3-IgSF locus.

In some aspects, the boundary between the transgene and the one or more homology arm sequences, is designed such that upon HDR and targeted integration of the transgene, the sequences within the transgene that encode one or more polypeptide, e.g., chain(s), domain(s) or region(s) of a chimeric receptor, e.g., miniCAR, is integrated in-frame with one or more exons of the open reading frame sequence at the endogenous invariant CD3-IgSF locus, and/or generates an in-frame fusion of the transgene that encode a polypeptide and one or more exons of the open reading frame sequence at the endogenous invariant CD3-IgSF locus. In some embodiments, all or a portion of the gene product of the invariant CD3-IgSF locus is encoded by the nucleic acid sequences of the endogenous open reading frame, and a portion of the miniCAR, e.g., the antigen-binding domain, is encoded by the integrated transgene, optionally separated by a multicistronic element, such as a 2A element.

In some embodiments, the one or more homology arm sequences include sequences that are homologous, substantially identical or identical to sequences that surround or flank the target site that are within an open reading frame sequence at the endogenous invariant CD3-IgSF locus. In some aspects, the one or more homology arm sequences contain introns and exons of a partial sequence of an open reading frame at the endogenous invariant CD3-IgSF locus. In some aspects, the boundary of the 5′ homology arm sequence and the transgene is such that, in a case of a transgene that does not contain a heterologous promoter, the coding portion of the transgene is fused in-frame with an upstream exon or a portion thereof, e.g., exon 1, 2, 3, 4 or 5, depending on the location of targeted integration, of the open reading frame of the endogenous invariant CD3-IgSF locus.

In some aspects, the boundary of the 5′ homology arm sequence and the transgene is such that, the upstream exons or a portion thereof, e.g., exons 1, 2, 3, 4 or 5, of the open reading frame of the endogenous invariant CD3-IgSF locus, is fused in-frame with the coding portions of the transgene. Thus, upon targeted integration, transcription and translation, the encoded miniCAR that is a contiguous polypeptide is produced, from a fusion DNA sequence of the transgene and an open reading frame sequence of the endogenous invariant CD3-IgSF locus. In some aspects, the upstream exons or a portion thereof encode all or a portion of the gene product of the invariant CD3-IgSF locus. In some aspects, upon targeted integration, a multicistronic element, e.g., a 2A element or an internal ribosome entry site (IRES) separates the open reading frame sequence of the endogenous invariant CD3-IgSF locus and the transgene encoding a portion of the miniCAR. In some aspects, when expressed and translated from the modified invariant CD3-IgSF locus, the polypeptide is cleaved to generate all or a portion of the polypeptide encoded by the endogenous invariant CD3-IgSF locus and a miniCAR.

In some embodiments, exemplary 5′ homology arm for targeting integration at the endogenous invariant CD3-IgSF locus CD3E comprises the sequence set forth in SEQ ID NO:4, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 4 or a partial sequence thereof.

In some embodiments, exemplary 3′ homology arm for targeting integration at the endogenous invariant CD3-IgSF locus CD3E comprises the sequence set forth in SEQ ID NO:5, or a sequence that exhibits at least 85%, 86%, 87%, 88%, 89%, 90%, 91%, 92%, 93%, 94%, 95%, 96%, 97%, 98%, 99% or more sequence identity to SEQ ID NO:5 or a partial sequence thereof.

In some aspects, the target site can determine the relative location and sequences of the homology arms. The homology arm can typically extend at least as far as the region in which end resection by the DNA repair mechanism can occur after the genetic disruption, e.g., DSB, is introduced, e.g., in order to allow the resected single stranded overhang to find a complementary region within the template polynucleotide. The overall length could be limited by parameters such as plasmid size, viral packaging limits or construct size limit.

In some embodiments, the homology arm comprises about 500 to 1000, e.g., 600 to 900 or 700 to 800, base pairs of homology on either side of the target site at the endogenous gene. In some embodiments, the homology arm comprises about at least or less than or about 200, 300, 400, 500, 600, 700, 800, 900 or 1000 base pairs homology 5′ of the target site, 3′ of the target site, or both 5′ and 3′ of the target site at invariant CD3-IgSF locus.

In some embodiments, the homology arm comprises at or about 10, 20, 30, 50, 100, 200, 300, 400, 500, 600, 700, 800, 900, 1000, 1500, 2000, 3000, 4000, or 5000 base pairs homology 3′ of the target site at invariant CD3-IgSF locus. In some embodiments, the homology arm comprises at or about 100 to 500, 200 to 400 or 250 to 350, base pairs homology 3′ of the transgene and/or target site at invariant CD3-IgSF locus. In some embodiments, the homology arm comprises less than about 100, 90, 80, 70, 60, 50, 40, 30, 20, 15, or 10 base pairs homology 5′ of the target site at invariant CD3-IgSF locus.

In some embodiments, the homology arm comprises at or about 10, 20, 30, 50, 100, 200, 300, 400, 500, 600, 700, 800, 900, 1000, 1500, 2000, 3000, 4000, or 5000 base pairs homology 5′ of the target site at invariant CD3-IgSF locus. In some embodiments, the homology arm comprises at or about 100 to 500, 200 to 400 or 250 to 350, base pairs homology of the transgene and/or target site at invariant CD3-IgSF locus. In some embodiments, the homology arm comprises less than about 100, 90, 80, 70, 60, 50, 40, 30, 20, 15, or 10 base pairs homology 3′ of the target site at invariant CD3-IgSF locus.

In some embodiments, the 3′ end of the 5′ homology arm is the position next to the 5′ end of the transgene. In some embodiments, the 5′ homology arm can extend at least at or about 10, 20, 30, 40, 50, 100, 200, 300, 400, 500, 600, 700, 800, 900, 1000, 1500, 2000, 3000, 4000, or 5000 nucleotides 5′ from the 5′ end of the transgene.

In some embodiments, the 5′ end of the 3′ homology arm is the position next to the 3′ end of the transgene. In some embodiments, the 3′ homology arm can extend at least at or about 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ the homology arms, may each comprise about 1000 base pairs (bp) of sequence flanking the most distal target sites (e.g., 1000 bp of sequence on either side of the mutation).

Exemplary homology arm lengths include at least at or about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include less than or less than about or is or is about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or 750 to at or about 1000 nucleotides.

In some of any such embodiments, the transgene is integrated by a template polynucleotide introduced into each of a plurality of T cells. In some of any embodiments, the template polynucleotide comprises the structure [5′ homology arm]-[transgene]-[3′ homology arm]. In certain embodiments, the 5′ homology arm and the 3′ homology arm comprises nucleic acid sequences homologous to nucleic acid sequences surrounding the at least at or about one target site. In some embodiments, the 5′ homology arm comprises nucleic acid sequences that are homologous to nucleic acid sequences 5′ of the target site. In some of any embodiments, the 3′ homology arm comprises nucleic acid sequences that are homologous to nucleic acid sequences 3′ of the target site. In certain embodiments, the 5′ homology arm and the 3′ homology arm independently are at least at or about or at least at or about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In some embodiments, the 5′ homology arm and the 3′ homology arm independently are between at or about 50 and at or about 100, 100 and at or about 250, 250 and at or about 500, 500 and at or about 750, 750 and at or about 1000, 1000 and at or about 2000 nucleotides. In some of any such embodiments, the 5′ homology arm and the 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

In some of any embodiments, the 5′ homology arm and the 3′ homology arm independently are from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides. In some of any embodiments, the 5′ homology arm and the 3′ homology arm independently are from at or about 100 to at or about at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length. In some embodiments, the 5′ homology arm and the 3′ homology arm independently are at or about 200, 300, 400, 500, 600, 700 or 800 nucleotides in length, or any value between any of the foregoing. In some embodiments, the 5′ homology arm and the 3′ homology arm independently are greater than at or about 300 nucleotides in length, optionally wherein the 5′ homology arm and the 3′ homology arm independently are at or about 400, 500 or 600 nucleotides in length or any value between any of the foregoing. In some embodiments, the 5′ homology arm and the 3′ homology arm independently are greater than at or about 300 nucleotides in length.

In some embodiments, one or more of the homology arms contain a sequence of nucleotides are homologous to sequences that encode a gene product of the invariant CD3-IgSF locus or a fragment thereof. In some embodiments, one or more homology arms are connected or linked in frame with the transgene encoding a portion, such as the antigen-binding fragment, of the miniCAR.

In some embodiments, alternative HDR is employed. In some embodiments, alternative HDR proceeds more efficiently when the template polynucleotide has extended homology 5′ to the target site (i.e., in the 5′ direction of the target site strand). Accordingly, in some embodiments, the template polynucleotide has a longer homology arm and a shorter homology arm, wherein the longer homology arm can anneal 5′ of the target site. In some embodiments, the arm that can anneal 5′ to the target site is at least 25, 50, 75, 100, 125, 150, 175, or 200, 300, 400, 500, 600, 700, 800, 900, 1000, 1500, 2000, 3000, 4000, or 5000 nucleotides from the target site or the 5′ or 3′ end of the transgene. In some embodiments, the arm that can anneal 5′ to the target site is at least 10%, 20%, 30%, 40%, or 50% longer than the arm that can anneal 3′ to the target site. In some embodiments, the arm that can anneal 5′ to the target site is at least 2×, 3×, 4×, or 5× longer than the arm that can anneal 3′ to the target site. Depending on whether a ssDNA template can anneal to the intact strand or the targeted strand, the homology arm that anneals 5′ to the target site may be at the 5′ end of the ssDNA template or the 3′ end of the ssDNA template, respectively.

Similarly, in some embodiments, the template polynucleotide has a 5′ homology arm, a transgene, and a 3′ homology arm, such that the template polynucleotide contains extended homology to the 5′ of the target site. For example, the 5′ homology arm and the 3′ homology arm may be substantially the same length, but the transgene may extend farther of the target site than 3′ of the target site. In some embodiments, the homology arm extends at least 10%, 20%, 30%, 40%, 50%, 2×, 3×, 4×, or 5× further to the 5′ end of the target site than the 3′ end of the target site.

In some embodiments alternative HDR proceeds more efficiently when the template polynucleotide is centered on the target site. Accordingly, in some embodiments, the template polynucleotide has two homology arms that are essentially the same size. In some embodiments, the first homology arm (e.g., 5′ homology arm) of a template polynucleotide may have a length that is within 10%, 9%, 8%, 7%, 6%, 5%, 4%, 3%, 2%, or 1% of the second homology arm (e.g., 3′ homology arm) of the template polynucleotide.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

In some embodiments, the length of the template polynucleotide, including the transgene and the one or more homology arms, is between or between about 1000 to about base pairs, such as about 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000 or 20000 base pairs. In some embodiments, the length of the template polynucleotide is limited by the maximum length of polynucleotide that can be prepared, synthesized or assembled and/or introduced into the cell or the capacity of the viral vector, and the type of polynucleotide or vector. In some aspects, the limited capacity of the template polynucleotide can determine the length of the transgene and/or the one or more homology arms. In some aspects, the combined total length of the transgene and the one or more homology arms must be within the maximum length or capacity of the polynucleotide or vector. For example, in some aspects, the transgene portion of the template polynucleotide is about 1000, 1500, 2000, 2500, 3000, 3500 or 4000 base pairs, and if the maximum length of the template polynucleotide is about 5000 base pairs, the remaining portion of the sequence can be divided among the one or more homology arms, e.g., such that the 3′ or 5′ homology arms can be approximately 500, 750, 1000, 1250, 1500, 1750 or 2000 base pairs.

c. Exemplary Template Polynucleotides

In some embodiments, an exemplary template polynucleotide includes, in 5′ to 3′ order, a 5′ homology arm, a signal sequence, a sequence of nucleotides encoding an antigen-binding domain, and a 3′ homology arm. In some embodiments, an exemplary template polynucleotide includes, in 5′ to 3′ order, a 5′ homology arm, a multicistronic element (e.g., 2A element), a signal sequence, a sequence of nucleotides encoding an antigen-binding domain, and a 3′ homology arm. In some embodiments, the transgene includes, in 5′ to 3′ order, a 5′ homology arm, a multicistronic element (e.g., 2A element), a signal sequence, a sequence of nucleotides encoding an antigen-binding domain, a sequence of nucleotides encoding a linker, and a 3′ homology arm.

In some embodiments, an exemplary template polynucleotide includes, in 5′ to 3′ order, a 5′ homology arm, a multicistronic element (e.g., 2A element), a sequence of nucleotides encoding one or more additional molecules, optionally a multicistronic element (e.g., 2A element), a signal sequence, a sequence of nucleotides encoding an antigen-binding domain, and a 3′ homology arm. In some embodiments, an exemplary template polynucleotide includes, in 5′ to 3′ order, a 5′ homology arm, a multicistronic element (e.g., 2A element), a sequence of nucleotides encoding one or more additional molecules, a multicistronic element (e.g., 2A element), a signal sequence, a sequence of nucleotides encoding an antigen-binding domain, a sequence of nucleotides encoding a linker, and a 3′ homology arm.

In some embodiments, an exemplary template polynucleotide includes, in 5′ to 3′ order, a 5′ homology arm, a heterologous regulatory element (e.g., a heterologous promoter), optionally a multicistronic element (e.g., 2A element), a signal sequence, a sequence of nucleotides encoding an antigen-binding domain, and a 3′ homology arm. In some embodiments, an exemplary template polynucleotide includes, in 5′ to 3′ order, a 5′ homology arm, a heterologous regulatory element (e.g., a heterologous promoter), a sequence of nucleotides encoding one or more additional molecules, optionally a multicistronic element (e.g., 2A element), a signal sequence, a sequence of nucleotides encoding an antigen-binding domain, and a 3′ homology arm. In some embodiments, an exemplary template polynucleotide includes, in 5′ to 3′ order, a 5′ homology arm, a heterologous regulatory element (e.g., a heterologous promoter), a sequence of nucleotides encoding one or more additional molecules, optionally a multicistronic element (e.g., 2A element), a signal sequence, a sequence of nucleotides encoding an antigen-binding domain, a sequence of nucleotides encoding a linker, and a 3′ homology arm.

In some aspects, exemplary sequence of 5′ homology arm, 3′ homology arm, nucleotides encoding an antigen-binding domain, sequence of nucleotides encoding linker, signal sequence, heterologous regulatory element (e.g., a heterologous promoter), multicistronic element, one or more additional molecules include any described herein.

3. Delivery of Template Polynucleotides

In some embodiments, the polynucleotide, such as a template polynucleotide containing transgene sequences encoding a portion, such as an antigen-binding domain, of a miniCAR (for example, described in Section I.B.2 herein), are introduced into the cells in nucleotide form, e.g., as a polynucleotide or a vector. In particular embodiments, the polynucleotide contains a transgene sequence that encodes a portion of a miniCAR and one or more homology arms, and can be introduced into the cell for homology-directed repair (HDR)-mediated integration of the transgene sequences.

In some aspects, the provided embodiments genetic engineering of cells, by the introduction of one or more agent(s) or components thereof capable of inducing a genetic disruption and a template polynucleotide, to induce HDR and targeted integration of the transgene sequences. In some aspects, the one or more agent(s) and the template polynucleotide are delivered simultaneously. In some aspects, the one or more agent(s) and the template polynucleotide are delivered sequentially. In some embodiments, the one or more agent(s) are delivered prior to the delivery of the polynucleotide.

In some embodiments, the template polynucleotide is introduced into the cell for engineering, in addition to the agent(s) capable of inducing a targeted genetic disruption, e.g., nuclease and/or gRNAs. In some embodiments, the template polynucleotide(s) may be delivered prior to, simultaneously or after one or more components of the agent(s) capable of inducing a targeted genetic disruption is introduced into a cell. In some embodiments, the template polynucleotide(s) are delivered simultaneously with the agents. In some embodiments, the template polynucleotides are delivered prior to the agents, for example, seconds to hours to days before the template polynucleotides, including, but not limited to, 1 to 60 minutes (or any time therebetween) before the agents, 1 to 24 hours (or any time therebetween) before the agents or more than 24 hours before the agents. In some embodiments, the template polynucleotides are delivered after the agents, seconds to hours to days after the template polynucleotides, including immediately after delivery of the agent, e.g., between 30 seconds to 4 hours, such as about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after delivery of the agents and/or preferably within 4 hours of delivery of the agents. In some embodiments, the template polynucleotide is delivered more than 4 hours after delivery of the agents. In some embodiments, the template polynucleotide is introduced at or about 2 hours after the introduction of the one or more agents.

In some embodiments, the template polynucleotides may be delivered using the same delivery systems as the agent(s) capable of inducing a targeted genetic disruption, e.g., nuclease and/or gRNAs. In some embodiments, the template polynucleotides may be delivered using different same delivery systems as the agent(s) capable of inducing a targeted genetic disruption, e.g., nuclease and/or gRNAs. In some embodiments, the template polynucleotide is delivered simultaneously with the agent(s). In other embodiments, the template polynucleotide is delivered at a different time, before or after delivery of the agent(s). Any of the delivery method described herein in Section I.A.3 (e.g., in Tables 6 and 7) for delivery of nucleic acids in the agent(s) capable of inducing a targeted genetic disruption, e.g., nuclease and/or gRNAs, can be used to deliver the template polynucleotide.

In some embodiments, the one or more agent(s) and the template polynucleotide are delivered in the same format or method. For example, in some embodiments, the one or more agent(s) and the template polynucleotide are both comprised in a vector, e.g., viral vector. In some embodiments, the template polynucleotide is encoded on the same vector backbone, e.g. AAV genome, plasmid DNA, as the Cas9 and gRNA. In some aspects, the one or more agent(s) and the template polynucleotide are in different formats, e.g., ribonucleic acid-protein complex (RNP) for the Cas9-gRNA agent and a linear DNA for the template polynucleotide, but they are delivered using the same method.

In some embodiments, the template polynucleotide is a linear or circular nucleic acid molecule, such as a linear or circular DNA or linear RNA, and can be delivered using any of the methods described in Section I.A.3 herein (e.g., Tables 6 and 7 herein) for delivering nucleic acid molecules into the cell.

In particular embodiments, the polynucleotide, e.g., the template polynucleotide, are introduced into the cells in nucleotide form, e.g., as or within a non-viral vector. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (such as described in Lee, et al. (2012) Nano Lett 12: 6322-27), lipid-mediated transfection, peptide-mediated delivery, e.g., cell-penetrating peptides, or a combination thereof. In some embodiments, the non-viral polynucleotide is delivered into the cell by a non-viral method described herein, such as a non-viral method listed in Table 7 herein.

In some embodiments, the template polynucleotide sequence can be comprised in a vector molecule containing sequences that are not homologous to the region of interest in the genomic DNA. In some embodiments, the virus is a DNA virus (e.g., dsDNA or ssDNA virus). In some embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein. A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In some embodiments, the template polynucleotide can be transferred into cells using recombinant infectious virus particles, such as, e.g., vectors derived from simian virus 40 (SV40), adenoviruses, adeno-associated virus (AAV). In some embodiments, the template polynucleotide is transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557 or HIV-1 derived lentiviral vectors.

### II. Nucleic Acids, Vectors and Delivery

In some embodiments, provided are polynucleotides, such as template polynucleotides for targeting a transgene to a specific genomic target location, such as at an invariant CD3-IgSF chain locus, e.g., CD3E, CD3D, or CD3G. In some embodiments, provided are any template polynucleotides described in Section I.B herein. In some embodiments, the template polynucleotide contains a transgene including nucleic acid sequences that encode a portion, such as an antigen-binding domain, of a miniCAR and optionally linkers, polypeptides and/or factors, and homology arms for targeted integration. In some embodiments, the template polynucleotide contains a transgene includes nucleic acid sequences that an antigen-binding domain of a miniCAR, and homology arms for targeted integration at an invariant CD3-IgSF chain locus. In some embodiments, the template polynucleotide can be contained in a vector.

In some embodiments, the polynucleotide, such as a template polynucleotide encoding a transgene as described herein, is introduced into the cells in nucleotide form, such as a polynucleotide or a vector. In particular embodiments, the polynucleotide contains a transgene including a sequence encoding a binding domain, e.g., antigen-binding domain. In certain embodiments, the one or more agent(s) or components thereof for genetic disruption are introduced into the cells in nucleic acid form, such as polynucleotides and/or vectors. In some embodiments, the components for engineering can be delivered in various forms using various delivery methods, including any suitable methods used for delivery of agent(s) as described in Section I.A.3 and Tables 6 and 7 herein. Also provided are one or more polynucleotides (such as nucleic acid molecules) encoding one or more components of the one or more agent(s) capable of inducing a genetic disruption (for example, any described in Section I.A herein). Also provided are one or more template polynucleotides containing the transgene sequences (for example, any described in Section I.B.2 herein). Also provided are vectors, such as vectors for genetically engineering cells for targeted integration of the transgene, that include one or more such polynucleotides, such as a template polynucleotide or a polynucleotide encoding one or more components of the one or more agent(s) capable of inducing a genetic disruption.

In some embodiments, agents capable of inducing a genetic disruption can be encoded in one or more polynucleotides. In some embodiments, the component of the agents, such as Cas9 molecule and/or a gRNA molecule, can be encoded in one or more polynucleotides, and introduced into the cells. In some embodiments, the polynucleotide encoding one or more component of the agents can be included in a vector.

In some embodiments, a vector may comprise a sequence that encodes a Cas9 molecule and/or a gRNA molecule and/or template polynucleotides. In some aspects, a vector may also comprise a sequence encoding a signal peptide (such as for nuclear localization, nucleolar localization, mitochondrial localization), fused, such as to a Cas9 molecule sequence. For example, a vector may comprise a nuclear localization sequence (such as from SV40) fused to the sequence encoding the Cas9 molecule. In some embodiments, provided are vectors for genetically engineering cells for targeted integration of the transgene sequences contained in the polynucleotides, such as the template polynucleotides described in Section I.B.2.

In particular embodiments, one or more regulatory/control elements, such as a promoter, an enhancer, an intron, a polyadenylation signal, a Kozak consensus sequence, internal ribosome entry sites (IRES), a 2A sequence, and splice acceptor or donor can be included in the vectors. In some embodiments, the promoter is selected from among an RNA pol I, pol II or pol III promoter. In some embodiments, the promoter is recognized by RNA polymerase II (such as a CMV, SV40 early region or adenovirus major late promoter). In another embodiment, the promoter is recognized by RNA polymerase III (such as a U6 or H1 promoter).

In certain embodiments, the promoter is a regulated promoter (such as inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (sequence set forth in SEQ ID NO:18 or 126; see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter (such as set forth in SEQ ID NO:77 or 118) or a modified form thereof (EF1α promoter with HTLV1 enhancer; such as set forth in SEQ ID NO:119) or the MND promoter (such as set forth in SEQ ID NO:131). In some embodiments, the polynucleotide and/or vector does not include a regulatory element, e.g. promoter.

In particular embodiments, the polynucleotide, e.g., the polynucleotide encoding the transgene, are introduced into the cells in nucleotide form, e.g., as or within a non-viral vector. In some embodiments, the polynucleotide is a DNA or an RNA polynucleotide. In some embodiments, the polynucleotide is a double-stranded or single-stranded polynucleotide. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (such as described in Lee, et al. (2012) Nano Lett 12: 6322-27), lipid-mediated transfection, peptide-mediated delivery, or a combination thereof. In some embodiments, the non-viral polynucleotide is delivered into the cell by a non-viral method described herein, such as a non-viral method listed in Table 7.

In some embodiments, the vector or delivery vehicle is a viral vector (e.g., for generation of recombinant viruses). In some embodiments, the virus is a DNA virus (e.g., dsDNA or ssDNA virus). In some embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein.

In some embodiments, the virus infects dividing cells. In another embodiment, the virus infects non-dividing cells. In another embodiment, the virus infects both dividing and non-dividing cells. In another embodiment, the virus can integrate into the host genome. In another embodiment, the virus is engineered to have reduced immunity, e.g., in human. In another embodiment, the virus is replication-competent. In another embodiment, the virus is replication-defective, e.g., having one or more coding regions for the genes necessary for additional rounds of virion replication and/or packaging replaced with other genes or deleted. In another embodiment, the virus causes transient expression of the Cas9 molecule and/or the gRNA molecule for the purposes of transient induction of genetic disruption. In another embodiment, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the polynucleotide containing the agent(s) and/or template polynucleotide is delivered by a recombinant retrovirus. In another embodiment, the retrovirus (e.g., Moloney murine leukemia virus) comprises a reverse transcriptase, e.g., that allows integration into the host genome. In some embodiments, the retrovirus is replication-competent. In another embodiment, the retrovirus is replication-defective, e.g., having one of more coding regions for the genes necessary for additional rounds of virion replication and packaging replaced with other genes, or deleted.

In some embodiments, the polynucleotide containing the agent(s) and/or template polynucleotide is delivered by a recombinant lentivirus. For example, the lentivirus is replication-defective, e.g., does not comprise one or more genes required for viral replication.

In some embodiments, the polynucleotide containing the agent(s) and/or template polynucleotide is delivered by a recombinant adenovirus. In another embodiment, the adenovirus is engineered to have reduced immunity in humans.

In some embodiments, the polynucleotide containing the agent(s) and/or template polynucleotide is delivered by a recombinant AAV. In some embodiments, the AAV can incorporate its genome into that of a host cell, e.g., a target cell as described herein. In another embodiment, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV7, AAV8, AAV 8.2, AAV9, AAV.rh10, modified AAV.rh10, AAV.rh32/33, modified AAV.rh32/33, AAV.rh43, modified AAV.rh43, AAV.rh64R1, modified AAV.rh64R1, and pseudotyped AAV, such as AAV2/8, AAV2/5 and AAV2/6 can also be used in the disclosed methods.

In some embodiments, the polynucleotide containing the agent(s) and/or template polynucleotide is delivered by a hybrid virus, e.g., a hybrid of one or more of the viruses described herein.

A packaging cell is used to form a virus particle that is capable of infecting a target cell. Such a cell includes a 293 cell, which can package adenovirus, and a ψ2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., Cas9. For example, an AAV vector used in gene therapy typically only possesses inverted terminal repeat (ITR) sequences from the AAV genome which are required for packaging and gene expression in the host or target cell. The missing viral functions are supplied in trans by the packaging cell line.

Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the viral vector has the ability of cell type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In some embodiments, the viral vector achieves cell type specific expression. For example, a tissue-specific promoter can be constructed to restrict expression of the agent capable of introducing a genetic disruption (e.g., Cas9 and gRNA) in only a specific target cell. The specificity of the vector can also be mediated by microRNA-dependent control of expression. In some embodiments, the viral vector has increased efficiency of fusion of the viral vector and a target cell membrane. For example, a fusion protein such as fusion-competent hemagglutinin (HA) can be incorporated to increase viral uptake into cells. In some embodiments, the viral vector has the ability of nuclear localization. For example, a virus that requires the breakdown of the nuclear membrane (during cell division) and therefore will not infect a non-diving cell can be altered to incorporate a nuclear localization peptide in the matrix protein of the virus thereby enabling the transduction of non-proliferating cells.

### III. Engineered Cells Expressing a Minicar and Cell Compositions

Provided herein are genetically engineered cells containing a modified locus encoding an invariant CD3 chain of the immunoglobulin superfamily (invariant CD3-IgSF chain locus). In some aspects, the endogenous invariant CD3-IgSF chain locus encodes an endogenous invariant CD3 chain of the immunoglobulin superfamily (invariant CD3-IgSF chain). In some embodiments, the modified invariant CD3-IgSF chain locus includes nucleic acid sequences encoding a chimeric receptor such as a mini chimeric antigen receptor, also referred to herein as a miniCAR. In some embodiments, the miniCAR is a fusion protein containing a heterologous antigen-binding domain and an endogenous invariant CD3-IgSF chain. In some aspects, the modified invariant CD3-IgSF chain locus in the genetically engineered cell contains exogenous nucleic acid sequences (e.g., transgene sequences) encoding one or more portions, regions or domains of a miniCAR, such as an antigen-binding domain, integrated into the endogenous invariant CD3-IgSF chain locus.

In some aspects, the provided engineered cells are produced using methods described herein, e.g., involving homology-dependent repair (HDR) by employing agent(s) for inducing a genetic disruption (for example, described in Section I.A) and template polynucleotides containing the transgene sequences as a template for repair (for example, described in Section I.B.2). In some aspects, the provided polynucleotides, such as any template polynucleotides described in Section I.B.2, can be targeted for integration at the endogenous invariant CD3-IgSF chain locus to generate a cell containing a modified invariant CD3-IgSF chain locus containing a nucleic acid sequence encoding a miniCAR. In some aspects, the encoded miniCAR includes a heterologous antigen-binding domain and an endogenous invariant CD3-IgSF chain. In some embodiments, the template polynucleotide that is integrated by HDR into the endogenous invariant CD3-IgSF chain locus includes a transgene sequence, for example as described in in Section I.B.2.a.

In some embodiments, the provided engineered cells express a mini chimeric antigen receptor (miniCAR). In some embodiments, the provided engineered cells contain a modified invariant CD3-IgSF chain locus, e.g., a modified CD3E locus, a modified CD3D locus or a modified CD3G locus, encoding a miniCAR. In some aspects, the cells are engineered to express a miniCAR, for example described in Section III.B. In some aspects, the miniCAR is encoded by the nucleic acid sequences present at the modified invariant CD3-IgSF chain locus in the engineered cells. In some aspects, the cells are generated by integrating transgene sequences encoding a portion of the miniCAR, e.g., an antigen-binding domain, via HDR. In some embodiments, the miniCAR contains a heterologous antigen-binding domain that binds to or recognizes an antigen (or a ligand), e.g., an antigen associated with a disease or disorder. In some embodiments, the antigen (or ligand) to which the heterologous antigen-binding domain binds may be referred to as a target antigen (or a target ligand).

In some aspects, the miniCAR contains all or a portion of an endogenous invariant CD3 chain of the immunoglobulin superfamily (invariant CD3-IgSF chain). In some embodiments, the invariant CD3-IgSF chain is the endogenous invariant CD3-IgSF chain encoded by the invariant CD3-IgSF chain locus into which the template polynucleotides are targeted for integration. As such, in some embodiments, the miniCAR is a fusion protein including a heterologous antigen-binding domain fused to all or a portion of the endogenous invariant CD3-IgSF chain.

In some embodiments, the miniCAR expressed by the cell contains a heterologous antigen-binding domain fused to all or a portion of the invariant CD3-IgSF chain. In some embodiments, the miniCAR expressed by the cell contains a heterologous antigen-binding domain fused at the N-terminus of the invariant CD3-IgSF chain. In some aspects, the nucleic acid sequences encoding the miniCAR at the modified invariant CD3-IgSF chain locus includes exogenous nucleic acid sequences fused, such as fused in-frame, with an open reading frame or a partial sequence thereof of an endogenous invariant CD3-IgSF chain locus that encodes an invariant CD3-IgSF chain. In some aspects, the encoded miniCAR comprises, at minimum, a heterologous extracellular antigen-binding domain, a transmembrane domain of an invariant CD3-IgSF chain and an intracellular region of an invariant CD3-IgSF chain. In some aspects, the encoded miniCAR comprises the heterologous extracellular antigen-binding domain that can bind to the target antigen, and following binding of the target antigen, a portion of the fused endogenous invariant CD3-IgSF chain, such as the intracellular region of the invariant CD3-IgSF contained in the miniCAR, induces or transmits a stimulatory or activation signal via the TCR/CD3 complex.

In some aspects, the miniCAR described herein assembles into a TCR/CD3 complex of an immune cell, e.g., T cell, in place of the corresponding endogenous invariant CD3-IgSF chain of the TCR/CD3 complex. In some embodiments, assembly of the miniCAR into the TCR/CD3 complex results in the antigen-binding domain of the miniCAR being present on the cell surface. In some embodiments, assembly of the miniCAR into the TCR/CD3 complex allows the intracellular domain or region of the miniCAR, e.g., the intracellular region of the invariant CD3-IgSF chain, to interact with the TCR/CD3 complex. In some embodiments, binding of the antigen-binding domain of the miniCAR to a target antigen or target ligand induces signaling via the TCR/CD3 complex into which the miniCAR is assembled. For example, binding of a target antigen by the binding domain of the miniCAR can induce TCR/CD3 complex signaling at least in part via the ITAM contained in the intracellular or cytoplasmic domain of the invariant CD3-IgSF chain, e.g., CD3e, CD3d, or CD3g chain. Thus, in some aspects, the miniCAR provided herein induces stimulating or activating signals, e.g., stimulating or activating T cell intracellular signaling cascades, through the TCR/CD3 complex. In some cases, the ability of the miniCAR to assemble into and induce signaling in a TCR/CD3 complex affords the engineered cell increased persistence and/or decreased tonic signaling.

In some aspects, compared to a conventional chimeric antigen receptor (CAR), which can comprises an extracellular antigen-binding domain, optionally a spacer, a transmembrane domain, and an intracellular domain comprising a CD3zeta (CD3ζ) signaling domain, and typically a co-stimulatory signaling domain, the miniCAR is smaller in size (minimally comprising an extracellular binding domain, a transmembrane region of an invariant CD3-IgSF chain and an intracellular region of an invariant CD3-IgSF chain), and does not require a co-stimulatory signaling domain. In some aspects, by virtue of binding of the target antigen to the extracellular antigen-binding domain, the intracellular region of an invariant CD3-IgSF chain can induce or transmit a signal through the TCR/CD3 complex, at least in part via the ITAM. As the miniCAR is assembled into the TCR/CD3 complex, the binding of the extracellular antigen-binding domain to the target antigen and the activating or stimulatory signal through the TCR/CD3 complex is directly coupled, and a co-stimulatory signal is not required.

In some embodiments, the methods, compositions, articles of manufacture, and/or kits provided herein are useful to generate, manufacture, or produce genetically engineered cells, e.g., genetically engineered T cells, that have or contain a modified invariant CD3-IgSF chain locus encoding a miniCAR. In particular embodiments, the methods provided herein result in genetically engineered cells that have or contain a modified invariant CD3-IgSF chain locus. In particular embodiments, the modified invariant CD3-IgSF chain locus is or contains a fusion of a transgene, e.g., a transgene described in Section I.B, and an open reading frame of the endogenous invariant CD3-IgSF chain gene. In certain embodiments, the transgene encodes an antigen-binding domain and is inserted in-frame into the open reading frame of the endogenous invariant CD3-IgSF chain gene, resulting in a modified locus that encodes a fusion protein containing the heterologous antigen-binding domain encoded by the inserted transgene, and the endogenous invariant CD3-IgSF chain encoded by invariant CD3-IgSF chain gene. Insertion or integration of the transgene in-frame may be accomplished according to the methods provided herein, such described in Section I.B.

In some aspects, the engineered cells are T cells. In some aspects, the T cells are engineered to express a miniCAR as described herein.

Also provided are compositions containing a plurality of the engineered cells. In some aspects, the compositions containing the engineered cells exhibit improved, uniform, homogeneous and/or stable expression and/or antigen binding by the encoded miniCAR compared to cells or cell compositions generated using other methods of engineering, such as methods in which the nucleic acid sequences encoding a chimeric receptor is introduced randomly into the genome of a cell. In some embodiments, the engineered cells exhibit increased persistence compared to T cells engineered with a chimeric receptor, e.g., a chimeric antigen receptor (CAR), that contains the same antigen-binding domain. In some embodiments, the engineered cells exhibit increased cytolytic activity compared to T cells engineered with a CAR that contains the same antigen-binding domain. In some embodiments, the engineered cells exhibit reduced tonic signaling via the endogenous TCR/CD3 complex compared to T cells engineered with a CAR that contains the same antigen-binding domain.

In some embodiments, the engineered cells or the composition comprising the engineered cells can be used in therapy, e.g., adoptive cell therapy. In some embodiments, the provided cells or cell compositions can be used in any of the methods of treatment described herein or for therapeutic uses described herein.

In some embodiments, the engineered cells can also express one or more additional molecules, e.g., a marker, an additional chimeric receptor polypeptides, an antibody or an antigen-binding fragment thereof, an immunomodulatory molecule, a ligand, a cytokine or a chemokine. In some aspects, the transgene sequence encoding a portion thereof the miniCAR, e.g., antigen-binding domain, contained in the polynucleotides, is integrated at the endogenous invariant CD3-IgSF chain locus, e.g., CD3E, CD3D, or CD3G locus, of the engineered cell, to result in a modified invariant CD3-IgSF chain locus, e.g., CD3E, CD3D, or CD3G locus, that encodes a miniCAR as described herein.

A. Modified Invariant CD3-IgSF Chain Locus

In some aspects, provided are genetically engineered cells comprising a modified invariant CD3-IgSF chain locus, e.g., CD3E, CD3D, or CD3G locus. In some embodiments, the modified invariant CD3-IgSF chain locus includes a heterologous nucleic acid sequence encoding a portion, such as an antigen-binding domain, of a miniCAR. In some embodiments, the nucleic acid sequence includes a transgene sequence encoding an antigen-binding domain, for example a transgene as described herein (see, e.g., Section I.B), the transgene sequence having been integrated at the endogenous invariant CD3-IgSF chain locus, optionally via homology directed repair (HDR). In some embodiments, the nucleic acid sequence includes a fusion of a transgene sequence encoding a heterologous antigen-binding domain and an open reading frame of the endogenous invariant CD3-IgSF chain locus.

In some aspects, the modified invariant CD3-IgSF chain locus is generated as a result of genetic disruption and integration of transgene sequences, e.g., as described in Section I.A, above, such as via HDR methods. In some aspects, the nucleic acid sequence present at the modified invariant CD3-IgSF chain locus includes a transgene sequence as described herein, integrated at a region in the endogenous invariant CD3-IgSF chain locus that is 5′ to all or a portion of the open reading frame sequences encoding the invariant CD3-IgSF chain. In some aspects, the nucleic acid sequence present at the modified invariant CD3-IgSF chain locus includes a transgene sequence as described herein, integrated at a region in the endogenous invariant CD3-IgSF chain locus that is 5′ to the sequences encoding a full length invariant CD3-IgSF chain, such as a full-length mature invariant CD3-IgSF chain. Thus, in some embodiments, a transgene sequence as described herein is integrated to avoid disrupting the sequences encoding the endogenous invariant CD3-IgSF chain. In some embodiments, a transgene sequence as described herein is integrated in-frame with the encoded sequence of the endogenous invariant CD3-IgSF chain. In some embodiments, a transgene sequence as described herein is integrated in-frame and upstream, e.g., 5′, to the sequences encoding the endogenous invariant CD3-IgSF chain. Thus, in some embodiments, the miniCAR fusion protein expressed from the modified invariant CD3-IgSF chain locus includes the expressed transgene fused to the N-terminus of the full length, optionally mature, invariant CD3-IgSF chain.

In some aspects, upon targeted integration of the transgene by HDR, the genome of the cell contains a modified invariant CD3-IgSF chain locus, containing a nucleic acid sequence encoding a fusion protein, e.g., miniCAR, that includes a heterologous antigen-binding domain and an endogenous invariant CD3-IgSF chain. In some embodiments, upon targeted integration, the modified invariant CD3-IgSF chain locus contains a fusion of the transgene, for example a transgene as described herein, and an open reading frame of an endogenous invariant CD3-IgSF chain locus. In some embodiments, upon targeted integration, the modified invariant CD3-IgSF chain locus contains a transgene as described herein, integrated into a site within the open reading frame of the endogenous invariant CD3-IgSF chain locus. In some embodiments, upon targeted integration, the modified invariant CD3-IgSF chain locus contains nucleic acid sequences, e.g., a DNA sequence, encoding an antigen-binding domain encoded the by a transgene as described herein, and the endogenous invariant CD3-IgSF chain encoded by the invariant CD3-IgSF chain locus.

In some embodiments, the integrated transgene comprises in order from 5′ to 3′ a sequence of nucleotides encoding one or more of a multicistronic element, an antigen-binding domain, and a linker. In some aspects, the integrated transgene encodes a multicistronic element and an antigen-binding domain. In some aspects, the integrated transgene encodes a multicistronic element, an antigen-binding domain, and a linker. In some aspects, the integrated transgene encodes an antigen-binding domain and a linker. In some embodiments, the multicistronic element is or includes a ribosome skip sequence. In some embodiments, the integrated transgene contains a ribosomal skipping element upstream, e.g., immediately upstream, of the sequence of nucleic acids encoding the antigen-binding domain. In some embodiments, the ribosome skip sequence is a T2A, a P2A, an E2A, or an F2A element. In some embodiments, the ribosome skip sequence is a P2A element.

In some embodiments, the integration of the transgene generates a gene fusion of transgene and endogenous sequences of the invariant CD3-IgSF chain locus, which together encode a miniCAR fusion protein containing an antigen-binding domain and an endogenous invariant CD3-IgSF chain, optionally a full length, optionally mature, invariant CD3-IgSF chain. In some embodiments, the mRNA transcribed from the modified invariant CD3-IgSF chain locus contains a 3′UTR that is encoded by the endogenous invariant CD3-IgSF chain locus and/or is identical to a 3′UTR of an mRNA that is transcribed from the endogenous invariant CD3-IgSF chain locus. In some embodiments, the mRNA transcribed from the transgene contains a 5′UTR that is encoded by the endogenous gene and/or is identical to a 5′UTR of an mRNA that is transcribed from the endogenous invariant CD3-IgSF chain locus.

In some embodiments, the modified invariant CD3-IgSF chain locus includes in order from 5′ to 3′, a sequence of nucleotides encoding a multicistronic element as described herein, optionally a P2A element; a sequence of nucleotides encoding an antigen-binding domain as described herein; and a sequence of nucleotides encoding an invariant CD3-IgSF chain, e.g., from the endogenous invariant CD3-IgSF locus. In some embodiments, the modified invariant CD3-IgSF chain locus includes in order from 5′ to 3′, a sequence of nucleotides encoding a multicistronic element as described herein, optionally a P2A element; a sequence of nucleotides encoding an antigen-binding domain as described herein; a linker as described herein; and a sequence of nucleotides encoding an invariant CD3-IgSF chain. In some embodiments, the modified invariant CD3-IgSF chain locus encodes a miniCAR that is a fusion protein containing, in order from N- to C-terminus, an antigen-binding domain as described herein and an invariant CD3-IgSF chain, such as a full length mature invariant CD3-IgSF chain. In some embodiments, the modified invariant CD3-IgSF chain locus encodes a miniCAR that is a fusion protein containing, in order from N- to C-terminus, an antigen-binding domain as described herein, a linker as described herein, and an invariant CD3-IgSF chain, such as a full length mature invariant CD3-IgSF chain.

In some embodiments, the miniCAR fusion protein by the modified invariant CD3-IgSF chain locus is functional, for example is capable of assembly into the TCR/CD3 complex, either spontaneously or following binding of the antigen to the antigen-binding domain, and transmitting or transducing a cellular signal, particularly following assembly into a TCR/CD3 complex. In some embodiments, the miniCAR assembles into a TCR/CD3 complex in place of the corresponding endogenous invariant CD3-IgSF chain of the TCR/CD3 complex. In some embodiments, a miniCAR encoded by the modified locus binds to a target antigen. In some embodiments, the target antigen is associated with, specific to, and/or expressed on a cell or tissue that is associated with a disease, disorder, or condition. In some embodiments, the miniCAR encoded by the modified invariant CD3-IgSF chain locus is a functional fusion protein that induces a primary activation signal in a T cell via the TCR/CD3 complex following binding of the antigen-binding domain of the miniCAR to a target antigen.

B. Encoded MiniCAR Fusion Proteins

In some embodiments, the chimeric receptors encoded by the engineered cells provided herein, or the engineered cells generated according to the methods provided herein, include a miniCAR, for example, that is a fusion proteins that contain a heterologous antigen-binding domain and all or a portion of an endogenous invariant CD3 chain of the immunoglobulin superfamily (invariant CD3-IgSF chain). In some aspects, at least a portion of the miniCAR is encoded by transgene sequences present in the polynucleotides provided herein, such as any template polynucleotides described in Section I.B.2 above. In some aspects, a transgene sequence encoding a portion of the miniCAR contained in the polynucleotides, e.g., an antigen-binding domain, is integrated at an endogenous invariant CD3-IgSF chain locus, e.g., CD3E, CD3D, or CD3G locus, of the engineered cell, to result in a modified invariant CD3-IgSF chain locus that encodes a miniCAR, such as any miniCAR described herein. In some aspects, the modified invariant CD3-IgSF chain locus includes a transgene as described herein and an open reading frame sequence of the endogenous invariant CD3-IgSF chain locus. In some embodiments, provided are engineered cells, such as T cells, that express one or more miniCAR fusion proteins.

In some embodiments, the antigen-binding domain contained in the miniCAR is or includes an antibody or an antigen-binding fragment thereof. In some embodiments, the antigen-binding domain is or includes a Fab fragment, a Fab₂ fragment, a single domain antibody, or a single chain variable fragment (scFv). In some embodiments, the antigen-binding domain is an antigen-binding domain as described herein, e.g., in Section III.B.1.

In some embodiments, the miniCAR encoded in the genetically engineered cells provided herein, generally contains an extracellular antigen-binding domain (encoded by the transgene), an extracellular region of the endogenous invariant CD3-IgSF chain (encoded by the endogenous invariant CD3-IgSF locus), for example, an endogenous CD3e, CD3d or CD3g, a transmembrane region of the endogenous invariant CD3-IgSF chain, and an intracellular region of the endogenous invariant CD3-IgSF chain. In some embodiments, the extracellular region, the transmembrane, and the intracellular region are the regions of an endogenous invariant CD3-IgSF chain, for example, an endogenous CD3e, CD3d or CD3g, and are encoded by the endogenous invariant CD3-IgSF locus. In some embodiments, the regions are the full length mature endogenous invariant CD3-IgSF chain regions, for example, full length mature endogenous CD3e, CD3d or CD3g. In some embodiments, the miniCAR encoded in the genetically engineered cells provided herein, generally contains various regions or domains such as one or more of an antigen-binding domain, a linker, an extracellular region of the endogenous invariant CD3-IgSF chain, a transmembrane region of the endogenous invariant CD3-IgSF chain, and an intracellular region of the endogenous invariant CD3-IgSF chain. In some embodiments, miniCAR includes an extracellular antigen-binding domain encoded by a transgene, a linker, an extracellular region, a transmembrane region, and an intracellular region.

In some embodiments, the antigen-binding domain of the miniCAR, encoded in the genetically engineered cells, is linked, directly or indirectly, to the extracellular domain of an endogenous invariant CD3-IgSF chain, for example, an endogenous CD3e, CD3d or CD3g. In some embodiments, the antigen-binding domain is indirectly linked to the extracellular domain of the endogenous invariant CD3-IgSF chain through a linker, e.g., a flexible linker as described herein (see, e.g., Section III.B.2). In some cases, the linker that separates or is positioned between the antigen-binding domain and the extracellular domain, e.g., the extracellular region of the endogenous invariant CD3-IgSF chain, thereby allowing the antigen-binding domain to avoid steric hindrance and attain its tertiary structure. In some aspects, the encoded miniCAR further contains other domains, linkers and/or regulatory elements.

In some embodiments, the encoded chimeric receptor is a miniCAR. An exemplary miniCAR sequence includes in order from N- to C-terminus: an antigen-binding domain, an extracellular region of the endogenous invariant CD3-IgSF chain, a transmembrane region of the endogenous invariant CD3-IgSF chain, and an intracellular region of the endogenous invariant CD3-IgSF chain. In some embodiments, an exemplary miniCAR sequence includes in order from N- to C-terminus: an antigen-binding domain, a linker, an extracellular region of the endogenous invariant CD3-IgSF chain, a transmembrane region of the endogenous invariant CD3-IgSF chain, and an intracellular region of the endogenous invariant CD3-IgSF chain. In some embodiments, the extracellular region, the transmembrane region, and the intracellular region are the regions or domains of an endogenous invariant CD3-IgSF chain, e.g., CD3e, CD3d or CD3g, optionally domains of the full length and mature endogenous invariant CD3-IgSF chain, encoded by the endogenous invariant CD3-IgSF chain locus into which a transgene as described herein is integrated.

In some embodiments, an exemplary encoded precursor miniCAR comprises, in its N- to C-terminus order: a signal peptide, an antigen-binding domain, an extracellular region of the endogenous invariant CD3-IgSF chain, a transmembrane region of the endogenous invariant CD3-IgSF chain, and an intracellular region of the endogenous invariant CD3-IgSF chain, wherein the nucleic acid sequence encoding the miniCAR is present in a modified invariant CD3-IgSF chain locus, e.g., CD3E, CD3D, or CD3G locus. In some embodiments, an exemplary encoded precursor miniCAR comprises, in its N- to C-terminus order: a signal peptide, an antigen-binding domain, a linker, an extracellular region of the endogenous invariant CD3-IgSF chain, a transmembrane region of the endogenous invariant CD3-IgSF chain, and an intracellular region of the endogenous invariant CD3-IgSF chain, wherein the nucleic acid sequence encoding the miniCAR is present in a modified invariant CD3-IgSF chain locus, e.g., CD3E, CD3D, or CD3G locus. In some embodiments, the extracellular region, the transmembrane region, and the intracellular region are the domains of the endogenous invariant CD3-IgSF chain encoded by the endogenous invariant CD3-IgSF chain locus, e.g., CD3E, CD3D, or CD3G locus.

In some embodiments, an exemplary encoded precursor miniCAR sequence comprises, in order from 5′ to 3′: a sequence of nucleotides encoding a signal peptide; a multicistronic element, optionally a ribosomal skip sequence, optionally a P2A sequence; an antigen-binding domain, optionally a single chain variable fragment (scFv); an extracellular region of the endogenous invariant CD3-IgSF chain, a transmembrane region of the endogenous invariant CD3-IgSF chain, and an intracellular region of the endogenous invariant CD3-IgSF chain. In some embodiments, an exemplary encoded precursor miniCAR sequence comprises, in order from 5′ to 3′: a sequence of nucleotides encoding a signal peptide; a multicistronic element, optionally a ribosomal skip sequence, optionally a P2A sequence; an antigen-binding domain, optionally a single chain variable fragment (scFv); a linker, optionally a linker having the sequence set forth by SEQ ID NO: 16; an extracellular domain; an extracellular region of the endogenous invariant CD3-IgSF chain, a transmembrane region of the endogenous invariant CD3-IgSF chain, and an intracellular region of the endogenous invariant CD3-IgSF chain. In some embodiments, the extracellular region, the transmembrane region, and the intracellular region are encoded by the invariant CD3-IgSF chain locus, e.g., CD3E, CD3D, or CD3G locus. The encoded precursor polypeptide, e.g., a precursor miniCAR, can include the signal peptide sequence, typically at the N-terminal of the encoded polypeptide. In the mature form of the expressed miniCAR, the signal sequence is cleaved from the remaining portions of the miniCAR.

1. Binding Domains

In some embodiments, the extracellular region of the encoded miniCAR includes a binding domain. In some embodiments, the binding domain is an extracellular binding domain. In some embodiments, the binding domain is or comprises a polypeptide, a ligand, a receptor, a ligand-binding domain, a receptor-binding domain, an antigen, an epitope, an antibody, an antigen-binding domain, an epitope-binding domain, an antibody-binding domain, a tag-binding domain or a fragment of any of the foregoing. In some embodiments, the binding domain is an antigen-binding domain or a ligand-binding domain. In some embodiments, the binding domain is an antigen-binding domain. In some aspects, as described above, the binding domain, e.g., antigen-binding domain, of the miniCAR is encoded by a transgene that is integrated at the invariant CD3-IgSF chain locus.

In some aspects, the extracellular binding domain, such as an antigen-binding domain, is linked or connected, either directly or indirectly to an extracellular region or domain of an invariant CD3-IgSF chain. In some embodiments, the antigen-binding domain of the miniCAR is linked to the extracellular region of the invariant CD3-IgSF chain via a linker. In some embodiments, the linker is flexible linker, for example a linker as described in Section III.B.2 below. In some embodiments, the antigen-binding domain is linked to a transmembrane region or domain and an intracellular region or domain(s) of the invariant CD3-IgSF chain through the extracellular region of the invariant CD3-IgSF chain. In some embodiments, the miniCAR includes a transmembrane region disposed between the extracellular region and the intracellular region. In some aspects, the binding domain, e.g., antigen-binding domain, is linked directly or indirectly, via a linker, to a full length, mature invariant CD3-IgSF chain.

In some embodiments, the antigen, e.g., an antigen (also called a “target antigen”) that binds the antigen-binding domain of the miniCAR, is a polypeptide. In some embodiments, the antigen is a carbohydrate or other molecule. In some embodiments, the antigen is selectively expressed or overexpressed on cells of the disease, disorder or condition, e.g., the tumor or pathogenic cells, as compared to normal or non-targeted cells or tissues, e.g., in healthy cells or tissues. In some embodiments, the disease, disorder or condition is an infectious disease or disorder, an autoimmune disease, an inflammatory disease, or a tumor or a cancer. In some embodiments, the antigen is expressed on normal cells and/or is expressed on the engineered cells. In some aspects, the miniCAR includes one or more regions or domains selected from an extracellular antigen-binding (or ligand-binding) or region or domains, e.g., any of the antibody or fragment described herein.

In some embodiments, the antigen-binding domain of the miniCAR includes an antigen-binding portion or portions of an antibody molecule, such as a single-chain antibody fragment (scFv) derived from the variable heavy (V_{H}) and variable light (V_{L}) chains of a monoclonal antibody (mAb), or a single domain antibody (sdAb).

In some embodiments, the antigen-binding domain is or comprises an antibody or an antigen-binding fragment (e.g. scFv) that specifically recognizes an antigen, such as an intact antigen, expressed on the surface of a cell. In some embodiments, the antigen, is a protein expressed on the surface of cells. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, among the antigens targeted by the miniCAR, e.g., via the antigen-binding domain, are those expressed in the context of a disease, condition, or cell type to be targeted via the adoptive cell therapy. Among the diseases and conditions are proliferative, neoplastic, and malignant diseases and disorders, including cancers and tumors, including hematologic malignancy, cancers of the immune system, such as lymphomas, leukemias, and/or myelomas, such as B, T, and myeloid leukemias, lymphomas, and multiple myelomas.

In some embodiments, the antigen or ligand is a tumor antigen or cancer marker. In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CDS, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19.

In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723. In some embodiments, exemplary antibody or antibody fragment include those described in U.S. Patent Publication No. WO 2014/031687, US 2016/0152723 and WO 2016/033570.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises a CDR-H1 and a CDR-H2 set forth in SEQ ID NOS: 97 and 98, respectively, and a CDR-H3 set forth in SEQ ID NO: 99 or 100; and a CDR-L1 set forth in SEQ ID NO: 101 and a CDR-L2 set forth in SEQ ID NO: 102 or 103 and a CDR-L3 set forth in SEQ ID NO: 104 or 105. In some embodiments, the FMC63 antibody comprises a heavy chain variable region (V_{H}) comprising the amino acid sequence of SEQ ID NO: 106 and a light chain variable region (V_{L}) comprising the amino acid sequence of SEQ ID NO: 107.

In some embodiments, the scFv comprises a variable light chain containing a CDR-L1 sequence of SEQ ID NO:101, a CDR-L2 sequence of SEQ ID NO:102, and a CDR-L3 sequence of SEQ ID NO:108 and/or a variable heavy chain containing a CDR-H1 sequence of SEQ ID NO:97, a CDR-H2 sequence of SEQ ID NO:98, and a CDR-H3 sequence of SEQ ID NO:99. In some embodiments, the scFv comprises a variable heavy chain region set forth in SEQ ID NO:106 and a variable light chain region set forth in SEQ ID NO:107. In some embodiments, the variable heavy and variable light chains are connected by a linker. In some embodiments, the linker is set forth in SEQ ID NO:109. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:110 or a sequence that exhibits at least 85%, 86%, 87%, 88%, 89%, 90%, 91%, 92%, 93%, 94%, 95%, 96%, 97%, 98%, or 99% sequence identity to SEQ ID NO:110. In some embodiments, the scFv comprises the sequence of amino acids set forth in SEQ ID NO:111 or a sequence that exhibits at least 85%, 86%, 87%, 88%, 89%, 90%, 91%, 92%, 93%, 94%, 95%, 96%, 97%, 98%, or 99% sequence identity to SEQ ID NO:111.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises a CDR-H1, a CDR-H2 and a CDR-H3 sequence set forth in SEQ ID NOS: 112-114, respectively, and a CDR-L1, a CDR-L2 and a CDR-L3 sequence set forth in SEQ ID NOS: 115-117, respectively. In some embodiments, the SJ25C1 antibody comprises a heavy chain variable region (V_{H}) comprising the amino acid sequence of SEQ ID NO: 118 and a light chain variable region (V_{L}) comprising the amino acid sequence of SEQ ID NO: 119.

In some embodiments, the scFv comprises a variable light chain containing a CDR-L1 sequence of SEQ ID NO:115, a CDR-L2 sequence of SEQ ID NO: 116, and a CDR-L3 sequence of SEQ ID NO:117 and/or a variable heavy chain containing a CDR-H1 sequence of SEQ ID NO:112, a CDR-H2 sequence of SEQ ID NO:113, and a CDR-H3 sequence of SEQ ID NO:114. In some embodiments, the scFv comprises a variable heavy chain region set forth in SEQ ID NO:118 and a variable light chain region set forth in SEQ ID NO:119. In some embodiments, the variable heavy and variable light chain are connected by a linker. In some embodiments, the linker is set forth in SEQ ID NO:16. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:120 or a sequence that exhibits at least 85%, 86%, 87%, 88%, 89%, 90%, 91%, 92%, 93%, 94%, 95%, 96%, 97%, 98%, or 99% sequence identity to SEQ ID NO:120.

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as is m971 scFv.

In some embodiments, the antigen is BCMA. In some embodiments, the scFv contains a V_{H} and a V_{L} derived from an antibody or an antibody fragment specific to BCMA. In some embodiments, the antibody or antibody fragment that binds BCMA is or contains a V_{H} and a V_{L} from an antibody or antibody fragment set forth in International Patent Applications, Publication Number WO 2016/090327, WO 2016/090320 and WO 2019/090003. In some embodiments, the antibody or antibody fragment that binds BCMA is or contains binding domains from an antibody or antibody fragment set forth in U.S. Ser. No. 10/072,088 and US 2017/0051068. In some embodiments, the antibody or antigen-binding domain can be any anti-BCMA antibody or antigen-binding fragment thereof described or derived from, for example, Carpenter et al., Clin Cancer Res., 2013, 19(8):2048-2060, WO 2016/090320, WO 2016/090327, WO 2010/104949, WO 2017/173256, WO 2017/031104, US 2020/0190205, WO2017/025038 and WO2019/000223.

In some embodiments, the antigen is ROR1. In some embodiments, the scFv contains a V_{H} and a V_{L} derived from an antibody or an antibody fragment specific to ROR1. In some embodiments, the antibody or antibody fragment that binds ROR1 is or contains a V_{H} and a V_{L} from an antibody or antibody fragment set forth in International Patent Applications, Publication Number WO 2014/031687, WO 2016/115559 and WO 2020/160050.

In some embodiments, the antigen is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Patent Applications, Publication Number WO 2016/090329, WO 2016/090312 and WO 2020/092854.

In some embodiments, the antigen is FcRL5. In some embodiments, the scFv contains a V_{H} and a V_{L} derived from an antibody or an antibody fragment specific to FcRL5. In some embodiments, the antibody or antibody fragment that binds FcRL5 is or contains a V_{H} and a V_{L} from an antibody or antibody fragment set forth in International Patent Applications, Publication Number WO 2016/090337 and WO 2017/096120.

In some embodiments, the antigen is mesothelin. In some embodiments, the scFv contains a V_{H} and a V_{L} derived from an antibody or an antibody fragment specific to mesothelin. In some embodiments, the antibody or antibody fragment that binds mesothelin is or contains a V_{H} and a V_{L} from an antibody or antibody fragment set forth in US2018/0230429.

In some embodiments, the antigen-binding domain is or comprises an antigen-binding portion or portions of an antibody molecule, such as a single-chain antibody fragment (scFv) derived from the variable heavy (V_{H}) and variable light (V_{L}) chains of a monoclonal antibody (mAb), or a single domain antibody (sdAb), such as sdFv, nanobody, V_{H}H and V_{NAR}. In some embodiments, an antigen-binding fragment comprises antibody variable regions joined by a flexible linker.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody, V_{H}H or V_{NAR}) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some aspects, the miniCAR is a bispecific miniCAR, e.g., containing two antigen-binding domains with different specificities.

In some embodiments, the antigen-binding domain of the miniCAR specifically recognizes the same antigen as a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In some embodiments, the antibody light chain constant region is chosen from, e.g., kappa or lambda, particularly kappa.

Among the binding domains of the encoded miniCAR are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAbs) are antibody fragments comprising all or a portion of the heavy chain variable domain or all or a portion of the light chain variable domain of an antibody. In certain embodiments, the single domain antibody (sdAb) is a human single domain antibody. In some embodiments, the miniCAR comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include nanobodies, camelid antibodies (e.g. V_{H}H), or shark antibodies (e.g. IgNAR). In some embodiments, a variable domain of a sdAb comprises three CDRs and four framework regions, designated FR1, CDR1, FR2, CDR2, FR3, CDR3, and FR4. In some embodiments, a sdAb variable domain may be truncated at the N-terminus or C-terminus such that it comprise only a partial FR1 and/or FR4, or lacks one or both of those framework regions, so long as the sdAb variable domain substantially maintains antigen binding and specificity. Exemplary sdAbs contemplated for use according to the compositions and methods described herein include sdAbs known to bind antigens associated with a disease, disorder, or condition, including sdAbs described in, for example, WO2017/025038 and WO2019/000223.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the encoded miniCARs, including TCR-like miniCARs, includes an extracellular portion containing an antibody or antibody fragment. In some embodiments, the antibody or fragment includes an scFv. In some aspects, the antibody or antigen-binding fragment can be obtained by screening a plurality, such as a library, of antigen-binding fragments or molecules, such as by screening an scFv library for binding to a specific antigen or ligand.

In some embodiments, the encoded miniCAR is a multi-specific CAR, e.g., contains a plurality of ligand- (e.g., antigen-) binding domains that can bind and/or recognize, e.g., specifically bind, a plurality of different antigens. In some aspects, the encoded miniCAR is a bispecific miniCAR, for example, targeting two antigens, such as by containing two antigen-binding domains with different specificities. In some embodiments, the miniCAR contains a bispecific binding domain, e.g., a bispecific antibody or fragment thereof, containing at least one antigen-binding domain binding to different surface antigens on a target cell, e.g., selected from any of the listed antigens as described herein, e.g. CD19 and CD22 or CD19 and CD20. In some embodiments, binding of the bispecific binding domain to each of its epitope or antigen can result in stimulation of function, activity and/or responses of the T cell, e.g., cytotoxic activity and subsequent lysis of the target cell. Among such exemplary bispecific binding domain can include tandem scFv molecules, in some cases fused to each other via, e.g. a flexible linker; diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; bispecific T cell engager (BiTE) molecules, which contain tandem scFv molecules fused by a flexible linker (see e.g. Nagorsen and Bauerle, Exp Cell Res 317, 1255-1260 (2011); or triomabs that include whole hybrid mouse/rat IgG molecules (Seimetz et al, Cancer Treat Rev 36, 458-467 (2010). Any of such binding domains can be contained in any of the miniCARs described herein.

In some aspects, the encoded miniCAR contains an antigen-binding domain that binds or recognizes, e.g., specifically binds, a universal tag or a universal epitope. In some aspects, the binding domain can bind a molecule, a tag, a polypeptide and/or an epitope that can be linked to a different binding molecule (e.g., antibody or antigen-binding fragment) that recognizes an antigen associated with a disease or disorder. Exemplary tag or epitope includes a dye (e.g., fluorescein isothiocyanate) or a biotin. In some aspects, a binding molecule (e.g., antibody or antigen-binding fragment) linked to a tag, that recognizes the antigen associated with a disease or disorder, e.g., tumor antigen, with an engineered cell expressing a miniCAR specific for the tag, to effect cytotoxicity or other effector function of the engineered cell. In some aspects, the specificity of the miniCAR to the antigen associated with a disease or disorder is provided by the tagged binding molecule (e.g., antibody), and different tagged binding molecule can be used to target different antigens. Exemplary binding domains specific for a universal tag or a universal epitope include those described, e.g., in U.S. Pat. No. 9,233,125, WO 2016/030414, Urbanska et al., (2012) Cancer Res 72: 1844-1852, and Tamada et al., (2012) Clin Cancer Res 18:6436-6445.

In some embodiments, the encoded miniCAR contains a TCR-like antibody, such as an antibody or an antigen-binding fragment (e.g. scFv) that specifically recognizes an intracellular antigen, such as a tumor-associated antigen, presented on the cell surface as a major histocompatibility complex (MHC)-peptide complex. In some embodiments, an antibody or antigen-binding portion thereof that recognizes an MHC-peptide complex can be expressed on cells as part of a miniCAR. In some embodiments, a miniCAR containing an antibody or antigen-binding fragment that exhibits TCR-like specificity directed against peptide-MHC complexes also may be referred to as a TCR-like miniCAR. In some embodiments, the miniCAR is a TCR-like miniCAR and the antigen is a processed peptide antigen, such as a peptide antigen of an intracellular protein, which, like a TCR, is recognized on the cell surface in the context of an MHC molecule. In some embodiments, the extracellular antigen-binding domain specific for an MHC-peptide complex of a TCR-like miniCAR is linked to one or more intracellular signaling components, in some aspects via linkers, extracellular regions or domains, and/or transmembrane regions or domain(s). In some embodiments, such molecules can typically mimic or approximate a signal through a natural antigen receptor, such as a TCR, and, optionally, a signal through such a receptor in combination with a costimulatory receptor.

In some embodiments, Major histocompatibility complex (MHC) includes a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated (32 microglobulin. Generally, MHC class II molecules are composed of two transmembrane glycoproteins, a and (3, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4⁺ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like miniCAR or antigen-binding portions thereof.

In some embodiments, a peptide, such as a peptide antigen or epitope, of a polypeptide can associate with an MHC molecule, such as for recognition by an antigen-binding domain. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like miniCAR, produces or triggers an activation signal to the T cell that induces a T cell response, such as T cell proliferation, cytokine production, a cytotoxic T cell response or other response.

In some embodiments, a TCR-like antibody or antigen-binding domain, are known or can be produced by known methods (see e.g. US Pat. App. Pub. Nos. US 2002/0150914; US 2003/0223994; US 2004/0191260; US 2006/0034850; US 2007/00992530; US20090226474; US20090304679; and International App. Pub. No. WO 03/068201).

In some embodiments, an antibody or antigen-binding domain thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described herein. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding domains thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US Pat. App. Pub. No. US20020150914, US20140294841; and Cohen CJ. et al. (2003) J Mol. Recogn. 16:324-332.

2. Linker

In some aspects, the encoded miniCAR includes an extracellular binding domain, e.g., antigen-binding domain, and an invariant CD3-IgSF chain including all or a portion of an extracellular region of the invariant CD3-IgSF chain. In some embodiments, the binding domain of the miniCAR, e.g., antigen-binding domain, is linked to the extracellular region of the invariant CD3-IgSF chain indirectly, e.g., via a linker. In some aspects, the inclusion of a linker improves binding of the antigen-binding domain to its target, e.g., target antigen. In some aspects, the inclusion of a linker allows for flexibility and/or conformational changes necessary to induce a stimulating or activating signal via the TCR/CD3 complex following binding of the binding domain, e.g., antigen-binding domain, to its target, e.g., target antigen.

In some embodiments, the linker is a polypeptide linker. In some embodiments, a short oligo- or polypeptide linker, for example, a polypeptide linker of between 2 and 25 amino acids in length, such as one containing glycines and serines, e.g., glycine-serine doublet, is present and forms a linkage between the binding domain, e.g., antigen-binding domain, and the extracellular domain or region of the invariant CD3-IgSF chain. In some embodiments, the linker is a flexible linker. In some embodiments, the linker is a peptide linker. The linker may be 2-25 amino acids in length, such as 2, 3, 4, 5, 6, 7, 8, 9, 10, 11, 12, 13, 14, 15, 16, 17, 18, 19, 20, 21, 22, 23, 24, or 25 amino acids. In some embodiments, the linker is a polypeptide that is 2, 3, 4, 5, 6, 7, 8, 9, 10, 11, 12, 13, 14, 15, 16, 17, 18, 19 or 20 amino acids in length. In some embodiments, the linker is a polypeptide that is 3 to 18 amino acids in length. In some embodiments, the linker is a polypeptide that is 12 to 18 amino acids in length. In some embodiments, the linker is a polypeptide that is 15 to 18 amino acids in length.

Various polypeptide linkers are known (see e.g. Chen et al. (2013) Adv. Drug. Deliv. 65:1357-1369; and International PCT publication No. WO 2014/099997, WO2000/24884; U.S. Pat. Nos. 5,258,498; 5,525,491; 5,525,491, 6,132,992).

The linkers can be naturally-occurring, synthetic or a combination of both. Particularly suitable linker polypeptides predominantly include amino acid residues selected from Glycine (Gly), Serine (Ser), Alanine (Ala), and Threonine (Thr). For example, the linker may contain at least 75% (calculated on the basis of the total number of residues present in the peptide linker), such as at least 80%, at least 85%, or at least 90% of amino acid residues selected from Gly, Ser, Ala, and Thr. The linker may also consist of Gly, Ser, Ala and/or Thr residues only. In some embodiments, the linker contains 1-25 glycine residues, 5-20 glycine residues, 5-15 glycine residues, or 8-12 glycine residues. In some aspects, suitable peptide linkers typically contain at least 50% glycine residues, such as at least 75% glycine residues. In some embodiments, a peptide linker comprises glycine residues only. In some embodiments, a peptide linker comprises glycine and serine residues only.

Among the linkers are those rich in glycine and serine and/or in some cases threonine. In some embodiments, the linker comprises 10 to 20 residues, such as at least or about 10, 15, or 20 residues. In some embodiments, the linker sequence comprises the amino acid sequence set forth in SEQ ID NO: 121 ((GGGGS)n), where n is an integer between 1 and inclusive. In some embodiments, the linker comprises GS, GGS, GGGGS (SEQ ID NO:122), GGGGGS (SEQ ID NO:128) and combinations thereof. In some embodiments, the linker comprises (GGS)n, wherein n is 1 to 10, (GGGGS)n (SEQ ID NO: 121), wherein n is 1 to or (GGGGGS)n (SEQ ID NO:129), wherein n is 1 to 4. In some embodiments, the linker is selected from among a linker that is or comprises GGS, is or comprises GGGGS (SEQ ID NO: 122), is or comprises GGGGGS (SEQ ID NO: 128), is or comprises (GGS)2 (SEQ ID NO: 130), is or comprises GGSGGSGGS (SEQ ID NO: 131), is or comprises GGSGGSGGSGGS (SEQ ID NO:132), is or comprises GGSGGSGGSGGSGGS (SEQ ID NO:133), is or comprises GGGGGSGGGGGSGGGGGS (SEQ ID NO:134), is or comprises GGSGGGGSGGGGSGGGGS (SEQ ID NO: 135), is or comprises and GGGGSGGGGSGGGGS (SEQ ID NO:16). In some embodiments, the linker sequence is the amino acid sequence set forth in SEQ ID NO:122 (GGGGS). In some embodiments, the linker sequence is the amino acid sequence set forth in SEQ ID NO: 123 (GGGGSGGGGS). In some embodiments, the linker sequence is the amino acid sequence set forth in SEQ ID NO: 16 (GGGGSGGGGSGGGGS). In some embodiments, the linker sequence is the amino acid sequence set forth in SEQ ID NO: 124 (GGGGSGGGGSGGGGSGGGGS). In some embodiments, the linker is (G₄S)₃₋₄ (SEQ ID NO:125). In some embodiments, the linker is (G₄S)₂₋₃ (SEQ ID NO:126) or GGGAS(G₄S)₂ (SEQ ID NO:127). In some embodiments, the linker sequence is encoded in a nucleic acid sequence having the sequence set forth in SEQ ID NO: 2. In some embodiments, the linker is GGGGG (SEQ ID NO:150). In some of any of the above examples, serine can be replaced with alanine (e.g., (Gly₄Ala) or (Gly₃Ala)).

In some embodiments, the linker includes a peptide linker having the amino acid sequence GlyₓXaa-Gly_{y}-Xaa-Gly_{z} (SEQ ID NO:151), wherein each Xaa is independently selected from Alanine (Ala), Valine (Val), Leucine (Leu), Isoleucine (Ile), Methionine (Met), Phenylalanine (Phe), Tryptophan (Trp), Proline (Pro), Glycine (Gly), Serine (Ser), Threonine (Thr), Cysteine (Cys), Tyrosine (Tyr), Asparagine (Asn), Glutamine (Gln), Lysine (Lys), Arginine (Arg), Histidine (His), Aspartate (Asp), and Glutamate (Glu), and wherein x, y, and z are each integers in the range from 1-5. In some embodiments, each Xaa is independently selected from the group consisting of Ser, Ala, and Thr. In a specific variation, each of x, y, and z is equal to 3 (thereby yielding a peptide linker having the amino acid sequence Gly-Gly-Gly-Xaa-Gly-Gly-Gly-Xaa-Gly-Gly-Gly (SEQ ID NO:152), wherein each Xaa is selected as above.

In some embodiments, the linker is serine-rich linkers based on the repetition of a (SSSSG)n (SEQ ID NO:153) motif where n is at least 1, though n can be 2, 3, 4, 5, 6, 7, 8 and 9.

In some cases, it may be desirable to provide some rigidity into the peptide linker. This may be accomplished by including proline residues in the amino acid sequence of the peptide linker. Thus, in some embodiments, a linker comprises at least one proline residue in the amino acid sequence of the peptide linker. For example, a peptide linker can have an amino acid sequence wherein at least 25% (e.g., at least 50% or at least 75%) of the amino acid residues are proline residues. In one particular embodiment, the peptide linker comprises proline residues only.

In some aspects, a peptide linker comprises at least one cysteine residue, such as one cysteine residue. For example, in some embodiments, a linker comprises at least one cysteine residue and amino acid residues selected from the group consisting of Gly, Ser, Ala, and Thr. In some such embodiments, a linker comprises glycine residues and cysteine residues, such as glycine residues and cysteine residues only. Typically, only one cysteine residue will be included per peptide linker. One example of a specific linker comprising a cysteine residue includes a peptide linker having the amino acid sequence Glyₘ-Cys-Glyₙ, wherein n and m are each integers from 1-12, e.g., from 3-9, from 4-8, or from 4-7. In a specific variation, such a peptide linker has the amino acid sequence GGGGG-C-GGGGG (SEQ ID NO:154).

3. Affinity Tag

In some aspects, the encoded miniCAR also includes an affinity tag. In some aspects, the affinity tag is positioned in the extracellular region of the encoded miniCAR. In some embodiments, the affinity tag is optional. In some aspects, the affinity tag is included in addition to the linker. In some aspects, the affinity tag is included in lieu of a linker. In some aspects, inclusion of the affinity tag allows the affinity tag of the miniCAR to be recognized by a binding molecule. In some aspects, the inclusion of an affinity tag and/or binding of the affinity tag to a binding molecule recognizing the affinity tag can facilitate detection, selection, separation and/or purification of cells, such as engineered T cells expressing the miniCAR.

In some aspects, the affinity tag is fused to the N-terminus of the extracellular binding domain, e.g., antigen-binding domain. In some aspects, the affinity tag is fused to the C-terminus of the extracellular binding domain, e.g., antigen-binding domain. In some aspects, the affinity tag is fused to the N-terminus of the linker, e.g., peptide linker. In some aspects, the affinity tag is fused to the C-terminus of the linker, e.g., peptide linker. In some aspects, the affinity tag is fused to the N-terminus of the invariant CD3-IgSF chain contained in the miniCAR, such as the extracellular region of the invariant CD3-IgSF chain. In some aspects, the affinity tag is fused to the N-terminus of the extracellular region of the invariant CD3-IgSF chain. In some aspects, the affinity tag is fused to the N-terminus of the transmembrane region of the invariant CD3-IgSF chain contained in the miniCAR.

In some embodiments, the affinity tag has enough residues to provide an epitope recognized by an antibody or by a non-antibody binding molecule, yet, in some aspects, is short enough such that it does not interfere with or sterically block an epitope of the target antigen of the miniCAR as described herein. Suitable tag polypeptides generally have at least 5 or 6 amino acid residues and usually between about 8-50 amino acid residues, typically between 9-30 residues.

In some embodiments, the affinity tag can be a streptavidin-binding peptide or other molecule that is able to specifically bind to streptavidin, a streptavidin mutein or analog, avidin or an avidin mutein or analog. In some embodiments, the affinity tag is a streptavidin binding peptide. In some embodiments, the affinity tag is recognized by a binding molecule that is or that comprises a streptavidin or a streptavidin mutein.

In some embodiments, the streptavidin binding peptide contains a sequence with the general formula set forth in SEQ ID NO: 138, such as contains the sequence set forth in SEQ ID NO: 139. In some embodiments, the peptide sequence has the general formula set forth in SEQ ID NO: 140, such as set forth in SEQ ID NO: 141. In one example, the peptide sequence is Trp-Arg-His-Pro-Gln-Phe-Gly-Gly (also called Strep-tag®, set forth in SEQ ID NO:136). In one example, the peptide sequence is Ser-Ala-Trp-Ser-His-Pro-Gln-Phe-Glu-Lys (SEQ ID NO:149) or the minimal sequence Trp-Ser-His-Pro-Gln-Phe-Glu-Lys (also called Strep-tag® II, set forth in SEQ ID NO:137). In some embodiments, the affinity tag contains a sequential arrangement of at least two streptavidin-binding peptide modules, wherein the distance between the two modules is at least 0 and not greater than 50 amino acids, wherein one binding module has 3 to 8 amino acids and contains at least the sequence His-Pro-Xaa (SEQ ID NO: 138), where Xaa is glutamine, asparagine, or methionine, and wherein the other binding module has the same or different streptavidin peptide ligand, such as set forth in SEQ ID NO: 140 (see e.g. International Published PCT Appl. No. WO 02/077018; U.S. Pat. No. 7,981,632). In some embodiments, the streptavidin binding peptide contains a sequence having the formula set forth in any of SEQ ID NO: 142 or 143. In some embodiments, the affinity tag can contain twin-strep-tags such as by the sequential arrangement of two streptavidin binding modules, such as is commercially available as Twin-Strep-tag® from IBA GmbH, Gottingen, Germany, for example, containing the sequence (SAWSHPQFEK(GGGS)₂GGSAWSHPQFEK)(SEQ ID NO: 145). In some embodiments, the streptavidin binding peptide has the sequence of amino acids set forth in any of SEQ ID NOS: 144-148. In most cases, all these streptavidin binding peptides bind to the same binding site, namely the biotin binding site of streptavidin. In some embodiments, exemplary affinity tag, such as a streptavidin binding peptide include those described, for example, in WO 2015/158868, WO 2017/068425, WO 2017/068419, WO 2017/068421, or WO 2018/134691.

In some embodiments, the streptavidin binding peptide is recognized by a binding molecule comprising streptavidin or streptavidin mutein, which exhibits binding affinity for the peptide. In some embodiments, the binding affinity of streptavidin or a streptavidin mutein for a streptavidin binding peptide is with an equilibrium binding constant (K_{D}) of less than 1×10⁻⁴ M, 5×10⁻⁴ M, 1×10⁻⁵ M, 5×10⁻⁵ M, 1×10⁻⁶ M, 5×10⁻⁶ M or 1×10⁻⁷ M, but generally greater than 1×10⁻¹³ M, 1×10⁻¹² M or 1×10⁻¹¹ M. For example, peptide sequences (Strep-tags), such as disclosed in U.S. Pat. No. 5,506,121, can act as biotin mimics and demonstrate a binding affinity for streptavidin, e.g., with a K_{D} of approximately between 10⁻⁴ M and 10⁻⁵ M. In some cases, the binding affinity can be further improved by making a mutation within the streptavidin molecule, see e.g. U.S. Pat. No. 6,103,493 or International published PCT App. No. WO 2014/076277. In some embodiments, binding affinity can be determined by known methods.

In some embodiments, the streptavidin binding peptide is recognized by a binding molecule that is or comprises a streptavidin, a streptavidin mutein or analog, avidin, an avidin mutein or analog (such as neutravidin) or a mixture thereof. In some embodiments, the binding molecule is or contains an analog or mutein of streptavidin or an analog or mutein of avidin that reversibly binds a streptavidin-binding peptide. In some embodiments, the binding molecule is or comprises an avidin that can be wild-type avidin or muteins or analogs of avidin such as neutravidin, a deglycosylated avidin with modified arginines that typically exhibits a more neutral pi and is available as an alternative to native avidin. Generally, deglycosylated, neutral forms of avidin include those commercially available forms such as “Extravidin”, available through Sigma Aldrich, or “NeutrAvidin” available from Thermo Scientific or Invitrogen, for example. In general, streptavidin naturally occurs as a tetramer of four identical subunits, i.e. it is a homo-tetramer, where each subunit contains a single binding site for biotin, a biotin derivative or analog or a biotin mimic. In some cases, streptavidin can exist as a monovalent tetramer in which only one of the four binding sites is functional (Howarth et al. (2006) Nat. Methods, 3:267-73; Zhang et al. (2015) Biochem. Biophys. Res. Commun., 463:1059-63)), a divalent tetramer in which two of the four binding sites are functional (Fairhead et al. (2013) J. Mol. Biol., 426:199-214), or can be present in monomeric or dimeric form (Wu et al. (2005) J. Biol. Chem., 280:23225-31; Lim et al. (2010) Biochemistry, 50:8682-91). In some embodiments, the affinity tag, such as a streptavidin binding peptide, is recognized by an exemplary binding molecule that is or comprises a streptavidin or a streptavidin mutein or analog described, for example, in WO 2015/158868, WO 2017/068425, WO 2017/068419, WO 2017/068421, U.S. Pat. Nos. 5,168,049; 5,506,121; 6,022,951; 6,156,493; 6,165,750; 6,103,493; U.S. or 6,368,813; or WO 2014/076277.

In some embodiments, the binding molecule is an oligomer or a polymer of one or more streptavidin or avidin or of any analog or mutein of streptavidin or an analog or mutein of avidin (e.g. neutravidin). In some embodiments, the oligomer is generated or produced from a plurality of individual molecules (e.g. a plurality of homo-tetramers) of the same streptavidin, streptavidin mutein, avidin or avidin mutein. In some embodiments the binding molecule is an oligomer or a polymer of one or more streptavidin or avidin or of any analog or mutein of streptavidin or an analog or mutein of avidin (e.g. neutravidin). In some embodiments, the oligomer is generated or produced from a plurality of individual molecules (e.g. a plurality of homo-tetramers) of the same streptavidin, streptavidin mutein, avidin or avidin mutein. Exemplary oligomeric binding molecule that can bind to the affinity tag of the miniCAR include those described in, for example, WO 2015/158868, WO 2017/068425, WO 2017/068419 or WO 2017/068421.

In some embodiments, a streptavidin binding peptide (e.g. Strep-tag, such as Strep-tag® II or twin-Strep-tag) can be recognized by a binding molecule that is an antibody or antigen-binding fragment. In some embodiments, the antibody contains at least one binding site that can specifically bind an epitope or region of the affinity tag of the encoded miniCAR. Antibodies against such streptavidin binding peptides are known, including antibodies against the peptide sequence SAWSHPQFEK (SEQ ID NO:149) or the minimal sequence WSHPQFEK (SEQ ID NO:137), such as present in Strep-tag® II or twin-strep-tag (Schmidt T. & Skerra A., Nature protocols, 2007; international patent application publication number WO 2015/067768). In some embodiments, a streptavidin binding peptide (e.g. Strep-tag, such as Strep-tag® II or twin-Strep-tag) can be detected using for example, the commercially available StrepMAB-Classic (IBA, Goettingen Germany), StrepMAB-lmmo (IBA), anti-Streptag II antibody (Genscript), or Strep-tag antibody (Qiagen).

In some embodiments, the binding molecule is labeled with one or more detectable marker, to facilitate purification, selection and/or detection of engineered cells. For example, separation may be based on binding to fluorescently labeled antibodies. In some embodiments, the binding molecule can be labeled with one or more detectable markers. In some embodiments, the binding molecule is labeled with a fluorescent marker. Exemplary labeled binding molecules are known or are commercially available including, for example, Strep-Tactin-HRP, Strep-Tactin AP, Strep-Tactin Chromeo 488, Strep-Tactin Chromeo 546, or Strep-Tactin Oyster 645, each available from IBA (Goettingen Germany).

4. Invariant CD3-IgSF Chains

The chimeric receptors, e.g., miniCARs, provided herein include all or a portion of an invariant CD3-IgSF chain. As described above, invariant CD3 chains of the immunoglobulin superfamily (invariant CD3-IgSF chains), e.g., CD3e, CD3d or CD3g, are highly related cell-surface proteins of the immunoglobulin superfamily containing a single extracellular immunoglobulin domain, and contain a single conserved ITAM, to generate a stimulating or activating signal. In some embodiments, the ITAM is contained in the intracellular or cytoplasmic region or domain of the invariant CD3-IgSF chain. Thus, in some embodiments, the invariant CD3-IgSF chain included in the miniCAR contains at least an intracellular region or an intracellular domain of an invariant CD3-IgSF chain or portion thereof including the ITAM.

In some embodiments, the invariant CD3-IgSF chain contained in the miniCAR includes an extracellular region or a portion thereof; a transmembrane region or a portion thereof; and an intracellular region or portion thereof, wherein the intracellular region or portion thereof includes an ITAM. In some embodiments, the intracellular region included in the miniCAR is a full length intracellular region of an invariant CD3-IgSF chain. In some embodiments, the invariant CD3-IgSF chain contained in the miniCAR is a full length invariant CD3-IgSF chain. In some embodiments, the invariant CD3-IgSF chain contained in the miniCAR is a mature invariant CD3-IgSF chain, for example, without a signal peptide or after cleavage of a signal peptide. In some embodiments, the invariant CD3-IgSF chain contained in the miniCAR is a CD3e, CD3d, or CD3g chain.

In some cases, for example when a transgene encoding the binding domain of a miniCAR as described herein is integrated at a CD3E locus, the invariant CD3-IgSF chain of the miniCAR is a CD3e chain. In some embodiments, the CD3e chain is a full length CD3e chain. In some embodiments, the CD3e chain is a mature CD3e chain.

In some embodiments, the encoded CD3e chain of the miniCAR contains an extracellular region or a portion thereof (e.g., amino acids 23-126, such as 32-112, of SEQ ID NO: 17), a transmembrane region or a portion thereof (e.g., amino acids 127-152 of SEQ ID NO: 17), and an intracellular region or a portion thereof (e.g., amino acids 153-207, such as 178-205, of SEQ ID NO: 17). In some embodiments, the intracellular region or portion thereof includes the sequence set forth by amino acids 178-205 of SEQ ID NO: 17.

In some embodiments, the encoded CD3e chain of the miniCAR includes or is the sequence set forth in amino acids 23-207 of SEQ ID NO: 17. In some embodiments, the encoded CD3e chain of the miniCAR is or includes a sequence that exhibits at least or about 85%, 90%, 92%, 95%, or 97% sequence identity to amino acids 23-207 of SEQ ID NO: 17. In some embodiments, the encoded CD3e chain of the miniCAR consists of or consists essentially of the sequence set forth by amino acids 23-207 of SEQ ID NO: 17. In some embodiments, the encoded CD3e chain of the miniCAR consists of or consists essentially of a sequence that exhibits at least or about 85%, 90%, 92%, 95%, or 97% sequence identity to the sequence set forth by amino acids 23-207 of SEQ ID NO: 17. In some embodiments, the encoded CD3e chain of the miniCAR is a functional variant of the sequence set forth in SEQ ID NO:17, or the amino acid sequence set forth by amino acids 23-207 of SEQ ID NO: 17, sufficient to induce stimulating or activating signals through the TCR/CD3 complex into which it assembles, following binding the miniCAR binding domain to a target antigen. In some embodiments, the functional variant has a sequence of amino acids that exhibits at least at or about 85%, at least at or about 90%, at least at or about 92%, at least at or about 95%, or at least at or about 98% sequence identity to the sequence of amino acids set forth in SEQ ID NO: 17 or the sequence set forth by amino acids 23-207 of SEQ ID NO: 17.

In some embodiments, the encoded CD3e chain of the miniCAR contains an extracellular region or a portion thereof (e.g., amino acids 22-120, such as 34-99, of SEQ ID NO: 19), a transmembrane region or a portion thereof (e.g., amino acids 121-145 of SEQ ID NO: 19), and an intracellular region or a portion thereof (e.g., amino acids 146-201 of SEQ ID NO: 19).

In some embodiments, the encoded CD3e chain of the miniCAR includes or is the sequence set forth in amino acids 22-201 of SEQ ID NO: 19. In some embodiments, the encoded CD3e chain of the miniCAR is or includes a sequence that exhibits at least or about 85%, 90%, 92%, 95%, or 97% sequence identity to amino acids 22-201 of SEQ ID NO: 19. In some embodiments, the encoded CD3e chain of the miniCAR consists of or consists essentially of the sequence set forth by amino acids 22-201 of SEQ ID NO: 19. In some embodiments, the encoded CD3e chain of the miniCAR consists of or consists essentially of a sequence that exhibits at least or about 85%, 90%, 92%, 95%, or 97% sequence identity to the sequence set forth by amino acids 22-201 of SEQ ID NO: 19. In some embodiments, the encoded CD3e chain of the miniCAR is a functional variant of the sequence set forth in SEQ ID NO:19, or the amino acid sequence set forth by amino acids 22-201 of SEQ ID NO: 19, sufficient to induce stimulating or activating signals through the TCR/CD3 complex into which it assembles, following binding the miniCAR binding domain to a target antigen. In some embodiments, the functional variant has a sequence of amino acids that exhibits at least at or about 85%, at least at or about 90%, at least at or about 92%, at least at or about 95%, or at least at or about 98% sequence identity to the sequence of amino acids set forth in SEQ ID NO: 19 or the sequence set forth by amino acids 22-201 of SEQ ID NO: 19.

In some cases, for example when a transgene encoding the binding domain of a miniCAR as described herein is integrated at a CD3D locus, the invariant CD3-IgSF chain of the miniCAR is a CD3d chain. In some embodiments, the CD3d chain is a full length CD3d chain. In some embodiments, the CD3d chain is a mature CD3d chain.

In some embodiments, the encoded CD3d chain of the miniCAR contains an extracellular region or a portion thereof (e.g., amino acids 22-105 of SEQ ID NO: 20), a transmembrane region or a portion thereof (e.g., amino acids 106-126 of SEQ ID NO: 20), and an intracellular region or a portion thereof (e.g., amino acids 127-171, such as 138-166, of SEQ ID NO: 20). In some embodiments, the intracellular region or portion thereof includes the sequence set forth by amino acids 138-166 of SEQ ID NO: 20.

In some embodiments, the encoded CD3d chain of the miniCAR includes or is the sequence set forth in amino acids 22-171 of SEQ ID NO: 20. In some embodiments, the encoded CD3d chain of the miniCAR is or includes a sequence that exhibits at least or about 85%, 90%, 92%, 95%, or 97% sequence identity to amino acids 22-171 of SEQ ID NO: 20. In some embodiments, the encoded CD3d chain of the miniCAR consists of or consists essentially of the sequence set forth by amino acids 22-171 of SEQ ID NO: 20. In some embodiments, the encoded CD3d chain of the miniCAR consists of or consists essentially of a sequence that exhibits at least or about 85%, 90%, 92%, 95%, or 97% sequence identity to the sequence set forth by amino acids 22-171 of SEQ ID NO: 20. In some embodiments, the encoded CD3d chain of the miniCAR is a functional variant of the sequence set forth in SEQ ID NO:20, or the amino acid sequence set forth by amino acids 22-171 of SEQ ID NO: 20, sufficient to induce stimulating or activating signals through the TCR/CD3 complex into which it assembles, following binding of the miniCAR binding domain to a target antigen. In some embodiments, the functional variant has a sequence of amino acids that exhibits at least at or about 85%, at least at or about 90%, at least at or about 92%, at least at or about 95%, or at least at or about 98% sequence identity to the sequence of amino acids set forth in SEQ ID NO: 20 or the sequence set forth by amino acids 22-171 of SEQ ID NO: 20.

In some embodiments, the encoded CD3d chain of the miniCAR includes or is the sequence set forth in amino acids 22-127 of SEQ ID NO: 22. In some embodiments, the encoded CD3d chain of the miniCAR is or includes a sequence that exhibits at least or about 85%, 90%, 92%, 95%, or 97% sequence identity to amino acids 22-127 of SEQ ID NO: 22. In some embodiments, the encoded CD3d chain of the miniCAR consists of or consists essentially of the sequence set forth by amino acids 22-127 of SEQ ID NO: 22. In some embodiments, the encoded CD3d chain of the miniCAR consists of or consists essentially of a sequence that exhibits at least or about 85%, 90%, 92%, 95%, or 97% sequence identity to the sequence set forth by amino acids 22-127 of SEQ ID NO: 22. In some embodiments, the encoded CD3d chain of the miniCAR is a functional variant of the sequence set forth in SEQ ID NO:22, or the amino acid sequence set forth by amino acids 22-127 of SEQ ID NO: 22, sufficient to induce stimulating or activating signals through the TCR/CD3 complex into which it assembles, following binding of the miniCAR binding domain to a target antigen. In some embodiments, the functional variant has a sequence of amino acids that exhibits at least at or about 85%, at least at or about 90%, at least at or about 92%, at least at or about 95%, or at least at or about 98% sequence identity to the sequence of amino acids set forth in SEQ ID NO: 22 or the sequence set forth by amino acids 22-127 of SEQ ID NO: 22.

In some embodiments, the encoded CD3d chain of the miniCAR contains an extracellular region or a portion thereof (e.g., amino acids 23-30 of SEQ ID NO: 24), a transmembrane region or a portion thereof (e.g., amino acids 31-53 of SEQ ID NO: 24), and an intracellular region or a portion thereof (e.g., amino acids 54-98 of SEQ ID NO: 24).

In some embodiments, the encoded CD3d chain of the miniCAR includes or is the sequence set forth in amino acids 23-98 of SEQ ID NO: 24. In some embodiments, the encoded CD3d chain of the miniCAR is or includes a sequence that exhibits at least or about 85%, 90%, 92%, 95%, or 97% sequence identity to amino acids 23-98 of SEQ ID NO: 24. In some embodiments, the encoded CD3d chain of the miniCAR consists of or consists essentially of the sequence set forth by amino acids 23-98 of SEQ ID NO: 24. In some embodiments, the encoded CD3d chain of the miniCAR consists of or consists essentially of a sequence that exhibits at least or about 85%, 90%, 92%, 95%, or 97% sequence identity to the sequence set forth by amino acids 23-98 of SEQ ID NO: 24. In some embodiments, the encoded CD3d chain of the miniCAR is a functional variant of the sequence set forth in SEQ ID NO:24, or the amino acid sequence set forth by amino acids 23-98 of SEQ ID NO: 24, sufficient to induce stimulating or activating signals through the TCR/CD3 complex into which it assembles, following binding of the miniCAR binding domain to a target antigen. In some embodiments, the functional variant has a sequence of amino acids that exhibits at least at or about 85%, at least at or about 90%, at least at or about 92%, at least at or about 95%, or at least at or about 98% sequence identity to the sequence of amino acids set forth in SEQ ID NO: 24 or the sequence set forth by amino acids 23-98 of SEQ ID NO: 24.

In some cases, for example when a transgene encoding the binding domain of a miniCAR is integrated at a CD3G locus, the invariant CD3-IgSF chain of the miniCAR is a CD3g chain. In some embodiments, the CD3g chain is a full length CD3g chain. In some embodiments, the CD3g chain is a mature CD3g chain.

In some embodiments, the encoded CD3g chain of the miniCAR contains an extracellular region or a portion thereof (e.g., amino acids 23-116, such as 37-94, of SEQ ID NO: 26), a transmembrane region or a portion thereof (e.g., amino acids 117-137 of SEQ ID NO: 26), and an intracellular region or a portion thereof (e.g., amino acids 138-182, such as 149-177, of SEQ ID NO: 26). In some embodiments, the intracellular region or portion thereof includes the sequence set forth by amino acids 149-177 of SEQ ID NO: 26.

In some embodiments, the encoded CD3g chain of the miniCAR includes or is the sequence set forth in amino acids 23-182 of SEQ ID NO: 26. In some embodiments, the encoded CD3g chain of the miniCAR is or includes a sequence that exhibits at least or about 85%, 90%, 92%, 95%, or 97% sequence identity to amino acids 23-182 of SEQ ID NO: 26. In some embodiments, the encoded CD3g chain of the miniCAR consists of or consists essentially of the sequence set forth by amino acids 23-182 of SEQ ID NO: 26. In some embodiments, the encoded CD3g chain of the miniCAR consists of or consists essentially of a sequence that exhibits at least or about 85%, 90%, 92%, 95%, or 97% sequence identity to the sequence set forth by amino acids 23-182 of SEQ ID NO: 26. In some embodiments, the encoded CD3g chain of the miniCAR is a functional variant of the sequence set forth in SEQ ID NO:26, or the amino acid sequence set forth by amino acids 23-182 of SEQ ID NO: 26, sufficient to induce stimulating or activating signals through the TCR/CD3 complex into which it assembles, following binding of the miniCAR binding domain to a target antigen. In some embodiments, the functional variant has a sequence of amino acids that exhibits at least at or about 85%, at least at or about 90%, at least at or about 92%, at least at or about 95%, or at least at or about 98% sequence identity to the sequence of amino acids set forth in SEQ ID NO: 26 or the sequence set forth by amino acids 23-182 of SEQ ID NO: 26.

In some embodiments, an extracellular region of a CD3e, CD3d, or CD3g chain as described above is linked directly to the binding domain, e.g., antigen-binding domain, of the miniCAR. In some embodiments, an extracellular region of a CD3e, CD3d, or CD3g chain as described above is linked indirectly to the binding domain, e.g., antigen-binding domain, of the miniCAR through a linker. In some embodiments, the linker is as set forth in Section III.B.2 above.

C. Cells and Preparation of Cells for Genetic Engineering

In some embodiments, provided are engineered cells, e.g., genetically engineered or modified cells, and methods of engineering cells, including genetically engineered cells comprising a modified invariant CD3-IgSF chain locus, e.g., CD3E, CD3D, CD3G locus, that comprises a transgene sequence encoding a portion, e.g., antigen-binding domain, of a chimeric receptor such as a miniCAR. In some embodiments, polynucleotides, e.g., template polynucleotides such as any of the template polynucleotides described herein, such as in Section I.B.2, containing nucleic acid sequences comprising transgene sequences encoding a portion of a miniCAR. and/or additional molecule(s), are introduced into one a cell for engineering, e.g., according to the methods of engineering described herein. In some aspects, the cells are engineered using any of the methods provided herein. In some embodiments, the engineered cells contain a modified invariant CD3-IgSF chain locus, e.g., CD3E, CD3D, CD3G locus, said modified invariant CD3-IgSF chain locus comprising a nucleic acid sequence encoding a miniCAR comprising an antigen-binding domain and all or a portion of an endogenous invariant CD3-IgSF chain, e.g., a CD3e, a CD3d, or CD3g chain. In some aspects, the modified invariant CD3-IgsF chain locus of the engineered cell include those described in Section III.A herein.

In some aspects, the transgene sequences (exogenous or heterologous nucleic acid sequences, such as any described in Section I.B.2 herein) in the polynucleotides (such as template polynucleotides, for example, described in Section I.B.2 herein) and/or portions thereof are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acid sequences are not naturally occurring, such as a nucleic acid sequences not found in nature or is modified from a nucleic acid sequence found in nature, including one comprising chimeric combinations of nucleic acids encoding various domains from multiple different cell types.

In some aspects, provided are method of producing a genetically engineered T cell, the method involving introducing any of the provided polynucleotides, e.g., described herein in Section I.B.2, into a T cell comprising a genetic disruption at an invariant CD3-IgSF chain locus. In some aspects, the genetic disruption is introduced by any agents or methods for introducing a targeted genetic disruption, including any described herein, such as in Section I.A. In some aspects, the method produces a modified invariant CD3-IgSF chain locus, said modified invariant CD3-IgSF chain locus comprising a nucleic acid sequence encoding the miniCAR, comprising a heterologous antigen-binding domain and an endogenous invariant CD3-IgSF chain. In some aspects, provided are methods of producing a genetically engineered T cell that involves introducing, into a T cell, one or more agent(s) capable of inducing a genetic disruption at a target site within an endogenous invariant CD3-IgSF chain locus, e.g., CD3E, CD3D or CD3G locus, of the T cell; and introducing any of the provided polynucleotides, e.g., described herein in Section I.B.2, into a T cell comprising a genetic disruption at an invariant CD3-IgSF chain locus, wherein the method produces a modified invariant CD3-IgSF chain locus, said modified invariant CD3-IgSF chain locus comprising a nucleic acid sequence encoding the miniCAR comprising an antigen-binding domain and the endogenous invariant CD3-IgSF chain. In some embodiments, the nucleic acid sequence comprises a transgene sequence encoding a portion of the miniCAR, e.g., an antigen-binding domain, and the transgene sequence is targeted for integration within the endogenous invariant CD3-IgSF chain locus via homology directed repair (HDR).

In some embodiments, provided are methods of producing a genetically engineered T cell that involve introducing, into a T cell, a polynucleotide comprising a nucleic acid sequence encoding a portion of a miniCAR said T cell having a genetic disruption within an invariant CD3-IgSF chain locus, e.g., CD3E, CD3D or CD3G locus, of the T cell, wherein the nucleic acid sequence encoding the portion of the miniCAR is targeted for integration within the endogenous invariant CD3-IgSF chain locus via homology directed repair (HDR). In some embodiments, the method produces a modified invariant CD3-IgSF chain locus, said modified invariant CD3-IgSF chain locus comprising a nucleic acid sequence encoding a miniCAR comprising a heterologous antigen-binding domain and an endogenous invariant CD3-IgSF chain. In some embodiments, the nucleic acid sequence comprises a transgene sequence encoding a portion of the miniCAR, such as any described herein, for example, in Section I.B.2.

In some embodiments, upon performance of the methods, all, e.g., the entire or full, invariant CD3-IgSF chain in the genetically engineered T cell is encoded by an open reading frame or a partial sequence thereof of the endogenous invariant CD3-IgSF chain locus. In some embodiments, the nucleic acid sequence comprises a transgene sequence encoding a portion of the miniCAR, said portion encoding an antigen-binding domain and optionally a linker, and wherein the open reading frame or a partial sequence thereof encodes the entire or full invariant CD3-IgSF chain. In some embodiments, at least a fragment of the invariant CD3-IgSF chain, optionally the entire mature invariant CD3-IgSF chain, of the encoded miniCAR is encoded by the open reading frame of the endogenous invariant CD3-IgSF chain locus or a partial sequence thereof.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as iPSCs. In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils. In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the antigen-binding domain, and optionally a linker, of the miniCAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of binding molecule, each specific for a marker targeted for negative selection. Likewise, multiple cell types can simultaneously be positively selected by incubating cells with a plurality of binding molecule expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood.1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the binding molecule, or molecules, such as secondary antibodies or other reagents, which specifically bind to such binding molecule, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Pat. App. Pub. No. WO2009/072003 or US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the binding molecule are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177, Klebanoff et al. (2012) J Immunother. 35(9): 651-660, Terakura et al. (2012) Blood.1:72-82, and/or Wang et al. (2012) J Immunother.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Various methods for the introduction of genetically engineered components, e.g., agents for inducing a genetic disruption and/or nucleic acids encoding portions of miniCARs, are known and may be used with the provided methods and compositions. Exemplary methods include those for transfer of nucleic acids encoding the polypeptides or receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell, or any delivery methods described in Section I.A herein. Other approaches and vectors for transfer of the nucleic acids encoding the recombinant products are those described, e.g., in WO2014055668 and U.S. Pat. No. 7,446,190.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in T cells include calcium phosphate transfection (such as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, it may be desired to safeguard against the potential that overexpression of a stimulatory factor (for example, a lymphokine or a cytokine) could potentially result in an unwanted outcome or lower efficacy in a subject, such as a factor associated with toxicity in a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, the cells, e.g., T cells, may be engineered either during or after expansion. This engineering for the introduction of the gene of the desired polypeptide or receptor can be carried out with any suitable retroviral vector, for example. The genetically modified cell population can then be liberated from the initial stimulus (the CD3/CD28 stimulus, for example) and subsequently be stimulated with a second type of stimulus (e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol, 2012; 907:645-66 or Barrett et al., Chimeric Antigen Receptor Therapy for Cancer Annual Review of Medicine Vol. 65: 333-347 (2014).

In some embodiments, the cells are expanded following engineering. In some embodiments, engineered cells may be cultured and then expanded using antigen-specific or anti-idiotype antibodies. In some aspects, antigen-specific expansion of engineered T cells may be useful for increasing the total number of cells expressing the miniCAR. In some embodiments, engineered cells are expanded using antigen-specific or anti-idiotype antibodies prior to formulation into a therapeutic composition.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

As described herein, in some embodiments, the cells are incubated and/or cultured prior to or in connection with or after genetic engineering. The incubation steps can include culture, cultivation, stimulation, activation, expansion and/or freezing for preservation, e.g. cryopreservation. In some aspects, the engineered population of T cells are cultivated under conditions for expansion, wherein the cultivating is subsequent to the introducing of the one or more agents and/or the introducing of the polynucleotide. In some embodiments, cultivating under conditions for expansion comprises incubating the population of T cells with the target antigen of the antigen-binding domain, target cells expressing the target antigen, or an anti-idiotype antibody that binds to the antigen-binding domain.

In some aspects, after introduction of the agent for genetic disruption and/or template polynucleotides containing the transgene, the cells are cultured or incubated for expansion. In some embodiments, the incubation may comprise adding non-dividing EBV-transformed lymphoblastoid cells (LCL) as feeder cells. LCL can be irradiated with gamma rays in the range of about 6000 to 10,000 rads. The LCL feeder cells in some aspects are provided in any suitable amount, such as a ratio of LCL feeder cells to initial T lymphocytes of at least about 10:1.

In some embodiments, the engineered cells expanded using antigen-specific expansion methods. For example, engineered cells may be co-cultured with cells, such as LCL cells, expressing, or engineered to express, the target antigen of the antigen-binding domain of the miniCAR, to selectively induce expansion of engineered cells expressing the miniCAR. In some embodiments, antigen-specific expansion can be induced by co-culturing engineered cells with antigen-expressing cells at an effector to target (E:T) ratio of 0.5:1, 1:1, 1:2, 1:3, 1:4, 1:5, 1:6, 1:7, 1:8, 1:9, 1:10, or any suitable E:T to induce selective expansion. In some embodiments, the E:T ratio for co-culturing 1:3.

In some embodiments, antigen-specific cell expansion is accomplished by cultivating or co-culturing the engineered cells with an anti-idiotype antibody that binds the binding domain, e.g., antigen-binding domain, of the miniCAR, and induces expansion of the miniCAR-expressing cells.

In some embodiments, cultivating cells under conditions for expansion of the engineered cells, such as expanding engineered cells, for example using antigen-specific expansion methods described herein, increases, optionally selectively increases, the number of engineered cells expressing the miniCAR. In some embodiments, the cultivation is performed until, or the cultivation ends, such as by harvesting cells, when cells achieve a threshold amount, concentration, and/or expansion. In some embodiments, the duration of co-culturing under conditions for expansion is at or about 24 hours, 36 hours, 48 hours, 3, 4, 5, 6, 7, 8, 9, 10, 11, 12, 13, 14, 15, 16, 17, 18, 19, 20 or 21 days. In some embodiments, the duration of co-culturing under antigen-specific expansion conditions is at or about 4 days, 5 days, 6 days, 7 days, or 8 days. In some embodiments, the engineered cell population may be cultured under conditions for expansion until a target number of miniCAR-positive cells, e.g., miniCAR+ cells, is reached. In some embodiments, the engineered cell population may be cultured under conditions for expansion until a target fold expansion miniCAR-positive cells, e.g., miniCAR+ cells, is reached. In some embodiments, the cultivation ends when the cell achieve or achieve about or at least a 1.5-fold expansion, a 2-fold expansion, a 2.5-fold expansion, a 3-fold expansion, a 3.5-fold expansion, a 4-fold expansion, a 4.5-fold expansion, a 5-fold expansion, a 6-fold expansion, a 7-fold expansion, a 8-fold expansion, a 9-fold expansion, a 10-fold expansion, 15-fold expansion, a 20-fold expansion, a 25-fold expansion, a 30-fold expansion, a 35-fold expansion, a expansion, a 45-fold expansion, a 50-fold expansion, a 60-fold expansion, a 70-fold expansion, a 80-fold expansion, a 90-fold expansion, a 100-fold expansion, or greater than a 100-fold expansion, e.g., with respect and/or in relation to the amount of density of the cells at the start or initiation of the cultivation. In some embodiments, the threshold expansion is a 30-fold expansion, e.g., with respect and/or in relation to the amount of density of the cells at the start or initiation of the cultivation or immediately prior to the start or initiation of the cultivation.

In some embodiments, the cultivation ends when the cell achieve or achieve a certain number or percentage of miniCAR-positive cells among a population of cells. In some embodiments, the cultivation is performed until, or the cultivation ends, such as by harvesting cells, when at least at or about 10%, 15%, 20%, 25%, 30%, 35%, 40%, 45%, 50%, 55%, 60%, 65%, 70%, 75%, 80%, 85%, 90% or more of the cells in the population of cells express the miniCAR, e.g., are miniCAR+ cells. In some embodiments, the desired or target number of miniCAR-positive cells is defined or determined as the number of miniCAR-expressing cells for formulation as therapeutic agent. In some embodiments, the target number of miniCAR-expressing cells is a number of cells defined herein, for example in Section IV below. In some embodiments, the cultivation is performed until, or the cultivation ends, such as by harvesting cells, when at or about 1×10⁶ to at or about 5×10⁸ miniCAR-expressing cells, such as at or about 2×10⁶, 5×10⁶, 1×10⁷, 5×10⁷, 1×10⁸, 1.5×10⁸, or 5×10⁸ total miniCAR-expressing cells, or the range between any two of the foregoing values, are achieved. In some embodiments, the cultivation is performed until, or the cultivation ends, such as by harvesting cells, when at or about 2×10⁹ total miniCAR-expressing cells, such as, in the range of at or about 2.5×10⁷ to at or about 1.2×10⁹ miniCAR-expressing cells, such as at or about 2.5×10⁷, 5×10⁷, 1×10⁸, 1.5×10⁸, 8×10⁸, or 1.2×10⁹ total miniCAR-expressing cells, or the range between any two of the foregoing values, is achieved.

In some embodiments, the cells, e.g., engineered cells, are cultivated under conditions for expansion, according to any of the methods of expansion described herein, may be enriched for miniCAR-expressing cells and/or specific cell subtypes thereof. In some embodiments, expanded cells may be enriched for miniCAR-expressing, e.g., miniCAR+, cells. In some embodiments, expanded cells may be enriched for subtypes of miniCAR-expressing, e.g., miniCAR+, cells. For example, expanded cells may be enriched for miniCAR+/CD3+, miniCAR+/CD4+, miniCAR+/CD8+, and subtypes thereof, for example as described in Section III.C. In some embodiments, selective enrichment of the expanded cells, e.g., for miniCAR+, miniCAR+/CD3+, miniCAR+/CD4+, miniCAR+/CD8+, and subtypes thereof, may be performed according to any cell selection techniques described herein, for example at Section III.C.

D. Composition of Cells

Also provided are a plurality or populations of engineered cells, compositions containing such cells and/or enriched for such cells. In some aspects, the provided engineered cells and/or composition of engineered cells include any described herein, e.g., comprising a modified invariant CD3-IgSF chain locus, e.g., CD3E, CD3D, CD3G locus, comprising a transgene sequence include nucleic acid sequences encoding an antigen-binding domain, for example as described herein, and/or are produced by the methods described herein. In some aspects, the plurality or population of engineered cells contains any of the engineered cells described herein, e.g., in Section III.C herein. In some aspects, the provided cells and cell compositions can be engineered using any of the methods described herein, e.g., using agent(s) or methods for introducing genetic disruption, for example, as described in Section I.A herein, and/or using polynucleotides, such as template polynucleotide descried herein, for example in Section I.B.2, via homology-directed repair (HDR). In some aspects, such cell populations and/or compositions provided herein are comprised in a pharmaceutical composition or a composition for therapeutic uses or methods, for example, as described in Section V herein.

In some embodiments, the provided cell population and/or compositions containing engineered cells include a cell population that exhibits more improved, uniform, homogeneous and/or stable expression and/or antigen binding by the miniCAR, e.g., exhibit reduced coefficient of variation, compared to the expression and/or antigen binding of cell populations and/or compositions generated using other methods. In some embodiments, the cell population and/or compositions exhibit at least at or about 100%, 95%, 90%, 80%, 70%, 60%, 50%, 40%, 30%, 20% or 10% lower coefficient of variation of expression of the miniCAR and/or antigen binding by the miniCAR compared to a respective population generated using other methods, e.g., random integration of sequences encoding the miniCAR. The coefficient of variation is defined as standard deviation of expression of the nucleic acid of interest (e.g., transgene sequences) within a population of cells, for example CD4+ and/or CD8+ T cells, divided by the mean of expression of the respective nucleic acid of interest in the respective population of cells. In some embodiments, the cell population and/or compositions exhibit a coefficient of variation that is lower than at or about 0.70, 0.65, 0.60, 0.55, 0.50, 0.45, 0.40, 0.35 or 0.30 or less, when measured among CD4+ and/or CD8+ T cell populations that have been engineered using the methods provided herein.

In some embodiments, the provided cell population and/or compositions containing engineered cells include a cell population that exhibits minimal or reduced random integration of the transgene. In some aspects, random integration of transgene into the genome of the cell can result in adverse effects or cell death due to integration of the transgene into undesired location in the genome, e.g., into an essential gene or a gene critical in regulating the activity of the cell, and/or unregulated or uncontrolled expression of the receptor. In some aspects, random integration of the transgene is reduced by at least at or about or greater than at or about 50%, 60%, 70%, 80%, 90%, 91%, 92%, 93%, 94%, 95%, 96%, 97%, 98%, 99%, or more compared to cell populations generated using other methods.

In some embodiments, at least at or about or greater than at or about 30%, 35%, 40%, 45%, 50%, 55%, 60%, 65%, 70%, 75%, 80%, or 90% of the cells in the composition and/or cells in the composition that contains a genetic disruption at the invariant CD3-IgSF chain locus include integration of the transgene at the invariant CD3-IgSF chain locus, e.g., CD3E, CD3D, or CD3G locus. In some embodiments, at least at or about or greater than at or about at or about 75%, 80%, or 90% of the cells in a plurality of engineered cells generated by the method comprise a genetic disruption of at least at or about one target site within the invariant CD3-IgsF chain locus.

In some embodiments, provided are cell population and/or compositions that include a plurality of engineered T cells expressing a miniCAR, wherein the nucleic acid sequence encoding the miniCAR is present at the invariant CD3-IgSF chain locus (e.g., CD3E, CD3D, or CD3G), e.g., by integration of a transgene at the invariant CD3-IgSF chain locus via homology directed repair (HDR).

In some embodiments, at least at or about or greater than at or about 1%, 2%, 4%, 8%, 10%, 15%, 20%, 25%, 35%, 40%, 45%, 50%, 55%, 60%, 65%, 70%, 75%, 80%, or 90% of the cells in the composition express the miniCAR. In some embodiments, at least at or about or greater than at or about 20%, 25%, 35%, 40%, 45%, 50%, 55%, 60%, 65%, 70%, 75%, 80%, or 90% of the cells in the composition express the miniCAR. In some embodiments, at least or greater than at or about 25%, 30%, 35%, 40%, 45%, 50%, 55%, 60%, 65%, 70%, 75%, 80%, 90%, 91%, 92%, 93%, 94%, 95%, 96%, 97%, 98%, 99% or more of the cells in a plurality of engineered cells generated by the method express the miniCAR. In some embodiments, at least or greater than at or about 25%, 30%, 35%, 40%, 45%, 50%, 55%, 60%, 65%, 70%, 75%, 80%, 90%, 91%, 92%, 93%, 94%, 95%, 96%, 97%, 98%, 99% or more of the cells in a plurality of engineered cells generated by the method express the miniCAR, after expansion and/or enrichment of cells expressing the miniCAR.

In some embodiments, the provided compositions containing cells such as in which cells expressing the miniCAR make up at least at or about 25%, 30%, 35%, 40%, 45%, 50%, 55%, 60%, 65%, 70%, 75%, 80%, 90%, 91%, 92%, 93%, 94%, 95%, 96%, 97%, 98%, 99%, or more of the total cells in the composition or cells of a certain type such as T cells or CD8+ or CD4+ cells. In some embodiments, the provided compositions containing cells such as in which cells expressing the miniCAR make up at least at or about 25%, 30%, 35%, 40%, 45%, 50%, 55%, 60%, 65%, 70%, 75%, 80%, 90%, 91%, 92%, 93%, 94%, 95%, 96%, 97%, 98%, 99%, or more of the total cells in the composition that contains a genetic disruption at the invariant CD3-IgSF chain locus, e.g., CD3E, CD3D, or CD3G locus.

In some embodiments, the provided compositions containing cells such as in which cells expressing the miniCAR make up at least at or about 25%, 30%, 35%, 40%, 45%, 50%, 55%, 60%, 65%, 70%, 75%, 80%, 90%, 91%, 92%, 93%, 94%, 95%, 96%, 97%, 98%, 99%, or more of the total cells in the composition or cells of a certain type such as T cells or CD8+ or CD4+ cells.

### IV. Methods of Treatment

Provided herein are methods of treatment, e.g., including administering any of the engineered cells or compositions containing the engineered cells described herein, for example, engineered cells comprising a modified invariant CD3-IgSF chain locus, e.g., CD3E, CD3D, or CD3G locus, comprising a transgene encoding a heterologous antigen-binding domain. In some aspects, also provided are methods of administering any of the engineered cells or compositions containing engineered cells described herein to a subject, such as a subject that has a disease or disorder. The engineered cells expressing a miniCAR as described herein, or compositions containing the same, are useful in a variety of therapeutic, diagnostic and prophylactic indications. For example, the engineered cells or compositions containing the engineered cells are useful in treating a variety of diseases and disorders in a subject. Such methods and uses include therapeutic methods and uses, for example, involving administration of the engineered cells, or compositions containing the same, to a subject having a disease, condition, or disorder, such as a tumor or cancer. In some embodiments, the engineered cells or compositions comprising the same are administered in an effective amount to effect treatment of the disease or disorder. Uses include uses of the engineered cells or compositions in such methods and treatments, and in the preparation of a medicament in order to carry out such therapeutic methods. In some embodiments, the methods are carried out by administering the engineered cells, or compositions comprising the same, to the subject having or suspected of having the disease or condition. In some embodiments, the methods thereby treat the disease or condition or disorder in the subject. Also provided are therapeutic methods for administering the cells and compositions to subjects, e.g., patients.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described herein. In particular embodiments, the antigen-binding domain of a miniCAR described herein specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or disorder is a multiple myeloma (MM). In some embodiments, administration of the provided cells, e.g., engineered cells containing a modified invariant CD3-IgSF chain locus, e.g., CD3E, CD3D, or CD3G locus, encoding a miniCAR described herein, can result in treatment of and/or amelioration of a disease or condition, such as a MM in the subject. In some embodiments, the subject has or is suspected of having a MM that is associated with expression of a tumor-associated antigen, such as a B cell maturation antigen (BCMA), G protein-coupled receptor class C group 5 member D (GPRC5D), or Fc receptor like 5 (FCRL5; also known as Fc receptor homolog 5 or FCRH5).

In some embodiments, the disease or disorder is a chronic lymphocytic leukemia (CLL). In some embodiments, administration of the provided cells, e.g., engineered cells containing a modified invariant CD3-IgSF chain locus, e.g., CD3E, CD3D, or CD3G locus, encoding a miniCAR described herein, can result in treatment of and/or amelioration of a disease or condition, such as a CLL in the subject. In some embodiments, the subject has or is suspected of having a CLL that is associated with expression of a tumor-associated antigen, such as a Receptor Tyrosine Kinase Like Orphan Receptor 1 (ROR1).

In some embodiments, the disease or disorder is a solid tumor, or a cancer associated with a non-hematological tumor. In some embodiments, the disease or disorder is a solid tumor, or a cancer associated with a solid tumor. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma. In some embodiments, the disease or disorder is a bladder, lung, brain, melanoma (e.g. small-cell lung, melanoma), breast, cervical, ovarian, colorectal, pancreatic, endometrial, esophageal, kidney, liver, prostate, skin, thyroid, or uterine cancers. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma.

In some embodiments, the disease or disorder is a non-small cell lung cancer (NSCLC). In some embodiments, administration of the provided cells, e.g., engineered cells containing a modified invariant CD3-IgSF chain locus, e.g., CD3E, CD3D, or CD3G locus, encoding a miniCAR described herein, can result in treatment of and/or amelioration of a disease or condition, such as a NSCLC in the subject. In some embodiments, the subject has or is suspected of having a NSCLC that is associated with expression of a tumor-associated antigen, such as a Receptor Tyrosine Kinase Like Orphan Receptor 1 (ROR1).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some aspects, the miniCAR, as described herein, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30, or combinations thereof.

In some embodiments, the disease or condition is a myeloma, such as a multiple myeloma. In some aspects, the antigen-binding domain of the miniCAR as described herein specifically binds to an antigen associated with the disease or condition or expressed in cells of the environment of a lesion associated with the multiple myeloma. Antigens targeted by the receptors in some embodiments include antigens associated with multiple myeloma. In some aspects, the antigen, e.g., the second or additional antigen, such as the disease-specific antigen and/or related antigen, is expressed on multiple myeloma, such as B cell maturation antigen (BCMA), G protein-coupled receptor class C group 5 member D (GPRC5D), CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, β2-Microglobulin, HM1.24, IGF-1R, IL-6R, TRAIL-R1, and the activin receptor type IIA (ActRIIA). See Benson and Byrd, J. Clin. Oncol. (2012) 30(16): 2013-15; Tao and Anderson, Bone Marrow Research (2011):924058; Chu et al., Leukemia (2013) 28(4):917-27; Garfall et al., Discov Med. (2014) 17(91):37-46. In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603,477, 8,008,450; U.S. Pub. No. US20120189622 or US20100260748; and/or International PCT Publication Nos. WO2006099875, WO2009080829 or WO2012092612 or WO2014210064. In some embodiments, such antibodies or antigen-binding fragments thereof (e.g. scFv) are contained in multispecific antibodies, multispecific chimeric receptors, such as multispecific CARs, and/or multispecific cells.

In some embodiments, the disease or disorder is associated with expression of G protein-coupled receptor class C group 5 member D (GPRC5D) and/or expression of B cell maturation antigen (BCMA).

In some embodiments, the disease or disorder is a B cell-related disorder. In some of any of the provided embodiments of the provided methods, the disease or disorder associated with BCMA is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

In some embodiments, the disease or disorder is a cancer. In some embodiments, the cancer is a GPRC5D-expressing cancer. In some embodiments, the cancer is a plasma cell malignancy and the plasma cell malignancy is multiple myeloma (MM) or plasmacytoma. In some embodiments, the cancer is multiple myeloma (MM). In some embodiments, the cancer is a relapsed/refractory multiple myeloma.

In some embodiments, the antigen is ROR1, and the disease or disorder is CLL. In some embodiments, the antigen is ROR1, and the disease or disorder is NSCLC.

In some embodiments, the antigen-binding domain, e.g., scFv, included in the miniCAR described herein specifically recognizes an antigen, such as CD19, BCMA, GPRC5D, ROR1 or FcRL5. In some embodiments, the antigen-binding domain, e.g., scFv, included in the miniCAR described herein is derived from, or is a variant of, antibodies or antigen-binding fragment that specifically binds to CD19, BCMA, GPRC5D, ROR1 or FcRL5, such as any described in Section III.B.1 above.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the prevention or treatment of disease, the appropriate dosage may depend on the type of disease to be treated, the type of cells or chimeric receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT). Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described herein, and fludarabine at any dose or administration schedule, such as those described herein. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered miniCAR expressed by the population can be conjugated either directly or indirectly through a linker to a targeting moiety. The practice of conjugating compounds, e.g., the CAR, to targeting moieties is known. See, e.g., Wadwa et al., J. Drug Targeting 3: 1 1 1 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, such values refer to numbers of miniCAR-expressing cells; in other embodiments, they refer to number of T cells or PBMCs or total cells administered.

In some embodiments, for example, where the subject is a human, the dose includes fewer than about 5×10⁸ total miniCAR-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total miniCAR-expressing-expressing cells, T cells, or peripheral blood mononuclear cells (PBMCs) and fewer than at or about 2×10⁹ total miniCAR-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁸, 8×10⁸, or 1.2×10⁹ total such cells, or the range between any two of the foregoing values.

In some embodiments, the dose of genetically engineered cells comprises from at or about 1×10⁵ to at or about 5×10⁸ total miniCAR-expressing (miniCAR+) T cells, from at or about 1×10⁵ to at or about 2.5×10⁸ total miniCAR+ T cells, from at or about 1×10⁵ to at or about 1×10⁸ total miniCAR+ T cells, from at or about 1×10⁵ to at or about 5×10⁷ total miniCAR+ T cells, from at or about 1×10⁵ to at or about 2.5×10⁷ total miniCAR+ T cells, from at or about 1×10⁵ to at or about 1×10⁷ total miniCAR+ T cells, from at or about 1×10⁵ to at or about 5×10⁶ total miniCAR+ T cells, from at or about 1×10⁵ to at or about 2.5×10⁶ total miniCAR+ T cells, from at or about 1×10⁵ to at or about 1×10⁶ total miniCAR+ T cells, from at or about 1×10⁶ to at or about 5×10⁸ total miniCAR+ T cells, from at or about 1×10⁶ to at or about 2.5×10⁸ total miniCAR+ T cells, from at or about 1×10⁶ to at or about 1×10⁸ total miniCAR+ T cells, from at or about 1×10⁶ to at or about 5×10⁷ total miniCAR+ T cells, from at or about 1×10⁶ to at or about 2.5×10⁷ total miniCAR+ T cells, from at or about 1×10⁶ to at or about 1×10⁷ total miniCAR+ T cells, from at or about 1×10⁶ to at or about 5×10⁶ total miniCAR+ T cells, from at or about 1×10⁶ to at or about 2.5×10⁶ total miniCAR+ T cells, from at or about 2.5×10⁶ to at or about 5×10⁸ total miniCAR+ T cells, from at or about 2.5×10⁶ to at or about 2.5×10⁸ total miniCAR+ T cells, from at or about 2.5×10⁶ to at or about 1×10⁸ total miniCAR+ T cells, from at or about 2.5×10⁶ to at or about 5×10⁷ total miniCAR+ T cells, from at or about 2.5×10⁶ to at or about 2.5×10⁷ total miniCAR+ T cells, from at or about 2.5×10⁶ to at or about 1×10⁷ total miniCAR+ T cells, from at or about 2.5×10⁶ to at or about 5×10⁶ total miniCAR+ T cells, from at or about 5×10⁶ to at or about 5×10⁸ total miniCAR+ T cells, from at or about 5×10⁶ to at or about 2.5×10⁸ total miniCAR+ T cells, from at or about 5×10⁶ to at or about 1×10⁸ total miniCAR+ T cells, from at or about 5×10⁶ to at or about 5×10⁷ total miniCAR+ T cells, from at or about 5×10⁶ to at or about 2.5×10⁷ total miniCAR+ T cells, from at or about 5×10⁶ to at or about 1×10⁷ total miniCAR+ T cells, from at or about 1×10⁷ to at or about 5×10⁸ total miniCAR+ T cells, from at or about 1×10⁷ to at or about 2.5×10⁸ total miniCAR+ T cells, from at or about 1×10⁷ to at or about 1×10⁸ total miniCAR+ T cells, from at or about 1×10⁷ to at or about 5×10⁷ total miniCAR+ T cells, from at or about 1×10⁷ to at or about 2.5×10⁷ total miniCAR+ T cells, from at or about 2.5×10⁷ to at or about 5×10⁸ total miniCAR+ T cells, from at or about 2.5×10⁷ to at or about 2.5×10⁸ total miniCAR+ T cells, from at or about 2.5×10⁷ to at or about 1×10⁸ total miniCAR+ T cells, from at or about 2.5×10⁷ to at or about 5×10⁷ total miniCAR+ T cells, from at or about 5×10⁷ to at or about 5×10⁸ total miniCAR+ T cells, from at or about 5×10⁷ to at or about 2.5×10⁸ total miniCAR+ T cells, from at or about 5×10⁷ to at or about 1×10⁸ total miniCAR+ T cells, from at or about 1×10⁸ to at or about 5×10⁸ total miniCAR+ T cells, from at or about 1×10⁸ to at or about 2.5×10⁸ total miniCAR+ T cells, from at or about or 2.5×10⁸ to at or about 5×10⁸ total miniCAR+ T cells. In some embodiments, the dose of genetically engineered cells comprises from or from about 2.5×10⁷ to at or about 1.5×10⁸ total miniCAR+ T cells, such as from or from about 5×10⁷ to or to about 1×10⁸ total miniCAR+ T cells.

In some embodiments, the dose of genetically engineered cells comprises at least at or about 1×10⁵ miniCAR⁺ cells, at least at or about 2.5×10⁵ miniCAR⁺ cells, at least at or about 5×10⁵ miniCAR⁺ cells, at least at or about 1×10⁶ miniCAR⁺ cells, at least at or about 2.5×10⁶ miniCAR⁺ cells, at least at or about 5×10⁶ miniCAR⁺ cells, at least at or about 1×10⁷ miniCAR⁺ cells, at least at or about 2.5×10⁷ miniCAR⁺ cells, at least at or about 5×10⁷ miniCAR⁺ cells, at least at or about 1×10⁸ miniCAR⁺ cells, at least at or about 1.5×10⁸ miniCAR⁺ cells, at least at or about 2.5×10⁸ miniCAR⁺ cells, or at least at or about 5×10⁸ miniCAR⁺ cells.

In some embodiments, the cell therapy comprises administration of a dose comprising a number of cell from or from about 1×10⁵ to or to about 5×10⁸ total miniCAR-expressing cells, total T cells, or total peripheral blood mononuclear cells (PBMCs), from or from about 5×10⁵ to or to about 1×10⁷ total miniCAR-expressing cells, total T cells, or total peripheral blood mononuclear cells (PBMCs) or from or from about 1×10⁶ to or to about 1×10⁷ total miniCAR-expressing cells, total T cells, or total peripheral blood mononuclear cells (PBMCs), each inclusive. In some embodiments, the cell therapy comprises administration of a dose of cells comprising a number of cells at least or at least about 1×10⁵ total miniCAR-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also miniCAR-expressing (e.g. miniCAR⁺) cells. In some embodiments, the cell therapy comprises administration of a dose comprising a number of cell from or from about 1×10⁵ to or to about 5×10⁸ CD3⁺ or CD8⁺ total T cells or CD3⁺ or CD8⁺ miniCAR-expressing cells, from or from about 5×10⁵ to or to about 1×10⁷ CD3⁺ or CD8⁺ total T cells or CD3⁺ or CD8⁺ miniCAR-expressing cells, or from or from about 1×10⁶ to or to about 1×10⁷ CD3⁺ or CD8⁺ total T cells or CD3⁺ or CD8⁺ miniCAR-expressing cells, each inclusive. In some embodiments, the cell therapy comprises administration of a dose comprising a number of cell from or from about 1×10⁵ to or to about 5×10⁸ total CD3⁺/miniCAR⁺ or CD8⁺/miniCAR⁺ cells, from or from about 5×10⁵ to or to about 1×10⁷ total CD3⁺/miniCAR⁺ or CD8⁺/miniCAR⁺ cells, or from or from about 1×10⁶ to or to about 1×10⁷ total CD3⁺/miniCAR⁺ or CD8⁺/miniCAR⁺ cells, each inclusive.

In some embodiments, the T cells of the dose include CD4+ T cells, CD8+ T cells or CD4+ and CD8+ T cells.

In some embodiments, for example, where the subject is human, the CD8⁺ T cells of the dose, including in a dose including CD4⁺ and CD8⁺ T cells, includes between at or about 1×10⁶ and at or about 5×10⁸ total miniCAR-expressing CD8⁺ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total miniCAR-expressing CD8+ T cells, from or from about 1×10⁷ to or to about 5×10⁷ total miniCAR-expressing CD8+ T cells, from or from about 1×10⁷ to or to about 0.25×10⁸ total miniCAR-expressing CD8+ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total miniCAR-expressing CD8⁺ T cells.

In some embodiments, the dose of cells, e.g., miniCAR-expressing T cells, is administered to the subject as a single dose or is administered only one time within a period of two weeks, one month, three months, six months, 1 year or more. In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the miniCAR. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a miniCAR to CD8+ cells expressing a miniCAR and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or miniCAR being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., miniCAR-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of miniCAR (e.g., miniCAR+)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or miniCARs being administered.

In some embodiments, the methods also include administering one or more additional doses of cells expressing a miniCAR and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells being administered.

### V. Pharmaceutical Composition and Formulation

Also provided are compositions, such as pharmaceutical compositions and formulations for administration, such as for adoptive cell therapy. In some aspects, the pharmaceutical compositions contain any of the engineered cells or compositions containing the engineered cells described herein, e.g., including a modified invariant CD3-IgSF chain locus, e.g., CD3E, CD3D, or CD3G locus, containing a transgene sequence as described herein. In some embodiments, the dose of cells comprising cells engineered with a miniCAR as described herein, is provided as a composition or formulation, such as a pharmaceutical composition or formulation. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or miniCAR, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. In some aspects, the cells are isolated from a subject, engineered, and administered to the same subject. In other aspects, they are isolated from one subject, engineered, and administered to another subject. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### VI. Kits and Articles of Manufacture

Also provided are articles of manufacture, systems, apparatuses, and kits useful in performing the provided embodiments. In some embodiments, the provided articles of manufacture or kits contain one or more components of the one or more agent(s) capable of inducing genetic disruption and/or template polynucleotide(s), e.g., template polynucleotides containing transgenes as provided herein. In some embodiments, the articles of manufacture or kits can be used in methods for engineering T cells to express a chimeric receptor, e.g., a miniCAR, and/or other molecules, such as via integration of transgene sequences by homology-dependent repair (HDR), for example, to generate the engineered cells comprising a modified invariant CD3-IgSF chain locus, e.g., CD3E, CD3D, or CD3G locus, containing a nucleic acid sequence encoding a miniCAR, wherein the miniCAR is a fusion protein including a heterologous antigen-binding domain and an endogenous invariant CD3-IgSF chain.

In some embodiments, the articles of manufacture or kits include polypeptides, nucleic acids, vectors and/or polynucleotides useful in performing the provided methods. In some embodiments, the articles of manufacture or kits include one or more agent(s) capable of inducing a genetic disruption, for example, at an invariant CD3-IgSF chain locus, e.g., CD3E, CD3D, or CD3G locus, (such as those described in Section LA herein). In some embodiments, the articles of manufacture or kits include one or more nucleic acid molecules, e.g., a plasmid or a DNA fragment, that encodes one or more components of the one or more agent(s) capable of inducing genetic disruption and/or comprises template polynucleotide(s), e.g., for use in targeting transgene sequences into the cell via HDR, such as those described in Section I.B.2 herein. In some embodiments, the articles of manufacture or kits provided herein contain control vectors.

In some embodiments, the articles of manufacture or kits provided herein contain one or more agent(s), wherein each of the one or more agent is independently capable of inducing a genetic disruption of a target site within an invariant CD3-IgSF chain locus, e.g., CD3E, CD3D, or CD3G locus; and a template polynucleotide comprising a transgene encoding antigen-binding receptor and optionally a linker, wherein the transgene is targeted for integration at or near the target site via homology directed repair (HDR). In some aspects, the one or more agent(s) capable of inducing a genetic disruption is any described herein. In some aspects, the one or more agent(s) is a ribonucleoprotein (RNP) complex comprising a Cas9/gRNA complex. In some aspects, the gRNA included in the RNP targets a target site in an invariant CD3-IgSF chain locus, such as any target site described herein. In some aspects, the template polynucleotide is any of the template polynucleotide described herein.

In some embodiments, the articles of manufacture or kits include one or more containers, typically a plurality of containers, packaging material, and a label or package insert on or associated with the container or containers and/or packaging, generally including instructions for use, e.g., instructions for introducing the components into the cells for engineering.

The articles of manufacture provided herein contain packaging materials. Packaging materials for use in packaging the provided materials are well known. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the one or more agent(s) capable of inducing genetic disruption and/or template polynucleotide(s) are packaged separately. In some embodiments, each container can have a single compartment. In some embodiments, other components of the articles of manufacture or kits are packaged separately, or together in a single compartment.

Also provided are articles of manufacture, systems, apparatuses, and kits useful in administering the provided cells and/or cell compositions, e.g., for use in therapy or treatment. In some embodiments, the articles of manufacture or kits provided herein contain T cells and/or T cell compositions, such as any T cells and/or T cell compositions described herein. In some aspects, the articles of manufacture or kits provided herein can be used for administration of the T cells or T cell compositions, and can include instructions for use.

In some embodiments, the articles of manufacture or kits provided herein contain T cells, and/or T cell compositions, such as any T cells, and/or T cell compositions described herein. In some embodiments, the T cells, and/or T cell compositions any of the modified T cells used the screening methods described herein. In some embodiments, the articles of manufacture or kits provided herein contain control or unmodified T cells and/or T cell compositions. In some embodiments, the article of manufacture or kits include one or more instructions for administration of the engineered cells and/or cell compositions for therapy.

The articles of manufacture and/or kits containing cells or cell compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may include (a) a first container with a composition contained therein, wherein the composition includes engineered cells expressing a miniCAR; and (b) a second container with a composition contained therein, wherein the composition includes the second agent. In some embodiments, the article of manufacture may include (a) a first container with a first composition contained therein, wherein the composition includes a subtype of engineered cells expressing a miniCAR; and (b) a second container with a composition contained therein, wherein the composition includes a different subtype of engineered cells expressing a miniCAR. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1%.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A.M., ed., Oxford University Press, New York, 1988; Biocomputing: Informatics and Genome Projects, Smith, D.W., ed., Academic Press, New York, 1993; Computer Analysis of Sequence Data, Part I, Griffin, A. M., and Griffin, H.G., eds., Humana Press, New.Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### VIII. Exemplary Embodiments

Among the provided embodiments are:
1. An engineered T cell, comprising a modified invariant CD3-immunoglobulin superfamily (invariant CD3-IgSF) chain locus comprising a nucleic acid sequence encoding a mini chimeric antigen receptor (miniCAR), wherein the miniCAR is a fusion protein comprising a heterologous antigen-binding domain and an endogenous invariant CD3 chain of the invariant CD3-IgSF chain, wherein:
the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain.
2. An engineered T cell expressing a mini chimeric antigen receptor (miniCAR), wherein the miniCAR is a fusion protein comprising a heterologous antigen-binding domain and an endogenous invariant CD3 chain of the immunoglobulin superfamily (invariant CD3-IgSF chain).
3. An engineered T cell comprising a transgene encoding an antigen-binding domain inserted in-frame with an open reading frame of a locus encoding an endogenous invariant CD3 chain of the immunoglobulin superfamily (invariant CD3-IgSF chain), wherein the engineered T cell expresses a miniCAR fusion protein comprising a heterologous antigen-binding domain and the endogenous invariant CD3-IgSF chain.
4. The engineered T cell of embodiment 2 or 3, wherein the miniCAR is expressed from a modified invariant CD3-immunoglobulin superfamily (invariant CD3-IgSF) chain locus comprising a nucleic acid sequence encoding the miniCAR, wherein:
the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain.
5. The engineered T cell of any of embodiments 1-4, wherein the invariant CD3-IgSF chain is a CD3 epsilon (CD3e) chain.
6. The engineered T cell of any of embodiments 1-4, wherein the invariant CD3-IgSF chain is a CD3 delta (CD3d) chain.
7. The engineered T cell of any of embodiments 1-4, wherein the invariant CD3-IgSF chain is a CD3 gamma (CD3g) chain.
8. The engineered T cell of embodiment 1 or 4, wherein the modified invariant CD3-IgSF chain locus is a modified CD3 epsilon (CD3E) locus encoding a CD3e chain, a modified CD3 delta (CD3D) locus encoding a CD3d chain, or a modified CD3 gamma (CD3G) locus encoding a CD3g chain.
9. The engineered T cell of any of embodiments 1, 4, 5 and 8, wherein the modified invariant CD3-IgSF chain locus is a modified CD3E locus encoding a CD3e chain.
10. The engineered T cell of any of embodiments 1, 4, 5 and 8, wherein the modified invariant CD3-IgSF chain locus is a modified CD3D locus encoding a CD3d chain.
11. The engineered T cell of any of embodiments 1, 4, 5 and 8, wherein the modified invariant CD3-IgSF chain locus is a modified CD3G locus encoding a CD3g chain.
12. An engineered T cell, comprising a modified CD3E locus comprising a nucleic acid sequence encoding a mini chimeric antigen receptor (miniCAR), wherein the miniCAR is a fusion protein comprising a heterologous antigen-binding domain and an endogenous CD3e chain, wherein:
the nucleic acid sequence comprises an in-frame fusion of (i) a transgene comprising a sequence encoding the antigen-binding domain and (ii) an open reading frame of the endogenous CD3E locus encoding the CD3e chain.
13. An engineered T cell expressing a mini chimeric antigen receptor (miniCAR), wherein the miniCAR is a fusion protein comprising a heterologous antigen-binding domain and an endogenous CD3e chain.
14. An engineered T cell comprising a transgene encoding an antigen-binding domain inserted in-frame with an open reading frame of a locus encoding an endogenous CD3e chain, wherein the engineered T cell expresses a miniCAR fusion protein comprising a heterologous antigen-binding domain and the endogenous CD3e chain.
15. The engineered T cell of embodiment 13 or 14, wherein the miniCAR is expressed from a modified CD3E chain locus comprising a nucleic acid sequence encoding the miniCAR, wherein:
the nucleic acid sequence comprises an in-frame fusion of (i) a transgene comprising a sequence encoding the antigen-binding domain and (ii) an open reading frame of the endogenous CD3E locus encoding the CD3e chain.
16. The engineered T cell of any of embodiments 1-15, wherein the antigen-binding domain is or comprises an antibody or an antigen-binding fragment thereof.
17. The engineered T cell of any of embodiments 1-16, wherein the antigen-binding domain is or comprises a Fab fragment, a Fab₂ fragment, a single domain antibody, or a single chain variable fragment (scFv).
18. The engineered T cell of any of embodiments 1-17, wherein the antigen-binding domain is an scFv.
19. The engineered T cell of 1, 4, 5 and 8, wherein the modified invariant CD3-IgSF chain locus comprises, in order from 5′ to 3′, a sequence of nucleotides encoding the heterologous antigen-binding domain and the endogenous invariant CD3-IgSF chain.
20. The engineered T cell of any of embodiments 9, 12 and 15, wherein the modified CD3E locus comprises, in order from 5′ to 3′, a sequence of nucleotides encoding the heterologous antigen-binding domain and the endogenous CD3e chain.
21. The engineered T cell of any of embodiments 1-11 and 16-19, wherein the heterologous antigen-binding domain and the invariant CD3-IgSF chain are directly linked.
22. The engineered T cell of any of embodiments 1-11 and 16-19, wherein the heterologous antigen-binding domain and the invariant CD3-IgSF chain are linked indirectly via a linker.
23. The engineered T cell of any of embodiments 12-18 and 20, wherein the heterologous antigen-binding domain and the CD3e chain are directly linked.
24. The engineered T cell of any of embodiments 12-18 and 20, wherein the heterologous antigen-binding domain and the CD3e chain are linked indirectly via a linker.
25. The engineered T cell of any of embodiments 1, 3-12 and 14-24, wherein the transgene further comprises a nucleic acid sequence encoding a linker.
26. The engineered T cell of embodiment 25, wherein the linker is positioned 3′ to the antigen-binding domain.
27. An engineered T cell, comprising a modified CD3E locus comprising a nucleic acid sequence encoding a miniCAR, the miniCAR comprising a heterologous antigen-binding domain and an endogenous CD3e chain, wherein:
the nucleic acid sequence comprises an in-frame fusion of (i) a transgene comprising a sequence encoding the antigen-binding domain, wherein the antigen-binding domain is an scFv, and a sequence encoding a linker, and (ii) an open reading frame of an endogenous CD3E locus encoding the CD3e chain.
28. The engineered T cell of any of embodiments 25-27, wherein the transgene sequence comprises, in order from 5′ to 3′, a sequence of nucleotides encoding the antigen-binding domain and a sequence of nucleotides encoding the linker.
29. The engineered T cell of any of embodiments 25 or 26, wherein the modified invariant CD3-IgSF chain locus comprises, in order from 5′ to 3′, a sequence of nucleotides encoding the antigen-binding domain, the linker, and the invariant CD3-IgSF chain.
30. The engineered T cell of any of embodiments 25-29, wherein the linker is a polypeptide linker.
31. The engineered T cell of any of embodiments 25-30, wherein the linker is a polypeptide that is 2, 3, 4, 5, 6, 7, 8, 9, 10, 11, 12, 13, 14, 15, 16, 17, 18, 19 or 20 amino acids in length.
32. The engineered T cell of any of embodiments 25-31, wherein the linker is a polypeptide that is 3 to 18 amino acids in length.
33. The engineered T cell of any of embodiments 25-31, wherein the linker is a polypeptide that is 12 to 18 amino acids in length.
34. The engineered T cell of any of embodiments 25-31, wherein the linker is a polypeptide that is 15 to 18 amino acids in length.
35. The engineered T cell of any of embodiments 25-31, wherein the linker comprises GS, GGS, GGGGS (SEQ ID NO:122), GGGGGS (SEQ ID NO:128) and combinations thereof.
36. The engineered T cell of any of embodiments 25-30, wherein the linker comprises (GGS)n, wherein n is 1 to 10, (GGGGS)n (SEQ ID NO: 121), wherein n is 1 to 10, or (GGGGGS)n (SEQ ID NO:129), wherein n is 1 to 4.
37. The engineered T cell of any of embodiments 25-30, wherein the linker is selected from among a linker that is or comprises GGS, is or comprises GGGGS (SEQ ID NO: 122), is or comprises GGGGGS (SEQ ID NO: 128), is or comprises (GGS)2 (SEQ ID NO: 130), is or comprises GGSGGSGGS (SEQ ID NO: 131), is or comprises GGSGGSGGSGGS (SEQ ID NO:132), is or comprises GGSGGSGGSGGSGGS (SEQ ID NO:133), is or comprises GGGGGSGGGGGSGGGGGS (SEQ ID NO:134), is or comprises GGSGGGGSGGGGSGGGGS (SEQ ID NO: 135), is or comprises and GGGGSGGGGSGGGGS (SEQ ID NO:16).
38. The engineered T cell of any of embodiments 25-31 and 37, wherein the linker is or comprises GGGGSGGGGSGGGGS (SEQ ID NO:16).
39. The engineered T cell of any of embodiments 1, 3-12 and 14-38, wherein the transgene further comprises a nucleic acid sequence encoding one or more multicistronic elements, optionally wherein the one or more multicistronic elements are or comprise a ribosome skip sequence, optionally wherein the ribosome skip sequence is a T2A, a P2A, an E2A, or an F2A element.
40. The engineered T cell of embodiment 39, wherein the P2A element comprises the sequence set forth in SEQ ID NO: 3.
41. The engineered T cell of embodiment 39 or 40, wherein at least one of the one or more multicistronic elements is positioned 5′ to the antigen-binding domain.
42. The engineered T cell of any of embodiments 39-41, wherein the transgene sequence comprises, in order from 5′ to 3′, a sequence of nucleotides encoding the multicistronic element, optionally the P2A element; the antigen-binding domain; and the linker.
43. The engineered T cell of any of embodiments 1, 3-12 and 14-42, wherein the transgene further comprises a nucleic acid sequence encoding an affinity tag.
44. The engineered T cell of embodiment 43, wherein the affinity tag is a streptavidin binding peptide.
45. The engineered T cell of embodiment 44, wherein the streptavidin binding peptide is or comprises the sequence Trp-Ser-His-Pro-Gln-Phe-Glu-Lys (SEQ ID NO: 137), Trp-Arg-His-Pro-Gln-Phe-Gly-Gly (SEQ ID NO:136), Trp-Ser-His-Pro-Gln-Phe-Glu-Lys-(GlyGlyGlySer)3-Trp-Ser-His-Pro-Gln-Phe-Glu-Lys (SEQ ID NO: 146), Trp-Ser-His-Pro-Gln-Phe-Glu-Lys-(GlyGlyGlySer)2-Trp-Ser-His-Pro-Gln-Phe-Glu-Lys (SEQ ID NO: 147) and Trp-Ser-His-Pro-Gln-Phe-Glu-Lys-(GlyGlyGlySer)2Gly-Gly-Ser-Ala-Trp-Ser-His-Pro-Gln-Phe-Glu-Lys (SEQ ID NO: 148).
46. The engineered T cell of any of embodiments 39-45, wherein the modified invariant CD3-IgSF chain locus comprises, in order from 5′ to 3′, a sequence of nucleotides encoding the multicistronic element, optionally a P2A element; the antigen-binding domain; the linker; and the invariant CD3-IgSF chain.
47. The engineered T cell of any of embodiments 39-45, wherein the modified CD3E locus comprises, in order from 5′ to 3′, a sequence of nucleotides encoding the multicistronic element, optionally a P2A element; the antigen-binding domain; the linker; and the CD3e chain.
48. The engineered T cells of any of embodiments 1, 3-11, 16-19, 21, 22, 25, 26 and 28-47, wherein the open reading frame of the endogenous invariant CD3-IgSF chain locus encodes a full length mature invariant CD3-IgSF chain.
49. The engineered T cell of any of embodiments 1, 4-11, 16-19, 21, 22, 25, 26 and 28-48, wherein the modified invariant CD3-IgSF chain locus comprises the promoter and/or regulatory or control element of the endogenous locus operably linked to control expression the nucleic acid sequence encoding the miniCAR.
50. The engineered T cell of any of embodiments 1, 4-11, 16-19, 21, 22, 25, 26 and 28-48, wherein the modified invariant CD3-IgSF chain locus comprises one or more heterologous regulatory or control elements operably linked to control expression of the miniCAR or a portion thereof.
51. The engineered T cell of any of embodiments 1-50, wherein the antigen-binding domain binds to a target antigen that is associated with, specific to, and/or expressed on a cell or tissue of a disease, disorder or condition.
52. The engineered T cell of embodiment 51, wherein the target antigen is a tumor antigen.
53. The engineered T cell of embodiment 51 or 52, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.
54. The engineered T cell of any of embodiments 1-53, wherein the miniCAR assembles into a TCR/CD3 complex in place of the corresponding endogenous invariant CD3-IgSF chain of the TCR/CD3 complex.
55. The engineered T cell of any of embodiments 5 and 8-54, wherein the miniCAR assembles into a TCR/CD3 complex in place of the corresponding endogenous invariant CD3-IgSF CD3e chain of the TCR/CD3 complex.
56. The engineered T cell of embodiment 54 or 55, wherein binding of a target antigen by the heterologous antigen-binding domain of the miniCAR induces antigen-dependent signaling via the TCR/CD3 complex.
57. The engineered T cell of any of embodiments 54-56, wherein the miniCAR exhibits reduced tonic signaling via the TCR/CD3 complex compared to T cells engineered with a chimeric antigen receptor (CAR) that comprises the same antigen-binding domain.
58. The engineered T cell of any of embodiments 1-57, wherein the engineered T cell exhibits increased persistence compared to T cells engineered with a chimeric antigen receptor (CAR) that comprises the same antigen-binding domain and a heterologous CD3zeta (CD3z) signaling domain, and optionally a costimulatory signaling domain.
59. The engineered T cell of any of embodiments 1-58, wherein the engineered T cell exhibits increased cytolytic activity compared to T cells engineered with a chimeric antigen receptor (CAR) that comprises the same antigen-binding domain and a heterologous CD3zeta (CD3z) signaling domain, and optionally a costimulatory signaling domain.
60. The engineered T cell of any of embodiments 1-59, wherein the T cell is a primary T cell derived from a subject.
61. The engineered T cells of embodiment 60, wherein the subject is a human.
62. The engineered T cell of any of embodiments 1-61, wherein the T cell is a CD8+ T cell or a subtype thereof, or a CD4+ T cell or a subtype thereof.
63. The engineered T cell of any of embodiments 1, 3-12 and 14-42, wherein the transgene is integrated at the endogenous invariant CD3-IgSF chain locus of a T cell via homology directed repair (HDR).
64. A polynucleotide, comprising:
(a) a nucleic acid sequence encoding an antigen-binding domain; and
(b) one or more homology arms linked to the nucleic acid sequence, wherein the one or more homology arms comprise a sequence homologous to one or more regions of an open reading frame of an invariant CD3 chain of the immunoglobulin superfamily (invariant CD3-IgSF chain) locus of a T cell, wherein the invariant CD3-IgSFchain locus encodes an invariant CD3-IgSF chain.
65. The polynucleotide of embodiment 64, wherein the one or more homology arms comprise a sequence homologous to one or more regions of an open reading frame of the invariant CD3-IgSF chain locus, wherein the invariant CD3-IgSF chain locus is a CD3E locus encoding a CD3e chain, a CD3D locus encoding a CD3d chain, or a CD3G locus encoding a CD3g chain.
66. The polynucleotide of embodiment 64 or 65, wherein the invariant CD3-IgSF chain locus is a CD3E locus encoding a CD3e chain.
67. The polynucleotide of embodiment 64 or 65, wherein the invariant CD3-IgSF chain locus is a CD3D locus encoding a CD3d chain.
68. The polynucleotide of embodiment 64 or 65, wherein the invariant CD3-IgSF chain locus is a CD3G locus encoding a CD3g chain.
69. A polynucleotide, comprising:
(a) a nucleic acid sequence encoding an antigen-binding domain; and
(b) one or more homology arms linked to the nucleic acid sequence encoding the transgene, wherein the one or more homology arms comprise a sequence homologous to one or more regions of an open reading frame of a CD3E locus encoding a CD3e chain.
70. The polynucleotide of any of embodiments 64-69, wherein the antigen-binding domain is or comprises an antibody or an antigen-binding fragment thereof.
71. The polynucleotide of any of embodiments 64-70, wherein the antigen-binding domain is or comprises a Fab fragment, a Fab₂ fragment, a single domain antibody, or a single chain variable fragment (scFv).
72. The polynucleotide of any of embodiments 64-71, wherein the antigen-binding domain is an scFv.
73. The polynucleotide of any of embodiments 64-72, wherein the nucleic acid sequence further comprises nucleotides encoding a linker operably connected to the encoded antigen-binding domain, wherein the linker is positioned 3′ to the antigen-binding domain.
74. A polynucleotide, comprising:
(a) a nucleic acid sequence encoding a single chain variable fragment (scFv) and a sequence encoding a linker; and
(b) one or more homology arms linked to the nucleic acid sequence, wherein the one or more homology arms comprise a sequence homologous to one or more regions of an open reading frame of a CD3E locus encoding a CD3e chain.
75. The polynucleotide of embodiment 73 or 74, wherein the encoded linker is a polypeptide encoded linker.
76. The polynucleotide of any of embodiments 73-75, wherein the encoded linker is a polypeptide that is 2, 3, 4, 5, 6, 7, 8, 9, 10, 11, 12, 13, 14, 15, 16, 17, 18, 19 or 20 amino acids in length.
77. The polynucleotide of any of embodiments 73-76, wherein the encoded linker is a polypeptide that is 3 to 18 amino acids in length.
78. The polynucleotide of any of embodiments 73-76, wherein the encoded linker is a polypeptide that is 12 to 18 amino acids in length.
79. The polynucleotide of any of embodiments 73-76, wherein the encoded linker is a polypeptide that is 15 to 18 amino acids in length.
80. The polynucleotide of any of embodiments 73-76, wherein the encoded linker comprises GS, GGS, GGGGS (SEQ ID NO:122), GGGGGS (SEQ ID NO:128) and combinations thereof.
81. The polynucleotide of any of embodiments 73-75, wherein the encoded linker comprises (GGS)n, wherein n is 1 to 10, (GGGGS)n (SEQ ID NO: 121), wherein n is 1 to 10, or (GGGGGS)n (SEQ ID NO:129), wherein n is 1 to 4.
82. The polynucleotide of any of embodiments 73-75, wherein the encoded linker is selected from among a encoded linker that is or comprises GGS, is or comprises GGGGS (SEQ ID NO: 122), is or comprises GGGGGS (SEQ ID NO: 128), is or comprises (GGS)2 (SEQ ID NO: 130), is or comprises GGSGGSGGS (SEQ ID NO: 131), is or comprises GGSGGSGGSGGS (SEQ ID NO:132), is or comprises GGSGGSGGSGGSGGS (SEQ ID NO:133), is or comprises GGGGGSGGGGGSGGGGGS (SEQ ID NO:134), is or comprises GGSGGGGSGGGGSGGGGS (SEQ ID NO: 135), is or comprises and GGGGSGGGGSGGGGS (SEQ ID NO:16).
83. The polynucleotide of any of embodiments 73-76 and 82, wherein the encoded linker is or comprises GGGGSGGGGSGGGGS (SEQ ID NO:16).
84. The polynucleotide of any of embodiments 73-83, wherein the nucleic acid sequence comprises, in order from 5′ to 3′, a sequence of nucleotides encoding the antigen-binding domain and a sequence of nucleotides encoding the linker.
85. The polynucleotide of any of embodiments 64-84, wherein the nucleic acid sequence further comprises nucleotides encoding one or more multicistronic elements, optionally wherein the one or more multicistronic elements are or comprise a ribosome skip sequence, optionally wherein the ribosome skip sequence is a T2A, a P2A, an E2A, or an F2A element.
86. The polynucleotide of embodiment 85, wherein the P2A element comprises the sequence set forth in SEQ ID NO: 3.
87. The polynucleotide of embodiment 85 or 86, wherein the nucleic acid sequence comprises, in order from 5′ to 3′, a sequence of nucleotides encoding the multicistronic element, optionally the P2A element; the antigen-binding domain; and the linker.
88. The polynucleotide of any of embodiments 64-87, wherein the nucleic acid sequence further comprises a nucleic acid sequence encoding an affinity tag.
89. The polynucleotide of embodiment 88, wherein the affinity tag is a streptavidin binding peptide.
90. The polynucleotide of embodiment 89, wherein the streptavidin binding peptide is or comprises the sequence Trp-Ser-His-Pro-Gln-Phe-Glu-Lys (SEQ ID NO: 137), Trp-Arg-His-Pro-Gln-Phe-Gly-Gly (SEQ ID NO:136), Trp-Ser-His-Pro-Gln-Phe-Glu-Lys-(GlyGlyGlySer)3-Trp-Ser-His-Pro-Gln-Phe-Glu-Lys (SEQ ID NO: 146), Trp-Ser-His-Pro-Gln-Phe-Glu-Lys-(GlyGlyGlySer)2-Trp-Ser-His-Pro-Gln-Phe-Glu-Lys (SEQ ID NO: 147) and Trp-Ser-His-Pro-Gln-Phe-Glu-Lys-(GlyGlyGlySer)2Gly-Gly-Ser-Ala-Trp-Ser-His-Pro-Gln-Phe-Glu-Lys (SEQ ID NO: 148).
91. The polynucleotide of any of embodiments 64-90, wherein the one or more homology arms comprise a 5′ homology arm and a 3′ homology arm and the polynucleotide comprises the structure [5′ homology arm]-[nucleic acid sequence of (a)]-[3′ homology arm].
92. The polynucleotide of embodiment 91, wherein the 5′ homology arm and the 3′ homology arm independently are at or about 100, 200, 300, 400, 500, 600, 700 or 800 nucleotides in length, or any value between any of the foregoing, or are greater than at or about 100 nucleotides in length, optionally at or about 100, 200 or 300 nucleotides in length, or any value between any of the foregoing.
93. The polynucleotide of embodiment 91 or 92, wherein the 5′ homology arm comprises (i) the sequence set forth in SEQ ID NO: 4, or (ii) a sequence that exhibits at least 85%, 86%, 87%, 88%, 89%, 90%, 91%, 92%, 93%, 94%, 95%, 96%, 97%, 98%, 99% or more sequence identity to SEQ ID NO: 4 or (ii) a partial sequence of (i) or (ii).
94. The polynucleotide of any of embodiments 91-93, wherein the 3′ homology arm comprises (i) the sequence set forth in SEQ ID NO: 5, or (ii) a sequence that exhibits at least 85%, 86%, 87%, 88%, 89%, 90%, 91%, 92%, 93%, 94%, 95%, 96%, 97%, 98%, 99% or more sequence identity to SEQ ID NO: 5 or (iii) a partial sequence of (i) or (ii).
95. The polynucleotide of any of embodiments 64-94, wherein the encoded antigen-binding domain binds to a target antigen that is associated with, specific to, and/or expressed on a cell or tissue of a disease, disorder or condition.
96. The polynucleotide of embodiment 95, wherein the target antigen is a tumor antigen.
97. The polynucleotide of embodiment 95 or 96, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.
98. The polynucleotide of any of embodiments 64-68 and 70-97, wherein introduction of the polynucleotide into a genome of a T cell generates a modified invariant CD3-IgSF chain locus encoding a mini chimeric antigen receptor (miniCAR), wherein the miniCAR is a fusion protein comprising the antigen-binding domain encoded by the nucleic acid of the polynucleotide and an endogenous invariant CD3-IgSF chain, and wherein the modified invariant CD3-IgSF chain locus comprises the nucleic acid encoding the antigen-binding domain in-frame with an open reading frame of the endogenous invariant CD3-IgSF chain locus encoding the invariant CD3-IgSF chain.
99. The polynucleotide of embodiment 98, wherein the endogenous invariant CD3-IgSF chain is a CD3e chain, a CD3d chain, or a CD3g chain.
100. The polynucleotide of embodiment 98 or 99, wherein the endogenous invariant CD3-IgSF chain is a CD3e chain.
101. The polynucleotide of embodiment 98 or 99, wherein the endogenous invariant CD3-IgSF chain is a CD3d chain.
102. The polynucleotide of embodiment 98 or 99, wherein the endogenous invariant CD3-IgSF chain is a CD3g chain.
103. The polynucleotide of any of embodiments 98-102, wherein the encoded miniCAR assembles into a TCR/CD3 complex in place of the corresponding endogenous invariant CD3-IgSF chain of the TCR/CD3 complex.
104. The polynucleotide of any of embodiments 64-103, that is a linear polynucleotide, optionally a double-stranded polynucleotide or a single-stranded polynucleotide.
105. The polynucleotide of any of embodiments 64-103, wherein the polynucleotide is comprised in a vector.
106. The polynucleotide of any of embodiments 64-105, wherein the polynucleotide is between at or about 500 and at or about 3000 nucleotides, at or about 1000 and at or about 2500 nucleotides, or at or about 1500 nucleotides and at or about 2000 nucleotides in length.
107. A vector comprising the polynucleotide of any of embodiments 64-103, 105 and 106.
108. The vector of embodiment 107, wherein the vector is a viral vector.
109. The vector of embodiment 108, wherein the viral vector is an AAV vector, optionally wherein the AAV vector is an AAV2 or AAV6 vector.
110. The vector of embodiment 108, wherein the viral vector is a retroviral vector, optionally a lentiviral vector.
111. A method of producing genetically engineered T cells, the method comprising introducing the polynucleotide of any of embodiments 64-106 into a population of T cells, where T cells of the population comprise a genetic disruption at an endogenous invariant CD3-IgSF chain locus, wherein the invariant CD3-IgSF chain locus encodes an invariant CD3-IgSF chain.
112. A method of producing genetically engineered T cells, the method comprising introducing the vector of any of embodiments 107-110 into a population of T cells, where T cells of the population comprise a genetic disruption at an endogenous invariant CD3-IgSF chain locus, wherein the invariant CD3-IgSF chain locus encodes an invariant CD3-IgSF chain.
113. A method of producing genetically engineered T cells, the method comprising:
(a) introducing, into a population of T cells, one or more agents capable of inducing a genetic disruption at a target site within an endogenous invariant CD3-IgSF chain locus of T cells in the population, wherein the invariant CD3-IgSF chain locus encodes an invariant CD3-IgSF chain; and
(b) introducing the polynucleotide of any of embodiments 64-106 into the population of T cells, wherein T cells in the population comprise a genetic disruption at the endogenous invariant CD3 IgSF chain locus.
114. A method of producing genetically engineered T cells, the method comprising:
(a) introducing, into a population of T cells, one or more agents capable of inducing a genetic disruption at a target site within an endogenous invariant CD3-IgSF chain locus of T cells in the population, wherein the invariant CD3-IgSF chain locus encodes an invariant CD3-IgSF chain; and
(b) introducing the vector of any of embodiments 107-110 into the population of T cells, wherein T cells in the population comprise a genetic disruption at the endogenous invariant CD3 IgSF chain locus.
115. The method of any of embodiments 111-114, wherein the nucleic acid sequence of the polynucleotide is integrated in the endogenous invariant CD3-IgSF chain locus via homology directed repair (HDR).
116. A method of producing genetically engineered T cells, the method comprising:
(a) introducing, into a population comprising T cells, one or more agents capable of inducing a genetic disruption at a target site within an endogenous CD3E locus; and
(b) introducing the polynucleotide of any of embodiments 66 and 69-106 into the population comprising T cells, wherein T cells in the population comprise a genetic disruption at the endogenous CD3E locus.
117. A method of producing genetically engineered T cells, the method comprising:
(a) introducing, into a population comprising T cells, one or more agents capable of inducing a genetic disruption at a target site within an endogenous CD3E locus; and
(b) introducing the vector of any of embodiments 107-110 into the population comprising T cells, wherein T cells in the population comprise a genetic disruption at the endogenous CD3E locus.
118. A method of producing genetically engineered T cells, the method comprising introducing into a population comprising T cells the polynucleotide of any of embodiments 66 and 69-106, wherein T cells of the population comprise a genetic disruption within an endogenous CD3E locus, wherein the transgene of the polynucleotide is integrated into the endogenous CD3E locus via homology directed repair (HDR).
119. A method of producing genetically engineered T cells, the method comprising introducing into a population comprising T cells the vector of any of embodiments 107-110 wherein T cells of the population comprise a genetic disruption within an endogenous CD3E locus, wherein the transgene of the polynucleotide is integrated into the endogenous CD3E locus via homology directed repair (HDR).
120. The method of any of embodiments 111-119, wherein the genetic disruption is carried out by introducing, into the population of T cells, one or more agents to induce a genetic disruption at a target site within an endogenous invariant CD3-IgSF chain locus of the T cell.
121. The method of any of embodiments 111-120, wherein the method produces a modified invariant CD3-IgSF chain locus in T cells of the population of T cells, said modified invariant CD3-IgSF chain locus comprising a nucleic acid sequence encoding a miniCAR, wherein the miniCAR is a fusion protein comprising the antigen-binding domain encoded by the introduced polynucleotide and the endogenous invariant CD3-IgSF chain.
122. The method of embodiment 121, wherein the encoded miniCAR assembles into a TCR/CD3 complex in place of the corresponding endogenous invariant CD3-IgSF chain of the TCR/CD3 complex.
123. The method of any of embodiments 113-122, wherein the one or more agents capable of inducing a genetic disruption comprises a DNA binding protein or DNA-binding nucleic acid, a fusion protein comprising a DNA-targeting protein and a nuclease, or an RNA-guided nuclease that specifically binds to or hybridizes to the target site, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site.
124. The method of any of embodiments 113-123, wherein each of the one or more agents comprise a guide RNA (gRNA) having a targeting domain that is complementary to the at least one target site.
125. The method of embodiment 124, wherein the one or more agents are introduced as a ribonucleoprotein (RNP) complex comprising the gRNA and a Cas9 protein, optionally wherein the RNP is introduced via electroporation, particle gun, calcium phosphate transfection, cell compression or squeezing, optionally via electroporation.
126. The method of embodiment 125, wherein the concentration of the RNP is at or about 0.1, 0.2, 0.3, 0.4, 0.5, 0.6, 0.7, 0.8, 0.9, 1, 2, 2.2, 2.5, 3, 4, 5 μg/10⁶ cells, or a range defined by any two of the foregoing values, optionally wherein the concentration of the RNP is at or about 1 μg/10⁶ cells.
127. The method of any of embodiments 124-126, wherein the molar ratio of the gRNA and the Cas9 molecule in the RNP is at or about at or about 5:1, 4:1, 3:1, 2:1, 1:1, 1:2, 1:3, 1:4 or 1:5, or a range defined by any two of the foregoing values, optionally wherein the molar ratio of the gRNA and the Cas9 molecule in the RNP is at or about 2:1.
128. The method of any of embodiments 124-127, wherein the gRNA has a targeting domain sequence UUGACAUGCCCUCAGUAUCC (SEQ ID NO: 8).
129. The method of any of embodiments 111-128, wherein the population of T cells comprise primary T cells derived from a subject, optionally wherein the subject is a human.
130. The method of any of embodiments 111-129, wherein the T cells comprise CD8+ T cell or subtypes thereof, or CD4+ T cells or subtypes thereof.
131. The method of any of embodiments 111, 113, 115, 116, 118 and 120-130, wherein the polynucleotide is a linear polynucleotide, optionally a double-stranded polynucleotide or a single-stranded polynucleotide.
132. The method of any of embodiments 111, 113, 115, 116, 118 and 120-130, wherein the polynucleotide is comprised in a vector.
133 The method of any of embodiments 113-132, wherein the one or more agent(s) and the polynucleotide or vector are introduced simultaneously or sequentially, in any order.
134. The method of any of embodiments 113-133, wherein the one or more agent(s) and the polynucleotide or vector are introduced simultaneously.
135. The method of any of embodiments 113-133, wherein the polynucleotide or vector is introduced after the introduction of the one or more agents.
136. The method of embodiment 135, wherein the polynucleotide or vector is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the one or more agents.
137. The method of any of embodiments 113-136, wherein prior to the introducing of the one or more agents and/or the introducing of the polynucleotide or vector, the method comprises incubating the population of T cells, in vitro with one or more stimulatory agents under conditions to stimulate or activate one or more T cells of the population, optionally wherein the one or more stimulatory agent(s) comprises and anti-CD3 and/or anti-CD28 antibodies, optionally anti-CD3/anti-CD28 beads, optionally wherein the bead to cell ratio is or is about 1:1, or oligomeric particle reagent comprising anti-CD3 and/or anti-CD28 antibodies.
138. The method of any of embodiments 113-137, wherein the method further comprises incubating the population of T cells prior to, during or subsequent to the introducing of the one or more agents and/or the introducing of the polynucleotide or vector with one or more recombinant cytokines, optionally wherein the one or more recombinant cytokines are selected from the group consisting of IL-2, IL-7, and IL-15, optionally wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.
139. The method of embodiment 137 or 138, wherein the incubation is carried out subsequent to the introducing of the one or more agents and the introducing of the polynucleotide or vector, and wherein the incubation is for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.
140. The method of any of embodiments 113-139, further comprising cultivating the population of T cells under conditions for expansion, wherein the cultivating is subsequent to the introducing of the one or more agents and/or the introducing of the polynucleotide or vector.
141. The method of embodiment 140, wherein the cultivating under conditions for expansion comprises incubating the population of T cells with the target antigen of the antigen-binding domain, target cells expressing the target antigen, or an anti-idiotype antibody that binds to the antigen-binding domain.
142. The method of embodiment 140 or 141, wherein the cultivating under conditions for expansion is carried out for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.
143. The method of any of embodiments 111-142, wherein the method results in at least or greater than at or about 75%, 80%, or 90% of the cells in the population of T cells comprise a genetic disruption of at least one target site within the invariant CD3-IgSF chain locus.
144. The method of any of embodiments 111-143, wherein the method results in at least or greater than at or about 25%, 30%, 35%, 40%, 45%, 50%, 55%, 60%, 65%, 70%, 75%, 80%, or 90% or more of T cells in the population of T cells generated by the method express the miniCAR.
145. A population comprising engineered T cells produced by the method of any of embodiments 111-144.
146. A T cell comprising a TCR/CD3 complex comprising a mini chimeric antigen receptor (CAR), wherein the miniCAR is a fusion protein comprising a heterologous antigen-binding domain and an endogenous invariant CD3 chain of the immunoglobulin superfamily (invariant CD3-IgSF chain).of the TCR/CD3 complex.
147. The T cell of embodiment 146, wherein the miniCAR is expressed from a modified invariant CD3-IgSF chain locus of the T cell, the modified invariant CD3-IgSF chain locus comprising a nucleic acid sequence encoding the miniCAR.
148. The T cell of embodiment 147, wherein the invariant CD3-IgSF chain locus is a CD3 epsilon (CD3E), a CD3 delta (CD3D), or a CD3 gamma (CD3G) locus.
149. The T cell of embodiment 147, wherein the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain
150. A T cell comprising a TCR/CD3 complex comprising a mini chimeric antigen receptor (miniCAR), wherein the miniCAR is a fusion protein comprising a heterologous antigen-binding domain and an endogenous CD3e chain of the TCR/CD3 complex.
151. The T cell of embodiment 150, wherein the miniCAR is expressed from a modified CD3E locus comprising a nucleic acid sequence encoding the miniCAR.
152. A composition, comprising the engineered T cell of any of embodiments 1-63, the population comprising engineered T cells of embodiment 145, or the T cell of any of embodiments 146-151.
153. A composition, comprising engineered T cells produced by the method of any of embodiments 111-144.
154. The composition of embodiment 152 or 153, wherein the composition comprises CD4+ T cells and/or CD8+ T cells.
155. The composition of embodiment 154, wherein the composition comprises CD4+ T cells and CD8+ T cells and the ratio of CD4+ to CD8+ T cells is from or from about 1:3 to 3:1, optionally 1:1.
156. The composition of any of embodiments 152-155, wherein the composition comprises a plurality of T cells expressing the miniCAR.
157. The composition of any of embodiments 152-156, wherein the composition comprises at or about 1×10⁶, 1.5×10⁶, 2.5×10⁶, 5×10⁶, 7.5×10⁶, 1×10⁷, 1.5×10⁷, 2×10⁷, 2.5×10⁷, 5×10⁷, 7.5×10⁷, 1×10⁸, 1.5×10⁸, 2.5×10⁸, or 5×10⁸ total T cells.
158. The composition of any of embodiments 152-157, wherein the composition comprises at or about 1×10⁵, 2.5×10⁵, 5×10⁵, 6.5×10⁵, 1×10⁶, 1.5×10⁶, 2×10⁶, 2.5×10⁶, 5×10⁶, 7.5×10⁶, 1×10⁷, 1.5×10⁷, 5×10⁷, 7.5×10⁷, 1×10⁸ or 2.5×10⁸ T cells expressing the miniCAR.
159. The composition of any of embodiments 156-158, wherein the frequency of T cells in the composition expressing the miniCAR is at or about 25%, 30%, 35%, 40%, 45%, 50%, 55%, 60%, 65%, 70%, 75%, 80%, or 90% or more of the total cells in the composition, or of the total CD4+ T cells or CD8+ T cells in the composition, or the total cells in the composition that comprises a genetic disruption within an endogenous invariant CD3-IgSF chain locus.
160 The composition of any of embodiments 152-159 that is a pharmaceutical composition.
161 The composition of any of embodiments 152-160, further comprising a pharmaceutically acceptable carrier.
162 The composition of any of embodiments 152-161, further comprising a cryoprotectant.
163. A method of treatment comprising administering the engineered T cell of any of embodiments 1-63, the population comprising engineered T cells of embodiment 145, the T cell of any of embodiments 146-151, or the composition of any of embodiments 153-162, to a subject having a disease or disorder.
164. Use of the engineered T cell of any of embodiments 1-63, the population comprising engineered T cells of embodiment 145, the T cell of any of embodiments 146-151, or the composition of any of embodiments 153-162 for the treatment of a disease or disorder.
165. Use of the engineered T cell of any of embodiments 1-63, the population comprising engineered T cells of embodiment 145, the T cell of any of embodiments 146-151, or the composition of any of embodiments 153-162 in the manufacture of a medicament for treating a disease or disorder.
166. The engineered T cell of any of embodiments 1-63, the population comprising engineered T cells of embodiment 145, the T cell of any of embodiments 146-151, or the composition of any of embodiments 153-162 for use in the treatment of a disease or disorder.
167. The method, the use, the engineered T cell, the population of engineered T cells, the T cell or the composition for use of any of embodiments 152-166, wherein cells or tissues associated with the disease or disorder express the target antigen recognized by the antigen binding domain.
168. The method, the use, the engineered T cell, the population of engineered T cells, the T cell or the composition for use of any of embodiments 152-167, wherein the disease or disorder is a cancer or a tumor.
169. The method, the use, the engineered T cell, the population of engineered T cells, the T cell or the composition for use of embodiment 168, wherein the cancer or the tumor is a hematologic malignancy, optionally a lymphoma, a leukemia, or a plasma cell malignancy.
170. The method, the use, the engineered T cell, the population of engineered T cells, the T cell or the composition for use of embodiment 168 or 169, wherein the cancer is a lymphoma and the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL).
171. The method, the use, the engineered T cell, the population of engineered T cells, the T cell or the composition for use of any of embodiments 168-170, wherein the cancer is a leukemia and the leukemia is chronic lymphocytic leukemia (CLL), plasma cell leukemia or acute lymphocytic leukemia (ALL).
172. The method, the use, the engineered T cell, the population of engineered T cells, the T cell or the composition for use of any of embodiments 168-170, wherein the cancer is a plasma cell malignancy and the plasma cell malignancy is multiple myeloma (MM).
173. The method, the use, the engineered T cell, the population of engineered T cells, the T cell or the composition for use of embodiment 168, wherein the cancer or the tumor is a solid tumor, optionally wherein the solid tumor is a non-small cell lung cancer (NSCLC) or a head and neck squamous cell carcinoma (HNSCC).
174. A kit comprising:
one or more agents capable of inducing a genetic disruption at a target site within an endogenous invariant CD3-IgSF chain locus of a T cell; and the polynucleotide of any of embodiments 64-106.
175. A kit, comprising:
one or more agents capable of inducing a genetic disruption at a target site within an endogenous invariant CD3-IgSF chain locus of a T cell; and
a polynucleotide of any of embodiments 64-106, wherein the polynucleotide is targeted for integration at or near the target site via homology directed repair (HDR); and instructions for carrying out the method of any of embodiments 56-88b.
176. The kit of embodiment 174 or 175, wherein the endogenous invariant CD3-IgSF chain locus is a CD3E locus encoding a CD3e chain, a CD3D locus encoding a CD3d chain, or a CD3G locus encoding an CD3g chain.
177. The kit of any of embodiments 174-176, wherein the endogenous invariant CD3-IgSF chain locus is a CD3E locus encoding a CD3e chain
178. The kit of any of embodiments 174-176, wherein the endogenous invariant CD3-IgSF chain locus is a CD3D locus encoding a CD3d chain
179. The kit of any of embodiments 174-176, wherein the endogenous invariant CD3-IgSF chain locus is a CD3G locus encoding a CD3g chain.
180. A kit comprising:
one or more agents capable of inducing a genetic disruption at a target site within a CD3E locus of a T cell; and
the polynucleotide of any of embodiments 64-106.
181. A kit, comprising:
one or more agents capable of inducing a genetic disruption at a target site within a CD3E locus of a T cell; and
a polynucleotide of any of embodiments 64-106, wherein the polynucleotide is targeted for integration at or near the target site via homology directed repair (HDR); and
instructions for carrying out the method of any of embodiments 111-144.
182. The kit of any of embodiments 174-181, wherein the one or more agents capable of inducing a genetic disruption comprises a DNA binding protein or DNA-binding nucleic acid that specifically binds to or hybridizes to the target site, a fusion protein comprising a DNA-targeting protein and a nuclease, or an RNA-guided nuclease, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site.
183. The kit of any of embodiments 174-182, wherein the each of the one or more agents comprise a guide RNA (gRNA) having a targeting domain that is complementary to the at least one target site.
184. The kit of embodiment 183, wherein the gRNA has a targeting domain sequence UUGACAUGCCCUCAGUAUCC (SEQ ID NO: 8).

### IX. Examples

The following examples are included for illustrative purposes only and are not intended to limit the scope of the invention.

### Example 1 Targeted Integration of Transgene Sequences Encoding an scFv at the Endogenous CD3 Epsilon (CD3E) Locus in a T Cell

Human T cells were engineered to express a mini chimeric antigen receptor (miniCAR) containing a heterologous antigen-binding domain, such as a single chain variable fragment (scFv), linked to an endogenous component of the CD3 complex. This example exemplifies generation of a miniCAR in which a heterologous scFv was indirectly linked to the endogenous CD3 epsilon chain of the T cell via a flexible peptide linker.

To generate the engineered T cells, a nucleic acid sequence encoding the heterologous scFv and peptide linker was targeted for integration at the endogenous locus encoding a subunit of the CD3 complex, for example the endogenous CD3 epsilon (CD3E) locus (encoding CD3e), via homology-dependent repair (HDR). The resulting engineered T cells contained a modified CD3E locus encoding a miniCAR fusion protein composed of the scFv and linker fused to CD3e.

A schematic showing an exemplary CD3 complex including the encoded miniCAR fusion protein from the resulting modified CD3E locus is depicted in FIG. 1A.

A. Generation of Engineered T Cells by HDR

Linear double stranded template polynucleotides containing nucleic acid sequences encoding an exemplary anti-CD19 scFv (SEQ ID NO: 1) and an exemplary peptide linker sequence (GGGGS)3 (SEQ ID NO:2), flanked by 5′ and 3′ homology sequences for targeted integration at the endogenous CD3 epsilon (CD3E) locus of a T cell were generated for HDR-mediated targeting. The encoded anti-CD19 scFv was derived from a murine antibody (variable region derived from FMC63, V_{L}-linker-V_{H} orientation). The polynucleotides also contained a P2A ribosome skip sequence (SEQ ID NO: 3) upstream of the scFv-encoding sequence to allow the inserted nucleic acid sequences to be expressed under the control of the endogenous CD3E promoter at the insertion site.

For targeting at the CD3E locus, the nucleic acid sequences were flanked by 5′ and 3′ homology arms of approximately 100 to 300 base pairs (set forth in SEQ ID NOS: 4 and 5, respectively).

The general structure of the exemplary linear template polynucleotide was as follows: [5′ homology arm]-[scFv]-[Linker]-[3′ homology arm]. An exemplary linear template polynucleotide is set forth in SEQ ID NO: 6.

Primary human CD4+ and CD8+ T cells were stimulated, cultured and subjected to a one-step electroporation with ribonucleoprotein (RNP) complexes containing CD3E-targeting gRNA (UUGACAUGCCCUCAGUAUCC, as set forth in SEQ ID NO: 8) and the exemplary linear template polynucleotides (SEQ ID NO: 6) for HDR-mediated targeting of the scFv-linker-encoding nucleic acid at the CD3E locus. Specifically, T cells were stimulated by incubation with a reagent containing anti-CD3/anti-CD28 Fab antibody fragments. Cells were washed and suspended in electroporation mix. Pre-assembled RNP complexes containing CD3E-targeting gRNA and a Cas9 protein (1m/1×10⁶ cells) were mixed with 0.7 μg, 1.2 μg or 1.4 μg of the exemplary linear polynucleotides, then added to the stimulated cell suspension. The cells were subject to electroporation, followed by incubation in culture medium for 5 days. Mock electroporated cells, cells electroporated with RNPs containing a gRNA targeting a T cell receptor alpha constant (TRAC) gene (AGAGUCUCUCAGCUGGUACA, as set forth in SEQ ID NO: 10; TRAC KO; resulting in absence of anti-CD3 antibody staining), or cells electroporated with RNPs containing gRNA targeting CD3E only (without template polynucleotides; CD3E KO) served as controls. Five days after electroporation (7 days after initial stimulation), the cells were assessed by flow cytometry after staining with an anti-CD3 antibody (OKT3 clone; Kjer-Nielsen et al., PNAS May 18, 2004 101 (20) 7675-7680;) targeting the CD3e chain, an anti-CD4 antibody, and an anti-idiotype antibody to detect expression of the exemplary anti-CD19 scFv, for example as described in International Patent Application Publication WO2018/023100.

B. Expression of Exemplary scFv

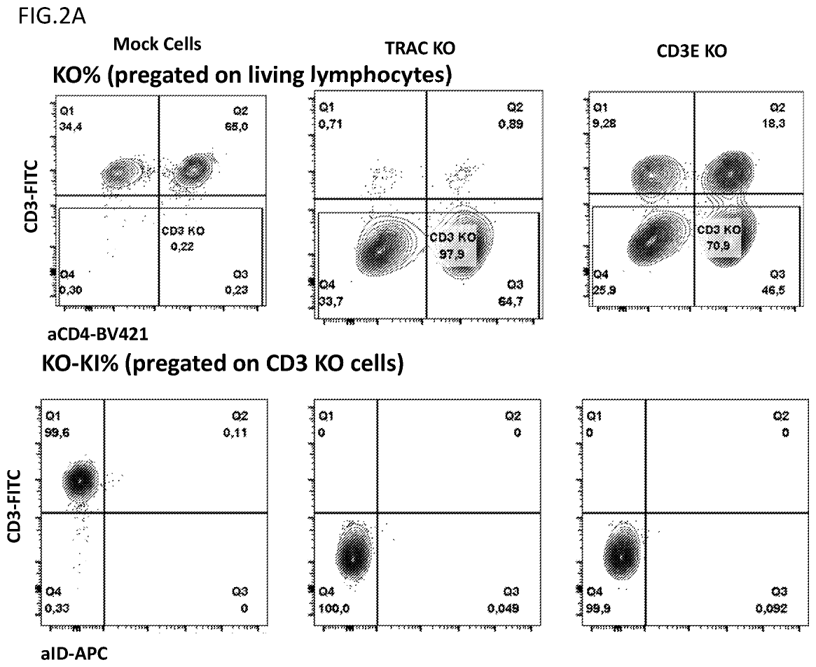

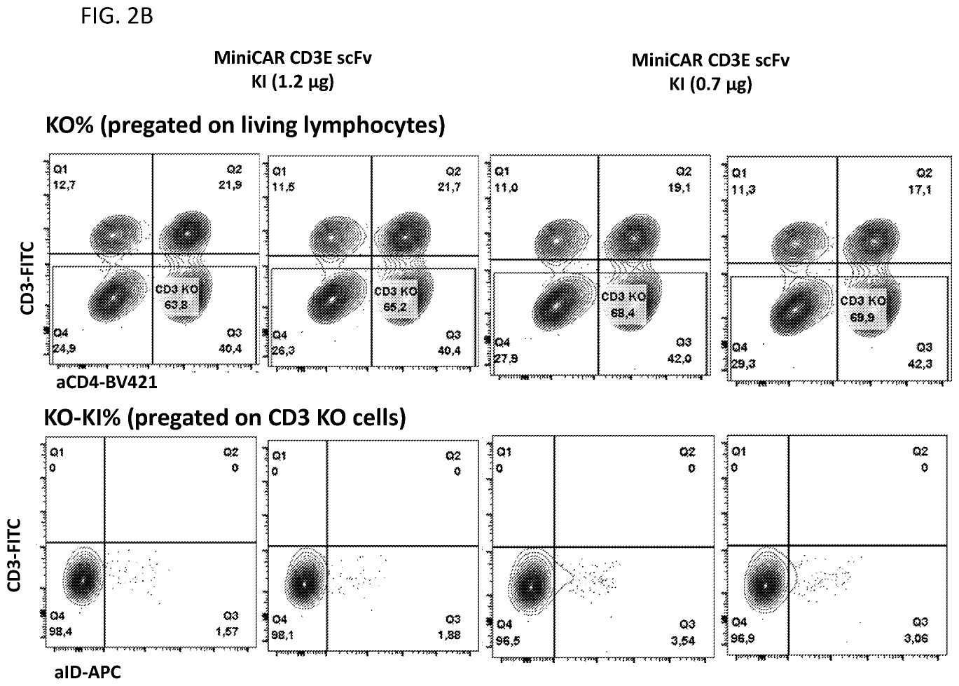

As shown in FIG. 2A, CD3 but not the scFv was expressed on the surface of mock transduced cells (FIG. 2A, left panels), TRAC KO cells showed substantially reduced expression of CD3 on the cell surface and no staining with the anti-idiotype antibody (FIG. 2A, middle panels), and CD3E KO cells showed reduced expression of CD3 on the cell surface and no staining with the anti-idiotype antibody (FIG. 2A, right panels). In contrast, the results in FIG. 2B showed that cells electroporated with RNP complexes targeting CD3E and exemplary template polynucleotides resulted in reduced cell surface expression of CD3 (FIG. 2B, top panels) and scFv-expressing cells, as observed by the presence of scFv⁺ cells in MiniCAR CD3E scFv KI groups (FIG. 2B, bottom panels), at 7 days after stimulation. FIG. 3A shows that electroporation with RNPs containing gRNA targeting CD3E and template polynucleotide (MiniCAR CD3E scFv KI) or with RNPs containing gRNA targeting CD3E only, without the template polynucleotide (CD3E KO), results in knock out of cell surface expression of CD3e in more than 70% of cells. FIG. 3B demonstrates that electroporated RNPs containing gRNA targeting CD3E and template polynucleotide (MiniCAR CD3E scFv KI) results in cell surface expression of the exemplary scFv, as evidenced by anti-idiotype antibody staining.

These results are consistent with the integration of transgene sequences encoding the exemplary scFv and linker by HDR into the CD3E locus, and expression of the scFv and linker as a fusion protein with CD3e on the surface of the engineered T cell.

C. Antigen-Specific Expansion and Enrichment of Engineered T Cells

To assess antigen-specific expansion of the engineered T cells expressing the anti-CD19 scFv from the modified CD3E locus, electroporated cells from each experimental group were expanded after co-culturing with irradiated CD19-expressing LCL cells at an effector to target ratio (E:T) of 1:3. Cells were co-cultured for 5 days and assessed by flow cytometry, and stained with an anti-CD3 antibody, an anti-CD4 antibody and an anti-idiotype antibody to detect expression of the exemplary anti-CD19 scFv.

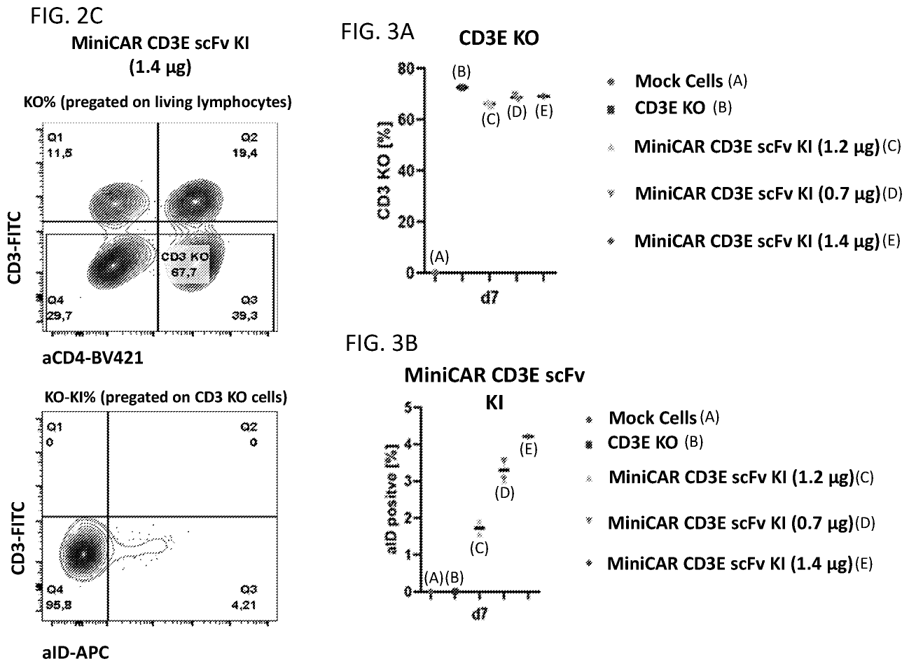

As shown in FIG. 4A, the percentage of cells that were absent (knocked-out) for expression of CD3 remained relative unchanged, with only a minimal decrease in the percentages of cells negative for CD3 after 5 days of expansion of the engineered cells. However, as shown in FIG. 4B, a striking increase in the percentage of scFv⁺ cells was observed, as evidenced by anti-idiotype antibody staining, 5 days after co-culturing, indicating an antigen-specific expansion of the miniCAR-engineered scFv⁺ cells. Results showed that there was no increase in the fold-expansion of cells that did not express the miniCAR (mock-transduced or CD3E KO), and an about 30-fold to about 40-fold increase in number of cells that had been engineered to express the engineered miniCAR containing the anti-CD19 scFv.

These results are consistent with the ability of the miniCAR fusion protein encoded by the modified CD3E locus encoding an exemplary scFv, to promote antigen-specific cellular expansion of the miniCAR engineered cells, and enrich for cells expressing the miniCAR in the expanded composition.

D. Cytolytic Activity of Engineered T Cells

The cytolytic activity of expanded engineered T cells expressing the exemplary miniCAR composed of the heterologous scFv linked via a linker to CD3e was assessed. Changes in impedance during co-culture with plate adherent target human embryonic kidney (HEK) cells expressing CD19 at an effector to target ratio (E:T) of 10:1 were measured over time. HEK-CD19+ cells only (without co-culture with engineered T cells), HEK-CD19+ cells co-cultured with mock T cells not engineered with the miniCAR, or media only were used as controls.

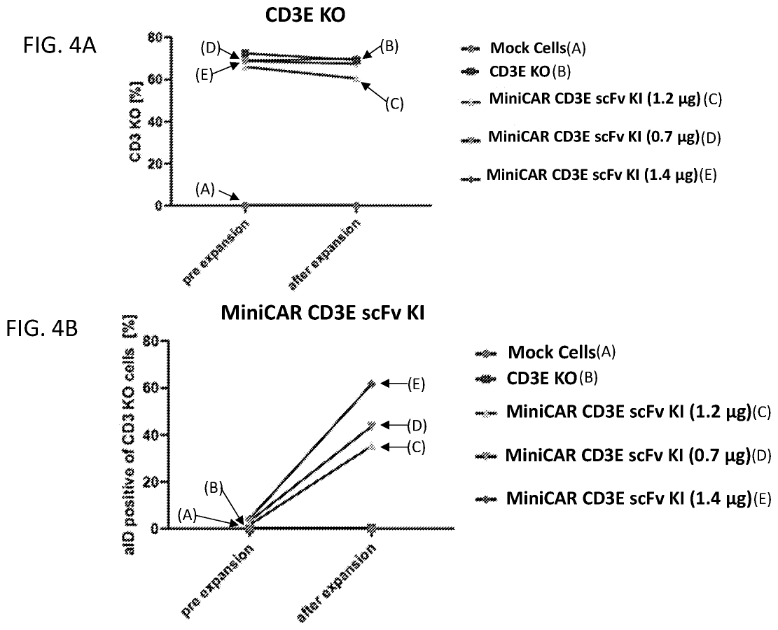

As shown in FIG. 5, miniCAR T cells exhibited substantial cytolytic activity, as evidenced by a decrease in impedance over time, compared to control groups.

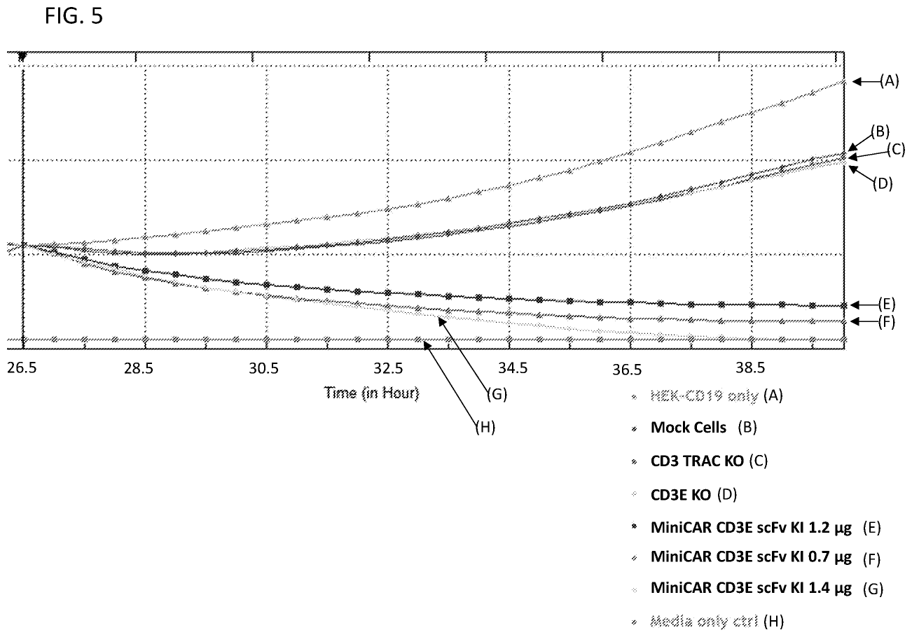

To further assess killing activity with increasing titrations of target cells, miniCAR T cells were co-cultured with HEK-CD19+ cells at E:T ratios of 10:1, 5:1, 2.5:1, and 1.25:1, and cytolytic activity was measured by impedance changes over time. As shown in FIG. 6, cytolytic activity was observed at each E:T ratio, as evidenced by a decrease in impedance over time, compared to HEK-CD19 cells cultured in the absence of engineered T cells; greatest killing activity was observed at the highest E:T ratios of 5:1 and 10:1.

These results are consistent with the ability of the engineered cells having a modified CD3E locus encoding a miniCAR composed of a heterologous scFv linked to CD3e, to kill antigen-expressing target cells.

### Example 2 Targeted Integration of Transgene Sequences Encoding an scFv at the Endogenous CD3 Epsilon (CD3E) or TCR Alpha Chain (TRAC) Locus in a T Cell

Human T cells were engineered to express a miniCAR composed of a heterologous single chain variable fragment (scFv) directly linked to an endogenous component of the CD3 complex, such as a CD3e, or a heterologous single chain variable fragment (scFv) directly linked to an endogenous TCR component, such as a TCR alpha chain.

The miniCAR was engineered into T cells similar to methods as described in Example 1, except that the scFv was integrated for direct fusion (without a linker) with the CD3e. A schematic showing an exemplary CD3 complex including the encoded miniCAR fusion protein from the resulting modified CD3E locus is depicted in FIG. 1B. The same exemplary scFv was integrated into the endogenous TRAC locus encoding the TCR alpha chain via HDR generally as described in Example 1, to generate a fusion protein where the scFv was fused directly (without a linker) with the TCR alpha chain. A schematic showing an exemplary TCR complex including the encoded fusion protein from the resulting modified TRAC locus is depicted in FIG. 1C. Expression of the encoded fusion proteins was assessed.

A. Generation of Engineered T Cells by HDR

T cells were engineered to express an exemplary anti-CD19 scFv from a modified CD3E locus, by targeted integration of the scFv-encoding nucleic acid sequence, generally as described in Example 1 above, with the following differences: the general structure of the exemplary linear template polynucleotide was as follows: [5′ homology arm]-[scFv]-[3′ homology arm]. An exemplary linear template polynucleotide for integration at the CD3E locus is set forth in SEQ ID NO: 7. The resulting engineered T cells contained a modified CD3E locus encoding a miniCAR fusion protein composed of the scFv directly fused to CD3e.

Integration of the scFv at the TRAC locus was accomplished by electroporating T cells with ribonucleoprotein (RNP) complexes containing TRAC-targeting gRNA (AGAGUCUCUCAGCUGGUACA, as set forth in SEQ ID NO: 10) and exemplary linear template polynucleotides encoding an exemplary anti-CD19 scFv (SEQ ID NO: 1), as described in Example 1. The exemplary linear polynucleotides included 5′ and 3′ homology arms for integration at the TRAC locus (SEQ ID NOS: 13 and 14, respectively). An exemplary linear template polynucleotide for integration at the TRAC locus is set forth in SEQ ID NO: 15. The resulting engineered T cells contained a modified TRAC locus encoding the scFv fused to TRAC.

Cells expressing an exemplary full length anti-CD19 chimeric antigen receptor (CAR) containing an scFv, a linker, transmembrane domain, a 4-1BB costimulatory domain and a CD3z domain. integrated via HDR at the endogenous TRAC locus served as a control. Control cells were generated by electroporation with ribonucleoprotein (RNP) complexes containing TRAC-targeting gRNA (AGAGUCUCUCAGCUGGUACA, as set forth in SEQ ID NO: 10) and exemplary linear template polynucleotides encoding a full length anti-CD19 CAR (SEQ ID NO: 12), generally as described in Example 1 above. The exemplary full length CAR sequence included The polynucleotides encoding the exemplary full length CAR included 5′ and 3′ homology arms for integration at the TRAC locus (SEQ ID NOS: 13 and 14, respectively).

Five days after electroporation (7 days after initial stimulation), the cells were assessed by flow cytometry after staining with an anti-idiotype antibody to detect expression of the anti-CD19 scFv, for example as described in International Patent Application Publication WO2018/023100.

B. Expression of Exemplary scFv

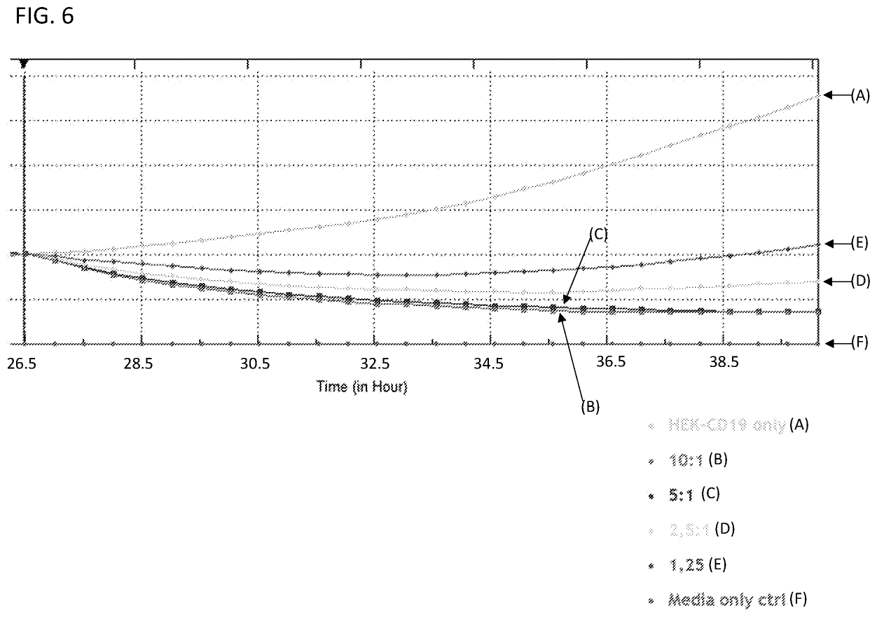

FIG. 7 shows the percentage of cells modified at the TRAC locus expressing the exemplary scFv on the cell surface, as evidenced by staining with anti-idiotype antibodies. The control cells with the full length CAR integrated at the TRAC locus (TRAC CAR) showed a higher percentage of cells expressing the exemplary scFv on the cell surface than cells electroporated with TRAC-targeting gRNA and template polynucleotides encoding an exemplary anti-CD19 scFv (TRAC scFv), control cells electroporated with TRAC-targeting gRNA only, or control cells electroporated with the exemplary CAR template only.

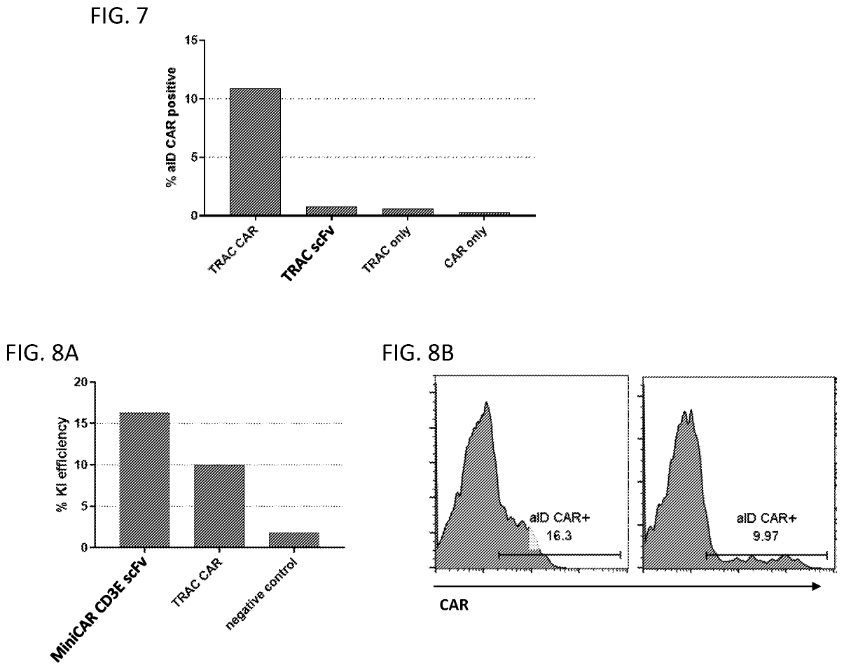

As shown in FIG. 8A, the percentage of cells expressing the exemplary miniCAR composed of a heterologous anti-CD19 scFv and CD3e (MiniCAR CD3E scFv) was higher compared to cells expressing the exemplary full length CAR comprising the same anti-CD19 scFv as the binding domain, expressed from a modified TRAC locus or Mock electroporated cells (negative control). FIG. 8B shows a representative histogram profile of the full length CAR expression from a modified TRAC locus (right panel) and the expression of the exemplary miniCAR from a modified CD3E locus (left panel), showing an increased cell surface expression of the exemplary miniCAR from a modified CD3E compared to the expression of the full length CAR expression from a modified TRAC locus.

These results are consistent with integration of transgene sequences encoding the exemplary scFv by HDR into the CD3E locus, for expression of a miniCAR composed of the scFv as a fusion protein with CD3e on engineered T cells. Together with the results in Example 1, these results demonstrate the feasibility of generating a miniCAR by direct or indirect (using a linker) fusion of the antigen-binding domain (e.g. scFv) to a CD3 component of the TCR complex, such as CD3e. Further, the provided results demonstrate improved expression of the miniCAR containing an extracellular scFv antigen-binding domain fused to a CD3 component of the TCR complex, such as CD3e, compared to alternative engineered cells expressing a full length CAR containing the same scFv, from a modified TRAC locus, or cells expressing the same scFv linked to TRAC.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ATGCTGCTGCTGGTGACCAGCCTGCTGCTGTGCGAGCTGCCCCACCCCGCCTTTCT | Anti-CD19 scFv |
| | GCTGATCCCCGACATCCAGATGACCCAGACCACCTCCAGCCTGAGCGCCAGCCTGG | |
| | GCGACCGGGTGACCATCAGCTGCCGGGCCAGCCAGGACATCAGCAAGTACCTGAAC | |
| | TGGTATCAGCAGAAGCCCGACGGCACCGTCAAGCTGCTGATCTACCACACCAGCAG | |
| | GTTGCACAGCGGCGTCCCCAGTCGCTTCTCAGGAAGTGGATCAGGGACCGATTACA | |
| | GTCTGACCATCTCCAACCTGGAACAGGAAGATATCGCCACCTACTTTTGCCAGCAG | |
| | GGCAACACACTGCCCTACACCTTTGGCGGCGGAACAAAGCTGGAAATCACCGGCAG | |
| | CACCTCCGGCAGCGGCAAGCCTGGCAGCGGCGAGGGCAGCACCAAGGGCGAGGTGA | |
| | AGCTGCAGGAAAGCGGCCCTGGCCTGGTGGCCCCCAGCCAGAGCCTGAGCGTGACC | |
| | TGCACCGTGAGCGGCGTGAGCCTGCCCGACTACGGCGTGAGCTGGATCAGGCAGCC | |
| | CCCCAGGAAGGGCCTGGAATGGCTGGGCGTGATCTGGGGCAGCGAGACCACCTACT | |
| | ACAACAGCGCCCTGAAGAGCCGGCTGACCATCATCAAGGACAACAGCAAGAGCCAG | |
| | GTGTTCCTGAAGATGAACAGCCTGCAGACCGACGACACCGCCATCTACTACTGCGC | |
| | CAAGCACTACTACTACGGCGGCAGCTACGCCATGGACTACTGGGGCCAGGGCACCA | |
| | GCGTGACCGTGAGCAGC | |
| 2 | GGTGGAGGAGGCTCTGGTGGAGGCGGTAGCGGAGGCGGAGGGTCG | G4Sx3 linker |
| 3 | GGAAGCGGAGAGGGCAGAGGAAGTCTTCTAACATGCGGTGACGTGGAGGAGAATCC | P2A |
| | CGGCCCA | |
| 4 | GTCACTAATTTGCCTTTTCTAAAATTGTCCTGGTTTCTTCTGCCAATTTCCCTTCT | 5′ Homology Arm |
| | TTCTCCCCAGCATATAAAGTCTCCATCTCTGGAACCACAGTAATATTGACATGCCC | |
| | TCAGTAT | |
| 5 | GATGGTAATGAAGAAATGGGAGGCATTACTCAGACACCATACAAGGTCAGTATCAG | 3′ Homology Arm |
| | TGGGACCACCGTAATCCTCACCTGCCCACAGTATCCTGGATCTGAAATACTATGGC | |
| | AACACAATGATAAAAACATAGGCGGTGATGAGGATGATAAAAACATAGGAAGCGAT | |
| | GAGGATCACCTGTCACTGAAGGAATTTTCAGAATTGGAGCAAAGTGGTTATTATGT | |
| | CTGCTACCCCAGAGGAAGCAAACCAGAAGATGCGAACTTTTAT | |
| 6 | GTCACTAATTTGCCTTTTCTAAAATTGTCCTGGTTTCTTCTGCCAATTTCCCTTCT | CD3e MiniCAR |
| | TTCTCCCCAGCATATAAAGTCTCCATCTCTGGAACCACAGTAATATTGACATGCCC | |
| | TCAGTATGGAAGCGGAGAGGGCAGAGGAAGTCTTCTAACATGCGGTGACGTGGAGG | |
| | AGAATCCCGGCCCAATGCTGCTGCTGGTGACCAGCCTGCTGCTGTGCGAGCTGCCC | |
| | CACCCCGCCTTTCTGCTGATCCCCGACATCCAGATGACCCAGACCACCTCCAGCCT | |
| | GAGCGCCAGCCTGGGCGACCGGGTGACCATCAGCTGCCGGGCCAGCCAGGACATCA | |
| | GCAAGTACCTGAACTGGTATCAGCAGAAGCCCGACGGCACCGTCAAGCTGCTGATC | |
| | TACCACACCAGCAGGTTGCACAGCGGCGTCCCCAGTCGCTTCTCAGGAAGTGGATC | |
| | AGGGACCGATTACAGTCTGACCATCTCCAACCTGGAACAGGAAGATATCGCCACCT | |
| | ACTTTTGCCAGCAGGGCAACACACTGCCCTACACCTTTGGCGGCGGAACAAAGCTG | |
| | GAAATCACCGGCAGCACCTCCGGCAGCGGCAAGCCTGGCAGCGGCGAGGGCAGCAC | |
| | CAAGGGCGAGGTGAAGCTGCAGGAAAGCGGCCCTGGCCTGGTGGCCCCCAGCCAGA | |
| | GCCTGAGCGTGACCTGCACCGTGAGCGGCGTGAGCCTGCCCGACTACGGCGTGAGC | |
| | TGGATCAGGCAGCCCCCCAGGAAGGGCCTGGAATGGCTGGGCGTGATCTGGGGCAG | |
| | CGAGACCACCTACTACAACAGCGCCCTGAAGAGCCGGCTGACCATCATCAAGGACA | |
| | ACAGCAAGAGCCAGGTGTTCCTGAAGATGAACAGCCTGCAGACCGACGACACCGCC | |
| | ATCTACTACTGCGCCAAGCACTACTACTACGGCGGCAGCTACGCCATGGACTACTG | |
| | GGGCCAGGGCACCAGCGTGACCGTGAGCAGCGGTGGAGGAGGCTCTGGTGGAGGCG | |
| | GTAGCGGAGGCGGAGGGTCGGATGGTAATGAAGAAATGGGAGGCATTACTCAGACA | |
| | CCATACAAGGTCAGTATCAGTGGGACCACCGTAATCCTCACCTGCCCACAGTATCC | |
| | TGGATCTGAAATACTATGGCAACACAATGATAAAAACATAGGCGGTGATGAGGATG | |
| | ATAAAAACATAGGAAGCGATGAGGATCACCTGTCACTGAAGGAATTTTCAGAATTG | |
| | GAGCAAAGTGGTTATTATGTCTGCTACCCCAGAGGAAGCAAACCAGAAGATGCGAA | |
| | CTTTTAT | |
| 7 | GTCACTAATTTGCCTTTTCTAAAATTGTCCTGGTTTCTTCTGCCAATTTCCCTTCT | CD3e minicar |
| | TTCTCCCCAGCATATAAAGTCTCCATCTCTGGAACCACAGTAATATTGACATGCCC | without linker from |
| | TCAGTATggaagcggagagggcagaggaagtcttctaacatgcggtgacgtggagg | |
| | agaatcccggcccaATGCTGCTGCTGGTGACCAGCCTGCTGCTGTGCGAGCTGCCC | |
| | CACCCCGCCTTTCTGCTGATCCCCGACATCCAGATGACCCAGACCACCTCCAGCCT | |
| | GAGCGCCAGCCTGGGCGACCGGGTGACCATCAGCTGCCGGGCCAGCCAGGACATCA | |
| | GCAAGTACCTGAACTGGTATCAGCAGAAGCCCGACGGCACCGTCAAGCTGCTGATC | |
| | TACCACACCAGCAGGTTGCACAGCGGCGTCCCCAGTCGCTTCTCAGGAAGTGGATC | |
| | AGGGACCGATTACAGTCTGACCATCTCCAACCTGGAACAGGAAGATATCGCCACCT | |
| | ACTTTTGCCAGCAGGGCAACACACTGCCCTACACCTTTGGCGGCGGAACAAAGCTG | |
| | GAAATCACCGGCAGCACCTCCGGCAGCGGCAAGCCTGGCAGCGGCGAGGGCAGCAC | |
| | CAAGGGCGAGGTGAAGCTGCAGGAAAGCGGCCCTGGCCTGGTGGCCCCCAGCCAGA | |
| | GCCTGAGCGTGACCTGCACCGTGAGCGGCGTGAGCCTGCCCGACTACGGCGTGAGC | |
| | TGGATCAGGCAGCCCCCCAGGAAGGGCCTGGAATGGCTGGGCGTGATCTGGGGCAG | |
| | CGAGACCACCTACTACAACAGCGCCCTGAAGAGCCGGCTGACCATCATCAAGGACA | |
| | ACAGCAAGAGCCAGGTGTTCCTGAAGATGAACAGCCTGCAGACCGACGACACCGCC | |
| | ATCTACTACTGCGCCAAGCACTACTACTACGGCGGCAGCTACGCCATGGACTACTG | |
| | GGGCCAGGGCACCAGCGTGACCGTGAGCAGCGGAGGCGGTTCTGGAGGTGGAAGCG | |
| | GTGGCTCTGATGGTAATGAAGAAATGGGAGGCATTACTCAGACACCATACAAGGTC | |
| | AGTATCAGTGGGACCACCGTAATCCTCACCTGCCCACAGTATCCTGGATCTGAAAT | |
| | ACTATGGCAACACAATGATAAAAACATAGGCGGTGATGAGGATGATAAAAACATAG | |
| | GCAGTGATGAGGATCACCTGTCACTGAAGGAATTTTCAGAATTGGAGCAAAGTGGT | |
| | TATTATGTCTGCTACCCCAGAGGAAGCAAACCAGAAGATGCGAACTTTTAT | |
| 8 | UUGACAUGCCCUCAGUAUCC | CD3E gRNA target |
| | | sequence 1 |
| 9 | TTGACATGCCCTCAGTATCC | CD3E gRNA |
| | | sequence 1 |
| 10 | AGAGUCUCUCAGCUGGUACA | TRAC gRNA target |
| | | sequence |
| 11 | AGAGTCTCTCAGCTGGTACA | TRAC gRNA |
| | | sequence |
| 12 | atgctgctgctggtgaccagcctgctgctgtgcgagctgccccaccccgcctttct | anti-CD19 CAR |
| | gctgatccccgacatccagatgacccagaccacctccagcctgagcgccagcctgg | (nt) |
| | gcgaccgggtgaccatcagctgccgggccagccaggacatcagcaagtacctgaac | |
| | tggtatcagcagaagcccgacggcaccgtcaagctgctgatctaccacaccagccg | |
| | gctgcacagcggcgtgcccagccggtttagcggcagcggctccggcaccgactaca | |
| | gcctgaccatctccaacctggaacaggaagatatcgccacctacttttgccagcag | |
| | ggcaacacactgccctacacctttggcggcggaacaaagctggaaatcaccggcag | |
| | cacctccggcagcggcaagcctggcagcggcgagggcagcaccaagggcgaggtga | |
| | agctgcaggaaagcggccctggcctggtggcccccagccagagcctgagcgtgacc | |
| | tgcaccgtgagcggcgtgagcctgcccgactacggcgtgagctggatcaggcagcc | |
| | ccccaggaagggcctggaatggctgggcgtgatctggggcagegagaccacctact | |
| | acaacagcgccctgaagagccggctgaccatcatcaaggacaacagcaagagccag | |
| | gtgttcctgaagatgaacagcctgcagaccgacgacaccgccatctactactgcgc | |
| | caagcactactactacggcggcagctacgccatggactactggggccagggcacca | |
| | gcgtgaccgtgagcagcgagagcaagaattggagccacccgcagttcgaaaaagga | |
| | ggtggaggttcaggtggtggaggctcttacggaccgaattggtctcatcctcagtt | |
| | cgagaaaggaggcggttctggaggtggaagcggtggctcttggagccacccacagt | |
| | ttgaaaagggaggcgggggctccggtggcggaggctcttccggatctccctgtcca | |
| | ccttgccctatgttctgggtgctggtagtggtaggtggagtgctggcctgctacag | |
| | cctgctggtgacagtggccttcatcatcttttgggtgaaacggggcagaaagaaac | |
| | tcctgtatatattcaaacaaccatttatgagaccagtacaaactactcaagaggaa | |
| | gatggctgtagctgccgatttccagaagaagaagaaggaggatgtgaactgcgggt | |
| | gaagttcagcagaagcgccgacgcacctgcctaccagcagggccagaatcagctgt | |
| | acaacgagctgaacctgggacgaagggaagagtacgacgtcctggataagcggaga | |
| | ggccgggaccctgagatgggcggcaagcctcggeggaagaacccccaggaaggcct | |
| | gtataacgaactgcagaaagacaagatggccgaggcctacagcgagatcggcatga | |
| | agggcgagcggaggcggggcaagggccacgacggcctgtatcagggcctgtccacc | |
| | gccaccaaggatacctacgacgccctgcacatgcaggccctgcccccaaggtga | |
| 13 | GGGAAATGAGATCATGTCCTAACCCTGATCCTCTTGTCCCACAGATATCCAGAACC | TRAC 5′ homology |
| | CTGACCCTGCCGTG | arm |
| 14 | TACCAGCTGAGAGACTCTAAATCCAGTGACAAGTCTGTCTGCCTATTCACCGATTT | TRAC 3′ homology |
| | TG | arm |
| 15 | gggaaatgagatcatgtcctaaccctgatcctcttgtcccacagatatccagaacc | TRAC minicar |
| | ctgaccctgccgtgggaagcggagagggcagaggaagtcttctaacatgcggtgac | without linker |
| | gtggaggagaatcccggcccaATGCTGCTGCTGGTGACCAGCCTGCTGCTGTGCGA | |
| | GCTGCCCCACCCCGCCTTTCTGCTGATCCCCGACATCCAGATGACCCAGACCACCT | |
| | CCAGCCTGAGCGCCAGCCTGGGCGACCGGGTGACCATCAGCTGCCGGGCCAGCCAG | |
| | GACATCAGCAAGTACCTGAACTGGTATCAGCAGAAGCCCGACGGCACCGTCAAGCT | |
| | GCTGATCTACCACACCAGCAGGTTGCACAGCGGCGTCCCCAGTCGCTTCTCAGGAA | |
| | GTGGATCAGGGACCGATTACAGTCTGACCATCTCCAACCTGGAACAGGAAGATATC | |
| | GCCACCTACTTTTGCCAGCAGGGCAACACACTGCCCTACACCTTTGGCGGCGGAAC | |
| | AAAGCTGGAAATCACCGGCAGCACCTCCGGCAGCGGCAAGCCTGGCAGCGGCGAGG | |
| | GCAGCACCAAGGGCGAGGTGAAGCTGCAGGAAAGCGGCCCTGGCCTGGTGGCCCCC | |
| | AGCCAGAGCCTGAGCGTGACCTGCACCGTGAGCGGCGTGAGCCTGCCCGACTACGG | |
| | CGTGAGCTGGATCAGGCAGCCCCCCAGGAAGGGCCTGGAATGGCTGGGCGTGATCT | |
| | GGGGCAGCGAGACCACCTACTACAACAGCGCCCTGAAGAGCCGGCTGACCATCATC | |
| | AAGGACAACAGCAAGAGCCAGGTGTTCCTGAAGATGAACAGCCTGCAGACCGACGA | |
| | CACCGCCATCTACTACTGCGCCAAGCACTACTACTACGGCGGCAGCTACGCCATGG | |
| | ACTACTGGGGCCAGGGCACCAGCGTGACCGTGAGCAGCGGAGGCGGTTCTGGAGGT | |
| | GGAAGCGGTGGCTCTATCCAGAACCCCGACCCTGCTGTTTATCAGCTCAGGGATtc | |
| | taaatccagtgacaagtctgtctgcctattcaccgattttg | |
| 16 | GGGGSGGGGSGGGGS | G4Sx3 linker |
| 17 | MQSGTHWRVLGLCLLSVGVWGQDGNEEMGGITQTPYKVSISGTTVILTCPQYPGSE | CD3epsilon (CD3e) |
| | ILWQHNDKNIGGDEDDKNIGSDEDHLSLKEFSELEQSGYYVCYPRGSKPEDANFYL | isoform 1 |
| | YLRARVCENCMEMDVMSVATIVIVDICITGGLLLLVYYWSKNRKAKAKPVTRGAGA | (NCBI: |
| | GGRQRGQNKERPPPVPNPDYEPIRKGQRDLYSGLNQRRI | NP_000724.1) |
| 18 | agaaaccctcctcccctcccagcctcaggtgcctgcttcagaaaatgaagtagtaa | CD3epsilon (CD3e) |
| | gtctgctggcctccgccatcttagtaaagtaacagtcccatgaaacaaagatgcag | (NCBI: |
| | tcgggcactcactggagagttctgggcctctgcctcttatcagttggcgtttgggg | NM_000733) |
| | gcaagatggtaatgaagaaatgggtggtattacacagacaccatataaagtctcca | |
| | tctctggaaccacagtaatattgacatgccctcagtatcctggatctgaaatacta | |
| | tggcaacacaatgataaaaacataggcggtgatgaggatgataaaaacataggcag | |
| | tgatgaggatcacctgtcactgaaggaattttcagaattggagcaaagtggttatt | |
| | atgtctgctaccccagaggaagcaaaccagaagatgcgaacttttatctctacctg | |
| | agggcaagagtgtgtgagaactgcatggagatggatgtgatgtcggtggccacaat | |
| | tgtcatagtggacatctgcatcactgggggcttgctgctgctggtttactactgga | |
| | gcaagaatagaaaggccaaggccaagcctgtgacacgaggagcgggtgctggcggc | |
| | aggcaaaggggacaaaacaaggagaggccaccacctgttcccaacccagactatga | |
| | gcccatccggaaaggccagcgggacctgtattctggcctgaatcagagacgcatct | |
| | gaccctctggagaacactgcctcccgctggcccaggtctcctctccagtccccctg | |
| | cgactccctgtttcctgggctagtcttggaccccacgagagagaatcgttcctcag | |
| | cctcatggtgaactcgcgccctccagcctgatcccccgctccctcctccctgcctt | |
| | ctctgctggtacccagtcctaaaatattgctgcttcctcttcctttgaagcatcat | |
| | cagtagtcacaccctcacagctggcctgccctcttgccaggatatttatttgtgct | |
| | attcactcccttccctttggatgtaacttctccgttcagttccctccttttcttgc | |
| | atgtaagttgtcccccatcccaaagtattccatctacttttctatcgccgtcccct | |
| | tttgcagccctctctggggatggactgggtaaatgttgacagaggccctgccccgt | |
| | tcacagatcctggccctgagccagccctgtgctcctccctcccccaacactcccta | |
| | ccaaccccctaatcccctactccctccaccccccctccactgtaggccactggatg | |
| | gtcatttgcatctccgtaaatgtgctctgctcctcagctgagagagaaaaaaataa | |
| | actgtatttggctgcaa | |
| 19 | MQSGTHWRVLGLCLLSVGVWGQDGNEEMAYKVSISGTTVILTCPQYPGSEILWQHN | CD3epsilon (CD3e) |
| | DKNIGGDEDDKNIGSDEDHLSLKEFSELEQSGYYVCYPRGSKPEDANFYLYLRARV | isoform 2 |
| | CENCMEMDVMSVATIVIVDICITGGLLLLVYYWSKNRKAKAKPVTRGAGAGGRQRG | (Uniprot: E9PSH8) |
| | QNKERPPPVPNPDYEPIRKGQRDLYSGLNQRRI | |
| 20 | MEHSTFLSGLVLATLLSQVSPFKIPIEELEDRVFVNCNTSITWVEGTVGTLLSDIT | CD3delta (CD3d) |
| | RLDLGKRILDPRGIYRCNGTDIYKDKESTVQVHYRMCQSCVELDPATVAGIIVTDV | isoform 1 |
| | IATLLLALGVFCFAGHETGRLSGAADTQALLRNDQVYQPLRDRDDAQYSHLGGNWA | (Uniprot: P04234-1) |
| | RNK | |
| 21 | agagaagcagacatcttctagttcctcccccactctcctctttccggtacctgtga | CD3delta (CD3d) |
| | gtcagctaggggagggcagctctcacccaggctgatagttcggtgacctggcttta | isoform 1 |
| | tctactggatgagttccgctgggagatggaacatagcacgtttctctctggcctgg | (GenBank: |
| | tactggctacccttctctcgcaagtgagccccttcaagatacctatagaggaactt | NM_000732.4) |
| | gaggacagagtgtttgtgaattgcaataccagcatcacatgggtagagggaacggt | |
| | gggaacactgctctcagacattacaagactggacctgggaaaacgcatcctggacc | |
| | cacgaggaatatataggtgtaatgggacagatatatacaaggacaaagaatctacc | |
| | gtgcaagttcattatcgaatgtgccagagctgtgtggagctggatccagccaccgt | |
| | ggctggcatcattgtcactgatgtcattgccactctgctccttgctttgggagtct | |
| | tctgctttgctggacatgagactggaaggctgtctggggctgccgacacacaagct | |
| | ctgttgaggaatgaccaggtctatcagcccctccgagatcgagatgatgctcagta | |
| | cagccaccttggaggaaactgggctcggaacaagtgaacctgagactggtggcttc | |
| | tagaagcagccattaccaactgtaccttcccttcttgctcagccaataaatatatc | |
| | ctctttcactcagaaaaaaaaaaaaaaaaaaaaaaaaaaaaaa | |
| 22 | MEHSTFLSGLVLATLLSQVSPFKIPIEELEDRVFVNCNTSITWVEGTVGTLLSDIT | CD3delta (CD3d) |
| | RLDLGKRILDPRGIYRCNGTDIYKDKESTVQVHYRTADTQALLRNDQVYQPLRDRD | isoform 2 |
| | DAQYSHLGGNWARNK | (Uniprot: P04234-2) |
| 23 | agagaagcagacatcttctagttcctcccccactctcctctttccggtacctgtga | CD3delta (CD3d) |
| | gtcagctaggggagggcagctctcacccaggctgatagttcggtgacctggcttta | isoform 2 |
| | tctactggatgagttccgctgggagatggaacatagcacgtttctctctggcctgg | (GenBank: |
| | tactggctacccttctctcgcaagtgagccccttcaagatacctatagaggaactt | NM_001040651.1) |
| | gaggacagagtgtttgtgaattgcaataccagcatcacatgggtagagggaacggt | |
| | gggaacactgctctcagacattacaagactggacctgggaaaacgcatcctggacc | |
| | cacgaggaatatataggtgtaatgggacagatatatacaaggacaaagaatctacc | |
| | gtgcaagttcattatcgaactgccgacacacaagctctgttgaggaatgaccaggt | |
| | ctatcagcccctccgagatcgagatgatgctcagtacagccaccttggaggaaact | |
| | gggctcggaacaagtgaacctgagactggtggcttctagaagcagccattaccaac | |
| | tgtaccttcccttcttgctcagccaataaatatatcctctttcactcagaaaaaaa | |
| | aaaaaaaaaaaaaaaaaaaaaaa | |
| 24 | MEHSTFLSGLVLATLLSQVCQSCVELDPATVAGIIVTDVIATLLLALGVFCFAGHE | CD3delta (CD3d) |
| | TGRLSGAADTQALLRNDQVYQPLRDRDDAQYSHLGGNWARNK | isoform 3 (Uniprot: |
| | | E9PMT5) |
| 25 | atggaacatagcacgtttctctctggcctggtactggctacccttctctcgcaagt | CD3delta (CD3d) |
| | gtgccagagctgtgtggagctggatccagccaccgtggctggcatcattgtcactg | isoform 3 |
| | atgtcattgccactctgctccttgctttgggagtcttctgctttgctggacatgag | (GenBank: |
| | actggaaggctgtctggggctgccgacacacaagctctgttgaggaatgaccaggt | JN392069.1) |
| | ctatcagcccctccgagatcgagatgatgctcagtacagccaccttggaggaaact | |
| | gggctcggaacaagtga | |
| 26 | MEQGKGLAVLILAIILLQGTLAQSIKGNHLVKVYDYQEDGSVLLTCDAEAKNITWF | CD3gamma (CD3g) |
| | KDGKMIGFLTEDKKKWNLGSNAKDPRGMYQCKGSQNKSKPLQVYYRMCQNCIELNA | isoform 1 |
| | ATISGFLFAEIVSIFVLAVGVYFIAGQDGVRQSRASDKQTLLPNDQLYQPLKDRED | (Uniprot: P09693) |
| | DQYSHLQGNQLRRN | |
| 27 | agtctagctgctgcacaggctggctggctggctggctgctaagggctgctccacgc | CD3delta (CD3d) |
| | ttttgccggaggacagagactgacatggaacaggggaagggcctggctgtcctcat | isoform 1 |
| | cctggctatcattcttcttcaaggtactttggcccagtcaatcaaaggaaaccact | (GenBank: |
| | tggttaaggtgtatgactatcaagaagatggttcggtacttctgacttgtgatgca | NM_NM_000073.2) |
| | gaagccaaaaatatcacatggtttaaagatgggaagatgatcggcttcctaactga | |
| | agataaaaaaaaatggaatctgggaagtaatgccaaggaccctcgagggatgtatc | |
| | agtgtaaaggatcacagaacaagtcaaaaccactccaagtgtattacagaatgtgt | |
| | cagaactgcattgaactaaatgcagccaccatatctggctttctctttgctgaaat | |
| | cgtcagcattttcgtccttgctgttggggtctacttcattgctggacaggatggag | |
| | ttcgccagtcgagagcttcagacaagcagactctgttgcccaatgaccagctctac | |
| | cagcccctcaaggatcgagaagatgaccagtacagccaccttcaaggaaaccagtt | |
| | gaggaggaattgaactcaggactcagagtagtccaggtgttctcctcctattcagt | |
| | tcccagaatcaaagcaatgcattttggaaagctcctagcagagagactttcagccc | |
| | taaatctagactcaaggttcccagagatgacaaatggagaagaaaggccatcagag | |
| | caaatttgggggtttctcaaataaaataaaaataaaaacaaatactgtgtttcaga | |
| | agcgccacctattggggaaaattgtaaaagaaaaatgaaaagatcaaataaccccc | |
| | tggatttgaatataattttttgtgttgtaatttttatttcgtttttgtataggtta | |
| | taattcacatggctcaaatattcagtgaaagctctccctccaccgccatcccctgc | |
| | tacccagtgaccctgttgccctcttcagagacaaattagtttctcttttttttttt | |
| | tttttttttttttttgagacagtctggctctgtcacccaggctgaaatgcagtggc | |
| | accatctcggctcactgcaacctctgcctcctgggttcaagcgattctcctgcctc | |
| | agcctcccgggcagctgggattacaggcacacactaccacacctggctaatttttg | |
| | tatttttagtagagacagggttttgctctgttggccaagctggtctcgaactcctg | |
| | acctcaagtgatccgcccgcctc | |
| 28 | TGCCATAGTATTTCAGATCC | CD3E gRNA target |
| | | sequence 1 |
| 29 | CTGGATTACCTCTTGCCCTC | CD3E gRNA target |
| | | sequence 2 |
| 30 | AGGGCATGTCAATATTACTG | CD3E gRNA target |
| | | sequence 3 |
| 31 | TATTATGTCTGCTACCCCAG | CD3E gRNA target |
| | | sequence 4 |
| 32 | AGATAAAAGTTCGCATCTTC | CD3E gRNA target |
| | | sequence 5 |
| 33 | AGATGCAGTCGGGCACTCAC | CD3E gRNA target |
| | | sequence 6 |
| 34 | TTACTTTACTAAGATGGCGG | CD3E gRNA target |
| | | sequence 7 |
| 35 | GATGGAGACTTTATATGCTG | CD3E gRNA target |
| | | sequence 8 |
| 36 | GATGTCCACTATGACAATTG | CD3E gRNA target |
| | | sequence 9 |
| 37 | CAACACAATGATAAAAACAT | CD3E gRNA target |
| | | sequence 10 |
| 38 | TGAGGATCACCTGTCACTGA | CD3E gRNA target |
| | | sequence 11 |
| 39 | UGCCAUAGUAUUUCAGAUCC | CD3E gRNA |
| | | sequence 1 |
| 40 | CUGGAUUACCUCUUGCCCUC | CD3E gRNA |
| | | sequence 2 |
| 41 | AGGGCAUGUCAAUAUUACUG | CD3E gRNA |
| | | sequence 3 |
| 42 | UAUUAUGUCUGCUACCCCAG | CD3E gRNA |
| | | sequence 4 |
| 43 | AGAUAAAAGUUCGCAUCUUC | CD3E gRNA |
| | | sequence 5 |
| 44 | AGAUGCAGUCGGGCACUCAC | CD3E gRNA |
| | | sequence 6 |
| 45 | UUACUUUACUAAGAUGGCGG | CD3E gRNA |
| | | sequence 7 |
| 46 | GAUGGAGACUUUAUAUGCUG | CD3E gRNA |
| | | sequence 8 |
| 47 | GAUGUCCACUAUGACAAUUG | CD3E gRNA |
| | | sequence 9 |
| 48 | CAACACAAUGAUAAAAACAU | CD3E gRNA |
| | | sequence 10 |
| 49 | UGAGGAUCACCUGUCACUGA | CD3E gRNA |
| | | sequence 11 |
| 50 | AAACGCATCCTGGACCCACG | CD3D gRNA target |
| | | sequence 1 |
| 51 | ACTTCGATAATGAACTTGCA | CD3D gRNA target |
| | | sequence 2 |
| 52 | TAGCCTTACCTTGCGAGAGA | CD3D gRNA target |
| | | sequence 3 |
| 53 | CCGACACACAAGCTCTGTTG | CD3D gRNA target |
| | | sequence 4 |
| 54 | CAACGCTCACCTGATAGACC | CD3D gRNA target |
| | | sequence 5 |
| 55 | GAACATAGCACGTTTCTCTC | CD3D gRNA target |
| | | sequence 6 |
| 56 | GACAATGATGCCAGCCACGG | CD3D gRNA target |
| | | sequence 7 |
| 57 | TTGCAATACCAGCATCACAT | CD3D gRNA target |
| | | sequence 8 |
| 58 | AAACGCAUCCUGGACCCACG | CD3D gRNA |
| | | sequence 1 |
| 59 | ACUUCGAUAAUGAACUUGCA | CD3D gRNA |
| | | sequence 2 |
| 60 | UAGCCUUACCUUGCGAGAGA | CD3D gRNA |
| | | sequence 3 |
| 61 | CCGACACACAAGCUCUGUUG | CD3D gRNA |
| | | sequence 4 |
| 62 | CAACGCUCACCUGAUAGACC | CD3D gRNA |
| | | sequence 5 |
| 63 | GAACAUAGCACGUUUCUCUC | CD3D gRNA |
| | | sequence 6 |
| 64 | GACAAUGAUGCCAGCCACGG | CD3D gRNA |
| | | sequence 7 |
| 65 | UUGCAAUACCAGCAUCACAU | CD3D gRNA |
| | | sequence 8 |
| 66 | TTACACTGATACATCCCTCG | CD3G gRNA target |
| | | sequence 1 |
| 67 | ACTTTGGCCCAGTCAATCAA | CD3G gRNA target |
| | | sequence 2 |
| 68 | GTGTATGACTATCAAGAAGA | CD3G gRNA target |
| | | sequence 3 |
| 69 | TTCTCCTACCTTTGATTGAC | CD3G gRNA target |
| | | sequence 4 |
| 70 | CTTGAAGAAGAATGATAGCC | CD3G gRNA target |
| | | sequence 5 |
| 71 | CAGAGACTGACATGGAACAG | CD3G gRNA target |
| | | sequence 6 |
| 72 | TACACTGATACATCCCTCGA | CD3G gRNA target |
| | | sequence 7 |
| 73 | CAGAAGCCAAAAATATCACA | CD3G gRNA target |
| | | sequence 8 |
| 74 | AAAGAGAAAGCCAGATATGG | CD3G gRNA target |
| | | sequence 9 |
| 75 | UUACACUGAUACAUCCCUCG | CD3G gRNA |
| | | sequence 1 |
| 76 | ACUUUGGCCCAGUCAAUCAA | CD3G gRNA |
| | | sequence 2 |
| 77 | GUGUAUGACUAUCAAGAAGA | CD3G gRNA |
| | | sequence 3 |
| 78 | UUCUCCUACCUUUGAUUGAC | CD3G gRNA |
| | | sequence 4 |
| 79 | CUUGAAGAAGAAUGAUAGCC | CD3G gRNA |
| | | sequence 5 |
| 80 | CAGAGACUGACAUGGAACAG | CD3G gRNA |
| | | sequence 6 |
| 81 | UACACUGAUACAUCCCUCGA | CD3G gRNA |
| | | sequence 7 |
| 82 | CAGAAGCCAAAAAUAUCACA | CD3G gRNA |
| | | sequence 8 |
| 83 | AAAGAGAAAGCCAGAUAUGG | CD3G gRNA |
| | | sequence 9 |
| 84 | atgcttctcctggtgacaagccttctgctctgtgagttaccacacccagcattcct | GMCSFR alpha |
| | cctgatccca | chain signal |
| | | sequence |
| 85 | MLLLVTSLLLCELPHPAFLLIP | GMCSFR alpha |
| | | chain signal |
| | | sequence |
| 86 | MALPVTALLLPLALLLHA | CD8 alpha signal |
| | | peptide |
| 87 | ATGCTGCTGCTGGTGACCAGCCTGCTGCTGTGCGAGCTGCCCCACCCCGCCTTTCT | GMCSFR alpha |
| | GCTGATCCCC | chain signal |
| | | sequence |
| 88 | LEGGGEGRGSLLTCGDVEENPGPR | T2A |
| 89 | EGRGSLLTCGDVEENPGP | T2A |
| 90 | GSGATNFSLLKQAGDVEENPGP | P2A |
| 91 | ATNFSLLKQAGDVEENPGP | P2A |
| 92 | QCTNYALLKLAGDVESNPGP | E2A |
| 93 | VKQTLNFDLLKLAGDVESNPGP | F2A |
| 94 | GSGEGRGSLLTCGDVEENPGP | P2A |
| 95 | CTGACCTCTTCTCTTCCTCCCACAG | human HBB gene |
| | | splice acceptor |
| 96 | TTTCTCTCCACAG | human IgG gene |
| | | splice acceptor |
| 97 | DYGVS | CDR H1 |
| 98 | VIWGSETTYYNSALKS | CDR H2 |
| 99 | YAMDYWG | CDR H3 |
| 100 | HYYYGGSYAMDY | HC-CDR3 |
| 101 | RASQDISKYLN | CDR L1 |
| 102 | SRLHSGV | CDR L2 |
| 103 | HTSRLHS | LC-CDR2 |
| 104 | GNTLPYTFG | CDR L3 |
| 105 | QQGNTLPYT | LC-CDR3 |
| 106 | EVKLQESGPGLVAPSQSLSVTCTVSGVSLPDYGVSWIRQPPRKGLEWLGVIWGSET | VH |
| | TYYNSALKSRLTIIKDNSKSQVFLKMNSLQTDDTAIYYCAKHYYYGGSYAMDYWGQ | |
| | GTSVTVSS | |
| 107 | DIQMTQTTSSLSASLGDRVTISCRASQDISKYLNWYQQKPDGTVKLLIYHTSRLHS | VL |
| | GVPSRFSGSGSGTDYSLTISNLEQEDIATYFCQQGNTLPYTFGGGTKLEIT | |
| 108 | GNTLPYTFG | CDR L3 |
| 109 | GSTSGSGKPGSGEGSTKG | Linker |
| 110 | gacatccagatgacccagaccacctccagcctgagcgccagcctgggcgaccgggt | Sequence encoding |
| | gaccatcagctgccgggccagccaggacatcagcaagtacctgaactggtatcagc | scFv |
| | agaagcccgacggcaccgtcaagctgctgatctaccacaccagccggctgcacagc | |
| | ggcgtgcccagccggtttagcggcagcggctccggcaccgactacagcctgaccat | |
| | ctccaacctggaacaggaagatatcgccacctacttttgccagcagggcaacacac | |
| | tgccctacacctttggcggcggaacaaagctggaaatcaccggcagcacctccggc | |
| | agcggcaagcctggcagcggcgagggcagcaccaagggcgaggtgaagctgcagga | |
| | aagcggccctggcctggtggcccccagccagagcctgagcgtgacctgcaccgtga | |
| | gcggcgtgagcctgcccgactacggcgtgagctggatccggcagccccccaggaag | |
| | ggcctggaatggctgggcgtgatctggggcagcgagaccacctactacaacagcgc | |
| | cctgaagagccggctgaccatcatcaaggacaacagcaagagccaggtgttcctga | |
| | agatgaacagcctgcagaccgacgacaccgccatctactactgcgccaagcactac | |
| | tactacggcggcagctacgccatggactactggggccagggcaccagcgtgaccgt | |
| | gagcagc | |
| 111 | DIQMTQTTSSLSASLGDRVTISCRASQDISKYLNWYQQKPDGTVKLLIYHTSRLHS | scFv |
| | GVPSRFSGSGSGTDYSLTISNLEQEDIATYFCQQGNTLPYTFGGGTKLEITGSTSG | |
| | SGKPGSGEGSTKGEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAIYYCAKHY | |
| | YYGGSYAMDYWGQGTSVTVSS | |
| 112 | SYWMN | CDR H1 |
| 113 | QIYPGDGDTNYNGKFKG | CDR H2 |
| 114 | KTISSVVDFYFDY | CDR H3 |
| 115 | KASQNVGTNVA | CDR L1 |
| 116 | SATYRNS | CDR L2 |
| 117 | QQYNRYPYT | CDR L3 |
| 118 | EVKLQQSGAELVRPGSSVKISCKASGYAFSSYWMNWVKQRPGQGLEWIGQIYPGDG | VH |
| | DTNYNGKFKGQATLTADKSSSTAYMQLSGLTSEDSAVYFCARKTISSVVDFYFDYW | |
| | GQGTTVTVSS | |
| 119 | DIELTQSPKFMSTSVGDRVSVTCKASQNVGTNVAWYQQKPGQSPKPLIYSATYRNS | VL |
| | GVPDRFTGSGSGTDFTLTITNVQSKDLADYFCQQYNRYPYTSGGGTKLEIKR | |
| 120 | EVKLQQSGAELVRPGSSVKISCKASGYAFSSYWMNWVKQRPGQGLEWIGQIYPGDG | scFv |
| | DTNYNGKFKGQATLTADKSSSTAYMQLSGLTSEDSAVYFCARKTISSVVDFYFDYW | |
| | GQGTTVTVSSGGGGSGGGGSGGGGSDIELTQSPKFMSTSVGDRVSVTCKASQNVGT | |
| | NVAWYQQKPGQSPKPLIYSATYRNSGVPDRFTGSGSGTDFTLTITNVQSKDLADYF | |
| | CQQYNRYPYTSGGGTKLEIKR | |
| 121 | (GGGGS)n | Linker; n is an |
| | | integer between 1 |
| | | and 10, inclusive |
| 122 | GGGGS | Linker |
| 123 | GGGGSGGGGS | Linker |
| 124 | GGGGSGGGGSGGGGSGGGGS | Linker |
| 125 | (G₄S)₃₋₄ | Linker |
| 126 | (G₄S)₂₋₃ | Linker |
| 127 | GGGAS(G₄S)₂ | Linker |
| 128 | GGGGGS | Linker |
| 129 | (GGGGGS)n | Linker; n is an |
| | | integer between 1 |
| | | and 4, inclusive |
| 130 | (GGS)₂ | Linker |
| 131 | GGSGGSGGS | Linker |
| 132 | GGSGGSGGSGGS | Linker |
| 133 | GGSGGSGGSGGSGGS | Linker |
| 134 | GGGGGSGGGGGSGGGGGS | Linker |
| 135 | GGSGGGGSGGGGSGGGGS | Linker |
| 136 | Trp-Arg-His-Pro-Gln-Phe-Gly-Gly | Streptavidin binding |
| | | peptide, Strep-tag® |
| 137 | WSHPQFEK | Strep-tag® II |
| 138 | His-Pro-Baa | Streptavidin |
| | | Binding peptide |
| | | Baa is selected from |
| | | glutamine, |
| | | asparagine and |
| | | methionine |
| 139 | His-Pro-Gln-Phe | Streptavidin- |
| | | binding peptide |
| 140 | Oaa-Xaa-His-Pro-Gln-Phe-Yaa-Zaa | Streptavidin- |
| | | binding peptide |
| | | Oaa is Trp, Lys or |
| | | Arg; |
| | | Xaa is any amino |
| | | acid; |
| | | Yaa is Gly or Glu |
| | | Zaa is Gly, Lys or |
| | | Arg |
| 141 | -Trp-Xaa-His-Pro-Gln-Phe-Yaa-Zaa- | Streptavidin- |
| | | binding peptide |
| | | Xaa is any amino |
| | | acid; |
| | | Yaa is Gly or Glu |
| | | Zaa is Gly, Lys or |
| | | Arg |
| 142 | Trp-Ser-His-Pro-Gln-Phe-Glu-Lys-(Xaa)n-Trp-Ser-His-Pro- | Sequential modules |
| | Gln-Phe-Glu-Lys- | of streptavidin- |
| | | binding peptide |
| | | Xaa is any amino |
| | | acid; |
| | | n is either 8 or 12 |
| 143 | Trp-Ser-His-Pro-Gln-Phe-Glu-Lys-(GlyGlyGlySer)n-Trp-Ser- | Sequential modules |
| | His-Pro-Gln-Phe-Glu-Lys | of streptavidin- |
| | | binding peptide |
| | | n is 2 or 3 |
| 144 | SAWSHPQFEKGGGSGGGSGGGSWSHPQFEK | Twin-Strep-tag |
| 145 | SAWSHPQFEKGGGSGGGSGGSAWSHPQFEK | Twin-Strep-tag |
| 146 | WSHPQFEKGGGSGGGSGGGSWSHPQFEK | Twin-Strep-tag |
| 147 | WSHPQFEKGGGSGGGSWSHPQFEK | Twin-Strep-tag |
| 148 | WSHPQFEKGGGSGGGSGGSAWSHPQFEK | Twin-Strep-tag |
| 149 | SAWSHPQFEK | Streptavidin binding |
| | | peptide, Strep-tag® |
| | | II |
| 150 | GGGGG | linker |
| 151 | GlyₓXaa-Gly_{y}-Xaa-Gly_{z} | linker, each Xaa is |
| | | independently |
| | | selected from |
| | | Alanine (Ala), |
| | | Valine (Val), |
| | | Leucine (Leu), |
| | | Isoleucine (Ile), |
| | | Methionine (Met), |
| | | Phenylalanine |
| | | (Phe), Tryptophan |
| | | (Trp), Proline (Pro), |
| | | Glycine (Gly), |
| | | Serine (Ser), |
| | | Threonine (Thr), |
| | | Cysteine (Cys), |
| | | Tyrosine (Tyr), |
| | | Asparagine (Asn), |
| | | Glutamine (Gln), |
| | | Lysine (Lys), |
| | | Arginine (Arg), |
| | | Histidine (His), |
| | | Aspartate (Asp), |
| | | and Glutamate |
| | | (Glu), and wherein |
| | | x, y, and z are each |
| | | integers in the range |
| | | from 1-5 |
| 152 | Gly-Gly-Gly-Xaa-Gly-Gly-Gly-Xaa-Gly-Gly-Gly | linker, each Xaa is |
| | | independently |
| | | selected from |
| | | Alanine (Ala), |
| | | Valine (Val), |
| | | Leucine (Leu), |
| | | Isoleucine (Ile), |
| | | Methionine (Met), |
| | | Phenylalanine |
| | | (Phe), Tryptophan |
| | | (Trp), Proline (Pro), |
| | | Glycine (Gly), |
| | | Serine (Ser), |
| | | Threonine (Thr), |
| | | Cysteine (Cys), |
| | | Tyrosine (Tyr), |
| | | Asparagine (Asn), |
| | | Glutamine (Gln), |
| | | Lysine (Lys), |
| | | Arginine (Arg), |
| | | Histidine (His), |
| | | Aspartate (Asp), |
| | | and Glutamate |
| | | (Glu) |
| 153 | (SSSSG)n | linker, n is at least 1 |
| 154 | GGGGG-C-GGGGG | linker |

1. An engineered T cell, comprising a modified invariant CD3-immunoglobulin superfamily (invariant CD3-IgSF) chain locus comprising a nucleic acid sequence encoding a mini chimeric antigen receptor (miniCAR), wherein the miniCAR is a fusion protein comprising a heterologous antigen-binding domain and an endogenous invariant CD3 chain of the invariant CD3-IgSF chain locus, wherein:
the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain.

2. An engineered T cell expressing a mini chimeric antigen receptor (miniCAR), wherein the miniCAR is a fusion protein comprising a heterologous antigen-binding domain and an endogenous invariant CD3 chain of the immunoglobulin superfamily (invariant CD3-IgSF chain).

3. An engineered T cell comprising a transgene encoding an antigen-binding domain inserted in-frame with an open reading frame of a locus encoding an endogenous invariant CD3 chain of the immunoglobulin superfamily (invariant CD3-IgSF chain), wherein the engineered T cell expresses a miniCAR fusion protein comprising a heterologous antigen-binding domain and the endogenous invariant CD3-IgSF chain.

4. The engineered T cell of claim 2 or 3, wherein the miniCAR is expressed from a modified invariant CD3-immunoglobulin superfamily (invariant CD3-IgSF) chain locus comprising a nucleic acid sequence encoding the miniCAR, wherein:
the nucleic acid sequence comprises an in-frame fusion of (i) a transgene comprising a sequence encoding the antigen-binding domain and (ii) an open reading frame of the endogenous invariant CD3-IgSF chain locus encoding the invariant CD3-IgSF chain.

5. The engineered T cell of claim 1 or 4, wherein the modified invariant CD3-IgSF chain locus is a modified CD3 epsilon (CD3E) locus encoding a CD3e chain, a modified CD3 delta (CD3D) locus encoding a CD3d chain, or a modified CD3 gamma (CD3G) locus encoding a CD3g chain.

6. The engineered T cell of any of claim 1, 4, or 5, wherein the modified invariant CD3-IgSF chain locus is a modified CD3E locus encoding a CD3e chain.

7. The engineered T cell of any of claims 1-6, wherein the antigen-binding domain comprises an antibody or an antigen-binding fragment thereof.

8. The engineered T cell of any of claims 1-7, wherein the antigen-binding domain comprises a Fab fragment, a Fab₂ fragment, a single domain antibody, or a single chain variable fragment (scFv).

9. The engineered T cell of any one of claim 1, or 5-8, wherein the modified invariant CD3-IgSF chain locus comprises, in order from 5′ to 3′, a sequence of nucleotides encoding the heterologous antigen-binding domain and the endogenous invariant CD3-IgSF chain.

10. The engineered T cell of any of claims 1-9, wherein the antigen-binding domain and the invariant CD3-IgSF chain are directly linked.

11. The engineered T cell of any of claims 1-9, wherein the antigen-binding domain and the invariant CD3-IgSF chain are linked indirectly via a linker.

12. The engineered T cell of any of claim 1, or 3-11, wherein the transgene further comprises a nucleic acid sequence encoding a linker.

13. The engineered T cell of claim 12, wherein the linker is positioned 3′ to the antigen-binding domain.

14. An engineered T cell, comprising a modified CD3E locus comprising a nucleic acid sequence encoding a miniCAR, the miniCAR comprising a heterologous antigen-binding domain and an endogenous CD3e chain, wherein:
the nucleic acid sequence comprises an in-frame fusion of (i) a transgene comprising a sequence encoding the antigen-binding domain, wherein the antigen-binding domain is an scFv, and a sequence encoding a linker, and (ii) an open reading frame of an endogenous CD3E locus encoding the CD3e chain.

15. The engineered T cell of any one of claims 12-14, wherein the transgene sequence comprises, in order from 5′ to 3′, a sequence of nucleotides encoding the antigen-binding domain and a sequence of nucleotides encoding the linker.

16. The engineered T cell of claim 15, wherein the modified invariant CD3-IgSF chain locus comprises, in order from 5′ to 3′, a sequence of nucleotides encoding the antigen-binding domain, the linker, and the invariant CD3-IgSF chain.

17. The engineered T cell of any of claims 12-16, wherein the linker is a polypeptide linker.

18. The engineered T cell of any of claims 12-17, wherein the linker is a polypeptide that is 3 to 18 amino acids in length.

19. The engineered T cell of any of claims 12-18, wherein the linker comprises GS, GGS, GGGGS (SEQ ID NO:122), GGGGGS (SEQ ID NO:128) and combinations thereof.

20. The engineered T cell of any of claims 12-18, wherein the linker comprises (GGS)n, wherein n is 1 to 10, (GGGGS)n (SEQ ID NO: 121), wherein n is 1 to 10, or (GGGGGS)n (SEQ ID NO:129), wherein n is 1 to 4.

21. The engineered T cell of any of claim 1, or 3-20, wherein the transgene further comprises a nucleic acid sequence encoding one or more multicistronic element.

22. The engineered T cell of claim 21, wherein the P2A element comprises the sequence set forth in SEQ ID NO: 3.

23. The engineered T cell of claim 21 or claim 22, wherein at least one of the one or more multicistronic elements is positioned 5′ to the antigen-binding domain.

24. The engineered T cell of any of claims 21-23, wherein the transgene sequence comprises, in order from 5′ to 3′, a sequence of nucleotides encoding the multicistronic element, optionally the P2A element; the antigen-binding domain; and the linker.

25. The engineered T cell of any of claim 1, or 3-24, wherein the transgene further comprises a nucleic acid sequence encoding an affinity tag.

26. The engineered T cell of claim 25, wherein the affinity tag is a streptavidin binding peptide.

27. The engineered T cell of any of claims 21-26, wherein the modified invariant CD3-IgSF chain locus comprises, in order from 5′ to 3′, a sequence of nucleotides encoding the multicistronic element; the antigen-binding domain; the linker; and the invariant CD3-IgSF chain.

28. The engineered T cells of any of claim 1, 3-13, or 15-27, wherein the open reading frame of the endogenous invariant CD3-IgSF chain locus encodes a full length mature invariant CD3-IgSF chain.

29. The engineered T cell of any of claim 1, 4-13, or 15-28, wherein the modified invariant CD3-IgSF chain locus comprises the promoter and/or regulatory or control element of the endogenous locus operably linked to control expression the nucleic acid sequence encoding the miniCAR.

30. The engineered T cell of any of claim 1, 4-13, or 15-28, wherein the modified invariant CD3-IgSF chain locus comprises one or more heterologous regulatory or control elements operably linked to control expression of the miniCAR or a portion thereof.

31. The engineered T cell of any of claims 1-30, wherein the antigen-binding domain binds to a target antigen that is associated with, specific to, and/or expressed on a cell or tissue of a disease, disorder or condition.

32. The engineered T cell of claim 31, wherein the target antigen is a tumor antigen.

33. The engineered T cell of claim 31 or 32, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

34. The engineered T cell of any of claims 1-33, wherein the miniCAR assembles into a TCR/CD3 complex in place of the corresponding endogenous invariant CD3-IgSF chain of the TCR/CD3 complex.

35. The engineered T cell of any of claims 5-34, wherein the miniCAR assembles into a TCR/CD3 complex in place of the corresponding endogenous invariant CD3-IgSF CD3e chain of the TCR/CD3 complex.

36. The engineered T cell of claim 34 or 35, wherein binding of a target antigen by the heterologous antigen-binding domain of the miniCAR induces antigen-dependent signaling via the TCR/CD3 complex.

37. The engineered T cell of any of claims 34-36, wherein the miniCAR exhibits reduced tonic signaling via the TCR/CD3 complex compared to T cells engineered with a chimeric antigen receptor (CAR) that comprises the same antigen-binding domain.

38. The engineered T cell of any of claims 1-37, wherein the engineered T cell exhibits increased persistence compared to T cells engineered with a chimeric antigen receptor (CAR) that comprises the same antigen-binding domain and a heterologous CD3zeta (CD3z) signaling domain.

39. The engineered T cell of any of claims 1-38, wherein the engineered T cell exhibits increased cytolytic activity compared to T cells engineered with a chimeric antigen receptor (CAR) that comprises the same antigen-binding domain and a heterologous CD3zeta (CD3z) signaling domain

40. The engineered T cell of any of claims 1-39, wherein the T cell is a primary T cell derived from a subject.

41. The engineered T cells of claim 40, wherein the subject is a human.

42. The engineered T cell of any of claims 1-41, wherein the T cell is a CD8+ T cell or a subtype thereof, or a CD4+ T cell or a subtype thereof.

43. The engineered T cell of any of claim 1, 2, or 4-42, wherein the transgene is integrated at the endogenous invariant CD3-IgSF chain locus of a T cell via homology directed repair (HDR).

44. A polynucleotide, comprising:
(a) a nucleic acid sequence encoding an antigen-binding domain; and
(b) one or more homology arms linked to the nucleic acid sequence, wherein the one or more homology arms comprise a sequence homologous to one or more regions of an open reading frame of an invariant CD3 chain of the immunoglobulin superfamily (invariant CD3-IgSF chain) locus of a T cell, wherein the invariant CD3-IgSFchain locus encodes an invariant CD3-IgSF chain.

45. The polynucleotide of claim 44, wherein the one or more homology arms comprise a sequence homologous to one or more regions of an open reading frame of the invariant CD3-IgSF chain locus, wherein the invariant CD3-IgSF chain locus is a CD3E locus encoding a CD3e chain, a CD3D locus encoding a CD3d chain, or a CD3G locus encoding a CD3g chain.

46. A polynucleotide, comprising:
(a) a nucleic acid sequence encoding an antigen-binding domain; and
(b) one or more homology arms linked to the nucleic acid sequence encoding the transgene, wherein the one or more homology arms comprise a sequence homologous to one or more regions of an open reading frame of a CD3E locus encoding a CD3e chain.

47. The polynucleotide of any of claims 44-46, wherein the antigen-binding domain comprises an antibody or an antigen-binding fragment thereof.

48. The polynucleotide of any of claims 44-47, wherein the antigen-binding domain comprises a Fab fragment, a Fab₂ fragment, a single domain antibody, or a single chain variable fragment (scFv).

49. The polynucleotide of any of claims 44-48, wherein the nucleic acid sequence further comprises nucleotides encoding a linker operably connected to the encoded antigen-binding domain, wherein the linker is positioned 3′ to the antigen-binding domain.

50. The polynucleotide of claim 49, wherein the encoded linker is a polypeptide that is 3 to 18 amino acids in length.

51. The polynucleotide of any of claim 49 or 50, wherein the encoded linker comprises GS, GGS, GGGGS (SEQ ID NO:122), GGGGGS (SEQ ID NO:128) and combinations thereof.

52. The polynucleotide of any of claims 49-51, wherein the encoded linker comprises (GGS)n, wherein n is 1 to 10, (GGGGS)n (SEQ ID NO: 121), wherein n is 1 to 10, or (GGGGGS)n (SEQ ID NO:129), wherein n is 1 to 4.

53. The polynucleotide of any of claims 49-52, wherein the encoded linker is selected from the group consisting of a encoded linker that comprises GGS, comprises GGGGS (SEQ ID NO: 122), comprises GGGGGS (SEQ ID NO: 128), comprises (GGS)2 (SEQ ID NO: 130), is or comprises GGSGGSGGS (SEQ ID NO: 131), comprises GGSGGSGGSGGS (SEQ ID NO:132), comprises GGSGGSGGSGGSGGS (SEQ ID NO:133), comprises GGGGGSGGGGGSGGGGGS (SEQ ID NO:134), comprises GGSGGGGSGGGGSGGGGS (SEQ ID NO: 135), comprises and GGGGSGGGGSGGGGS (SEQ ID NO:16).

54. The polynucleotide of any of claims 49-53, wherein the nucleic acid sequence comprises, in order from 5′ to 3′, a sequence of nucleotides encoding the antigen-binding domain and a sequence of nucleotides encoding the linker.

55. The polynucleotide of any of claims 44-54, wherein the nucleic acid sequence further comprises nucleotides encoding one or more multicistronic elements.

56. The polynucleotide of claim 55, wherein the multicistronic element comprises a P2A element, wherein the P2A element comprises the sequence set forth in SEQ ID NO: 3.

57. The polynucleotide of claim 55 or 56, wherein the nucleic acid sequence comprises, in order from 5′ to 3′, a sequence of nucleotides encoding the multicistronic element, optionally the P2A element; the antigen-binding domain; and the linker.

58. The polynucleotide of any of claims 44-57, wherein the one or more homology arms comprise a 5′ homology arm and a 3′ homology arm and the polynucleotide comprises the structure [5′ homology arm]-[nucleic acid sequence of (a)]-[3′ homology arm].

59. The polynucleotide of claim 58, wherein the 5′ homology arm and the 3′ homology arm independently are at or about 100, 200, 300, 400, 500, 600, 700 or 800 nucleotides in length, or any value between any of the foregoing, or are greater than at or about 100 nucleotides in length, optionally at or about 100, 200 or 300 nucleotides in length, or any value between any of the foregoing.

60. The polynucleotide of claim 58 or 59, wherein the 5′ homology arm comprises a sequence that exhibits at least 85%

61. The polynucleotide of any of claims 58-60, wherein the 3′ homology arm comprises a sequence that exhibits at least 85% sequence identity to SEQ ID NO: 5.

62. The polynucleotide of any of claims 44-61, wherein the encoded antigen-binding domain binds to a target antigen that is associated with, specific to, and/or expressed on a cell or tissue of a disease, disorder or condition.

63. The polynucleotide of claim 62, wherein the target antigen is a tumor antigen.

64. The polynucleotide of claim 62 or 63, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

65. The polynucleotide of any of claims 44-45 and 47-64, wherein introduction of the polynucleotide into a genome of a T cell generates a modified invariant CD3-IgSF chain locus encoding a mini chimeric antigen receptor (miniCAR), wherein the miniCAR is a fusion protein comprising the antigen-binding domain encoded by the nucleic acid of the polynucleotide and an endogenous invariant CD3-IgSF chain, and wherein the modified invariant CD3-IgSF chain locus comprises the nucleic acid encoding the antigen-binding domain in-frame with an open reading frame of the endogenous invariant CD3-IgSF chain locus encoding the invariant CD3-IgSF chain.

66. The polynucleotide of claim 65, wherein the endogenous invariant CD3-IgSF chain is a CD3e chain, a CD3d chain, or a CD3g chain.

67. The polynucleotide of any of claims 65-66, wherein the encoded miniCAR assembles into a TCR/CD3 complex in place of the corresponding endogenous invariant CD3-IgSF chain of the TCR/CD3 complex.

68. The polynucleotide of any of claims 44-67, that is a linear polynucleotide.

69. The polynucleotide of any of claims 44-68, wherein the polynucleotide is comprised in a vector.

70. The polynucleotide of any of claims 44-69, wherein the polynucleotide is between about 500 and about 3000 nucleotides, about 1000 and about 2500 nucleotides, or about 1500 nucleotides and about 2000 nucleotides in length.

71. A vector comprising the polynucleotide of any of claims 44-67 and 69-70.

72. The vector of claim 71, wherein the vector is a viral vector.

73. The vector of claim 72, wherein the viral vector is a retroviral vector.

74. A method of producing genetically engineered T cells, the method comprising introducing the polynucleotide of any of claims 44-73 into a population of T cells, wherein T cells of the population comprise a genetic disruption at an endogenous invariant CD3-IgSF chain locus, wherein the invariant CD3-IgSF chain locus encodes an invariant CD3-IgSF chain.

75. A method of producing genetically engineered T cells, the method comprising introducing the vector of any of claims 71-73 into a population of T cells, wherein T cells of the population comprise a genetic disruption at an endogenous invariant CD3-IgSF chain locus, wherein the invariant CD3-IgSF chain locus encodes an invariant CD3-IgSF chain.

76. A method of producing genetically engineered T cells, the method comprising:
(a) introducing, into a population of T cells, one or more agents capable of inducing a genetic disruption at a target site within an endogenous invariant CD3-IgSF chain locus of T cells in the population, wherein the invariant CD3-IgSF chain locus encodes an invariant CD3-IgSF chain; and
(b) introducing the polynucleotide of any of claims 44-70 into the population of T cells, wherein T cells in the population comprise a genetic disruption at the endogenous invariant CD3 IgSF chain locus.

77. A method of producing genetically engineered T cells, the method comprising:
(a) introducing, into a population of T cells, one or more agents capable of inducing a genetic disruption at a target site within an endogenous invariant CD3-IgSF chain locus of T cells in the population, wherein the invariant CD3-IgSF chain locus encodes an invariant CD3-IgSF chain; and
(b) introducing the vector of any of claims 71-73 into the population of T cells, wherein T cells in the population comprise a genetic disruption at the endogenous invariant CD3 IgSF chain locus.

78. The method of any of claims 74-77, wherein the nucleic acid sequence of the polynucleotide is integrated in the endogenous invariant CD3-IgSF chain locus via homology directed repair (HDR).

79. The method of any one of claims 74-78, wherein the invariant CD3-IgSF chain locus is a CD3 epsilon (CD3E) locus encoding a CD3e chain, a CD3 delta (CD3D) locus encoding a CD3d chain, or a CD3 gamma (CD3G) locus encoding a CD3g chain.

80. The method of any of claims 74-79, wherein the genetic disruption is carried out by introducing into the population of T cells, one or more agents to induce a genetic disruption at a target site within an endogenous invariant CD3-IgSF chain locus of the T cell.

81. The method of any of claims 76-80, wherein the one or more agents capable of inducing a genetic disruption comprises a DNA binding protein or DNA-binding nucleic acid, a fusion protein comprising a DNA-targeting protein and a nuclease, or an RNA-guided nuclease that specifically binds to or hybridizes to the target site, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site.

82. The method of any of claims 76-81, wherein each of the one or more agents comprise a guide RNA (gRNA) having a targeting domain that is complementary to the at least one target site.

83. The method of claim 82, wherein the one or more agents are introduced as a ribonucleoprotein (RNP) complex comprising the gRNA and a Cas9 protein, optionally wherein the RNP is introduced via electroporation, particle gun, calcium phosphate transfection, cell compression or squeezing, optionally via electroporation.

84. The method of any of claims 82-83, wherein the gRNA has a targeting domain sequence UUGACAUGCCCUCAGUAUCC (SEQ ID NO: 8).

85. The method of any of claims 74-84, wherein the population of T cells comprise primary T cells derived from a subject.

86. The method of any of claims 74-85, wherein the T cells comprise CD8+ T cell or subtypes thereof, or CD4+ T cells or subtypes thereof.

87. The method of any of claims 74, and 76-86, wherein the polynucleotide is a linear polynucleotide.

88. The method of any of claims 74, and 76-86, wherein the polynucleotide is comprised in a vector.

89. The method of any of claims 76-88, wherein the one or more agent(s) and the polynucleotide or vector are introduced simultaneously or sequentially, in any order.

90. The method of claim 89, wherein the polynucleotide or vector is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of the one or more agents.

91. The method of any of claims 76-90, wherein prior to the introducing of the one or more agents and/or the introducing of the polynucleotide or vector, the method comprises incubating the population of T cells, in vitro with one or more stimulatory agents under conditions to stimulate or activate one or more T cells of the population.

92. The method of any of claims 76-91, wherein the method further comprises incubating the population of T cells prior to, during or subsequent to the introducing of the one or more agents and/or the introducing of the polynucleotide or vector with one or more recombinant cytokines.

92. The method of claim 91 or 92, wherein the incubation is carried out subsequent to the introducing of the one or more agents and the introducing of the polynucleotide or vector, and wherein the incubation is for up to 21 days, optionally up to or about 7 days.

93. The method of any of claims 76-92, further comprising cultivating the population of T cells under conditions for expansion, wherein the cultivating is subsequent to the introducing of the one or more agents and/or the introducing of the polynucleotide or vector.

94. The method of claim 93, wherein the cultivating under conditions for expansion comprises incubating the population of T cells with the target antigen of the antigen-binding domain, target cells expressing the target antigen, or an anti-idiotype antibody that binds to the antigen-binding domain.

95. The method of claim 93 or 94, wherein the cultivating under conditions for expansion is carried out for up 21 days.

96. The method of any of claims 74-95, wherein the method results in at least 75% of the cells in the population of T cells comprise a genetic disruption of at least one target site within the invariant CD3-IgSF chain locus.

97. The method of any of claims 74-96, wherein the method results in at least or greater than 25% or of the T cells in the population of T cells generated by the method express the miniCAR.

98. A population comprising engineered T cells produced by the method of any of claims 74-97.

99. A T cell comprising a TCR/CD3 complex comprising a mini chimeric antigen receptor (CAR), wherein the miniCAR is a fusion protein comprising a heterologous antigen-binding domain and an endogenous invariant CD3 chain of the immunoglobulin superfamily (invariant CD3-IgSF chain) of the TCR/CD3 complex.

100. The T cell of claim 99, wherein the miniCAR is expressed from a modified invariant CD3-IgSF chain locus of the T cell, the modified invariant CD3-IgSF chain locus comprising a nucleic acid sequence encoding the miniCAR.

101. The T cell of claim 100, wherein the invariant CD3-IgSF chain locus is a CD3 epsilon (CD3E), a CD3 delta (CD3D), or a CD3 gamma (CD3G) locus.

102. A composition, comprising the engineered T cell of any of claims 1-44, the population comprising engineered T cells of claim 98, or the T cell of any of claims 99-101.

103. A composition, comprising engineered T cells produced by the method of any of claims 74-97.

104. The composition of claim 102 or claim 103, wherein the composition comprises CD4+ T cells and/or CD8+ T cells.

105. The composition of claim 104, wherein the composition comprises CD4+ T cells and CD8+ T cells and the ratio of CD4+ to CD8+ T cells is from about 1:3 to 3:1.

106. The composition of any of claims 102-105, wherein the composition comprises a plurality of T cells expressing the miniCAR.

107. The composition of any of claims 102-106, wherein the composition comprises about 1×10⁶, 1.5×10⁶, 2.5×10⁶, 5×10⁶, 7.5×10⁶, 1×10⁷, 1.5×10⁷, 2×10⁷, 2.5×10⁷, 5×10⁷, 7.5×10⁷, 1×10⁸, 1.5×10⁸, 2.5×10⁸, or 5×10⁸ total T cells.

108. The composition of any of claims 102-107, wherein the composition comprises about 1×10⁵, 2.5×10⁵, 5×10⁵, 6.5×10⁵, 1×10⁶, 1.5×10⁶, 2×10⁶, 2.5×10⁶, 5×10⁶, 7.5×10⁶, 1×10⁷, 1.5×10⁷, 5×10⁷, 7.5×10⁷, 1×10⁸ or 2.5×10⁸ T cells expressing the miniCAR.

109. The composition of any of claims 102-108, wherein the frequency of T cells in the composition expressing the miniCAR is at or about 25%, 30%, 35%, 40%, 45%, 50%, 55%, 60%, 65%, 70%, 75%, 80%, or 90% or more of the total cells in the composition, or of the total CD4+ T cells or CD8+ T cells in the composition, or the total cells in the composition that comprises a genetic disruption within an endogenous invariant CD3-IgSF chain locus.

110. The composition of any of claims 102-109 that is a pharmaceutical composition.

111. A method of treatment comprising administering the engineered T cell of any of claims 1-43, the population comprising engineered T cells of claim 98, the T cell of any of claims 99-101, or the composition of any of claims 102-110, to a subject having a disease or disorder.

112. Use of the engineered T cell of any of claims 1-43, the population comprising engineered T cells of claim 98, the T cell of any of claims 99-101, or the composition of any of claims 102-110 for the treatment of a disease or disorder.

113. Use of the engineered T cell of any of claims 1-43, the population comprising engineered T cells of claim 98, the T cell of any of claims 99-101, or the composition of any of claims 102-110 in the manufacture of a medicament for treating a disease or disorder.

114. The engineered T cell of any of claims 1-43, the population comprising engineered T cells of claim 98, the T cell of any of claims 99-101, or the composition of any of claims 102-110 for use in the treatment of a disease or disorder.

115. The method, the use, the engineered T cell, the population of engineered T cells, the T cell or the composition for use of any of claims 110-114, wherein cells or tissues associated with the disease or disorder express the target antigen recognized by the antigen binding domain.

116. The method, the use, the engineered T cell, the population of engineered T cells, the T cell or the composition for use of any of claims 110-115, wherein the disease or disorder is a cancer or a tumor.

117. The method, the use, the engineered T cell, the population of engineered T cells, the T cell or the composition for use of claim 116, wherein the cancer or the tumor is a a lymphoma, a leukemia, or a plasma cell malignancy.

118. The method, the use, the engineered T cell, the population of engineered T cells, the T cell or the composition for use of claim 116 or 117, wherein the cancer is a lymphoma and the lymphoma is Burkitt's lymphoma, non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL).

119. The method, the use, the engineered T cell, the population of engineered T cells, the T cell or the composition for use of any of claims 117-118, wherein the cancer is a leukemia and the leukemia is chronic lymphocytic leukemia (CLL), plasma cell leukemia or acute lymphocytic leukemia (ALL).

120. The method, the use, the engineered T cell, the population of engineered T cells, the T cell or the composition for use of any of claims 117-118, wherein the cancer is a plasma cell malignancy and the plasma cell malignancy is multiple myeloma (MM).

121. The method, the use, the engineered T cell, the population of engineered T cells, the T cell or the composition for use of claim 116, wherein the cancer or the tumor is a solid tumor, optionally wherein the solid tumor is a non-small cell lung cancer (NSCLC) or a head and neck squamous cell carcinoma (HNSCC).

122. A kit comprising:
one or more agents capable of inducing a genetic disruption at a target site within an endogenous invariant CD3-IgSF chain locus of a T cell; and
the polynucleotide of any of claims 44-70.

123. A kit, comprising:
one or more agents capable of inducing a genetic disruption at a target site within an endogenous invariant CD3-IgSF chain locus of a T cell; and
a polynucleotide of any of claims 44-70, wherein the polynucleotide is targeted for integration at or near the target site via homology directed repair (HDR); and
instructions for carrying out the method of any of claims 71-96.

124. The kit of claim 122 or 123, wherein the endogenous invariant CD3-IgSF chain locus is a CD3E locus encoding a CD3e chain, a CD3D locus encoding a CD3d chain, or a CD3G locus encoding an CD3g chain.

125. The kit of any of claims 122-124, wherein the one or more agents capable of inducing a genetic disruption comprises a DNA binding protein or DNA-binding nucleic acid that specifically binds to or hybridizes to the target site, a fusion protein comprising a DNA-targeting protein and a nuclease, or an RNA-guided nuclease, optionally wherein the one or more agent(s) comprises a zinc finger nuclease (ZFN), a TAL-effector nuclease (TALEN), or and a CRISPR-Cas9 combination that specifically binds to, recognizes, or hybridizes to the target site.

126. The kit of any of claims 122-125, wherein the each of the one or more agents comprise a guide RNA (gRNA) having a targeting domain that is complementary to the at least one target site.

127. The kit of claim 126, wherein the gRNA has a targeting domain sequence UUGACAUGCCCUCAGUAUCC (SEQ ID NO: 8).


SEQUENCE LISTING (154 sequences)

SEQ ID NO: 1 - DNA, 801 bp, Artificial Sequence
  Anti-CD19 scFv
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagcaggttgcacagcggcgtccccagtcgcttctcaggaagtggatcagggaccgattacagtctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 2 - DNA, 45 bp, Artificial Sequence
  G4Sx3 linker
ggtggaggaggctctggtggaggcggtagcggaggcggagggtcg

SEQ ID NO: 3 - DNA, 63 bp, Artificial Sequence
  P2A
ggaagcggagagggcagaggaagtcttctaacatgcggtgacgtggaggagaatcccggccca

SEQ ID NO: 4 - DNA, 119 bp, Artificial Sequence
  Synthetic construct
gtcactaatttgccttttctaaaattgtcctggtttcttctgccaatttcccttctttctccccagcatataaagtctccatctctggaaccacagtaatattgacatgccctcagtat

SEQ ID NO: 5 - DNA, 267 bp, Artificial Sequence
  Synthetic construct
gatggtaatgaagaaatgggaggcattactcagacaccatacaaggtcagtatcagtgggaccaccgtaatcctcacctgcccacagtatcctggatctgaaatactatggcaacacaatgataaaaacataggcggtgatgaggatgataaaaacataggaagcgatgaggatcacctgtcactgaaggaattttcagaattggagcaaagtggttattatgtctgctaccccagaggaagcaaaccagaagatgcgaacttttat

SEQ ID NO: 6 - DNA, 1295 bp, Artificial Sequence
  CD3e MiniCAR
gtcactaatttgccttttctaaaattgtcctggtttcttctgccaatttcccttctttctccccagcatataaagtctccatctctggaaccacagtaatattgacatgccctcagtatggaagcggagagggcagaggaagtcttctaacatgcggtgacgtggaggagaatcccggcccaatgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagcaggttgcacagcggcgtccccagtcgcttctcaggaagtggatcagggaccgattacagtctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggtggaggaggctctggtggaggcggtagcggaggcggagggtcggatggtaatgaagaaatgggaggcattactcagacaccatacaaggtcagtatcagtgggaccaccgtaatcctcacctgcccacagtatcctggatctgaaatactatggcaacacaatgataaaaacataggcggtgatgaggatgataaaaacataggaagcgatgaggatcacctgtcactgaaggaattttcagaattggagcaaagtggttattatgtctgctaccccagaggaagcaaaccagaagatgcgaacttttat

SEQ ID NO: 7 - DNA, 1283 bp, Artificial Sequence
  CD3e MiniCAR
gtcactaatttgccttttctaaaattgtcctggtttcttctgccaatttcccttctttctccccagcatataaagtctccatctctggaaccacagtaatattgacatgccctcagtatggaagcggagagggcagaggaagtcttctaacatgcggtgacgtggaggagaatcccggcccaatgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagcaggttgcacagcggcgtccccagtcgcttctcaggaagtggatcagggaccgattacagtctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggaggcggttctggaggtggaagcggtggctctgatggtaatgaagaaatgggaggcattactcagacaccatacaaggtcagtatcagtgggaccaccgtaatcctcacctgcccacagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttat

SEQ ID NO: 8 - RNA, 20 nt, Artificial Sequence
  CD3E gRNA target sequence 1
uugacaugcccucaguaucc

SEQ ID NO: 9 - DNA, 20 bp, Artificial Sequence
  CD3E gRNA sequence 1
ttgacatgccctcagtatcc

SEQ ID NO: 10 - RNA, 20 nt, Artificial Sequence
  TRAC gRNA target sequence
agagucucucagcugguaca

SEQ ID NO: 11 - DNA, 20 bp, Artificial Sequence
  TRAC gRNA sequence
agagtctctcagctggtaca

SEQ ID NO: 12 - DNA, 1566 bp, Artificial Sequence
  anti-CD19 CAR
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgagagcaagaattggagccacccgcagttcgaaaaaggaggtggaggttcaggtggtggaggctcttacggaccgaattggtctcatcctcagttcgagaaaggaggcggttctggaggtggaagcggtggctcttggagccacccacagtttgaaaagggaggcgggggctccggtggcggaggctcttccggatctccctgtccaccttgccctatgttctgggtgctggtagtggtaggtggagtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcacctgcctaccagcagggccagaatcagctgtacaacgagctgaacctgggacgaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 13 - DNA, 70 bp, Artificial Sequence
  TRAC 5′ homology arm
gggaaatgagatcatgtcctaaccctgatcctcttgtcccacagatatccagaaccctgaccctgccgtg

SEQ ID NO: 14 - DNA, 58 bp, Artificial Sequence
  TRAC 3′ homology arm
taccagctgagagactctaaatccagtgacaagtctgtctgcctattcaccgattttg

SEQ ID NO: 15 - DNA, 1049 bp, Artificial Sequence
  TRAC minicar without linker
gggaaatgagatcatgtcctaaccctgatcctcttgtcccacagatatccagaaccctgaccctgccgtgggaagcggagagggcagaggaagtcttctaacatgcggtgacgtggaggagaatcccggcccaatgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagcaggttgcacagcggcgtccccagtcgcttctcaggaagtggatcagggaccgattacagtctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggaggcggttctggaggtggaagcggtggctctatccagaaccccgaccctgctgtttatcagctcagggattctaaatccagtgacaagtctgtctgcctattcaccgattttg

SEQ ID NO: 16 - protein, 15 aa, Artificial Sequence
  G4Sx3 linker
GGGGSGGGGSGGGGS

SEQ ID NO: 17 - protein, 207 aa, Artificial Sequence
  CD3epsilon CD3e isoform 1 NCBI NP_000724.1
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 18 - DNA, 1361 bp, Artificial Sequence
  CD3epsilon CD3e NCBI NM_000733
agaaaccctcctcccctcccagcctcaggtgcctgcttcagaaaatgaagtagtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaccccccctccactgtaggccactggatggtcatttgcatctccgtaaatgtgctctgctcctcagctgagagagaaaaaaataaactgtatttggctgcaa

SEQ ID NO: 19 - protein, 201 aa, Artificial Sequence
  CD3epsilon CD3e isoform 2 Uniprot E9PSH8
MQSGTHWRVLGLCLLSVGVWGQDGNEEMAYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 20 - protein, 171 aa, Artificial Sequence
  CD3delta CD3d isoform 1 Uniprot P04234-1
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 21 - DNA, 771 bp, Artificial Sequence
  CD3delta CD3d isoform 1 GenBank NM_000732.4
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 22 - protein, 127 aa, Artificial Sequence
  CD3delta CD3d isoform 2 Uniprot P04234-2
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 23 - DNA, 639 bp, Artificial Sequence
  CD3delta CD3d isoform 2 GenBank NM_001040651.1
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaactgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 24 - protein, 98 aa, Artificial Sequence
  CD3delta CD3d isoform 3 Uniprot E9PMT5
MEHSTFLSGLVLATLLSQVCQSCVELDPATVAGIIVTDVIATLLLALGVFCFAGHETGRLSGAADTQALLRNDQVYQPLRDRDDAQYSHLGGNWARNK

SEQ ID NO: 25 - DNA, 297 bp, Artificial Sequence
  CD3delta CD3d isoform 3 GenBank JN392069.1
atggaacatagcacgtttctctctggcctggtactggctacccttctctcgcaagtgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtga

SEQ ID NO: 26 - protein, 182 aa, Artificial Sequence
  CD3gamma CD3g isoform 1 Uniprot P09693
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 27 - DNA, 1311 bp, Artificial Sequence
  CD3delta CD3d isoform 1 GenBank NM_NM_000073.2
agtctagctgctgcacaggctggctggctggctggctgctaagggctgctccacgcttttgccggaggacagagactgacatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattgaactcaggactcagagtagtccaggtgttctcctcctattcagttcccagaatcaaagcaatgcattttggaaagctcctagcagagagactttcagccctaaatctagactcaaggttcccagagatgacaaatggagaagaaaggccatcagagcaaatttgggggtttctcaaataaaataaaaataaaaacaaatactgtgtttcagaagcgccacctattggggaaaattgtaaaagaaaaatgaaaagatcaaataaccccctggatttgaatataattttttgtgttgtaatttttatttcgtttttgtataggttataattcacatggctcaaatattcagtgaaagctctccctccaccgccatcccctgctacccagtgaccctgttgccctcttcagagacaaattagtttctcttttttttttttttttttttttttttgagacagtctggctctgtcacccaggctgaaatgcagtggcaccatctcggctcactgcaacctctgcctcctgggttcaagcgattctcctgcctcagcctcccgggcagctgggattacaggcacacactaccacacctggctaatttttgtatttttagtagagacagggttttgctctgttggccaagctggtctcgaactcctgacctcaagtgatccgcccgcctc

SEQ ID NO: 28 - DNA, 20 bp, Artificial Sequence
  CD3E gRNA target sequence 1
tgccatagtatttcagatcc

SEQ ID NO: 29 - DNA, 20 bp, Artificial Sequence
  CD3E gRNA target sequence 2
ctggattacctcttgccctc

SEQ ID NO: 30 - DNA, 20 bp, Artificial Sequence
  CD3E gRNA target sequence 3
agggcatgtcaatattactg

SEQ ID NO: 31 - DNA, 20 bp, Artificial Sequence
  CD3E gRNA target sequence 4
tattatgtctgctaccccag

SEQ ID NO: 32 - DNA, 20 bp, Artificial Sequence
  CD3E gRNA target sequence 5
agataaaagttcgcatcttc

SEQ ID NO: 33 - DNA, 20 bp, Artificial Sequence
  CD3E gRNA target sequence 6
agatgcagtcgggcactcac

SEQ ID NO: 34 - DNA, 20 bp, Artificial Sequence
  CD3E gRNA target sequence 7
ttactttactaagatggcgg

SEQ ID NO: 35 - DNA, 20 bp, Artificial Sequence
  CD3E gRNA target sequence 8
gatggagactttatatgctg

SEQ ID NO: 36 - DNA, 20 bp, Artificial Sequence
  CD3E gRNA target sequence 9
gatgtccactatgacaattg

SEQ ID NO: 37 - DNA, 20 bp, Artificial Sequence
  CD3E gRNA target sequence 10
caacacaatgataaaaacat

SEQ ID NO: 38 - DNA, 20 bp, Artificial Sequence
  CD3E gRNA target sequence 11
tgaggatcacctgtcactga

SEQ ID NO: 39 - RNA, 20 nt, Artificial Sequence
  CD3E gRNA sequence 1
ugccauaguauuucagaucc

SEQ ID NO: 40 - RNA, 20 nt, Artificial Sequence
  CD3E gRNA sequence 2
cuggauuaccucuugcccuc

SEQ ID NO: 41 - RNA, 20 nt, Artificial Sequence
  CD3E gRNA sequence 3
agggcaugucaauauuacug

SEQ ID NO: 42 - RNA, 20 nt, Artificial Sequence
  CD3E gRNA sequence 4
uauuaugucugcuaccccag

SEQ ID NO: 43 - RNA, 20 nt, Artificial Sequence
  CD3E gRNA sequence 5
agauaaaaguucgcaucuuc

SEQ ID NO: 44 - RNA, 20 nt, Artificial Sequence
  CD3E gRNA sequence 6
agaugcagucgggcacucac

SEQ ID NO: 45 - RNA, 20 nt, Artificial Sequence
  CD3E gRNA sequence 7
uuacuuuacuaagauggcgg

SEQ ID NO: 46 - RNA, 20 nt, Artificial Sequence
  CD3E gRNA sequence 8
gauggagacuuuauaugcug

SEQ ID NO: 47 - RNA, 20 nt, Artificial Sequence
  CD3E gRNA sequence 9
gauguccacuaugacaauug

SEQ ID NO: 48 - RNA, 20 nt, Artificial Sequence
  CD3E gRNA sequence 10
caacacaaugauaaaaacau

SEQ ID NO: 49 - RNA, 20 nt, Artificial Sequence
  CD3E gRNA sequence 11
ugaggaucaccugucacuga

SEQ ID NO: 50 - DNA, 20 bp, Artificial Sequence
  CD3D gRNA target sequence 1
aaacgcatcctggacccacg

SEQ ID NO: 51 - DNA, 20 bp, Artificial Sequence
  CD3D gRNA target sequence 2
acttcgataatgaacttgca

SEQ ID NO: 52 - DNA, 20 bp, Artificial Sequence
  CD3D gRNA target sequence 3
tagccttaccttgcgagaga

SEQ ID NO: 53 - DNA, 20 bp, Artificial Sequence
  CD3D gRNA target sequence 4
ccgacacacaagctctgttg

SEQ ID NO: 54 - DNA, 20 bp, Artificial Sequence
  CD3D gRNA target sequence 5
caacgctcacctgatagacc

SEQ ID NO: 55 - DNA, 20 bp, Artificial Sequence
  CD3D gRNA target sequence 6
gaacatagcacgtttctctc

SEQ ID NO: 56 - DNA, 20 bp, Artificial Sequence
  CD3D gRNA target sequence 7
gacaatgatgccagccacgg

SEQ ID NO: 57 - DNA, 20 bp, Artificial Sequence
  CD3D gRNA target sequence 8
ttgcaataccagcatcacat

SEQ ID NO: 58 - DNA, 20 bp, Artificial Sequence
  CD3D gRNA sequence 1
aaacgcauccuggacccacg

SEQ ID NO: 59 - RNA, 20 nt, Artificial Sequence
  CD3D gRNA sequence 2
acuucgauaaugaacuugca

SEQ ID NO: 60 - RNA, 20 nt, Artificial Sequence
  CD3D gRNA sequence 3
uagccuuaccuugcgagaga

SEQ ID NO: 61 - RNA, 20 nt, Artificial Sequence
  CD3D gRNA sequence 4
ccgacacacaagcucuguug

SEQ ID NO: 62 - RNA, 20 nt, Artificial Sequence
  CD3D gRNA sequence 5
caacgcucaccugauagacc

SEQ ID NO: 63 - RNA, 20 nt, Artificial Sequence
  CD3D gRNA sequence 6
gaacauagcacguuucucuc

SEQ ID NO: 64 - RNA, 20 nt, Artificial Sequence
  CD3D gRNA sequence 7
gacaaugaugccagccacgg

SEQ ID NO: 65 - RNA, 20 nt, Artificial Sequence
  CD3D gRNA sequence 8
uugcaauaccagcaucacau

SEQ ID NO: 66 - DNA, 20 bp, Artificial Sequence
  CD3G gRNA target sequence 1
ttacactgatacatccctcg

SEQ ID NO: 67 - DNA, 20 bp, Artificial Sequence
  CD3G gRNA target sequence 2
actttggcccagtcaatcaa

SEQ ID NO: 68 - DNA, 20 bp, Artificial Sequence
  CD3G gRNA target sequence 3
gtgtatgactatcaagaaga

SEQ ID NO: 69 - DNA, 20 bp, Artificial Sequence
  CD3G gRNA target sequence 4
ttctcctacctttgattgac

SEQ ID NO: 70 - DNA, 20 bp, Artificial Sequence
  CD3G gRNA target sequence 5
cttgaagaagaatgatagcc

SEQ ID NO: 71 - DNA, 20 bp, Artificial Sequence
  CD3G gRNA target sequence 6
cagagactgacatggaacag

SEQ ID NO: 72 - DNA, 20 bp, Artificial Sequence
  CD3G gRNA target sequence 7
tacactgatacatccctcga

SEQ ID NO: 73 - DNA, 20 bp, Artificial Sequence
  CD3G gRNA target sequence 8
cagaagccaaaaatatcaca

SEQ ID NO: 74 - DNA, 20 bp, Artificial Sequence
  CD3G gRNA target sequence 9
aaagagaaagccagatatgg

SEQ ID NO: 75 - RNA, 20 nt, Artificial Sequence
  CD3G gRNA sequence 1
uuacacugauacaucccucg

SEQ ID NO: 76 - RNA, 20 nt, Artificial Sequence
  CD3G gRNA sequence 2
acuuuggcccagucaaucaa

SEQ ID NO: 77 - RNA, 20 nt, Artificial Sequence
  CD3G gRNA sequence 3
guguaugacuaucaagaaga

SEQ ID NO: 78 - RNA, 20 nt, Artificial Sequence
  CD3G gRNA sequence 4
uucuccuaccuuugauugac

SEQ ID NO: 79 - RNA, 20 nt, Artificial Sequence
  CD3G gRNA sequence 5
cuugaagaagaaugauagcc

SEQ ID NO: 80 - RNA, 20 nt, Artificial Sequence
  CD3G gRNA sequence 6
cagagacugacauggaacag

SEQ ID NO: 81 - RNA, 20 nt, Artificial Sequence
  CD3G gRNA sequence 7
uacacugauacaucccucga

SEQ ID NO: 82 - RNA, 20 nt, Artificial Sequence
  CD3G gRNA sequence 8
cagaagccaaaaauaucaca

SEQ ID NO: 83 - RNA, 20 nt, Artificial Sequence
  CD3G gRNA sequence 9
aaagagaaagccagauaugg

SEQ ID NO: 84 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 85 - protein, 22 aa, Artificial Sequence
  GMCSFR alpha chain signal sequence
MLLLVTSLLLCELPHPAFLLIP

SEQ ID NO: 86 - protein, 18 aa, Artificial Sequence
  CD8 alpha signal peptide
MALPVTALLLPLALLLHA

SEQ ID NO: 87 - DNA, 66 bp, Artificial Sequence
  GMCSFR alpha chain signal sequence
atgctgctgctggtgaccagcctgctgctgtgcgagctgccccaccccgcctttctgctgatcccc

SEQ ID NO: 88 - protein, 24 aa, Artificial Sequence
  T2A peptide
LEGGGEGRGSLLTCGDVEENPGPR

SEQ ID NO: 89 - protein, 18 aa, Artificial Sequence
  T2A peptide
EGRGSLLTCGDVEENPGP

SEQ ID NO: 90 - protein, 22 aa, Artificial Sequence
  P2A peptide
GSGATNFSLLKQAGDVEENPGP

SEQ ID NO: 91 - protein, 19 aa, Artificial Sequence
  P2A peptide
ATNFSLLKQAGDVEENPGP

SEQ ID NO: 92 - protein, 20 aa, Artificial Sequence
  E2A peptide
QCTNYALLKLAGDVESNPGP

SEQ ID NO: 93 - protein, 22 aa, Artificial Sequence
  F2A peptide
VKQTLNFDLLKLAGDVESNPGP

SEQ ID NO: 94 - protein, 21 aa, Artificial Sequence
  P2A peptide
GSGEGRGSLLTCGDVEENPGP

SEQ ID NO: 95 - DNA, 25 bp, Artificial Sequence
  human HBB gene splice acceptor
ctgacctcttctcttcctcccacag

SEQ ID NO: 96 - DNA, 13 bp, Artificial Sequence
  human IgG gene splice acceptor
tttctctccacag

SEQ ID NO: 97 - protein, 5 aa, Artificial Sequence
  CDR H1
DYGVS

SEQ ID NO: 98 - protein, 16 aa, Artificial Sequence
  CDR H2
VIWGSETTYYNSALKS

SEQ ID NO: 99 - protein, 7 aa, Artificial Sequence
  CDR H3
YAMDYWG

SEQ ID NO: 100 - protein, 12 aa, Artificial Sequence
  HC-CDR3
HYYYGGSYAMDY

SEQ ID NO: 101 - protein, 11 aa, Artificial Sequence
  CDR L1
RASQDISKYLN

SEQ ID NO: 102 - protein, 7 aa, Artificial Sequence
  CDR L2
SRLHSGV

SEQ ID NO: 103 - protein, 7 aa, Artificial Sequence
  LC-CDR2
HTSRLHS

SEQ ID NO: 104 - protein, 9 aa, Artificial Sequence
  CDR L3
GNTLPYTFG

SEQ ID NO: 105 - protein, 9 aa, Artificial Sequence
  LC-CDR3
QQGNTLPYT

SEQ ID NO: 106 - protein, 120 aa, Artificial Sequence
  VH peptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 107 - protein, 107 aa, Artificial Sequence
  VL peptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 108 - protein, 9 aa, Artificial Sequence
  CDR L3
GNTLPYTFG

SEQ ID NO: 109 - protein, 18 aa, Artificial Sequence
  Linker
GSTSGSGKPGSGEGSTKG

SEQ ID NO: 110 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 111 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 112 - protein, 5 aa, Artificial Sequence
  CDR H1
SYWMN

SEQ ID NO: 113 - protein, 17 aa, Artificial Sequence
  CDR H2
QIYPGDGDTNYNGKFKG

SEQ ID NO: 114 - protein, 13 aa, Artificial Sequence
  Synthetic construct
KTISSVVDFYFDY

SEQ ID NO: 115 - protein, 11 aa, Artificial Sequence
  CDR L1
KASQNVGTNVA

SEQ ID NO: 116 - protein, 7 aa, Artificial Sequence
  CDR L2
SATYRNS

SEQ ID NO: 117 - protein, 9 aa, Artificial Sequence
  CDR L3
QQYNRYPYT

SEQ ID NO: 118 - protein, 122 aa, Artificial Sequence
  VH peptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 119 - protein, 108 aa, Artificial Sequence
  VL peptide
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 120 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 121 - protein, 5 aa, Artificial Sequence
  Linker
  VARIANT (1)...(5): Can be present in repeats of any integer up to and including 10
GGGGS

SEQ ID NO: 122 - protein, 5 aa, Artificial Sequence
  Linker
GGGGS

SEQ ID NO: 123 - protein, 10 aa, Artificial Sequence
  Linker
GGGGSGGGGS

SEQ ID NO: 124 - protein, 20 aa, Artificial Sequence
  Linker
GGGGSGGGGSGGGGSGGGGS

SEQ ID NO: 125 - protein, 5 aa, Artificial Sequence
  Linker
  VARIANT (1)...(5): Can be present in repeats of 3 or 4
GGGGS

SEQ ID NO: 126 - protein, 5 aa, Artificial Sequence
  Linker
  VARIANT (1)...(5): Can be present in repeats of 2 or 3
GGGGS

SEQ ID NO: 127 - protein, 15 aa, Artificial Sequence
  Linker
GGGASGGGGSGGGGS

SEQ ID NO: 128 - protein, 6 aa, Artificial Sequence
  Linker
GGGGGS

SEQ ID NO: 129 - protein, 6 aa, Artificial Sequence
  Linker
  VARIANT (1)...(6): Can be present in repeats of any integer up to and including 4
GGGGGS

SEQ ID NO: 130 - protein, 6 aa, Artificial Sequence
  Linker
GGSGGS

SEQ ID NO: 131 - protein, 9 aa, Artificial Sequence
  Linker
GGSGGSGGS

SEQ ID NO: 132 - protein, 12 aa, Artificial Sequence
  Linker
GGSGGSGGSGGS

SEQ ID NO: 133 - protein, 15 aa, Artificial Sequence
  Linker
GGSGGSGGSGGSGGS

SEQ ID NO: 134 - protein, 18 aa, Artificial Sequence
  Linker
GGGGGSGGGGGSGGGGGS

SEQ ID NO: 135 - protein, 18 aa, Artificial Sequence
  Linker
GGSGGGGSGGGGSGGGGS

SEQ ID NO: 136 - protein, 8 aa, Artificial Sequence
  Streptavidin binding peptide
WRHPQFGG

SEQ ID NO: 137 - protein, 8 aa, Artificial Sequence
  Streptavidin binding peptide II
WSHPQFEK

SEQ ID NO: 138 - protein, 3 aa, Artificial Sequence
  Streptavidin binding peptide
  VARIANT 3: Xaa = Glu, Asp or Met
HPX

SEQ ID NO: 139 - protein, 4 aa, Artificial Sequence
  Streptavidin-binding peptide
HPQF

SEQ ID NO: 140 - protein, 8 aa, Artificial Sequence
  Streptavidin-binding peptide
  VARIANT 1: Xaa = Trp, Lys or Arg
  VARIANT 2: Xaa = any amino acid
  VARIANT 7: Xaa = GLy or Glu
  VARIANT 8: Xaa = Gly, Lys or Arg
XXHPQFXX

SEQ ID NO: 141 - protein, 8 aa, Artificial Sequence
  Streptavidin-binding peptide
  VARIANT 2: Xaa = any amino acid
  VARIANT 7: Xaa = Gly or Glu
  VARIANT 8: Xaa = Gly, Lys or Arg
WXHPQFXX

SEQ ID NO: 142 - protein, 17 aa, Artificial Sequence
  Sequential modules of streptavidin-binding peptide
  VARIANT 9: Xaa = any amino acid
  VARIANT 9: Xaa is present in numbers of 8 or 12
WSHPQFEKXWSHPQFEK

SEQ ID NO: 143 - protein, 21 aa, Artificial Sequence
  Sequential modules of streptavidin-binding peptide
  VARIANT 9-12: Is present in repeats of 2 or 3
WSHPQFEKGGGSWSHPEPFEK

SEQ ID NO: 144 - protein, 30 aa, Artificial Sequence
  Twin-Strep-tag
SAWSHPQFEKGGGSGGGSGGGSWSHPQFEK

SEQ ID NO: 145 - protein, 30 aa, Artificial Sequence
  Twin-Strep-tag
SAWSHPQFEKGGGSGGGSGGSAWSHPQFEK

SEQ ID NO: 146 - protein, 28 aa, Artificial Sequence
  Twin-Strep-tag
WSHPQFEKGGGSGGGSGGGSWSHPQFEK

SEQ ID NO: 147 - protein, 24 aa, Artificial Sequence
  Twin-Strep-tag
WSHPQFEKGGGSGGGSWSHPQFEK

SEQ ID NO: 148 - protein, 28 aa, Artificial Sequence
  Twin-Strep-tag
WSHPQFEKGGGSGGGSGGSAWSHPQFEK

SEQ ID NO: 149 - protein, 10 aa, Artificial Sequence
  Streptavidin binding peptide II
SAWSHPQFEK

SEQ ID NO: 150 - protein, 5 aa, Artificial Sequence
  linker
GGGGG

SEQ ID NO: 151 - protein, 5 aa, Artificial Sequence
  linker
  VARIANT 2, 4: Xaa = Ala, Val, Leu, Ile, Met, Phe, Trp, Pro, Gly, Ser, Thr, Cys, Tyr, Asn, Gln, Lys, Arg, His, Asp and Glu
  VARIANT 1, 3, 5: Can be present in repeats of any integer up to and including 5
GXGXG

SEQ ID NO: 152 - protein, 11 aa, Artificial Sequence
  Linker
  VARIANT 4, 8: Xaa = Ala, Val, Leu, Ile, Met, Phe, Trp, Pro, Gly, Ser, Thr, Cys, Tyr, Asn, Gln, Lys, Arg, His, Asp and Glu
GGGXGGGXGGG

SEQ ID NO: 153 - protein, 5 aa, Artificial Sequence
  Linker
  VARIANT (1)...(5): Can be present in repeats of any integer
SSSSG

SEQ ID NO: 154 - protein, 11 aa, Artificial Sequence
  Linker
GGGGGCGGGGG